(19) United States
(12) Patent Application Publication
(10) Pub. No.: US 2026/0365641 A1
(43) Pub. Date: Nov. 19, 2026

(54) Aromatic Acetylene Derivative, Preparation Method Therefor, And Use Thereof

(71) Applicants: Zhejiang Hisun Pharmaceutical Co., Ltd., Taizhou (CN); Shanghai Aryl Pharmtech Co., Ltd., Shanghai (CN)

(72) Inventors: Xiangui Huang, Shanghai (CN); Weiwei Liao, Shanghai (CN); Chengfei Wu, Shanghai (CN); Cheng Ye, Shanghai (CN); Daiwang Xu, Shanghai (CN); Xiaojie Xu, Taizhou (CN); Houjiang Zhou, Taizhou (CN)

(73) Assignees: Zhejiang Hisun Pharmaceutical Co., Ltd., Taizhou (CN); Shanghai Aryl Pharmtech Co., Ltd., Shanghai (CN)

(21) Appl. No.: 19/114,529

(22) PCT Filed: Sep. 28, 2023
(86) PCT No.: PCT/CN2023/122622

§ 371 Date: Mar. 24, 2025

(30) Foreign Application Priority Data
Sep. 28, 2022 (CN) 202211187568.X
Jan. 19, 2023 (CN) 202310062329.X
Jun. 14, 2023 (CN) 202310702568.7

(51) Int. Cl. C07D 413/14; A61K 31/422; A61K 31/438; A61K 31/4439; A61K 31/444; A61K 31/454; A61K 31/497; A61K 31/501; A61K 31/506; A61K 31/5377; A61P 31/04

(52) U.S. Cl. CPC C07D 413/14; A61K 31/422; A61K 31/438; A61K 31/4439; A61K 31/444; A61K 31/454; A61K 31/497; A61K 31/501; A61K 31/506; A61K 31/5377; A61P 31/04

(57) ABSTRACT

The present invention relates to an aromatic acetylene derivative, a preparation method therefor, and medical use of a pharmaceutical composition containing the derivative. Specifically, the invention relates to an aromatic acetylene derivative as represented by general formula (I), a preparation method therefor, and a pharmaceutically acceptable salt thereof, and use of the aromatic acetylene derivative and the pharmaceutically acceptable salt thereof as a therapeutic agent, in particular, as an LPXC inhibitor, wherein the definition of substituents in general formula (I) is the same as that in the specification.

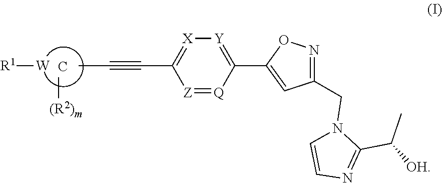

### TECHNICAL FIELD

The present invention relates to an aromatic acetylene derivative, a preparation method therefor, a pharmaceutical composition containing the derivative, and use of the pharmaceutical composition as a therapeutic agent, in particular as an LPXC inhibitor.

### BACKGROUND

The 1930s to 1960s were the golden age for the development of antibiotics. Since then, antibiotics have been widely used all over the world, but drug resistance problems of bacteria have also emerged one after another, and drug-resistant bacteria have become a major problem threatening human health. Multi-drug-resistant Gram-negative bacterium is one of the main pathogens of infection. At present, there is a serious shortage of drugs used to treat the infection of the multi-drug-resistant Gram-negative bacterium in clinic, and more toxic drugs are still used.

In recent years, bacterial drug resistance has been a hot topic in the international medical field, but the research and development speed has been slow, and few compounds have entered clinical research at home and abroad. Therefore, it is an important problem to find a new antibacterial drug for Gram-negative bacterium.

UDP-3-O—(R-3-hyrdoxymyristoyl)-N-acetyl glucosamine (LPXC) is a Zn⁺ dependent metalloenzyme, which is a first rate-limiting enzyme for the synthesis of a lipid A, while the lipid A is an important component of an outer membrane of the Gram-negative bacterium, which can anchor lipopolysaccharide to the outer membrane, maintain the integrity of its own cells and serve as an external barrier for hydrophobicity to protect external factors such as antibiotics from entering the cells and protects the bacteria from invasion. In addition, the lipid A is also an active component of bacterial endotoxin, which enters blood through intestinal mucosa, activates an immune response of a human body, and even causes severe septic shock, which is also the reason why the Gram-negative bacterium has pathogenic infection. Therefore, the inhibition of LPXC can inhibit the biosynthesis of the lipid A of the Gram-negative bacterium, thus effectively controlling the infection of the Gram-negative bacterium.

At present, further understanding of structure and characteristics of LPXC is mostly obtained through separation, purification and analytical identification of LPXC crystals of Escherichia coli, Pseudomonas aeruginosa and thermophilic bacteria. The structures of LPXC from the three different sources are highly similar, which all contain two domains, wherein an active region is located at a junction of the two domains. Each domain contains α helix and β sheet, and β sheet contains α helix, forming a sandwich structure of “β-α-α-β”. Although amino acid sequences of the two domains are slightly different, the two domains have the same spatial structure. Moreover, each domain has a corresponding insertion region, which is composed of p sheet, forming different functional regions. Studies have shown that LPXC has high homology in Gram-negative bacterium, and has no common sequence with various mammalian enzyme systems. From a biological point of view, because of unique advantages of broad spectrum and low toxicity of LPXC, inhibition of LPXC will be an ideal direction to study antibacterial drugs.

At present, there are no new drugs on the market for LPXC inhibitors. The small-molecule compound RC-01 developed by Toyama Chemical Co. Ltd has entered the first clinical phase, and the research on LPXC by other pharmaceutical companies such as Novartis and Taisho Pharmaceutical (Taiwan) Co., Ltd. is in the preclinical stage. Therefore, the research and application of LPXC inhibitors have made some progress, but the room for improvement is still huge, and it is still necessary to continue research and development of new LPXC inhibitors.

### SUMMARY

In view of the above technical problems, the present invention provides an aromatic acetylene derivative represented by general formula (I) or a stereoisomer, a tautomer or a pharmaceutically acceptable salt thereof:

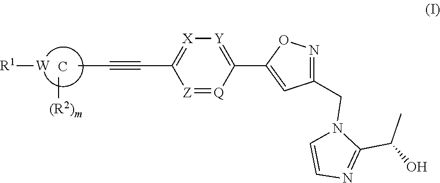

wherein:
W is selected from O, S(O)ᵣ or N;
ring C is selected from 5- to 10-membered spiro-heterocyclyl or monocyclic heterocyclyl, wherein the monocyclic heterocyclyl contains one double bond;
X, Y, Z and Q are each independently selected from CR^{A} or N atoms, and at most two of X, Y, Z and Q are N atoms at the same time;
R^{A} is selected from hydrogen atom, halogen, hydroxyl, cyano, alkyl or alkoxy; wherein the alkyl or the alkoxy is optionally further substituted by one or more substituents selected from halogen, hydroxyl, cyano, alkyl or alkoxy;
R¹ does not exist or is selected from hydrogen atom, hydroxyl, cyano, halogen, alkyl, alkoxy, cycloalkyl, heterocyclyl, aryl, heteroaryl, —C(O)R³, —C(O)NR⁶R⁷ or —S(O)ᵣR⁴; wherein the alkoxy, the alkyl, the cycloalkyl, the heterocyclyl, the aryl, or the heteroaryl is optionally further substituted by one or more R^{B};
R³ and R⁴ are each independently selected from hydrogen atom, hydroxyl, alkyl, cycloalkyl, aryl, heteroaryl or heterocyclyl; wherein the alkyl, the cycloalkyl, the aryl, the heteroaryl or the heterocyclyl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R^{B} is each independently selected from cyano, halogen, alkyl, hydroxyl, cycloalkyl, heterocyclyl, aryl, heteroaryl, —OR⁵, —C(O)R⁵, —C(O)OR⁵, —NHC(O)R⁵, —NHC(O)OR⁵, —NR⁶R⁷, —C(O)NR⁶R⁷, —CH₂NHC(O)OR⁵, —CH₂NR⁶R⁷ or —S(O)ᵣR⁵; wherein the alkyl, the cycloalkyl, the heterocyclyl, the aryl or the heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
alternatively, two R^{B} form one —C(═O)— together with the same carbon atom to which the two R^{B} are connected;
each R² is the same or different, and is independently selected from hydroxyl, cyano, halogen, alkyl or alkoxy; wherein the alkyl or the alkoxy is optionally further substituted by one or more substituents selected from halogen, hydroxyl, cyano, alkyl or alkoxy;
each R⁵ is independently selected from hydrogen atom, alkyl, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁶ and R⁷ are each independently selected from hydrogen atom, hydroxyl, halogen, alkyl, alkoxy, cycloalkyl, heterocyclyl, aryl or heteroaryl, wherein the alkyl, the alkoxy, the cycloalkyl, the heterocyclyl, the aryl or the heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
alternatively, R⁶ and R⁷ form a 4- to 8-membered heterocyclyl together with atoms to which R⁶ and R⁷ are connected, wherein the 4- to 8-membered heterocyclyl contains one or more N, O or S(O)ᵣ, and the 4- to 8-membered heterocyclyl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁸, R⁹ and R¹⁰ are independently selected from hydrogen atom, alkyl, amino, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate;
m is 0, 1 or 2; and m is preferably 0; and
r is each independently 0, 1 or 2.

A preferred solution of the present invention provides a compound represented by general formula (I) or a stereoisomer or a tautomer thereof or a pharmaceutically acceptable salt thereof, which is a compound represented by general formula (II) or a stereoisomer or a tautomer thereof, or a pharmaceutically acceptable salt thereof,

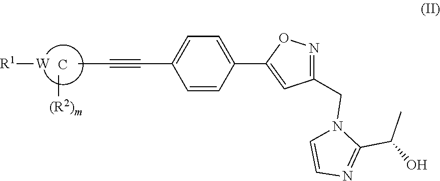

ring C, R¹, R², W and m are as defined in general formula (I).

A preferred solution of the present invention provides a compound represented by general formula (I) or general formula (II) or a stereoisomer or a tautomer thereof or a pharmaceutically acceptable salt thereof, wherein W is N.

A preferred solution of the present invention provides a compound represented by general formula (I) or general formula (II) or a stereoisomer or a tautomer thereof or a pharmaceutically acceptable salt thereof, wherein ring C is selected from:

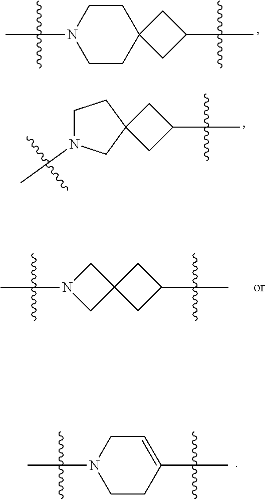

A preferred solution of the present invention provides a compound represented by general formula (I) or general formula (II) or a stereoisomer or a tautomer thereof or a pharmaceutically acceptable salt thereof, wherein
R¹ is selected from hydrogen atom, hydroxyl, alkyl, cycloalkyl, heterocyclyl, —C(O)R³, —C(O)NR⁶R⁷ or —S(O)ᵣR⁴; wherein the alkyl or the cycloalkyl is optionally further substituted by one or more R^{B};
each R^{B} is independently selected from cyano, hydroxyl, heterocyclyl, aryl, heteroaryl, —OR⁵, —C(O)OR⁵, —NR⁶R⁷, —C(O)NR⁶R⁷ or —S(O)ᵣR⁵; wherein the heterocyclyl or the heteroaryl is optionally further substituted by one or more substituents selected from hydroxyl, cyano, alkoxy, haloalkyl, ═O, —C(O)OR⁸ or —C(O)NR⁹R¹⁰;
alternatively, two R^{B} form one —C(═O)— together with the same carbon atom to which the two R^{B} are connected;
R³ is selected from hydrogen atom, alkyl, cycloalkyl, heteroaryl or heterocyclyl; wherein the alkyl, the cycloalkyl, the heteroaryl or the heterocyclyl is optionally further substituted by one or more substituents selected from hydroxyl, alkoxy, cyano, heteroaryl, ═O, —NR⁹R¹⁰, —C(O)OR⁸ or —C(O)NR⁹R¹⁰;
R⁴ is hydrogen atom or hydroxyl;
each R⁵ is independently selected from hydrogen atom, alkyl or hydroxyalkyl;
R6 and R7 are each independently selected from hydrogen atom or alkyl, wherein the alkyl is optionally further substituted by one or more substituents selected from hydroxyl, cyano, halogen or alkoxy;
R⁸, R⁹ and R¹⁰ are each independently selected from hydrogen atom or alkyl; the alkyl is preferably methyl; and
r is 2.

In a preferred solution of the present invention, the compound represented by general formula (I) is selected from:

| No. of | | |
|---|---|---|
| com- | | |
| pound | Structure | Name |
| Example 1 | 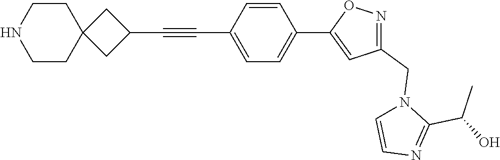 [SMILES: C[C@H](O)c1nccn1Cc1cc(-c2ccc(C#CC3CC4(CCNCC4)C3)cc2)on1] | (S)-1-(1-((5-(4-((7-azaspiro[3.5] nonan-2-yl)ethynyl)phenyl) isoxazol-3-yl)methyl)-1H- imidazol-2-yl)ethan-1-ol |
| Example 2 | 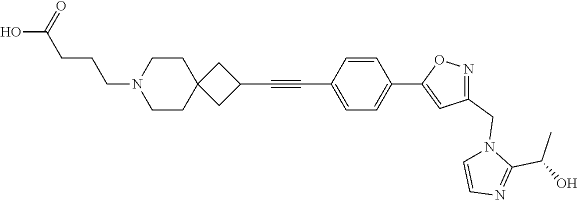 [SMILES: C[C@H](O)c1nccn1Cc1cc(-c2ccc(C#CC3CC4(CCN(CCCC(=O)O)CC4)C3)cc2)on1] | (S)-4-(2-((4-(3-((2-(1- hydroxyethyl)-1H- imidazol-1-yl)methyl)isoxazol- 5-yl)phenyl)ethynyl)-7- azaspiro[3.5]nonan-7-yl) butanoic acid |
| Example 3 | 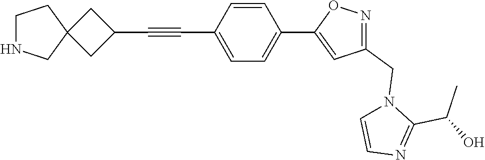 [SMILES: C[C@H](O)c1nccn1Cc1cc(-c2ccc(C#CC3CC4(CCNC4)C3)cc2)on1] | (S)-1-(1-((5-(4-((6-azaspiro[3.4] octan-2-yl)ethynyl)phenyl) isoxazol-3-yl)methyl)-1H- imidazol-2-yl)ethan-1-ol |
| Example 4 | 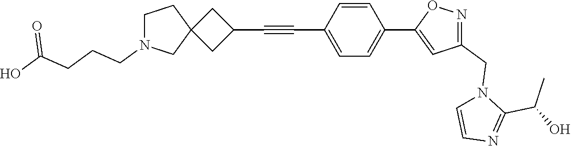 [SMILES: C[C@H](O)c1nccn1Cc1cc(-c2ccc(C#CC3CC4(CCN(CCCC(=O)O)C4)C3)cc2)on1] | (S)-4-(2-((4-(3-((2-(1- hydroxyethyl)-1H-imidazol-1-yl) methyl)isoxazol-5-yl)phenyl) ethynyl)-6-azaspiro[3.4]octan- 6-yl)butanoic acid |
| Example 5 | 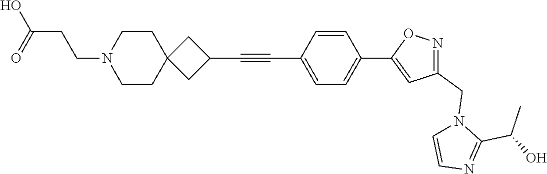 [SMILES: C[C@H](O)c1nccn1Cc1cc(-c2ccc(C#CC3CC4(CCN(CCC(=O)O)CC4)C3)cc2)on1] | (S)-3-(2-((4-(3-((2-(1- hydroxyethyl)-1H-imidazol-1-yl) methyl)isoxazol-5-yl)phenyl) ethynyl)-7-azaspiro[3.5] nonan-7-yl)propanoic acid |
| Example 6 | 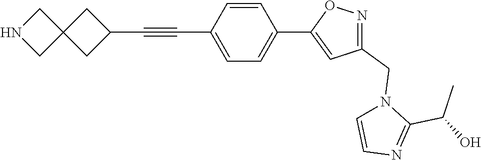 [SMILES: C[C@H](O)c1nccn1Cc1cc(-c2ccc(C#CC3CC4(CNC4)C3)cc2)on1] | (S)-1-(1-((5-(4-((2-azaspiro[3.3] heptan-6-yl)ethynyl)phenyl) isoxazol-3-yl)methyl)-1H- imidazol-2-yl)ethan-1-ol |
| Example 7 | 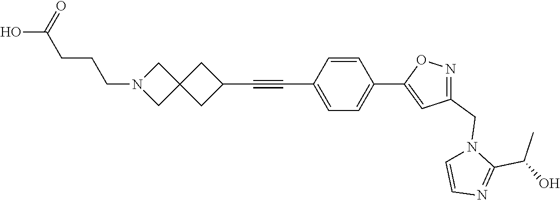 [SMILES: C[C@H](O)c1nccn1Cc1cc(-c2ccc(C#CC3CC4(C3)CN(CCCC(=O)O)C4)cc2)on1] | (S)-4-(6-((4-(3-((2-(1- hydroxyethyl)-1H-imidazol-1-yl) methyl)isoxazol-5-yl)phenyl) ethynyl)-2-azaspiro[3.3]heptan- 2-yl)butanoic acid |
| Example 8 | 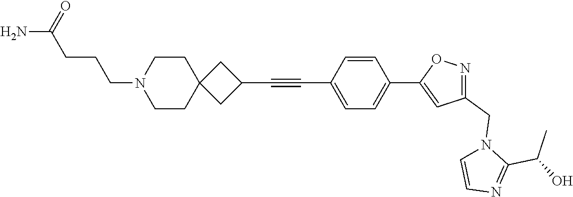 [SMILES: C[C@H](O)c1nccn1Cc1cc(-c2ccc(C#CC3CC4(CCN(CCCC(N)=O)CC4)C3)cc2)on1] | (S)-4-(2-((4-(3-((2-(1- hydroxyethyl)-1H-imidazol- 1-yl)methyl)isoxazol-5-yl) phenyl)ethynyl)-7-azaspiro[3.5] nonan-7-yl)butanamide |
| Example 9 | 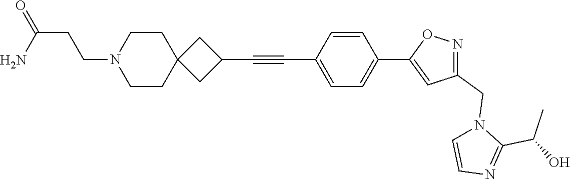 [SMILES: C[C@H](O)c1nccn1Cc1cc(-c2ccc(C#CC3CC4(CCN(CCC(N)=O)CC4)C3)cc2)on1] | (S)-3-(2-((4-(3-((2-(1- hydroxyethyl)-1H-imidazol- 1-yl)methyl)isoxazol-5-yl) phenyl)ethynyl)-7-azaspiro[3.5] nonan-7-yl)propanamide |
| Example 10 | 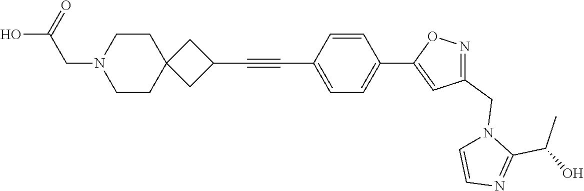 [SMILES: C[C@H](O)c1nccn1Cc1cc(-c2ccc(C#CC3CC4(CCN(CC(=O)O)CC4)C3)cc2)on1] | (S)-2-(2-((4-(3-((2-(1- hydroxyethyl)-1H-imidazol- 1-yl)methyl)isoxazol-5-yl) phenyl)ethynyl)-7- azaspiro[3.5]nonan-7-yl)acetic acid |
| Example 11 | 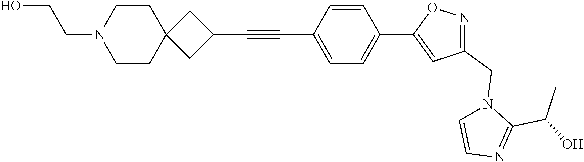 [SMILES: C[C@H](O)c1nccn1Cc1cc(-c2ccc(C#CC3CC4(CCN(CCO)CC4)C3)cc2)on1] | (S)-2-(2-((4-(3-((2-(1- hydroxyethyl)-1H-imidazol- 1-yl)methyl)isoxazol-5-yl) phenyl)ethynyl)-7-azaspiro[3.5] nonan-7-yl)ethan-1-ol |
| Example 12 | 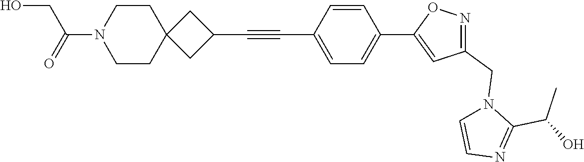 [SMILES: C[C@H](O)c1nccn1Cc1cc(-c2ccc(C#CC3CC4(CCN(C(=O)CO)CC4)C3)cc2)on1] | (S)-2-hydroxy-1-(2-((4-(3-((2- (1-hydroxyethyl)-1H-imidazol- 1-yl)methyl)isoxazol-5-yl) phenyl)ethynyl)-7-azaspiro[3.5] nonan-7-yl)ethan-1-one |
| Example 13 | 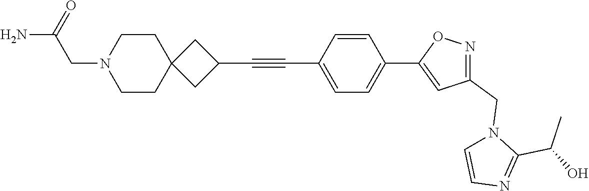 [SMILES: C[C@H](O)c1nccn1Cc1cc(-c2ccc(C#CC3CC4(CCN(CC(N)=O)CC4)C3)cc2)on1] | (S)-2-(2-((4-(3-((2-(1- hydroxyethyl)-1H-imidazol- 1-yl)methyl)isoxazol-5-yl) phenyl)ethynyl)-7-azaspiro[3.5] nonan-7-yl)acetamide |
| Example 14 | 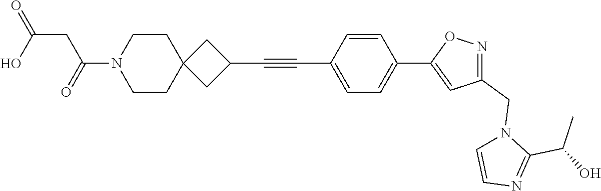 [SMILES: C[C@H](O)c1nccn1Cc1cc(-c2ccc(C#CC3CC4(CCN(C(=O)CC(=O)O)CC4)C3)cc2)on1] | (S)-3-(2-((4-(3-((2-(1- hydroxyethyl)-1H-imidazol- 1-yl)methyl)isoxazol-5-yl) phenyl)ethynyl)-7-azaspiro[3.5] nonan-7-yl)-3-oxopropanoic acid |
| Example 15 | 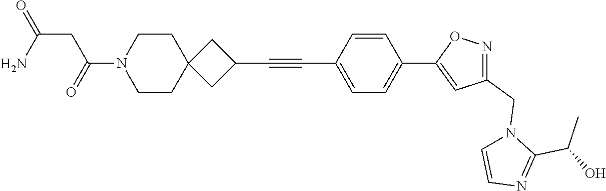 [SMILES: C[C@H](O)c1nccn1Cc1cc(-c2ccc(C#CC3CC4(CCN(C(=O)CC(N)=O)CC4)C3)cc2)on1] | (S)-3-(2-((4-(3-((2-(1- hydroxyethyl)-1H-imidazol- 1-yl)methyl)isoxazol-5-yl) phenyl)ethynyl)-7-azaspiro[3.5] nonan-7-yl)-3-oxopropanamide |
| Example 16 | 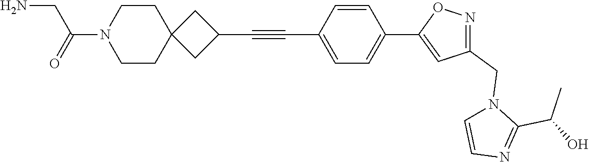 [SMILES: C[C@H](O)c1nccn1Cc1cc(-c2ccc(C#CC3CC4(CCN(C(=O)CN)CC4)C3)cc2)on1] | (S)-2-amino-1-(2-((4-(3-((2-(1- hydroxyethyl)-1H-imidazol-1-yl) methyl)isoxazol-5-yl)phenyl) ethynyl)-7-azaspiro[3.5]nonan- 7-yl)ethan-1-one |
| Example 17 | 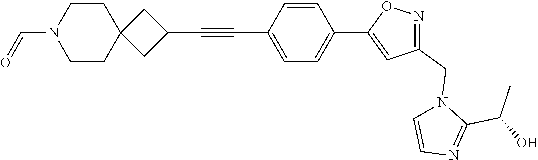 [SMILES: C[C@H](O)c1nccn1Cc1cc(-c2ccc(C#CC3CC4(CCN(C=O)CC4)C3)cc2)on1] | (S)-2-((4-(3-((2-(1-hydroxyethyl)- 1H-imidazol-1-yl)methyl) isoxazol-5-yl)phenyl)ethynyl)-7- azaspiro[3.5]nonane-7- carbaldehyde |
| Example 18 | 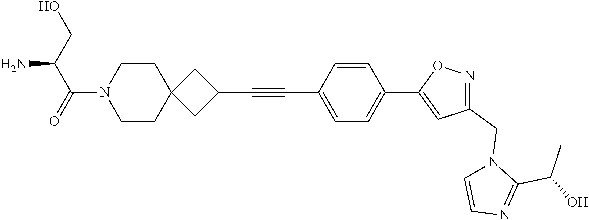 [SMILES: C[C@H](O)c1nccn1Cc1cc(-c2ccc(C#CC3CC4(CCN(C(=O)[C@@H](N)CO)CC4)C3)cc2)on1] | (S)-2-amino-3-hydroxy-1-(2-((4- (3-((2-((S)-1-hydroxyethyl)-1H- imidazol-1-yl)methyl)isoxazol-5- yl)phenyl)ethynyl)-7-azaspiro [3.5]nonan-7-yl)propan-1-one |
| Example 19 | 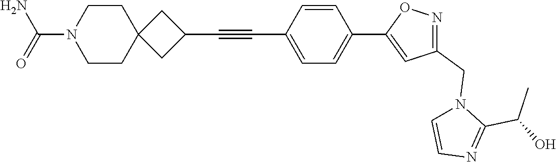 [SMILES: C[C@H](O)c1nccn1Cc1cc(-c2ccc(C#CC3CC4(CCN(C(N)=O)CC4)C3)cc2)on1] | (S)-2-((4-(3-((2-(1-hydroxyethyl)- 1H-imidazol-1-yl)methyl) isoxazol-5-yl)phenyl)ethynyl)-7- azaspiro[3.5]nonane-7- carboxamide |
| Example 20 | 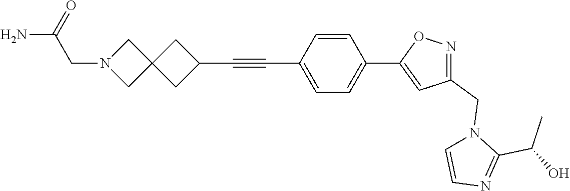 [SMILES: C[C@H](O)c1nccn1Cc1cc(-c2ccc(C#CC3CC4(C3)CN(CC(N)=O)C4)cc2)on1] | (S)-2-(6-((4-(3-((2-(1- hydroxyethyl)-1H-imidazol- 1-yl)methyl)isoxazol-5-yl) phenyl)ethynyl)-2-azaspiro[3.3] heptan-2-yl)acetamide |
| Example 21 | 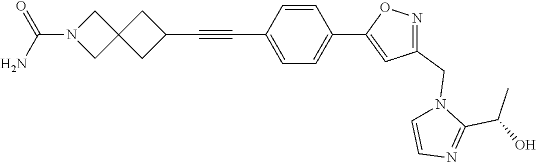 [SMILES: C[C@H](O)c1nccn1Cc1cc(-c2ccc(C#CC3CC4(C3)CN(C(N)=O)C4)cc2)on1] | (S)-6-((4-(3-((2-(1-hydroxyethyl)- 1H-imidazol-1-yl)methyl) isoxazol-5-yl)phenyl) ethynyl)-2-azaspiro[3.3 ]heptane- 2-carboxamide |
| Example 22 | 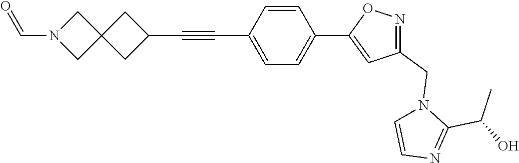 [SMILES: C[C@H](O)c1nccn1Cc1cc(-c2ccc(C#CC3CC4(C3)CN(C=O)C4)cc2)on1] | (S)-6-((4-(3-((2-(1-hydroxyethyl)- 1H-imidazol-1-yl)methyl) isoxazol-5-yl)phenyl) ethynyl)-2-azaspiro[3.3 ] heptane-2-carbaldehyde |
| Example 23 | 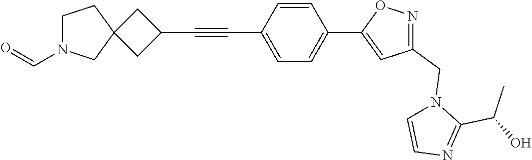 [SMILES: C[C@H](O)c1nccn1Cc1cc(-c2ccc(C#CC3CC4(CCN(C=O)C4)C3)cc2)on1] | (S)-2-((4-(3-((2-(1-hydroxyethyl)- 1H-imidazol-1-yl)methyl) isoxazol-5-yl)phenyl) ethynyl)-6-azaspiro[3.4]octane- 6-carbaldehyde |
| Example 24 | 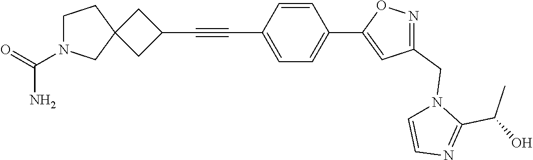 [SMILES: C[C@H](O)c1nccn1Cc1cc(-c2ccc(C#CC3CC4(CCN(C(N)=O)C4)C3)cc2)on1] | (S)-2-((4-(3-((2-(1-hydroxyethyl)- 1H-imidazol-1-yl)methyl) isoxazol-5-yl)phenyl) ethynyl)-6-azaspiro[3.4]octane- 6-carboxamide |
| Example 25 | 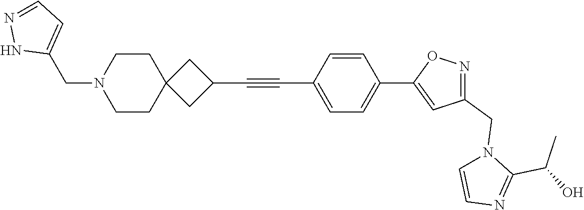 [SMILES: C[C@H](O)c1nccn1Cc1cc(-c2ccc(C#CC3CC4(CCN(Cc5ccn[nH]5)CC4)C3)cc2)on1] | (S)-1-(1-((5-(4-((7-((1H-pyrazol- 5-yl)methyl)-7-azaspiro[3.5] nonan-2-yl)ethynyl)phenyl) isoxazol-3-yl)methyl)-1H- imidazol-2-yl)ethan-1-ol |
| Example 26 | 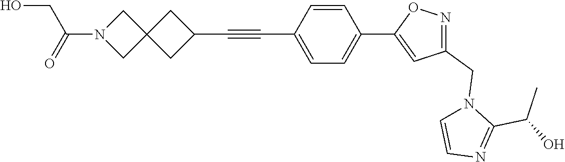 [SMILES: C[C@H](O)c1nccn1Cc1cc(-c2ccc(C#CC3CC4(C3)CN(C(=O)CO)C4)cc2)on1] | (S)-2-hydroxy-1-(6-((4-(3-((2-(1- hydroxyethyl)-1H-imidazol-1- yl)methyl)isoxazol-5-yl)phenyl) ethynyl)-2-azaspiro[3.3 ]heptan- 2-yl)ethan-1-one |
| Example 27 | 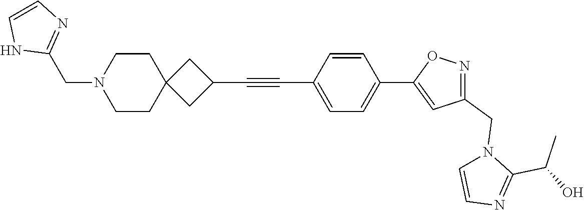 [SMILES: C[C@H](O)c1nccn1Cc1cc(-c2ccc(C#CC3CC4(CCN(Cc5ncc[nH]5)CC4)C3)cc2)on1] | (S)-1-(1-((5-(4-((7-((1H-imidazol- 2-yl)methyl)-7-azaspiro[3.5] nonan-2-yl)ethynyl)phenyl) isoxazol-3-yl)methyl)-1H- imidazol-2-yl)ethan-1-ol |
| Example 28 | 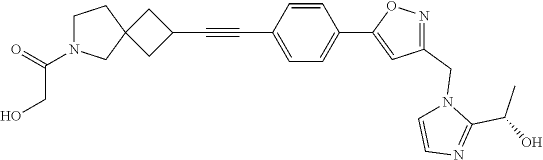 [SMILES: C[C@H](O)c1nccn1Cc1cc(-c2ccc(C#CC3CC4(CCN(C(=O)CO)C4)C3)cc2)on1] | (S)-2-hydroxy-1-(2-((4-(3-((2-(1- hydroxyethyl)-1H-imidazol-1-yl) methyl)isoxazol-5-yl)phenyl) ethynyl)-6-azaspiro[3.4]octan- 6-yl)ethan-1-one |
| Example 29 | 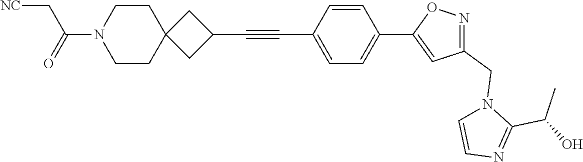 [SMILES: C[C@H](O)c1nccn1Cc1cc(-c2ccc(C#CC3CC4(CCN(C(=O)CC#N)CC4)C3)cc2)on1] | (S)-3-(2-((4-(3-((2-(1- hydroxyethyl)-1H-imidazol- 1-yl)methyl)isoxazol-5-yl) phenyl)ethynyl)-7-azaspiro [3.5]nonan-7-yl)-3- oxopropanenitrile |
| Example 30 | 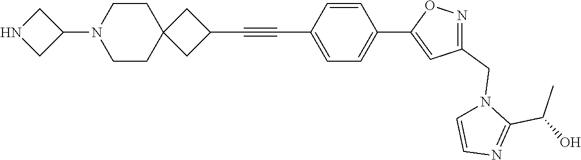 [SMILES: C[C@H](O)c1nccn1Cc1cc(-c2ccc(C#CC3CC4(CCN(C5CNC5)CC4)C3)cc2)on1] | (S)-1-(1-((5-(4-((7-(azetidin-3-yl)- 7-azaspiro[3.5]nonan-2-yl) ethynyl)phenyl)isoxazol-3-yl) methyl)-1H-imidazol- 2-yl)ethan-1-ol |
| Example 31 | 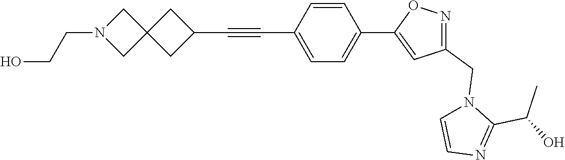 [SMILES: C[C@H](O)c1nccn1Cc1cc(-c2ccc(C#CC3CC4(C3)CN(CCO)C4)cc2)on1] | (S)-2-(6-((4-(3-((2-(1- hydroxyethyl)-1H-imidazol- 1-yl)methyl)isoxazol-5-yl) phenyl)ethynyl)-2-azaspiro[3.3] heptan-2-yl)ethan-1-ol |
| Example 32 | 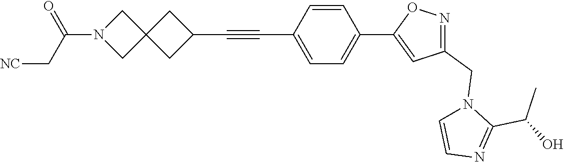 [SMILES: C[C@H](O)c1nccn1Cc1cc(-c2ccc(C#CC3CC4(C3)CN(C(=O)CC#N)C4)cc2)on1] | (S)-3-(6-((4-(3-((2-(1- hydroxyethyl)-1H-imidazol- 1-yl)methyl)isoxazol-5-yl) phenyl)ethynyl)-2-azaspiro[3.3 ] heptan-2-yl)-3- oxopropanenitrile |
| Example 33 | 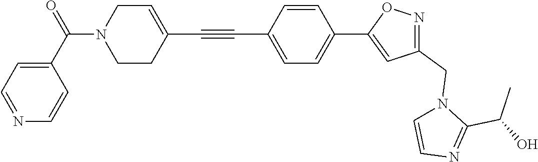 [SMILES: C[C@H](O)c1nccn1Cc1cc(-c2ccc(C#CC3=CCN(C(=O)c4ccncc4)CC3)cc2)on1] | (S)-(4-((4-(3-((2-(1- hydroxyethyl)- 1H-imidazol-1-yl)methyl) isoxazol-5-yl)phenyl)ethynyl)- 3,6-dihydropyridin-1(2H)- yl)(pyridin-4-yl)methanone |
| Example 34 | 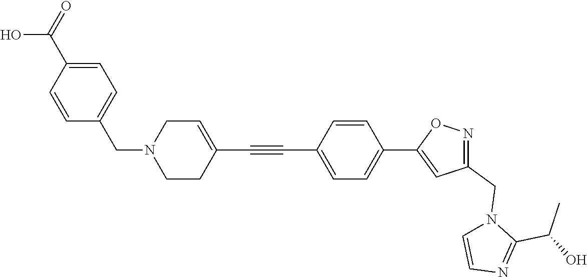 [SMILES: C[C@H](O)c1nccn1Cc1cc(-c2ccc(C#CC3=CCN(Cc4ccc(C(=O)O)cc4)CC3)cc2)on1] | (S)-4-((4-((4-(3-((2-(1- hydroxyethyl)-1H-imidazol- 1-yl)methyl)isoxazol-5-yl) phenyl)ethynyl)-3,6- dihydropyridin-1(2H)-yl) methyl)benzoic acid |
| Example 35 | 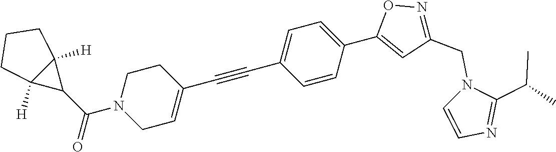 | ((1R,5S)-bicyclo[3.1.0]hexan-6- yl)(4-((4-(3-((2-((S)-1- hydroxyethyl)-1H-imidazol-1-yl) methyl)isoxazol-5-yl) phenyl)ethynyl)-3,6- dihydropyridin-1(2H)-yl) methanone |
| Example 36 | 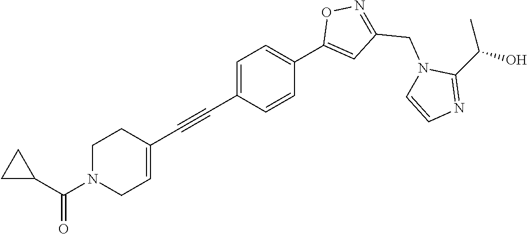 [SMILES: C[C@H](O)c1nccn1Cc1cc(-c2ccc(C#CC3=CCN(C(=O)C4CC4)CC3)cc2)on1] | (S)-cyclopropyl(4-((4-(3-((2-(1- hydroxyethyl)-1H-imidazol-1-yl) methyl)isoxazol-5-yl)phenyl) ethynyl)-3,6-dihydropyridin-1 (2H)-yl)methanone |
| Example 37 | 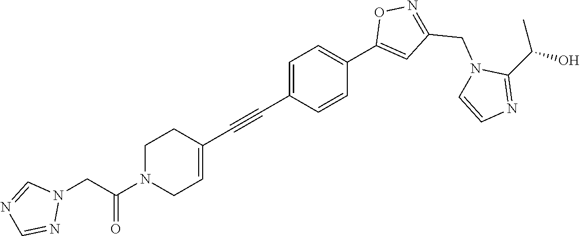 [SMILES: C[C@H](O)c1nccn1Cc1cc(-c2ccc(C#CC3=CCN(C(=O)Cn4cncn4)CC3)cc2)on1] | (S)-1-(4-((4-(3-((2-(1- hydroxyethyl)-1H-imidazol- 1-yl)methyl)isoxazol-5-yl) phenyl)ethynyl)-3,6- dihydropyridin-1(2H)-yl)-2-(1H- 1,2,4-triazol-1-yl)ethan-1-one |
| Example 38 | 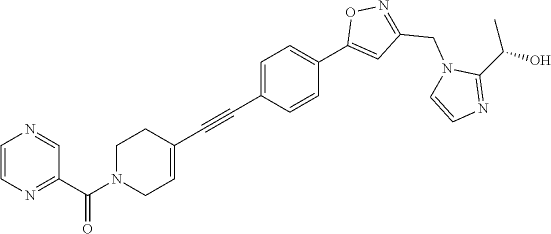 [SMILES: C[C@H](O)c1nccn1Cc1cc(-c2ccc(C#CC3=CCN(C(=O)c4cnccn4)CC3)cc2)on1] | (S)-(4-((4-(3-((2-(1- hydroxyethyl)-1H-imidazol- 1-yl)methyl)isoxazol-5-yl) phenyl)ethynyl)-3,6- dihydropyridin-1(2H)-yl)(pyrazin- 2-yl)methanone |
| Example 39 | 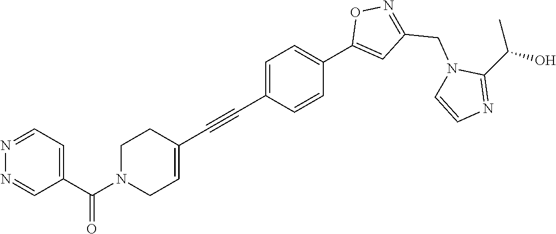 [SMILES: C[C@H](O)c1nccn1Cc1cc(-c2ccc(C#CC3=CCN(C(=O)c4ccnnc4)CC3)cc2)on1] | (S)-(4-((4-(3-((2-(1-hydroxyethyl)- 1H-imidazol-1-yl)methyl) isoxazol-5-yl)phenyl)ethynyl)- 3,6-dihydropyridin-1(2H)- yl)(pyridazin-4-yl)methanone |
| Example 40 | 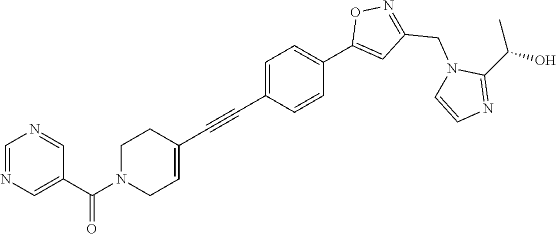 [SMILES: C[C@H](O)c1nccn1Cc1cc(-c2ccc(C#CC3=CCN(C(=O)c4cncnc4)CC3)cc2)on1] | (S)-(4-((4-(3-((2-(1-hydroxyethyl)- 1H-imidazol-1-yl)methyl) isoxazol-5-yl)phenyl) ethynyl)-3,6-dihydropyridin- 1(2H)-yl)(pyrimidin-5-yl) methanone |
| Example 41 | 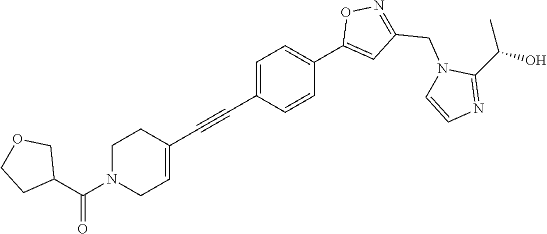 [SMILES: C[C@H](O)c1nccn1Cc1cc(-c2ccc(C#CC3=CCN(C(=O)C4CCOC4)CC3)cc2)on1] | (4-((4-(3-((2-((S)-1-hydroxyethyl)- 1H-imidazol-1-yl)methyl) isoxazol-5-yl)phenyl) ethynyl)-3,6-dihydropyridin- 1(2H)-yl)(tetrahydrofuran- 3-yl)methanone |
| Example 42 | 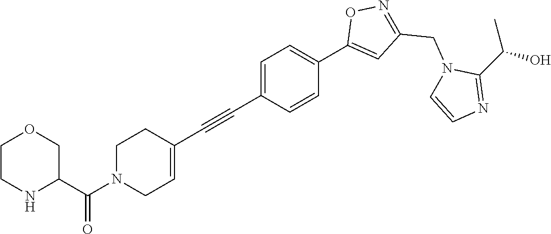 [SMILES: C[C@H](O)c1nccn1Cc1cc(-c2ccc(C#CC3=CCN(C(=O)C4COCCN4)CC3)cc2)on1] | (4-((4-(3-((2-((S)-1-hydroxyethyl)- 1H-imidazol-1-yl)methyl) isoxazol-5-yl)phenyl) ethynyl)-3,6-dihydropyridin- 1(2H)-yl)(morpholin-3- yl)methanone |
| Example 43 | 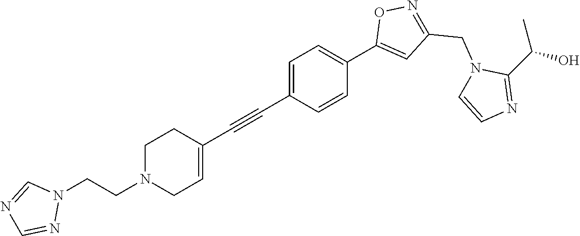 [SMILES: C[C@H](O)c1nccn1Cc1cc(-c2ccc(C#CC3=CCN(CCn4cncn4)CC3)cc2)on1] | (S)-1-(1-((5-(4-((1-(2-(1H-1,2,4- triazol-1-yl)ethyl)-1,2,3,6- tetrahydropyridin-4-yl) ethynyl)phenyl)isoxazol-3-yl) methyl)-1H-imidazol-2-yl) ethan-1-ol |
| Example 44 | 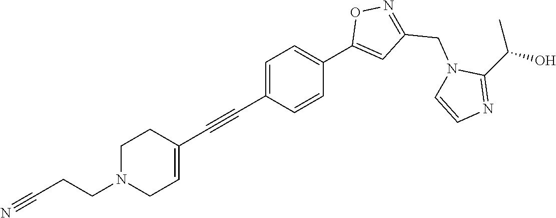 [SMILES: C[C@H](O)c1nccn1Cc1cc(-c2ccc(C#CC3=CCN(CCC#N)CC3)cc2)on1] | (S)-3-(4-((4-(3-((2-(1- hydroxyethyl)-1H-imidazol- 1-yl)methyl)isoxazol-5-yl) phenyl)ethynyl)-3,6- dihydropyridin-1(2H)-yl) propanenitrile |
| Example 45 | 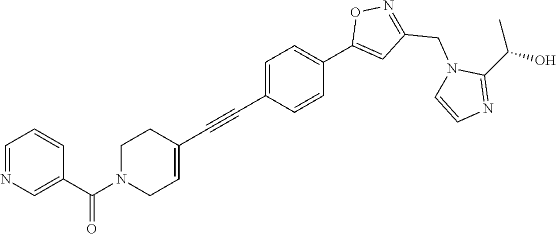 [SMILES: C[C@H](O)c1nccn1Cc1cc(-c2ccc(C#CC3=CCN(C(=O)c4cccnc4)CC3)cc2)on1] | (S)-(4-((4-(3-((2-(1-hydroxyethyl)- 1H-imidazol-1-yl)methyl) isoxazol-5-yl)phenyl) ethynyl)-3,6-dihydropyridin- 1(2H)-yl)(pyridin- 3-yl)methanone |
| Example 46 | 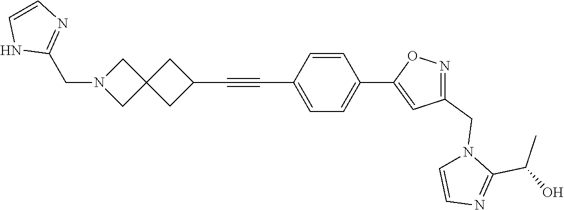 [SMILES: C[C@H](O)c1nccn1Cc1cc(-c2ccc(C#CC3CC4(C3)CN(Cc3ncc[nH]3)C4)cc2)on1] | (S)-1-(1-((5-(4-((2-((1H-imidazol- 2-yl)methyl)-2-azaspiro[3.3] heptan-6-yl)ethynyl)phenyl) isoxazol-3-yl)methyl)-1H- imidazol-2-yl)ethan-1-ol |
| Example 47 | 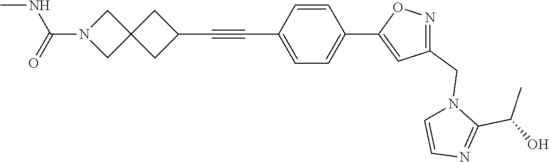 [SMILES: CNC(=O)N1CC2(CC(C#Cc3ccc(-c4cc(Cn5ccnc5[C@H](C)O)no4)cc3)C2)C1] | (S)-6-((4-(3-((2-(1-hydroxyethyl)- 1H-imidazol-1-yl)methyl) isoxazol-5-yl)phenyl) ethynyl)-N-methyl-2-azaspiro [3.3 ]heptane-2-carboxamide |
| Example 48 | 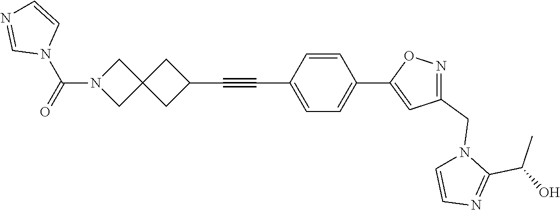 [SMILES: C[C@H](O)c1nccn1Cc1cc(-c2ccc(C#CC3CC4(C3)CN(C(=O)n3ccnc3)C4)cc2)on1] | (S)-(6-((4-(3-((2-(1-hydroxyethyl)- 1H-imidazol-1-yl)methyl) isoxazol-5-yl)phenyl) ethynyl)-2-azaspiro[3.3 ] heptan-2-yl)(1H-imidazol- 1-yl) methanone |
| Example 49 | 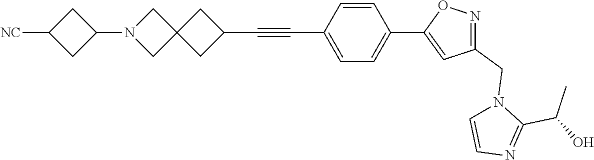 [SMILES: C[C@H](O)c1nccn1Cc1cc(-c2ccc(C#CC3CC4(C3)CN(C3CC(C#N)C3)C4)cc2)on1] | (S)-3-(6-((4-(3-((2-(1- hydroxyethyl)-1H-imidazol- 1-yl)methyl)isoxazol-5-yl)phenyl) ethynyl)-2-azaspiro[3.3] heptan-2-yl)cyclobutane- 1-carbonitrile |
| Example 50 | 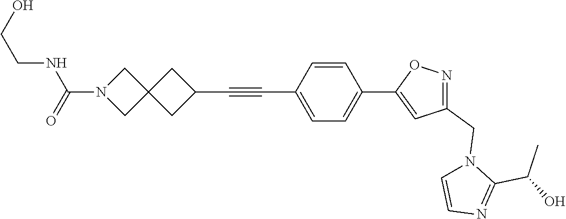 [SMILES: C[C@H](O)c1nccn1Cc1cc(-c2ccc(C#CC3CC4(C3)CN(C(=O)NCCO)C4)cc2)on1] | (S)-N-(2-hydroxyethyl)-6-((4-(3- ((2-(1-hydroxyethyl)-1H- imidazol-1-yl)methyl)isoxazol- 5-yl)phenyl)ethynyl)-2-azaspiro [3.3]heptane-2-carboxamide |
| Example 51 | 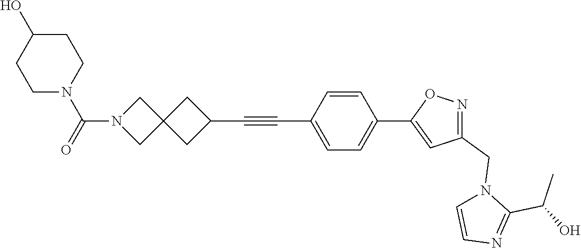 [SMILES: C[C@H](O)c1nccn1Cc1cc(-c2ccc(C#CC3CC4(C3)CN(C(=O)N3CCC(O)CC3)C4)cc2)on1] | (S)-(6-((4-(3-((2-(1-hydroxyethyl)- 1H-imidazol-1-yl)methyl) isoxazol-5-yl)phenyl) ethynyl)-2-azaspiro[3.3 ]heptan- 2-yl)(4-hydroxy piperidin-1-yl)methanone |
| Example 52 | 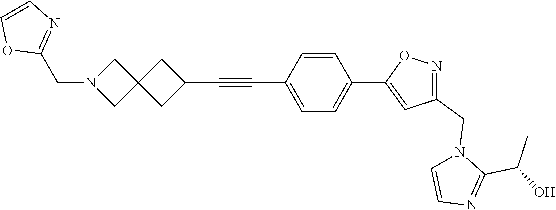 [SMILES: C[C@H](O)c1nccn1Cc1cc(-c2ccc(C#CC3CC4(C3)CN(Cc3ncco3)C4)cc2)on1] | (S)-1-(1-((5-(4-((2-(oxazol-2-yl methyl)-2-azaspiro[3.3]heptan-6- yl)ethynyl)phenyl)isoxazol-3-yl) methyl)-1H-imidazol-2-yl)ethan- 1-ol |
| Example 53 | 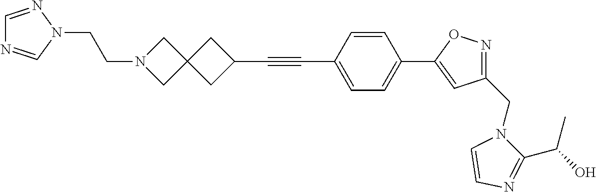 [SMILES: C[C@H](O)c1nccn1Cc1cc(-c2ccc(C#CC3CC4(C3)CN(CCn3cncn3)C4)cc2)on1] | (S)-1-(1-((5-(4-((2-(2-(1H-1,2,4- triazol-1-yl)ethyl)-2-azaspiro[3.3] heptan-6-yl)ethynyl)phenyl) isoxazol-3-yl)methyl)-1H- imidazol-2-yl)ethan-1-ol |
| Example 54 | 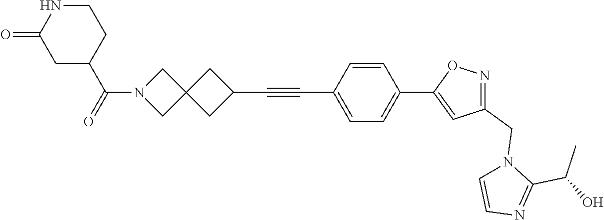 [SMILES: C[C@H](O)c1nccn1Cc1cc(-c2ccc(C#CC3CC4(C3)CN(C(=O)C3CCNC(=O)C3)C4)cc2)on1] | 4-(6-((4-(3-((2-((S)-1- hydroxyethyl)-1H-imidazol- 1-yl)methyl)isoxazol-5-yl) phenyl)ethynyl)-2-azaspiro [3.3]heptane-2-carbonyl) piperidin-2-one |
| Example 55 | 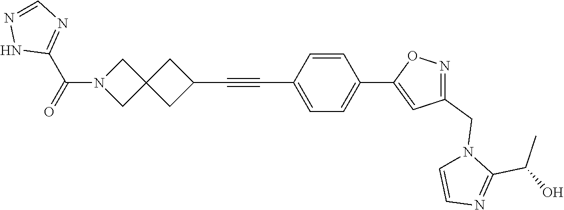 [SMILES: C[C@H](O)c1nccn1Cc1cc(-c2ccc(C#CC3CC4(C3)CN(C(=O)c3ncn[nH]3)C4)cc2)on1] | (S)-(6-((4-(3-((2-(1- hydroxyethyl)-1H-imidazol- 1-yl)methyl)isoxazol-5-yl) phenyl)ethynyl)-2-azaspiro [3.3]heptan-2-yl)(1H-1,2,4- triazol-5-yl)methanone |
| Example 56 | 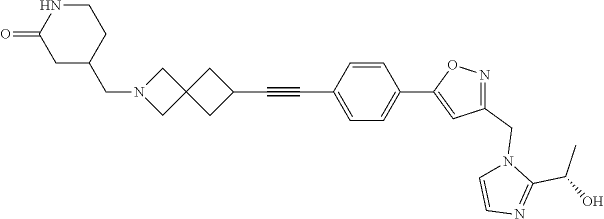 [SMILES: C[C@H](O)c1nccn1Cc1cc(-c2ccc(C#CC3CC4(C3)CN(CC3CCNC(=O)C3)C4)cc2)on1] | 4-((6-((4-(3-((2-((S)-1- hydroxyethyl)-1H-imidazol- 1-yl)methyl)isoxazol-5-yl) phenyl)ethynyl)-2-azaspiro [3.3]heptan-2-yl)methyl) piperidin-2-one |
| Example 57 | 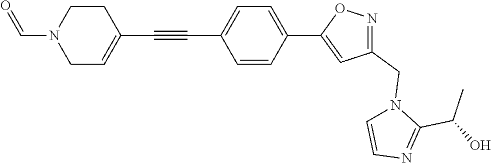 [SMILES: C[C@H](O)c1nccn1Cc1cc(-c2ccc(C#CC3=CCN(C=O)CC3)cc2)on1] | (S)-4-((4-(3-((2-(1-hydroxyethyl)- 1H-imidazol-1-yl)methyl) isoxazol-5-yl)phenyl)ethynyl)- 3,6-dihydropyridine- 1(2H)-carbaldehyde |
| Example 58 | 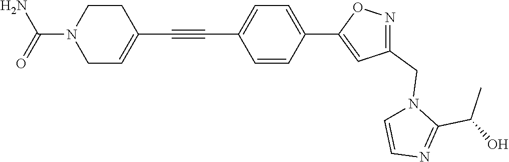 [SMILES: C[C@H](O)c1nccn1Cc1cc(-c2ccc(C#CC3=CCN(C(N)=O)CC3)cc2)on1] | (S)-4-((4-(3-((2-(1-hydroxyethyl)- 1H-imidazol-1-yl)methyl) isoxazol-5-yl)phenyl)ethynyl)- 3,6-dihydropyridine- 1(2H)-carboxamide |
| Example 59 | 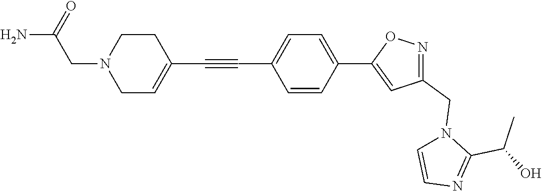 [SMILES: C[C@H](O)c1nccn1Cc1cc(-c2ccc(C#CC3=CCN(CC(N)=O)CC3)cc2)on1] | (S)-2-(4-((4-(3-((2-(1- hydroxyethyl)-1H-imidazol- 1-yl)methyl)isoxazol-5-yl)phenyl) ethynyl)-3,6-dihydropyridin- 1(2H)-yl)acetamide |
| Example 60 | 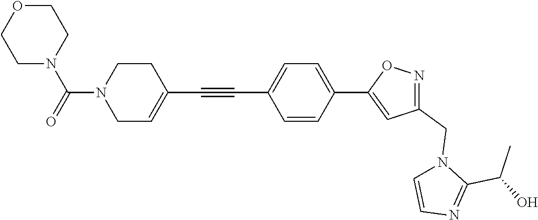 [SMILES: C[C@H](O)c1nccn1Cc1cc(-c2ccc(C#CC3=CCN(C(=O)N4CCOCC4)CC3)cc2)on1] | (S)-(4-((4-(3-((2-(1- hydroxyethyl)-1H-imidazol- 1-yl)methyl)isoxazol- 5-yl)phenyl)ethynyl)-3,6- dihydropyridin-1(2H)-yl) (morpholino)methanone |
| Example 61 | 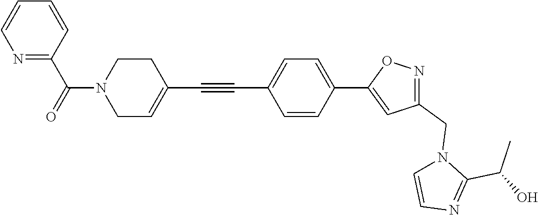 [SMILES: C[C@H](O)c1nccn1Cc1cc(-c2ccc(C#CC3=CCN(C(=O)c4ccccn4)CC3)cc2)on1] | (S)-(4-((4-(3-((2-(1- hydroxyethyl)-1H-imidazol- 1-yl)methyl)isoxazol-5-yl)phenyl) ethynyl)-3,6-dihydropyridin- 1(2H)-yl)(pyridin- 2-yl)methanone |
| Example 62 | 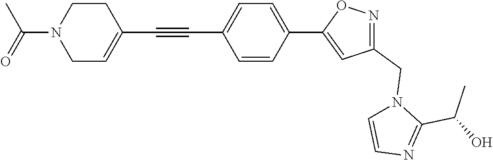 [SMILES: CC(=O)N1CC=C(C#Cc2ccc(-c3cc(Cn4ccnc4[C@H](C)O)no3)cc2)CC1] | (S)-1-(4-((4-(3-((2-(1- hydroxyethyl)-1H-imidazol- 1-yl)methyl)isoxazol-5-yl)phenyl) ethynyl)-3,6-dihydropyridin- 1(2H)-yl)ethan-1-one |
| Example 63 | 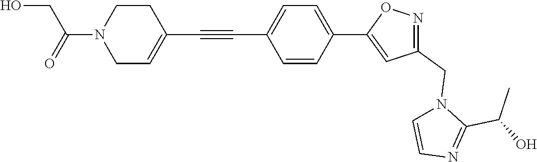 [SMILES: C[C@H](O)c1nccn1Cc1cc(-c2ccc(C#CC3=CCN(C(=O)CO)CC3)cc2)on1] | (S)-2-hydroxy-1-(4-((4-(3-((2-(1- hydroxyethyl)-1H-imidazol-1-yl) methyl)isoxazol-5-yl)phenyl) ethynyl)-3,6-dihydropyridin-1 (2H)-yl)ethan-1-one |
| Example 64 | 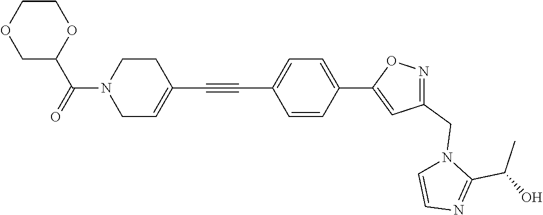 [SMILES: C[C@H](O)c1nccn1Cc1cc(-c2ccc(C#CC3=CCN(C(=O)C4COCCO4)CC3)cc2)on1] | (1,4-dioxan-2-yl)(4-((4-(3-((2- ((S)-1-hydroxyethyl)-1H- imidazol-1-yl)methyl)isoxazol- 5-yl)phenyl)ethynyl)-3,6- dihydropyridin-1(2H)-yl) methanone |
| Example 65 | 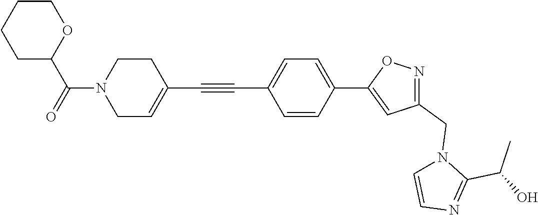 [SMILES: C[C@H](O)c1nccn1Cc1cc(-c2ccc(C#CC3=CCN(C(=O)C4CCCCO4)CC3)cc2)on1] | (4-((4-(3-((2-((S)-1- hydroxyethyl)-1H-imidazol- 1-yl)methyl)isoxazol-5-yl) phenyl)ethynyl)-3,6- dihydropyridin-1(2H)-yl) (tetrahydro-2H-pyran-2-yl) methanone |
| Example 66 | 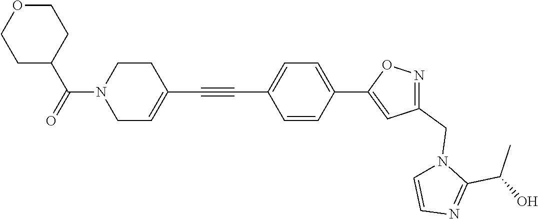 [SMILES: C[C@H](O)c1nccn1Cc1cc(-c2ccc(C#CC3=CCN(C(=O)C4CCOCC4)CC3)cc2)on1] | (S)-(4-((4-(3-((2-(1-hydroxyethyl)- 1H-imidazol-1-yl)methyl) isoxazol-5-yl)phenyl) ethynyl)-3,6-dihydropyridin- 1(2H)-yl)(tetrahydro- 2H-pyran-4-yl)methanone |
| Example 67 | 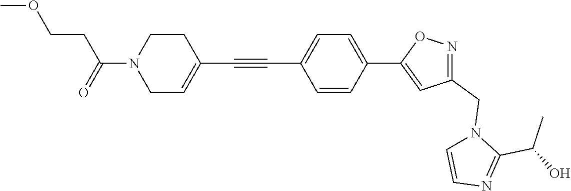 [SMILES: COCCC(=O)N1CC=C(C#Cc2ccc(-c3cc(Cn4ccnc4[C@H](C)O)no3)cc2)CC1] | (S)-1-(4-((4-(3-((2-(1- hydroxyethyl)-1H-imidazol- 1-yl)methyl)isoxazol-5-yl) phenyl)ethynyl)-3,6- dihydropyridin-1(2H)-yl)-3- methoxypropan-1-one |
| Example 68 | 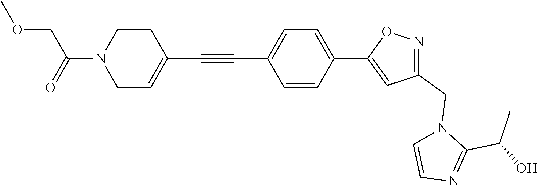 [SMILES: COCC(=O)N1CC=C(C#Cc2ccc(-c3cc(Cn4ccnc4[C@H](C)O)no3)cc2)CC1] | (S)-1-(4-((4-(3-((2-(1- hydroxyethyl)-1H-imidazol- 1-yl)methyl)isoxazol-5-yl) phenyl)ethynyl)-3,6- dihydropyridin-1(2H)-yl)-2- methoxyethan-1-one |
| Example 69 | 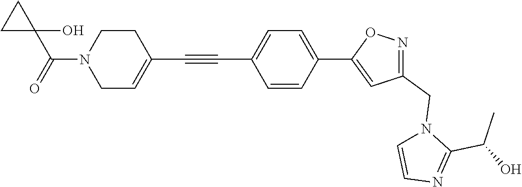 [SMILES: C[C@H](O)c1nccn1Cc1cc(-c2ccc(C#CC3=CCN(C(=O)C4(O)CC4)CC3)cc2)on1] | (S)-(1-hydroxycyclopropyl)(4- ((4-(3-((2-(1-hydroxyethyl)-1H- imidazol-1-yl)methyl)isoxazol-5- yl)phenyl)ethynyl)-3,6- dihydropyridin-1(2H)-yl) methanone |

or a stereoisomer, a tautomer thereof or a pharmaceutically acceptable salt thereof.

Note: if there is a difference between the drawn structure and the given name of the structure, the drawn structure will be given greater weight.

Still further, the present invention provides a pharmaceutical composition, and the pharmaceutical composition contains an effective dose of a compound represented by general formula (I) or (II), or a stereoisomer or a tautomer thereof, or a pharmaceutically acceptable salt thereof, and a pharmaceutically acceptable carrier, an excipient or a combination thereof.

The present invention provides use of the compound represented by general formula (I) or (II), or the stereoisomer or the tautomer thereof, or the pharmaceutically acceptable salt thereof, or the pharmaceutical composition thereof (for example, the pharmaceutical composition according to the foregoing technical solution) in preparing an LPXC inhibitor.

The present invention further provides use of the compound represented by general formula (I) or (II), or the stereoisomer or the tautomer thereof, or the pharmaceutically acceptable salt thereof, or the pharmaceutical composition thereof (for example, the pharmaceutical composition according to the foregoing technical solution) in preparing a medicament for treating a disease mediated by LPXC, wherein the disease mediated by LPXC is selected from a bacterial infection caused by a Gram-negative bacterium; wherein the disease mediated by LPXC is more preferably selected from bacterial infection caused by a Gram-negative bacterium selected from Escherichia coli, Pseudomonas aeruginosa, Proteusbacillus vulgaris, Shigella dysenteriae, Pneumobacillus, Brucella, Typhoid Bacillus, Acinetobacter, Yersinia, Legionella pneumophila, Bordetella pertussis, Shigella, Pasteurella, Vibrio cholerae or Neisseria Meningitidis.

The present invention further provides use of the compound represented by general formula (I) or (II), or the stereoisomer or the tautomer thereof, or the pharmaceutically acceptable salt thereof, or the pharmaceutical composition thereof (for example, the pharmaceutical composition according to the foregoing technical solution) in preparing a medicament for treating a bacterial infection caused by a Gram-negative bacterium.

The present invention provides use of the compound represented by general formula (I) or (II), or the stereoisomer or the tautomer thereof, or the pharmaceutically acceptable salt thereof, or the pharmaceutical composition thereof (for example, the pharmaceutical composition according to the foregoing technical solution) in preparing a medicament for treating bacterial infection, wherein the bacterial infection is caused by Gram-negative bacterium selected from Escherichia coli, Pseudomonas aeruginosa, Proteusbacillus vulgaris, Shigella dysenteriae, Pneumobacillus, Brucella, Typhoid Bacillus, Acinetobacter, Yersinia, Legionella pneumophila, Bordetella pertussis, Shigella, Pasteurella, Vibrio cholerae or Neisseria Meningitidis.

The present invention provides a method for preventing and/or treating a disease mediated by LPXC, which comprises administering to a patient a therapeutically effective dose of the compound represented by general formula (I) or (II), or the stereoisomer or the tautomer thereof, or the pharmaceutically acceptable salt thereof the pharmaceutical composition thereof (for example, the pharmaceutical composition according to the foregoing technical solution), wherein the disease mediated by LPXC is preferably a bacterial infection caused by a Gram-negative bacterium; wherein the disease mediated by LPXC is more preferably selected from a bacterial infection caused by a Gram-negative bacterium selected from Escherichia coli, Pseudomonas aeruginosa, Proteusbacillus vulgaris, Shigella dysenteriae, Pneumobacillus, Brucella, Typhoid Bacillus, Acinetobacter; Yersinia, Legionella pneumophila, Bordetella pertussis, Shigella, Pasteurella, Vibrio cholerae or Neisseria Meningitidis.

The present invention further provides a method for preventing and/or treating a bacterial infection caused by a Gram-negative bacterium, which comprises administering to a patient a therapeutically effective dose of the compound represented by general formula (I) or (II), or the stereoisomer or the tautomer thereof, or the pharmaceutically acceptable salt thereof the pharmaceutical composition thereof (for example, the pharmaceutical composition according to the foregoing technical solution).

The present invention further provides a method for preventing and/or treating a bacterial infection caused by a Gram-negative bacterium such as Escherichia coli, Pseudomonas aeruginosa, Proteusbacillus vulgaris, Shigella dysenteriae, Pneumobacillus, Brucella, Typhoid Bacillus, Acinetobacter; Yersinia, Legionella pneumophila, Bordetella pertussis, Shigella, Pasteurella, Vibrio cholerae or Neisseria Meningitidis, which comprises administering to a patient a therapeutically effective dose of the compound represented by general formula (I) or (II), or the stereoisomer or the tautomer thereof, or the pharmaceutically acceptable salt thereof, the pharmaceutical composition thereof (for example, the pharmaceutical composition according to the foregoing technical solution).

### DETAILED DESCRIPTION OF THE INVENTION

Unless stated to the contrary, some terms used in the specification and the claims of the present invention are defined as follows.

“Alkyl” refers to an aliphatic hydrocarbon group comprising a C₁-C₂₀ straight chain or having a branched chain when taken as one group or a part of one group. Preferably, the alkyl is C₁-C₁₀ alkyl, and more preferably, the alkyl is C₁-C₆ alkyl. Examples of an alkyl group comprise, but are not limited to, methyl, ethyl, n-propyl, isopropyl, n-butyl, isobutyl, tert-butyl, sec-butyl, n-pentyl, 1,1-dimethyl propyl, 1,2-dimethyl propyl, 2,2-dimethyl propyl, 1-ethyl propyl, 2-methyl butyl, 3-methyl butyl, n-hexyl, 1-ethyl-2-methyl propyl, 1,1,2-trimethyl propyl, 1,1-dimethyl butyl, 1,2-dimethyl butyl, 2,2-dimethyl butyl, 1,3-dimethyl butyl, 2-ethyl butyl, 2-methyl pentyl, 3-methyl pentyl, 4-methyl pentyl, 2,3-dimethyl butyl, and the like. The alkyl can be substituted or unsubstituted.

“Cycloalkyl” refers to a non-aromatic cyclic alkyl in which one or more ring-forming atoms are carbon atoms, comprising a monocyclic ring, a polycyclic ring, a fused ring, a bridged ring and a spirocyclic ring. Preferably, the cycloalkyl comprises a 5- to 7-membered monocyclic ring or a 7- to 10-membered bicyclic or tricyclic ring. Examples of “the cycloalkyl” comprise, but are not limited to, cyclopropyl, cyclopentyl and cyclobutyl. The cycloalkyl can be substituted or unsubstituted.

“Spiroalkyl” refers to a 5- to 18-membered polycyclic group with two or more cyclic structures, and single rings share one carbon atom (called a spiro atom) with each other, and the ring contains one or more double bonds, but no ring has a fully conjugated π electron aromatic system. Preferably, the spiroalkyl is 6- to 14-membered, and more preferably, the spiroalkyl is 7- to 10-membered. The spiroalkyl is classified into mono-, di- or multi-spiroalkyl according to a number of shared carbon atoms between rings, is preferably the mono- and di-spiroalkyl, and preferably is 4 membered/5 membered, 4 membered/6 membered, 5 membered/5 membered or 5 membered/6 membered. Non-limiting examples of the “spiroalkyl” comprise, but are not limited to, spiro[4.5]decyl, spiro[4.4]nonyl, spiro[3.5]nonyl and spiro[2.4]heptyl.

“Fused cycloalkyl” refers to a 5- to 18-membered full-carbon polycyclic group with two or more cyclic structures sharing one pair of carbon atoms, and one or more rings can contain one or more double bonds, but no ring has a fully conjugated π electron aromatic system. Preferably, the fused cycloalkyl is 6- to 12-membered, and more preferably, the fused cycloalkyl is 7- to 10-membered. The fused cycloalkyl may be classified into bicyclic, tricyclic, tetracyclic or polycyclic fused cycloalkyl according to a number of constituent rings, is preferably bicyclic or tricyclic fused cycloalkyl, and is more preferably bicycloalkyl as 5 membered/5 membered or 5 membered/6 membered. Non-limiting examples of the “fused cycloalkyl” comprise, but are not limited to,

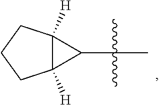 [SMILES: *C1[C@H]2CCC[C@@H]12]

bicyclo[3.1.0]hexyl, bicyclo[3.2.0]heptyl-1-alkenyl, bicyclo[3.2.0]heptyl, decahydronaphthyl or tetradecahydrophenanthryl.

“Bridged cycloalkyl” refers to a 5- to 18-membered full-carbon polycyclic group with two or more cyclic structures sharing two carbon atoms not directly connected with each other, and one or more rings may contain one or more double bonds, but no ring has a fully conjugated 71 electron aromatic system. Preferably, the bridged cycloalkyl is 6- to 12-membered, and more preferably, the bridged cycloalkyl is 7- to 10-membered. Preferably, the bridged cycloalkyl is 6- to 14-membered, and more preferably, the bridged cycloalkyl is 7- to 10-membered. The bridged cycloalkyl may be classified into bicyclic, tricyclic, tetracyclic or polycyclic bridged cycloalkyl according to a number of constituent rings, is preferably the bicyclic, tricyclic or tetracyclic bridged cycloalkyl, and is more preferably the bicyclic or tricyclic bridged cycloalkyl. Non-limiting examples of the “bridged cycloalkyl” comprise, but are not limited to, (1s, 4s)-bicyclo[2.2.1]heptyl, bicycle[3.2.1]octyl, (1s,5s)-bicyclo[3.3.1]nonyl, bicyclo[2.2.2]octyl, and (1r,5r)-bicyclo[3.3.2]decyl.

“Heterocyclyl”, “heterocycloalkyl”, “heterocycle” or “heterocyclic” may be used interchangeably in the present application, and all refers to non-aromatic heterocyclyl, wherein one or more ring-forming atoms are selected from heteroatoms of nitrogen, oxygen, or S(O)ᵣ (wherein r is selected from 0, 1 or 2), comprising a monocyclic ring, a polycyclic ring, a fused ring, a bridged ring and a spirocyclic ring. The heterocyclyl can be substituted or unsubstituted.

Examples of “monocyclic heterocyclyl” comprise, but are not limited to, morpholinyl, oxetanyl, thiomorpholinyl, tetrahydrofuranyl, tetrahydropyranyl, 1,1-dioxo-thiomorpholinyl, piperidinyl, 2-oxo-piperidinyl, pyrrolidinyl, 2-oxo-pyrrolidinyl, piperazine-2-one, 8-oxa-3-aza-bicyclo[3.2.1]octyl, piperazinyl, hexahydropyrimidine, 1,4-dioxane, or

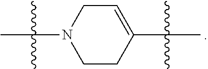 [SMILES: *C1=CCN(*)CC1 ; "."]

“Spiro-heterocyclyl” refers to a 5- to 18-membered polycyclic group with two or more cyclic structures, and single rings share one atom with each other, and the ring contains one or more double bonds, but no ring has a fully conjugated 71 electron aromatic system, wherein one or more ring atoms are selected from heteroatoms of nitrogen, oxygen or S(O)ᵣ (wherein r is selected from 0, 1 or 2), and the remaining ring atoms are carbon. Preferably, the spiro-heterocyclyl is 5- to 14-membered, more preferably, the spiro-heterocyclyl is 6- to 10-membered, and most preferably, the spiro-heterocyclyl is 7- to 10-membered. The spiro-heterocyclyl is divided into mono-, di- or multi-spiro-heterocyclyl according to a number of shared spiro atoms between rings, is preferably the mono- and di-spiro-heterocyclyl, and is more preferably a 4 membered/4 membered, 4 membered/5 membered, 4 membered/6 membered, 5 membered/5 membered or 5 membered/6 membered mono-spiro-heterocyclyl. Non-limiting examples of the “spiro-heterocyclyl” comprise, but are not limited to, 1,7-dioxane[4.5]decyl, 2-oxa-7-azaspiro[4.4]nonyl, 7-oxaspiro[3.5]nonyl, 5-oxaspiro[2.4]heptyl,

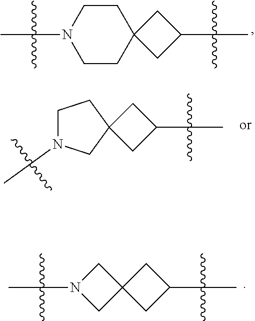

“Fused heterocyclyl” refers to a full carbon polycyclic group with two or more cyclic structures sharing one pair of atoms with each other, and one or more rings can contain one or more double bonds, but no ring has a fully conjugated π electron aromatic system, wherein one or more ring atoms are selected from heteroatoms of nitrogen, oxygen or S(O)ᵣ (wherein r is selected from 0, 1 or 2), and the remaining ring atoms are carbon. Preferably, the fused heterocyclyl is 6- to 14-membered, and more preferably, the fused heterocyclyl is 7- to 10-membered. The fused heterocyclyl may be divided into bicyclic, tricyclic, tetracyclic or polycyclic fused heterocyclyl according to a number of constituent rings, is preferably a bicyclic or tricyclic fused heterocyclyl, and is more preferably a 5 membered/5 membered or 5 membered/6 membered bicyclic fused heterocyclyl. Non-limiting examples of the “fused heterocyclyl” comprise, but are not limited to, octahydropyrrolo[3,4-c]pyrrolyl, octahydro-1H-isoindolyl, 3-azabicyclo[3.1.0]hexyl, and octahydrobenzo[b][1,4]dioxine.

“Bridged heterocyclyl” refers to a 5- to 14-membered or 5- to 18-membered polycyclic group containing two or more cyclic structures sharing two atoms not directly connected with each other, and one or more rings may contain one or more double bonds, but no ring has a fully conjugated π electron aromatic system, wherein one or more ring atoms are selected from heteroatoms of nitrogen, oxygen or S(O)ᵣ (wherein r is selected from 0, 1 or 2), and the remaining ring atoms are carbon. Preferably, the bridged heterocyclyl is 6- to 14-membered, and more preferably, the bridged heterocyclyl is 7- to 10-membered. The bridged heterocyclyl may be divided into bicyclic, tricyclic, tetracyclic or polycyclic bridged heterocyclyl according to a number of constituent rings, is preferably a bicyclic, tricyclic or tetracyclic bridged heterocyclyl, and is more preferably a bicyclic or tricyclic bridged heterocyclyl. Non-limiting examples of the “bridged heterocyclyl” comprise, but are not limited to, 2-azabicyclo[2.2.1]heptyl, 2-azabicyclo[2.2.2]octyl and 2-azabicyclo[3.3.2]decyl.

“Aryl” refers to a carbocyclic aromatic system containing one or two rings, wherein the rings may be connected together in a fused manner. The term “aryl” comprises monocyclic or bicyclic aryl, such as aromatic groups of phenyl, naphthyl and tetrahydronaphthyl. Preferably, the aryl is C₆-C₁₀ aryl, more preferably, the aryl is the phenyl and the naphthyl, and most preferably, the aryl is the naphthyl. The aryl can be substituted or unsubstituted.

“Heteroaryl” refers to a 5- to 6-membered aromatic monocyclic ring or a 8- to 10-membered aromatic bicyclic ring, which may contain 1 to 4 atoms selected from nitrogen, oxygen and/or sulfur. Examples of the “heteroaryl” comprise, but are not limited to, furyl, pyridyl, 2-oxo-1,2-dihydropyridyl, pyridazinyl, pyrimidinyl, pyrazinyl, thienyl, isoxazolyl, oxazolyl, oxadiazolyl, imidazolyl, pyrrolyl, pyrazolyl, triazolyl, tetrazolyl, thiazolyl, isothiazolyl, 1,2,3-thiadiazolyl, benzodioxolyl, benzothiophenyl, benzimidazolyl, indolyl, isoindolyl, 1,3-dioxo-isoindolyl, quinolyl, indazolyl, benzisothiazolyl, benzoxazolyl, benzisoxazolyl, isothiazolyl, 1H-1,2,4-triazolyl, 4H-1,2,4-triazolyl, pyridyl, pyrimidinyl, pyrazine-2(1H)-keto, pyrimidine-4(3H)-keto, pyridazine-3(2H)-keto, 1H-indolyl, 1H-benzo[d]imidazolyl, 1H-pyrrolo[2,3-c]pyridyl, 3H-imidazo[4,5-c]pyridyl, isoquinolinyl, quinazolinyl, 2H-isoindolyl, furan[3,2-b]pyridyl, furan[2,3-c]pyridyl, thieno[2,3-c]pyridyl, benzofuranyl, benzo[b]thienyl, 1H-pyrrolo[3,2-b]pyridyl, and 2H-pyrrolo[3,4-c]pyridyl. The heteroaryl can be substituted or unsubstituted.

“Alkoxy” refers to a group of (alkyl-O—), wherein the alkyl is defined herein. C₁-C₆ alkoxy is preferred. Examples of alkoxy comprise, but are not limited to, methoxy, ethoxy, n-propoxy, isopropoxy, n-butoxy, isobutoxy, tert-butoxy, and the like.

“Nitro” refers to an —NO₂ group.

“Hydroxyl” refers to an —OH group.

“Halogen” refers to fluorine, chlorine, bromine and iodine.

“Amino” refers to —NH₂.

“Cyano” refers to —CN.

“Benzyl” refers to —CH₂-phenyl.

“Carboxyl” refers to —C(O)OH.

“Carboxylate group” refers to —C(O)O-alkyl or —C(O)O-cycloalkyl, wherein the alkyl and the cycloalkyl are defined as above.

“Hydroxy alkyl” or “hydroxyalkyl” refers to alkyl substituted by hydroxyl, wherein the alkyl is defined as above.

“Aminoalkyl” refers to alkyl substituted by amino, wherein the alkyl is defined as above.

“Haloalkyl” refers to alkyl substituted by halogens, wherein the alkyl is defined herein.

“Haloalkoxy” refers to alkoxy substituted by halogens, wherein the alkoxy is defined herein.

“DMSO” refers to dimethyl sulfoxide.

“BOC” refers to tert-butoxycarbonyl.

“Bn” refers to benzyl.

“THP” refers to 2-tetrahydropyranyl.

“TFA” refers to trifluoroacetic acid.

“Ts” refers to p-toluenesulfonyl.

“Leaving group”, is an atom or functional group separated from a larger molecule in chemical reaction, which is a term used in nucleophilic substitution reaction and elimination reaction. In nucleophilic substitution reaction, a reactant attacked by a nucleophilic reagent is called substrate, while an atom or atomic group broken away with a pair of electrons in the substrate molecule is called leaving group. A group that accepts electrons easily and has a strong ability to bear negative charges is a good leaving group. When pKa of a conjugate acid of the leaving group is smaller, it is easier for the leaving group to separate from other molecules. The reason is that when the pKa of the conjugated acid of the leaving group is smaller, the corresponding leaving group does not need to be combined with other atoms, and the tendency to exist in the form of anions (or electrically neutral leaving group) is enhanced. Common leaving groups comprise, but are not limited to, halogen, mesyl, —OTs or —OH.

“Substituted” refers to that one or more hydrogen atoms, preferably at most 5 hydrogen atoms, and more preferably 1 to 3 hydrogen atoms, in a group, are independently substituted by a corresponding number of substituents. It goes without saying that the substituents are only in their possible chemical positions, and those skilled in the art can determine (by experiment or theory) possible or impossible substitutions without excessive efforts. For example, amino or hydroxyl with free hydrogen may be unstable when combined with carbon atoms with unsaturated (such as olefinic) bonds.

“Substitution” or “substituted” in the specification, unless otherwise specified, refers to that the group may be substituted by one or more groups selected from the following substituents: alkyl, alkenyl, alkynyl, alkoxy, alkyl sulphanyl, alkyl amino, halogen, sulfhydryl, hydroxyl, nitro, cyano, cycloalkyl, heterocyclyl, aryl, heteroaryl, cycloalkoxy, heterocycloalkoxy, cycloalkthiol, heterocycloalkthiol, amino, haloalkyl, hydroxyalkyl, carboxyl, carboxylate group, ═O, —OR⁵, —C(O)R⁵, —C(O)OR⁵, —NHC(O)R⁵, —NHC(O)OR⁵, —NR⁶R⁷, —C(O)NR⁶R⁷, —CH₂NHC(O)OR⁵, —CH₂NR⁶R⁷ or —S(O)ᵣR⁵.

R⁶ is selected from hydrogen atom, alkyl, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰.

R⁶ and R⁷ are each independently selected from hydrogen atom, hydroxyl, halogen, alkyl, alkoxy, cycloalkyl, heterocyclyl, aryl or heteroaryl, wherein the alkyl, the alkoxy, the cycloalkyl, heterocyclyl, the aryl or the heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰.

Alternatively, R⁶ and R⁷ form a 4- to 8-membered heterocyclyl together with atoms to which R⁶ and R⁷ are connected, wherein the 4- to 8-membered heterocyclyl contains one or more N, O or S(O)ᵣ, and the 4- to 8-membered heterocyclyl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰.

R⁸, R⁹ and R¹⁰ are each independently selected from hydrogen atom, alkyl, amino, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate.

r is selected from 0, 1 or 2.

The compound of the present invention may contain an asymmetric center or a chiral center, thus existing in different stereoisomeric forms. It is expected that all stereoisomeric forms of the compound of the present invention comprise, but are not limited to, a diastereomer, an enantiomer, and an atropisomer and a geometric (conformational) isomer, and a mixture thereof, such as a racemic mixture, which are all within the scope of the present invention.

Unless otherwise specified, the structure described in the present invention further comprises all isomers of this structure (such as forms of a diastereomer, an enantiomer, and an atropisomer and a geometric (conformational) isomer: such as R and S configurations of asymmetric centers, (Z) and (E) double-bond isomers, and (Z) and (E) conformational isomers). Therefore, a single stereoisomer, and an enantiomeric mixture, a diastereomeric mixture and a geometric (conformational) isomer mixture of the compound of the present invention are all within the scope of the present invention.

“Pharmaceutically acceptable salt” refers to some salts of the above compound capable of maintaining original biological activity and suitable for medical use. The pharmaceutically acceptable salt of the compound represented by Formula (I) can be a metal salt or an amine salt formed with a suitable acid.

“Pharmaceutical composition” refers to a mixture containing one or more compounds described herein or their physiologically acceptable salts or prodrugs and other chemical components, and other components such as physiologically acceptable carriers and excipients.

The purpose of the pharmaceutical composition is to promote the administration to organisms, which is beneficial for the absorption of active ingredients, thus exerting biological activity.

### Method for Synthesizing the Compounds of the Present Invention Invention

In order to achieve the objects of the present invention, the following technical solutions are adopted by the present invention:

The present invention provides a preparation method for a compound represented by general formula (I) or a stereoisomer, a tautomer or a pharmaceutically acceptable salt thereof, wherein the method comprises the following steps of:
when W is N:

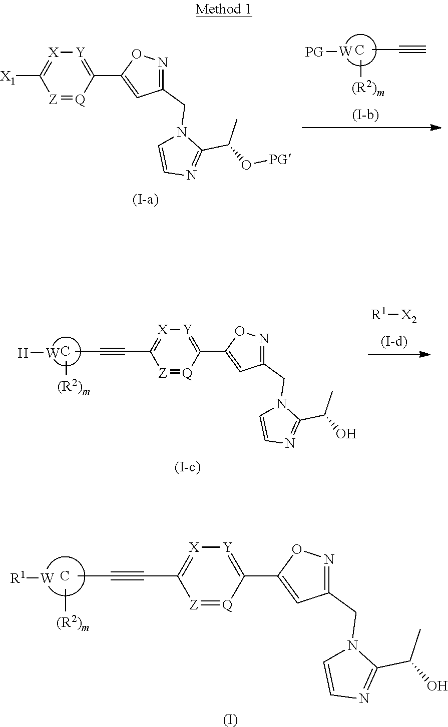

subjecting the compound represented by general formula (I-a) and the compound represented by general formula (I-b) to a coupling reaction under the action of a catalyst, and optionally further carrying out a deprotection reaction to obtain the compound represented by general formula (I-c), and optionally further subjecting the compound represented by general formula (I-c) and the compound represented by general formula (I-d) to substitution reaction to obtain the compound represented by general formula (I).

Wherein:
X₁ and X₂ are selected from halogen;
PG is an amino protecting group, and is preferably tert-butoxycarbonyl;
PG′ is a hydroxyl protecting group, and is preferably 2-tetrahydropyranyl; and
rings C, X, Y, Z, Q, R), R² and m are as defined in general formula (I).

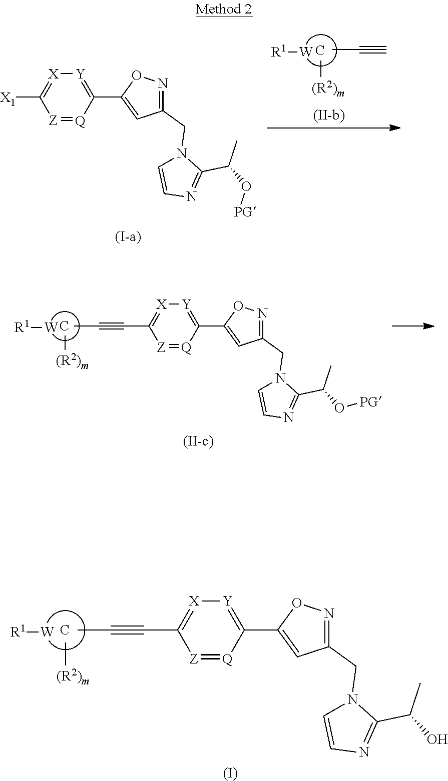

subjecting the compound represented by general formula (I-a) and the compound represented by general formula (II-b) to a coupling reaction under the action of a catalyst to obtain the compound represented by general formula (II-c), and optionally further carrying out a deprotection reaction on the compound represented by general formula (II-c) to obtain the compound represented by general formula (I).

Wherein:
X₁ is selected from halogen;
PG′ is a hydroxyl protecting group, and is preferably 2-tetrahydropyranyl; and
rings C, X, Y, Z, Q, R), R² and m are as defined in general formula (I).

### EMBODIMENT

The following examples are used to further describe the present invention, but these examples do not limit the scope of the present invention.

### Examples

The examples show the preparation of representative compounds represented by formula (I) and related structural identification data. It should be noted that the following examples are only used to illustrate the present invention, but not to limit the present invention. ¹H NMR spectrum was measured by Bruker instrument (400 MHz), and chemical shift was expressed in ppm, employing tetramethylsilane internal reference (0.00 ppm). ¹H NMR was expressed as follows: s=singlet, d=doublet, t=triplet, m=multiplet, br=broadened, dd=doublet of doublets, dt=doublet of triplets, and dq=double of quartets. If a coupling constant was provided, it was in the unit of Hz.

A mass spectrum was determined by LC/MS, and an ionization method may be ESI or APCI.

Yantai Huanghai HSGF254 or Qingdao GF254 silica gel plate was used as silica gel plates for thin layer chromatography. The silica gel plates used for thin layer chromatography (TLC) had a specification of 0.15 mm to 0.2 mm, and products separated and purified by TLC had a specification of 0.4 mm to 0.5 mm.

In general, Yantai Huanghai silica gel with 200-300 meshes was used as a carrier for column chromatography.

In the following examples, unless otherwise specified, all temperatures are in Celsius. Unless otherwise specified, various starting materials and reagents are commercially available or synthesized according to known methods, and the commercially available materials and reagents are directly used without further purification. Unless otherwise specified, the commercially available manufacturers include but are not limited to Aldrich Chemical Company, ABCR GmbH & Co. KG, Acros Organics, Guangzan Chemical Science and Technology Ltd., Jingyan Chemical Science and Technology Ltd., and the like.

DMSO-d₆ referred to deuterated dimethyl sulfoxide.

Argon atmosphere referred to that a reaction flask was connected with an argon balloon with a volume of about 1 L.

Unless otherwise specified in the examples, a solution in the reaction referred to an aqueous solution.

The compounds were purified by a silica gel column chromatography eluent system and thin layer chromatography, wherein the eluent system was selected from: A: petroleum ether and ethyl acetate system; B: dichloromethane and methanol system; C: dichloromethane and ethyl acetate system. The volume ratio of the solvent varied according to the polarity of the compound, and may also be adjusted by adding a small amount of acidic or basic reagents, such as acetic acid or triethylamine, or the like.

### Example 1

### (S)-1-(1-((5-(4-((7-azaspiro[3.5]nonan-2-yl)ethynyl)phenyl)isoxazol-3-yl)methyl)-1H-imidazol-2-yl)ethan-1-ol

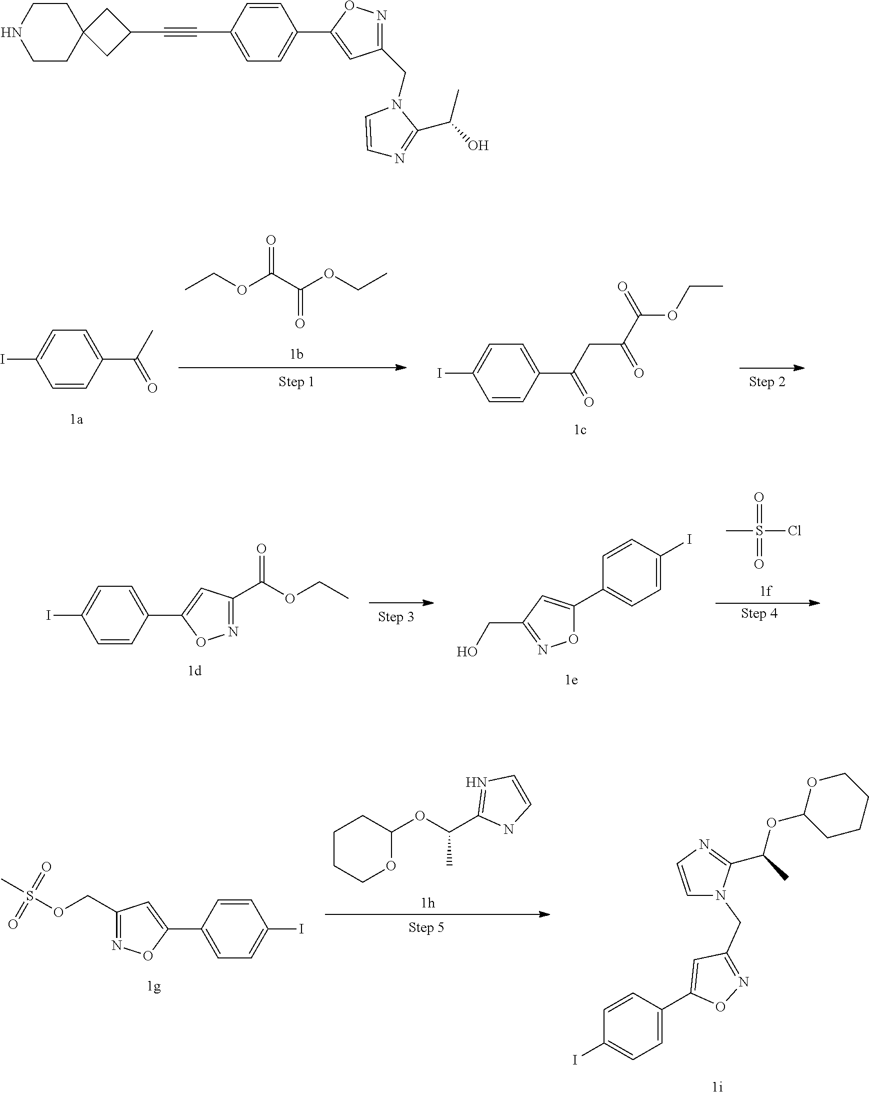

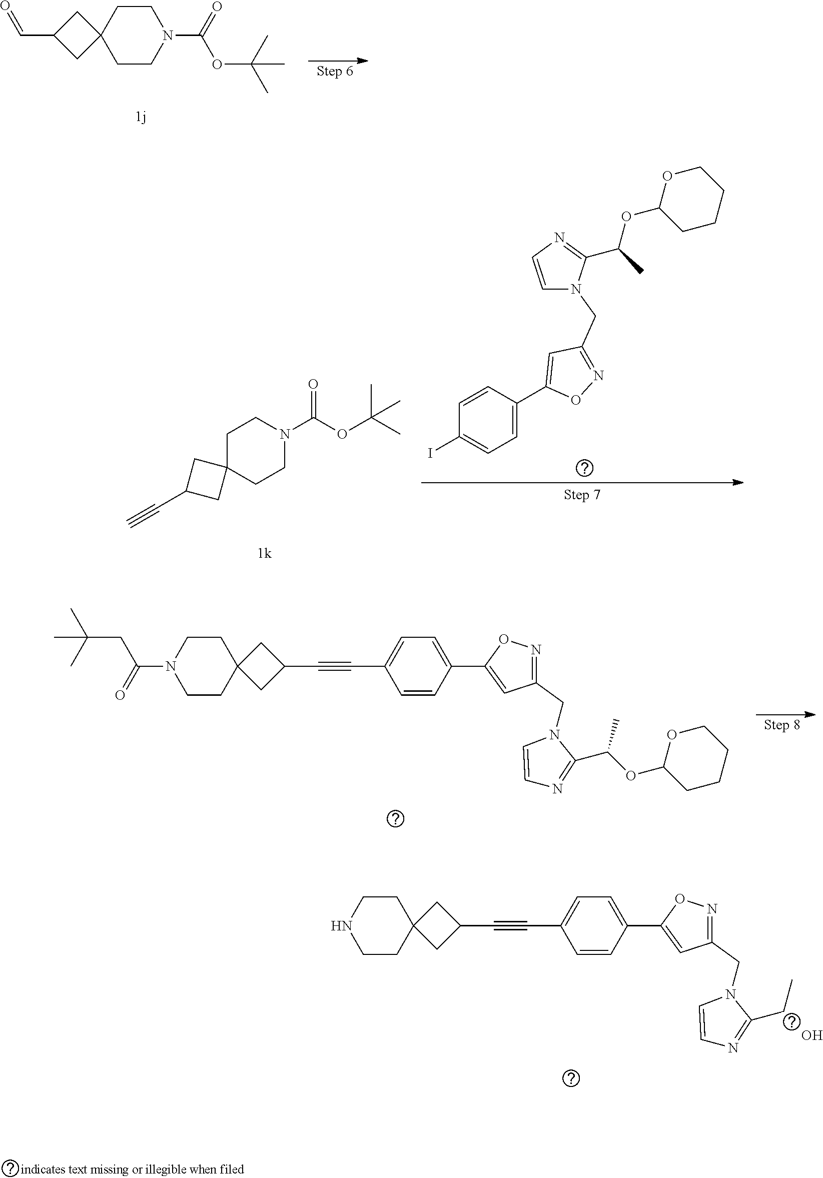

### Step 1

### Ethyl 4-(4-iodophenyl)-2,4-dioxobutanoate

Sodium hydride (1.63 g, 40.64 mmol, 60% dispersed in mineral oil) was added to toluene (20 mL), added with 1-(4-iodophenyl)ethan-1-one 1a (5 g, 20.32 mmol), heated to 50 °C, and dropwise added with a solution of diethyl oxalate 1b (4.45 g, 30.48 mmol) in toluene (20 mL), heated to 50 °C, and reacted for 2 hours. The reaction solution was cooled and then poured into ice water, adjusted to be acidic with 1 M hydrochloric acid, and extracted with ethyl acetate (100 mL×2). The combined organic phases were washed with saturated salt solution (100 mL×3), dried with anhydrous sodium sulfate, and concentrated under reduced pressure. The obtained residue was purified by silicagel column chromatography (eluent: system A) to obtain ethyl 4-(4-iodophenyl)-2,4-dioxobutanoate 1c (2.8 g) with a yield of 39.81%.

MS m/z (ESI): 347.0 [M+1].

### Step 2

### Ethyl 5-(4-iodophenyl)isoxazol-3-carboxylate

Ethyl 4-(4-iodophenyl)-2,4-dioxobutanoate 1c (2.8 g, 8.09 mmol) and hydroxylamine hydrochloride (1.69 g, 24.27 mmol) were added to ethanol (25 mL), heated and refluxed for 1 hour. After the reaction, the reaction solution was concentrated under reduced pressure, and added with ethyl acetate (100 mL) for dissolution. The organic phases were washed with saturated salt solution (100 mL), dried with anhydrous sodium sulfate and concentrated under reduced pressure.

The obtained residue was purified by silica gel column chromatography (eluent: A system) to obtain ethyl 5-(4-iodophenyl)isoxazol-3-carboxylate 1d (2.3 g) with a yield of 82.86%.

MS m/z (ESI): 343.8 [M+1].

### Step 3

### (5-(4-iodophenyl)isoxazol-3-yl)methanol

Ethyl 5-(4-iodophenyl)isoxazol-3-carboxylate 1d (1.20 g, 3.50 mmol) was added into methanol (25 mL), added with sodium borohydride (198.46 mg, 5.25 mmol) in batches, heated to 80 °C, and reacted for 4 hours. After the reaction was completed, the reaction solution was quenched by adding ice water, and extracted with ethyl acetate (50 mL×2). The organic phases were washed with saturated salt solution (50 mL), dried with anhydrous sodium sulfate, and concentrated under reduced pressure to obtain (5-(4-iodophenyl)isoxazol-3-yl)methanol 1e (0.36 g) with a yield of 34.19%.

MS m/z (ESI): 302.0 [M+1].

### Step 4

### (5-(4-iodophenyl)isoxazol-3-yl)methyl methanesulfonate

(5-(4-iodophenyl)isoxazol-3-yl)methanol 1e (0.36 g, 1.20 mmol) and triethylamine (241.99 mg, 2.39 mmol, 333.31 μL) were added to dichloromethane (5 mL), cooled to 0 °C, dropwise added with methanesulfonyl chloride if (205.45 mg, 1.79 mmol), heated to room temperature and reacted for 4 hours. After the reaction was completed, the reaction solution was quenched by adding water, and extracted with dichloromethane (50 mL×3). The organic phases were combined, and concentrated under reduced pressure. The obtained residue was purified by silica gel column chromatography (eluent: A system) to obtain (5-(4-iodophenyl)isoxazol-3-yl)methyl methanesulfonate 1g (0.45 g) with a yield of 99.26%.

MS m/z (ESI): 379.8 [M+1].

### Step 5

### 5-(4-iodophenyl)-3-((2-((1S)-1-((tetrahydro-2H-pyran-2-yl)oxy)ethyl)-1H-imidazol-1-yl)methyl)isoxazole

2-((1S)-1-((tetrahydro-2H-pyran-2-yl)oxy)ethyl)-1H-imidazole 1h (100 mg, 509.57 μmol) was added in N,N-dimethylformamide (2 mL), cooled to 0 °C, added with sodium hydride (50.96 mg, 764.35 μmol, 60% dispersed in mineral oil) in batches, heated to room temperature and reacted for 1 hour, added with (5-(4-iodophenyl)isoxazol-3-yl)methyl methanesulfonate 1g (193.21 mg, 509.57 μmol), and continuously reacted at room temperature for 4 hours. After the reaction was completed, the reaction solution was quenched by adding water, and extracted with ethyl acetate (10 mL×3). The organic phases were combined, and concentrated under reduced pressure. The obtained residue was purified by silica gel column chromatography (eluent: A system) to obtain 5-(4-iodophenyl)-3-((2-((1S)-1-((tetrahydro-2H-pyran-2-yl)oxy)ethyl)-1H-imidazol-1-yl)methyl)isoxazole 1i (0.12 g) with a yield of 49.13%.

MS m/z (ESI): 480.1 [M+1].

### Step 6

### Tert-butyl 2-ethynyl-7-azaspiro[3.5]nonane-7-carboxylate

In an ice bath, dimethyl (1-diazo-2-oxopropyl)phosphonate (1.36 g, 7.11 mmol) was added to potassium carbonate (1.64 g, 11.8 mmol) and tert-butyl 2-formyl-7-azaspiro[3.5]nonane-7-carboxylate 1j (1.5 g, 5.92 mmol, commercially available) in methanol (15 mL), and reacted for 24 hours at room temperature. After the reaction was completed, the reaction solution was quenched by adding water (50 mL), and extracted with ethyl acetate (50 mL×3). The combined organic phases were washed with saturated salt solution (100 mL), and concentrated under reduced pressure. The obtained residue was purified by silica gel column chromatography (eluent: A system) to obtain tert-butyl 2-ethynyl-7-azaspiro[3.5]nonane-7-carboxylate 1k (1.3 g) with a yield of 88.1%.

MS m/z (ESI): 194.1 [M+1-56].

### Step 7

### Tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-7-azaspiro[3.5]nonane-7-carboxylate

5-(4-iodophenyl)-3-((2-((1S)-1-((tetrahydro-2H-pyran-2-yl)oxy)ethyl)-1H-imidazol-1-yl)methyl)isoxazol 1i (1.3 g, 2.71 mmol), tert-butyl 2-ethynyl-7-azaspiro[3.5]nonane-7-carboxylate 1k (1.26 g, 3.53 mmol), bis(triphenylphosphine)palladium(II) chloride (190 mg, 271 μmol), cuprous iodide (51.8 mg, 271 μmol) and triethylamine (823 mg, 8.14 mmol, 1.13 mL) were added to N,N-dimethylformamide (13.87 mL), replaced with argon gas thrice, and reacted overnight at room temperature. After the reaction was completed, the reaction solution was quenched by adding water (30 mL), and extracted with ethyl acetate (100 mL×3). The combined organic phases were washed with saturated salt solution (100 mL), and concentrated under reduced pressure. The obtained residue was purified by silica gel column chromatography (eluent: B system) to obtain tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-7-azaspiro[3.5]nonane-7-carboxylate 1l (1.25 g) with a yield of 76.7%.

MS m/z (ESI): 601.1 [M+1].

### Step 8

### (S)-1-(1-((5-(4-((7-azaspiro[3.5]nonan-2-yl)ethynyl)phenyl)isoxazol-3-yl)methyl)-1H-imidazol-2-yl)ethan-1-ol

Tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-7-azaspiro[3.5]nonane-7-carboxylate 1l (1.2 g, 2.00 mmol) was dissolved in dichloromethane (30 mL), slowly added with trifluoroacetic acid (228 mg, 2.00 mmol), and reacted at room temperature for 4 hours. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(1-((5-(4-((7-azaspiro[3.5]nonan-2-yl)ethynyl)phenyl)isoxazol-3-yl)methyl)-1H-imidazol-2-yl)ethan-1-ol 1 (0.85 g) with a yield of 76.2%.

MS m/z (ESI): 417.1 [M+1].

### Example 2

### (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoic acid

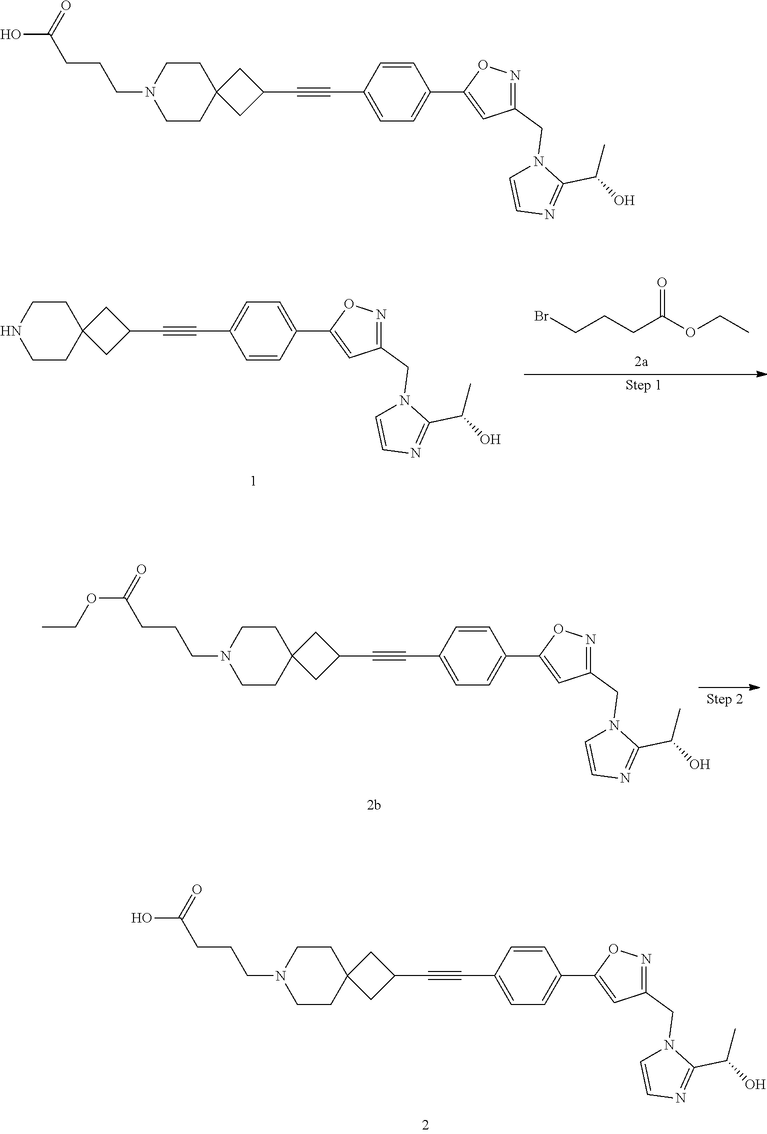

### Step 1

### Ethyl (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoate

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (100 mg, 240 μmol), ethyl 4-bromobutanoate 2a (70.3 mg, 360 μmol, commercially available), potassium carbonate (332 mg, 2.40 mmol) and sodium iodide (119 mg, 792 μmol) were added to N,N-dimethylformamide (3 mL) sequentially, heated to 70 °C and reacted for 4 hours. After the reaction was completed, the reaction solution was quenched by adding water (30 mL), and extracted with ethyl acetate (50 mL×3). The combined organic phases were washed with saturated salt solution (100 mL), and concentrated under reduced pressure. The obtained residue was purified by silica gel column chromatography (eluent: B system) to obtain ethyl (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoate 2b (65 mg) with a yield of 51.0%.

MS m/z (ESI): 531.1 [M+1].

### Step 2

### (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoic acid

Ethyl (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoate 2b (30 mg, 56.5 μmol) was dissolved in a mixed solvent of methanol (0.5 mL), tetrahydrofuran (0.5 mL) and water (0.5 mL), added with sodium hydroxide (2.26 mg, 56.5 μmol), heated to 70 °C and reacted for 4 hours. After the reaction was completed, the reaction solution was added with a small amount of diluted hydrochloric acid to adjust pH to about 4, and then concentrated to remove the organic solvent. The prepared solution was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoic acid 2 (1.8 mg) with a yield of 4.9%.

MS m/z (ESI): 503.1 [M+1].

### Example 3

### (S)-1-(1-((5-(4-((6-azaspiro[3.4]octan-2-yl)ethynyl)phenyl)isoxazol-3-yl)methyl)-1H-imidazol-2-yl)ethan-1-ol

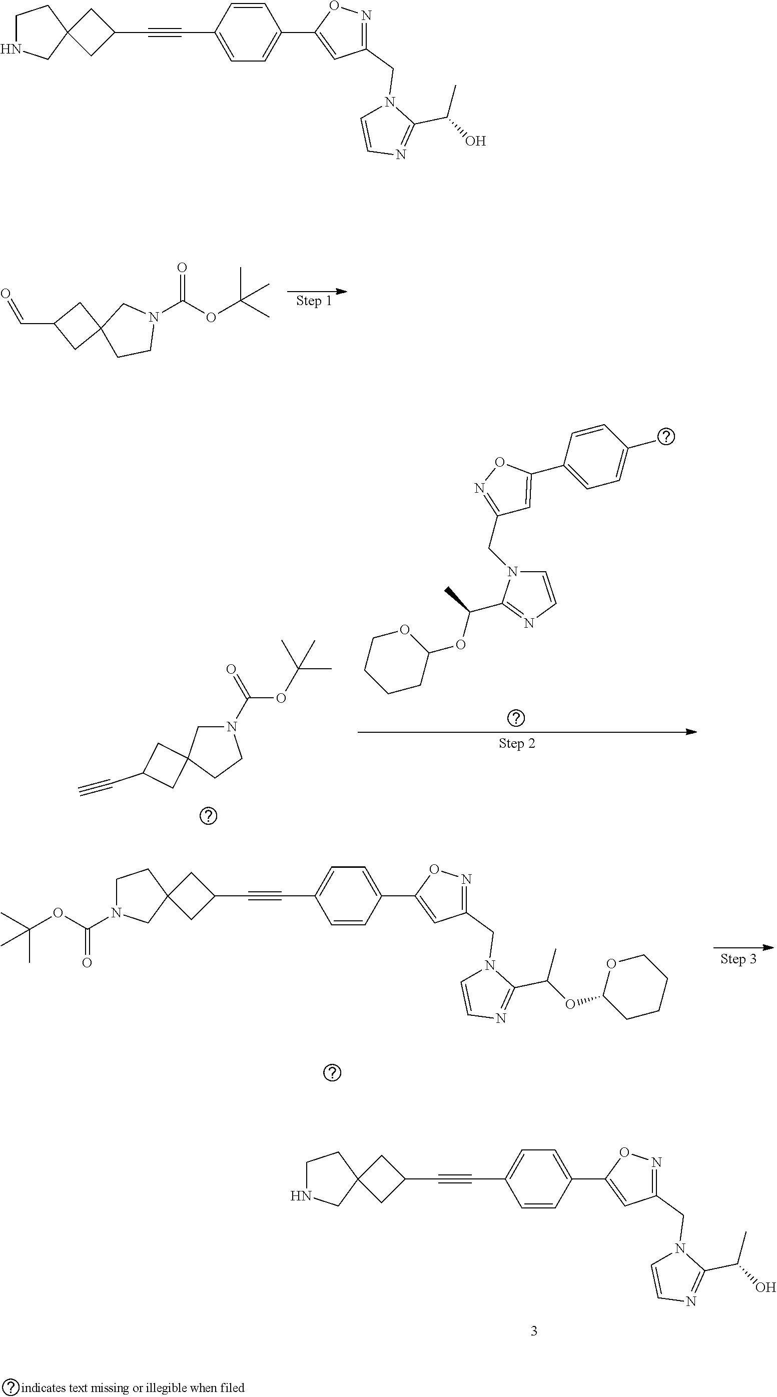

### Step 1

### Tert-butyl 2-ethynyl-6-azaspiro[3.4]octane-6-carboxylate

Under argon gas protection, in an ice bath, dimethyl (1-diazo-2-oxopropyl)phosphonate (241 mg, 1.25 mmol) was added to potassium carbonate (289 mg, 2.09 mmol) and tert-butyl 2-formyl-6-azaspiro[3.4]octane-6-carboxylate 3a (250 mg, 1.04 mmol) in methanol (4 mL), and reacted for 24 hours at room temperature. After the reaction was completed, the reaction solution was diluted by adding water (20 mL), and extracted with ethyl acetate (50 mL×3). The combined organic phases were washed with saturated salt solution (50 mL), and concentrated under reduced pressure. The obtained residue was purified by silica gel column chromatography (eluent: A system) to obtain tert-butyl 2-ethynyl-6-azaspiro[3.4]octane-6-carboxylate 3b (180 mg) with a yield of 73.2%.

MS m/z (ESI): 170.1 [M−55].

### Step 2

### Tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-6-azaspiro[3.4]octane-6-carboxylate

5-(4-iodophenyl)-3-((2-((1S)-1-((tetrahydro-2H-pyran-2-yl)oxy)ethyl)-1H-imidazol-1-yl)methyl)isoxazole 1i (0.4 g, 835 μmol), tert-butyl 2-ethynyl-6-azaspiro[3.4]octane-6-carboxylate 3b (365 mg, 1.08 mmol), Bis(triphenylphosphine)palladium(II) chloride (58.5 mg, 83.5 μmol), cuprous iodide (15.9 mg, 83.5 μmol) and triethylamine (253 mg, 2.50 mmol, 348 μL) were mixed with N,N-dimethylformamide (4.65 mL), replaced with argon gas thrice, and reacted overnight at room temperature. After the reaction was completed, the reaction solution was quenched by adding water (20 mL), and extracted with ethyl acetate (30 mL×3). The combined organic phases were washed with saturated salt solution (50 mL), and concentrated under reduced pressure. The obtained residue was purified by silica gel column chromatography (eluent: B system) to obtain ethyl tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-6-azaspiro[3.4]octane-6-carboxylate 3c (0.4 g) with a yield of 81.7%.

MS m/z (ESI): 587.3 [M+1].

### Step 3

### (S)-1-(1-((5-(4-((6-azaspiro[3.4]octan-2-yl)ethynyl)phenyl)isoxazol-3-yl)methyl)-1H-imidazol-2-yl)ethan-1-ol

Tert-butyl 2-((4-(3-((2-((1S)-1-((tetrahydro-2H-pyran-2-yl)oxy)ethyl)-1H-imidazol-1-yl)methyl)isoxazol-5-yl)phenyl)ethynyl)-6-azaspiro[3.4]octane-6-carboxylate 3c (0.3 g, 511 μmol) was dissolved in dichloromethane (5 mL), slowly dropwise added with trifluoroacetic acid (58 mg, 511 μmol), and reacted at room temperature for 4 hours. After the reaction was completed, the reaction solution was concentrated, subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(1-((5-(4-((6-azaspiro[3.4]octan-2-yl)ethynyl)phenyl)isoxazol-3-yl)methyl)-1H-imidazol-2-yl)ethan-1-ol 3 (120 mg) with a yield of 43.2%.

MS m/z (ESI): 403.1 [M+1].

### Example 4

### (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoic acid

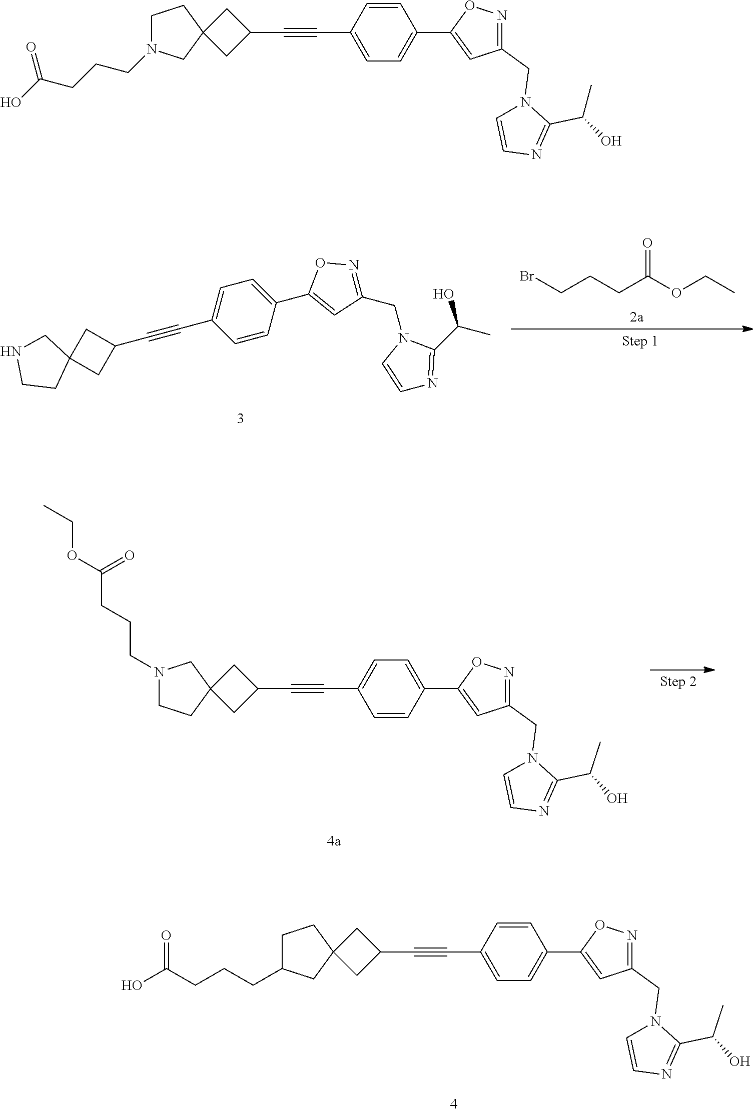

### Step 1

### Ethyl (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoate

(S)-1-(1-((5-(4-((6-azaspiro[3.4]octan-2-yl)ethynyl)phenyl)isoxazol-3-yl)methyl)-1H-imidazol-2-yl)ethan-1-ol 3 (100 mg, 248 μmol), ethyl 4-bromobutanoate 2a (72.7 mg, 373 μmol), potassium carbonate (343 mg, 2.48 mmol) and sodium iodide (74.5 mg, 497 μmol) were added to N,N-dimethylformamide (3 mL), heated to 70 °C and reacted for 4 hours. After the reaction was completed, the reaction solution was quenched by adding water (20 mL), and extracted with ethyl acetate (50 mL×3). The combined organic phases were washed with saturated salt solution (50 mL), and concentrated under reduced pressure. The obtained residue was purified by silica gel column chromatography (eluent: B system) to obtain ethyl (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoate 4a (60 mg) with a yield of 46.7%.

MS m/z (ESI): 517.1 [M+1].

### Step 2

### (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoic acid

Ethyl (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoate 4a (60 mg, 116 μmol) was dissolved in a mixed solvent of methanol (1 mL), tetrahydrofuran (1 mL) and water (1 mL), added with sodium hydroxide (23 mg, 580 μmol), heated to 70 °C and reacted for 4 hours. After the reaction was completed, the reaction solution was added with a small amount of diluted hydrochloric acid to adjust pH to about 4. The reaction solution was concentrated, most of the organic solvent was removed, and the prepared solution was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-(2-((4-(3-((2-(1-hydroxyethyl)-1H-imidazol-1-yl)methyl)isoxazol-5-yl)phenyl)ethynyl)-6-azaspiro[3.4]octan-6-yl)butanoic acid 4 (30 mg) with a yield of 40.7%.

MS m/z (ESI): 489.1 [M+1].

### Example 5

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoic acid

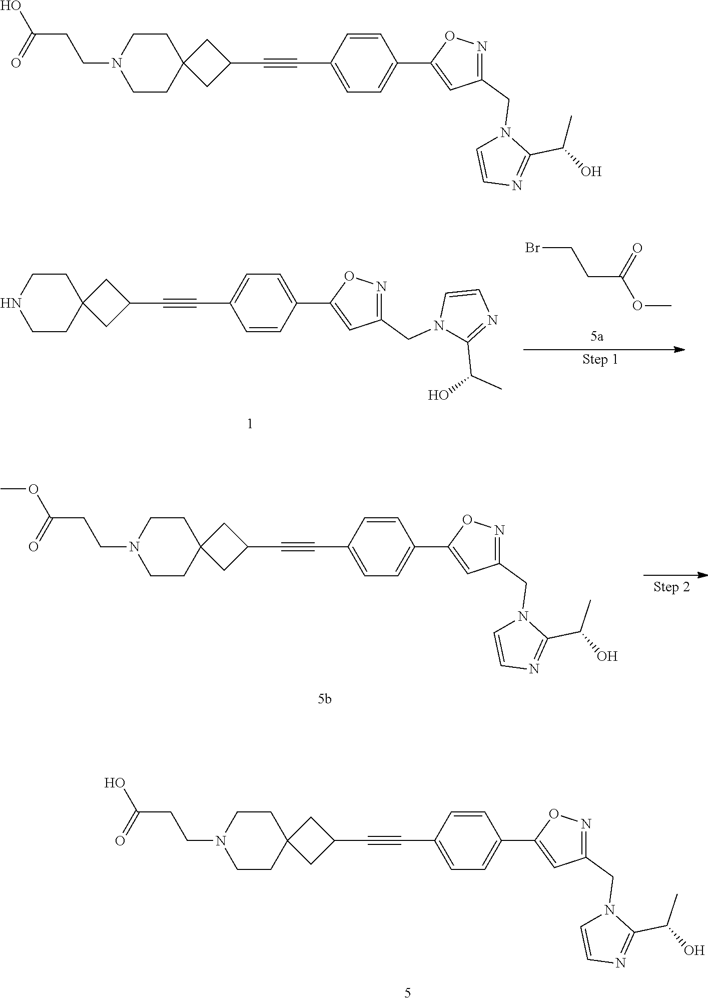

### Step 1

### Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoate

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 120 μmol), methyl 3-bromopropanoate 5a (30 mg, 180 μmol), potassium carbonate (166 mg, 1.20 mmol) and sodium iodide (59.4 mg, 396 μmol) were added to N,N-dimethylformamide (3 mL), heated to 70 °C and reacted for 4 hours. After the reaction was completed, the reaction solution was added with water (20 mL) for dilution, and extracted with ethyl acetate (30 mL×3). The combined organic phases washed with saturated salt solution (50 mL), and concentrated under reduced pressure. The obtained residue was purified by silica gel column chromatography (eluent: A system) to obtain methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoate 5b (40 mg), with a yield of 66.7%.

MS m/z (ESI): 503.1 [M+1].

### Step 2

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoic acid

Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoate 5b (80 mg, 159 μmol) was dissolved in a mixed solvent of methanol (1 mL), tetrahydrofuran (1 mL) and water (1 mL), added with sodium hydroxide (6.4 mg, 159 μmol), heated to 70 °C and reacted for 4 hours. After the reaction was completed, the reaction solution was added with a small amount of diluted hydrochloric acid to adjust pH to about 4, and concentrated to remove most organic solvent. The prepared solution was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoic acid 5 (20 mg) with a yield of 19.8%.

MS m/z (ESI): 489.1 [M+1].

### Example 6

### (S)-1-(1-((5-(4-((2-azaspiro[3.3]heptan-6-yl)ethynyl)phenyl)isoxazol-3-yl)methyl)-1H-imidazol-2-yl)ethan-1-ol

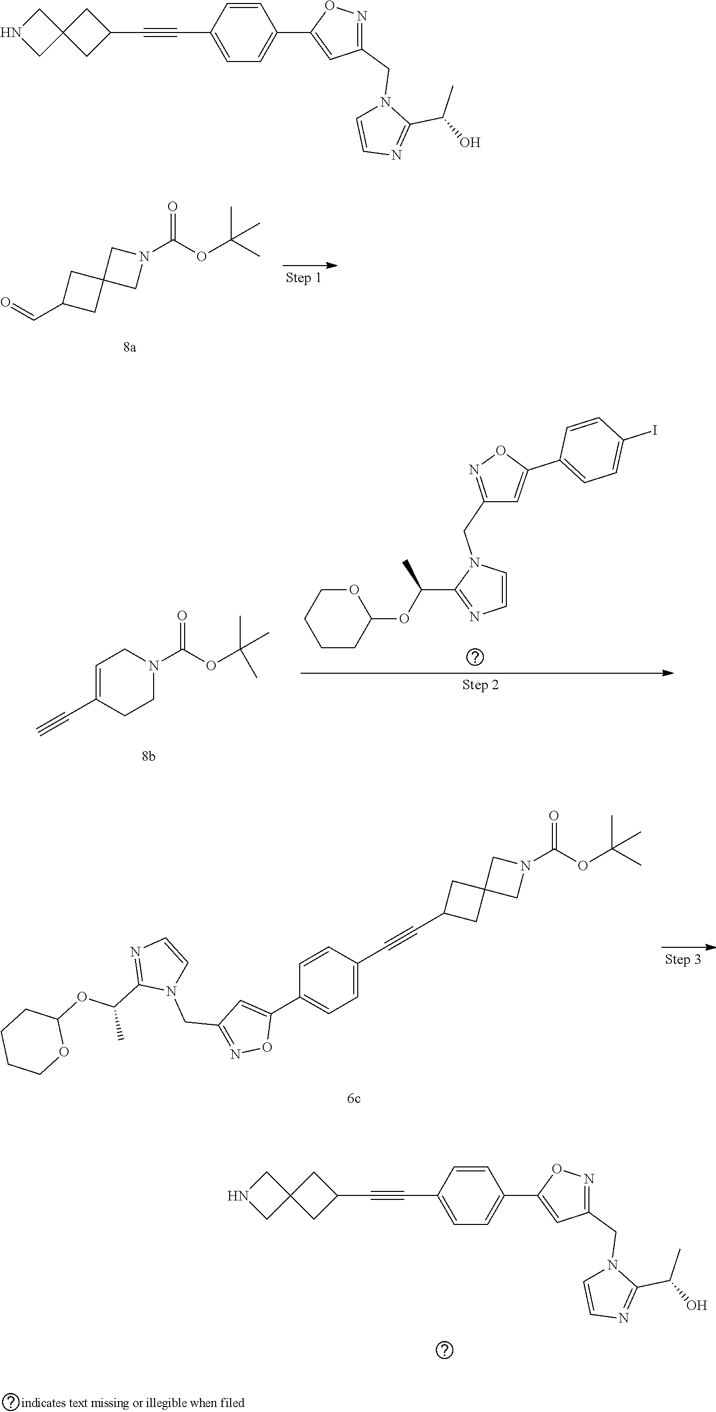

### Step 1

### Tert-butyl 6-ethynyl-2-azaspiro[3.3]heptane-2-carboxylate

Under argon gas protection, in an ice bath, dimethyl (1-diazo-2-oxopropyl)phosphonate (614 mg, 3.20 mmol) was slowly added to potassium carbonate (736 mg, 5.33 mmol) and tert-butyl 6-formyl-2-azaspiro[3.3]heptane-2-carboxylate 6a (0.6 g, 2.66 mmol) in methanol (8 mL), and reacted overnight at room temperature. After the reaction was completed, the reaction solution was added with water (20 mL) for dilution, and extracted with ethyl acetate (30 mL×3). The combined organic phases washed with saturated salt solution (50 mL), and concentrated under reduced pressure. The obtained residue was purified by silica gel column chromatography (eluent: A system) to obtain tert-butyl 6-ethynyl-2-azaspiro[3.3]heptane-2-carboxylate 6b (0.455 g), with a yield of 77.4%.

MS m/z (ESI): 166.1 [M−55].

### Step 2

### Tert-butyl 6-((4-(3-((2-((1S)-1-((tetrahydro-2H-pyran-2-yl)oxy)ethyl)-1H-imidazol-1-yl)methyl)isoxazol-5-yl)phenyl)ethynyl)-2-azaspiro[3.3]heptane-2-carboxylate

5-(4-iodophenyl)-3-((2-((1S)-1-((tetrahydro-2H-pyran-2-yl)oxy)ethyl)-1H-imidazol-1-yl)methyl)isoxazole 1i (0.6 g, 1.25 mmol), tert-butyl 6-ethynyl-2-azaspiro[3.3]heptane-2-carboxylate 6b (455 mg, 1.44 mmol), bis(triphenylphosphine)palladium(II) chloride (87.8 mg, 125 μmol), cuprous iodide (23.9 mg, 125 μmol) and triethylamine (380 mg, 3.76 mmol, 522 μL) were added to N,N-dimethylformamide (4 mL) sequentially, replaced with argon gas, and reacted overnight at room temperature. After the reaction was completed, the reaction solution was added with water (20 mL) for dilution, and extracted with ethyl acetate (100 mL×3). The combined organic phases were washed with saturated salt solution (100 mL), and concentrated under reduced pressure. The obtained residue was purified by silica gel column chromatography (eluent: B system) to obtain tert-butyl 6-((4-(3-((2-((1S)-1-((tetrahydro-2H-pyran-2-yl)oxy)ethyl)-1H-imidazol-1-yl)methyl)isoxazol-5-yl)phenyl)ethynyl)-2-azaspiro[3.3]heptane-2-carboxylate 6c (0.65 g), with a yield of 90.7%.

MS m/z (ESI): 573.1 [M+1].

### Step 3

### (S)-1-(1-((5-(4-((2-azaspiro[3.3]heptan-6-yl)ethynyl)phenyl)isoxazol-3-yl)methyl)-1H-imidazol-2-yl)ethan-1-ol

Tert-butyl 6-((4-(3-((2-((1S)-1-((tetrahydro-2H-pyran-2-yl)oxy)ethyl)-1H-imidazol-1-yl)methyl)isoxazol-5-yl)phenyl)ethynyl)-2-azaspiro[3.3]heptane-2-carboxylate 6c (20 mg, 34.92 μmol) was dissolved in dichloromethane (1 mL), slowly dropwise added with trifluoroacetic acid (46 mg, 405 μmol), and reacted at room temperature for 3 hours. After the reaction was completed, the reaction solution was concentrated. The residue was separated (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(1-((5-(4-((2-azaspiro[3.3]heptan-6-yl)ethynyl)phenyl)isoxazol-3-yl)methyl)-1H-imidazol-2-yl)ethan-1-ol 6 (5 mg) with a yield of 28.5%.

MS m/z (ESI): 389.1 [M+1].

### Example 7

### (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoic acid

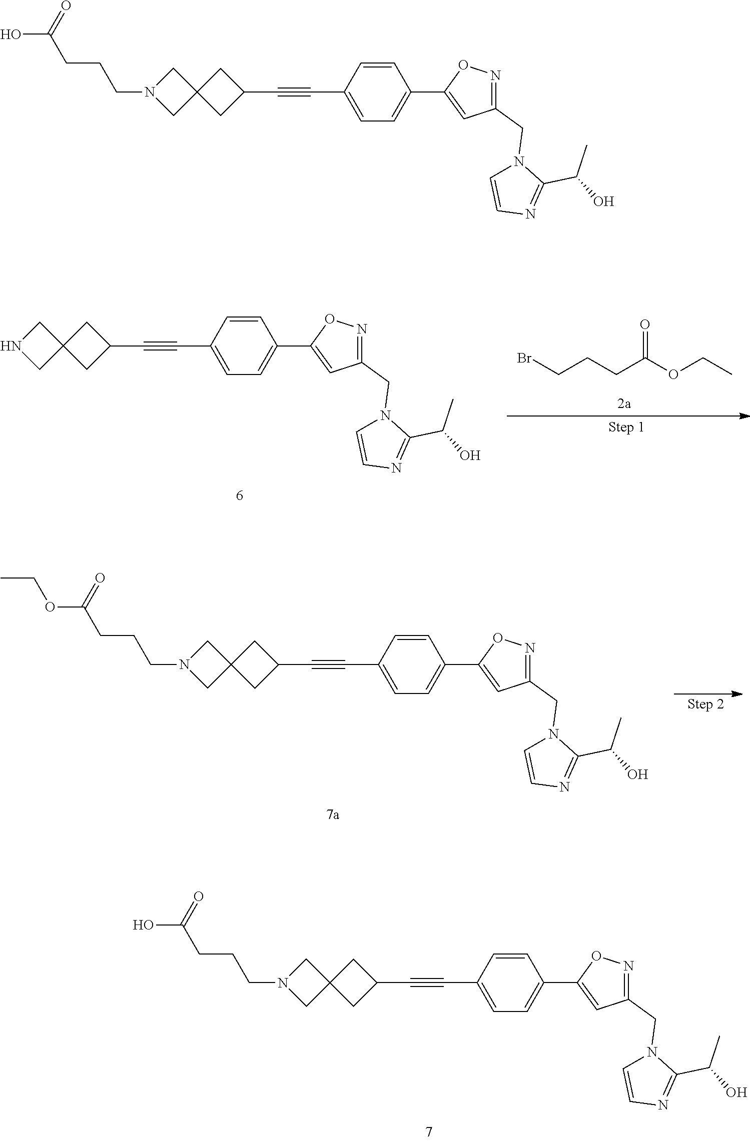

### Step 1

### Ethyl (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoate

(S)-1-(1-((5-(4-((2-azaspiro[3.3]heptan-6-yl)ethynyl)phenyl)isoxazol-3-yl)methyl)-1H-imidazol-2-yl)ethan-1-ol 6 (135 mg, 348 μmol), ethyl 4-bromobutanoate 2a (102 mg, 521 μmol, commercially available), potassium carbonate (480 mg, 3.48 mmol) and sodium iodide (172 mg, 1.15 mmol) were dissolved into N,N-dimethylformamide (3 mL), heated to 70 °C and reacted for 3 hours. After the reaction was completed, the reaction solution was added with water (20 mL) for dilution, and extracted with ethyl acetate (50 mL×3). The combined organic phases were washed with saturated salt solution (50 mL), and concentrated under reduced pressure. The obtained residue was purified by silica gel column chromatography (eluent: B system) to obtain ethyl (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoate 7a (50 mg), with a yield of 28.6%.

MS m/z (ESI): 503.1 [M+1].

### Step 2

### (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoic acid

Ethyl (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoate 7a (30 mg, 59.69 μmol) was dissolved in a mixed solvent of methanol (0.5 mL), tetrahydrofuran (0.5 mL) and water (0.5 mL), added with sodium hydroxide (2.4 mg, 59.7 μmol), heated to 70 °C and reacted for 3 hours. After the reaction was completed, the reaction solution was added with a proper amount of diluted hydrochloric acid to adjust pH to about 4, and concentrated to remove organic solvent. The prepared solution was subjected to preparative liquid separation (separation column AKZONOBEL Kromasil; 250×21.2 mm ID; 5 m, 20 mL/min; mobile phase A: 0.05% TFA+H₂O, mobile phase B: CH₃CN) to obtain (S)-4-(6-((4-(3-((2-(1-hydroxyethyl)-1H-imidazol-1-yl)methyl)isoxazol-5-yl)phenyl)ethynyl)-2-azaspiro[3.3]heptan-2-yl)butanoic acid 7 (10 mg) with a yield of 27.0%.

MS m/z (ESI): 475.1 [M+1].

### Example 8

### (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanamide

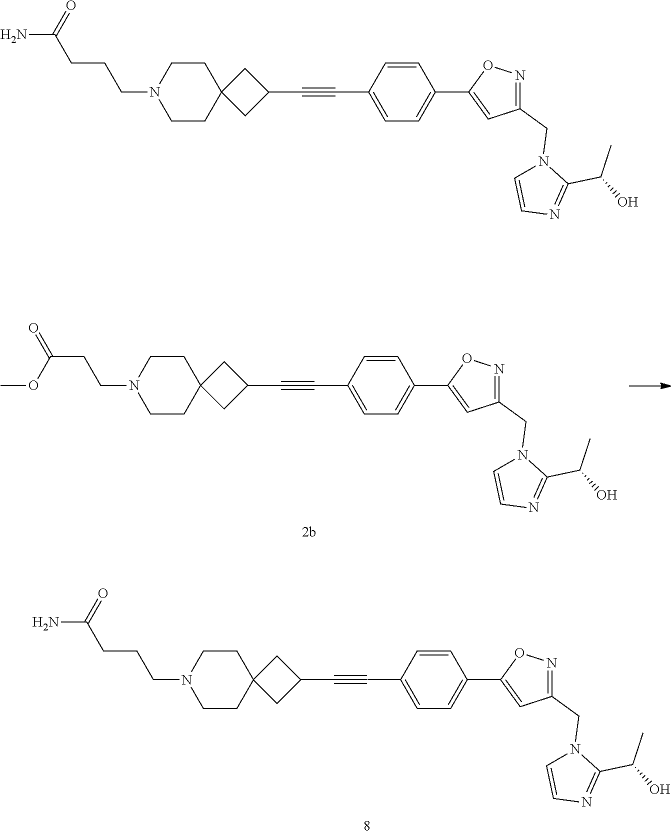 [SMILES: C[C@H](O)c1nccn1Cc1cc(-c2ccc(C#CC3CC4(CCN(CCCC(N)=O)CC4)C3)cc2)on1 ; COC(=O)CCN1CCC2(CC1)CC(C#Cc1ccc(-c3cc(Cn4ccnc4[C@H](C)O)no3)cc1)C2 ; C[C@H](O)c1nccn1Cc1cc(-c2ccc(C#CC3CC4(CCN(CCCC(N)=O)CC4)C3)cc2)on1]

Ethyl (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanoate 2b (30 mg, 56.53 μmol) was dissolved in ethanol (1.5 mL), dropwise added with ammonia water (1 mL), and reacted overnight at room temperature. After the reaction was completed, the reaction solution was concentrated, subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-(2-((4-(3-((2-(1-hydroxyethyl)-1H-imidazol-1-yl)methyl)isoxazol-5-yl)phenyl)ethynyl)-7-azaspiro[3.5]nonan-7-yl)butanamide 8 (5 mg) with a yield of 13.65%.

MS m/z (ESI): 502.1 [M+1].

### Example 9

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanamide

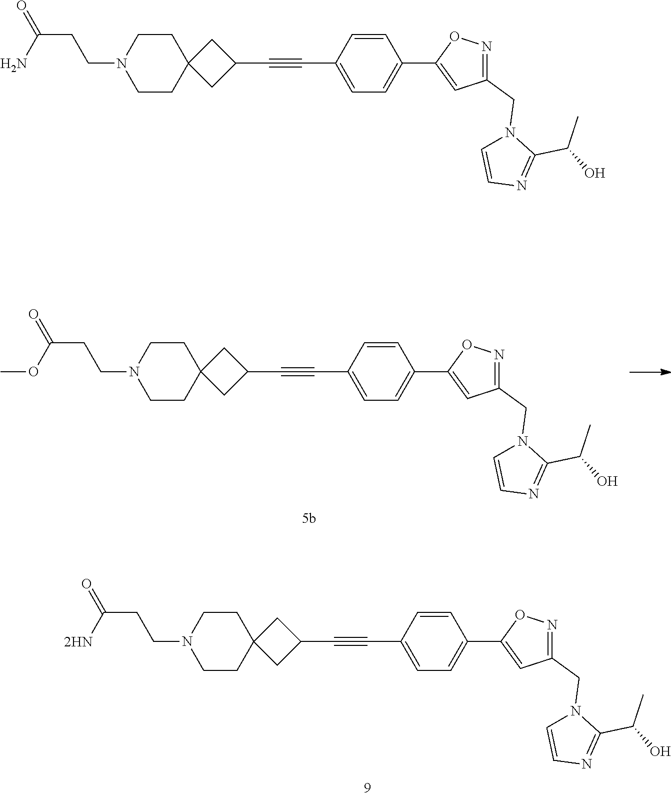

Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanoate 5b (60 mg, 119.38 μmol) was dissolved in ethanol (2 mL), dropwise added with ammonia water (1 mL), and reacted overnight at room temperature. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)propanamide 9 (10 mg) with a yield of 7.18%.

MS m/z (ESI): 488.1 [M+1].

### Example 10

### (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetic acid

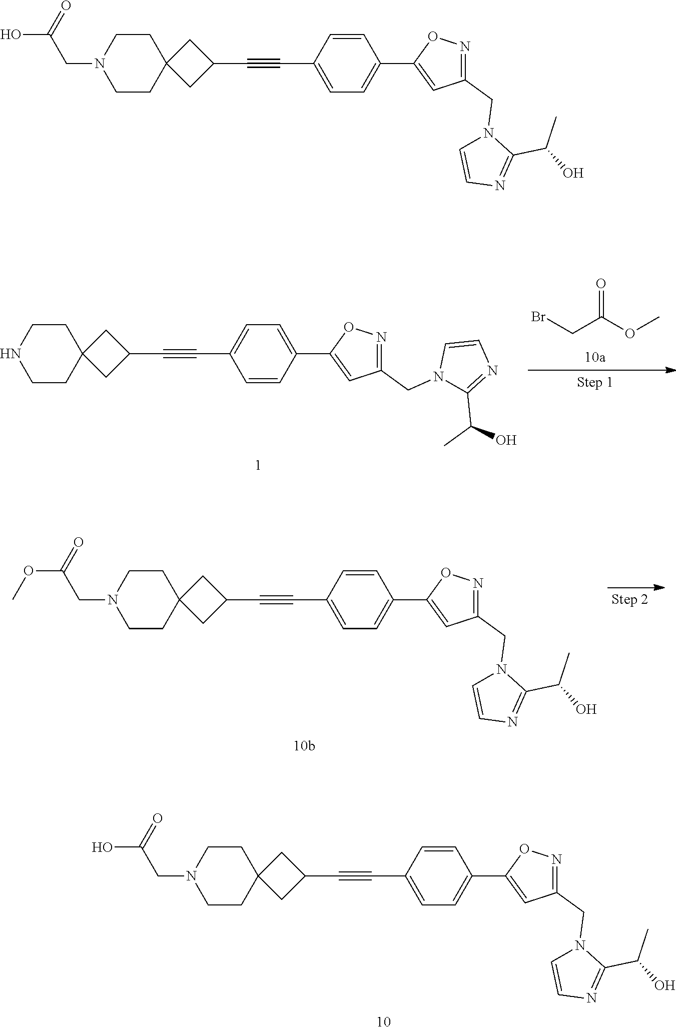

### Step 1

### Methyl (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetate

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (0.1 g, 240.09 μmol), methyl 2-bromoacetate 10a (55.1 mg, 360.13 μmol, commercially available), and potassium carbonate (332 mg, 2.40 mmol) were sequentially added to N,N-dimethylformamide (2 mL), and reacted at 80 °C for 4 hours. After the reaction was basically completed, the reaction solution was quenched by adding water (50 mL), extracted with ethyl acetate (50 mL×3) thrice, and then the organic phases were combined and concentrated under reduced pressure. The crude product was separated and purified by silicagel column chromatography (eluent: system B) to obtain methyl (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetate 10b (20 mg) with a yield of 17.05%.

MS m/z (ESI): 489.3 [M+1].

### Step 2

### (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetic acid

Methyl (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetate 10b (15 mg, 30.70 μmol) was dissolved in a mixed solvent of methanol (0.5 mL), tetrahydrofuran (0.5 mL) and water (0.5 mL), added with sodium hydroxide (6.14 mg, 153.51 μmol) and reacted at 80 °C for 8 hours. After the reaction was completed, the reaction solution was added with 1 M diluted hydrochloric acid (3 mL) and water (50 ml), and extracted with ethyl acetate (50 mL×3). The organic phases were combined and concentrated under reduced pressure, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetic acid 10 (2.90 mg) with a yield of 18.91%.

MS m/z (ESI): 475.0 [M+1].

### Example 11

### (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)ethan-1-ol

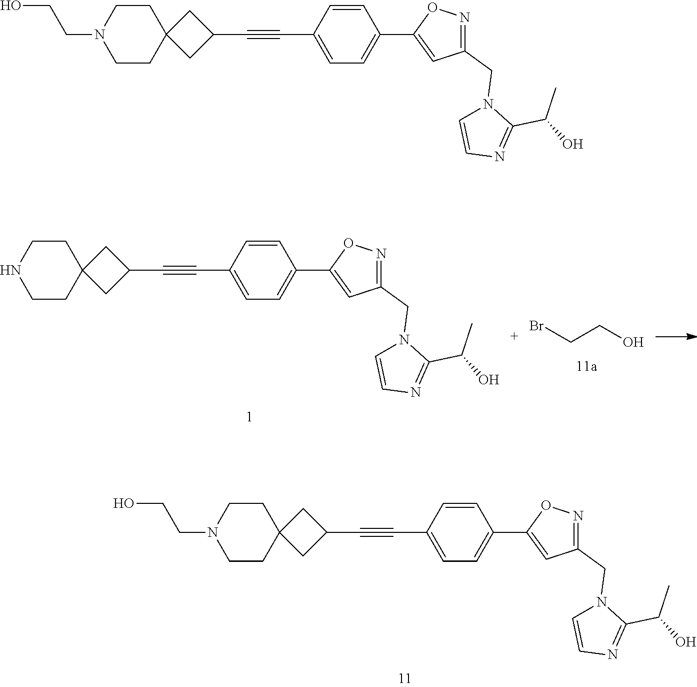

2-bromoethanol 11a (30 mg, 240.07 μmol, commercially available), (S)-1-(1-((5-(4-((7-azaspiro[3.5]nonan-2-yl)ethynyl)phenyl)isoxazol-3-yl)methyl)-1H-imidazol-2-yl)ethan-1-ol 1 (100 mg, 240.07 μmol), sodium iodide (216 mg, 1.44 mmol), and cesium carbonate (78.2 mg, 240.07 μmol) were respectively added to N,N-dimethylformamide (2 mL). After reacting at 80 °C for 6 hours, the reaction was basically completed. The reaction solution was quenched by adding water (50 mL), and extracted with ethyl acetate (50 mL×3) thrice. The combined organic phases were washed with saturated salt solution (50 mL), concentrated under reduced pressure, and purified by silica gel column chromatography (eluent: system B) to obtain (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)ethan-1-ol 11 (10 mg) with a yield of 6.89%.

MS m/z (ESI): 461.0 [M+1].

### Example 12

### (S)-2-hydroxy-1-(2-((4-(3-((2-(1-hydroxyethyl)-1H-imidazol-1-yl)methyl)isoxazol-5-yl)phenyl)ethynyl)-7-azaspiro[3.5]nonan-7-yl)ethan-1-one

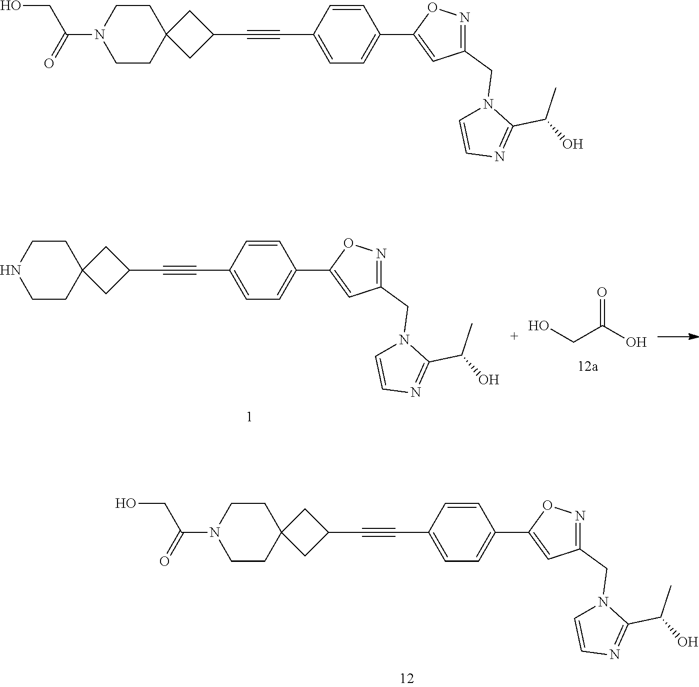

2-glycolic acid 12a (10 mg, 131.49 μmol, commercially available), (S)-1-(1-((5-(4-((7-azaspiro[3.5]nonan-2-yl)ethynyl)phenyl)isoxazol-3-yl)methyl)-1H-imidazol-2-yl)ethan-1-ol 1 (54.8 mg, 131.49 μmol), N,N-diisopropylethylamine (51.0 mg, 394.47 μmol), and O-(7-azabenzotriazole-1-yl)-N,N,N′,N′-tetramethyluronium hexafluorophosphate (100 mg, 262.98 μmol) were sequentially added to N,N-dimethylformamide (1 mL), and reacted at room temperature for 16 hours. After the reaction was basically completed, the reaction solution was quenched by adding water (50 mL), and extracted with ethyl acetate (50 mL×3) thrice. The combine organic phases were then washed with saturated salt solution (50 mL), and subjected to preparative liquid separation (separation column AKZONOBEL Kromasil; 250×21.2 mm ID; 5 m, 20 mL/min; mobile phase A: 0.05% TFA+H₂O, mobile phase B: CH₃CN) to obtain (S)-2-hydroxy-1-(2-((4-(3-((2-(1-hydroxyethyl)-1H-imidazol-1-yl)methyl)isoxazol-5-yl)phenyl)ethynyl)-7-azaspiro[3.5]nonan-7-yl)ethan-1-one 12 (10 mg) with a yield of 12.28%.

MS m/z (ESI): 475.1 [M+1].

### Example 13

### (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetamide

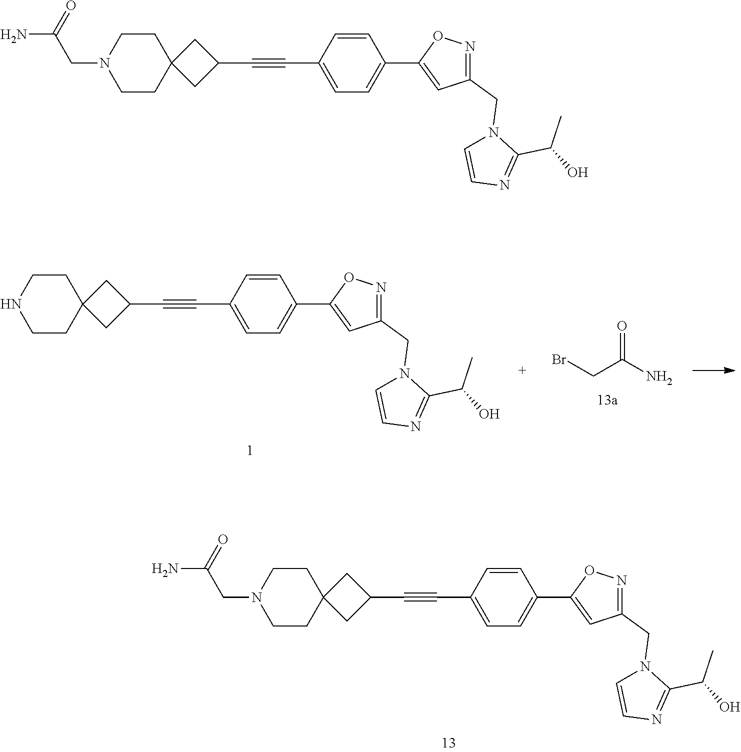

2-bromoacetamide 13a (10 mg, 72.48 μmol, commercially available), (S)-1-(1-((5-(4-((7-azaspiro[3.5]nonan-2-yl)ethynyl)phenyl)isoxazol-3-yl)methyl)-1H-imidazol-2-yl)ethan-1-ol 1 (30.2 mg, 72.48 μmol), potassium carbonate (30.1 mg, 217.45 μmol), and sodium iodide (10.9 mg, 72.48 μmol) were sequentially added to N,N-dimethylformamide (1 mL), and reacted at 80 °C for 4 hours. After the reaction was basically completed, the reaction solution was quenched by adding water (50 mL), and extracted with ethyl acetate (50 mL×3) thrice. The organic phases were combined and washed with saturated salt solution (50 mL), and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-(2-((4-(3-((2-(1-hydroxyethyl)-1H-imidazol-1-yl)methyl)isoxazol-5-yl)phenyl)ethynyl)-7-azaspiro[3.5]nonan-7-yl)acetamide 13 (5 mg) with a yield of 13.84%.

MS m/z (ESI): 474.0 [M+1].

### Example 14

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoic acid

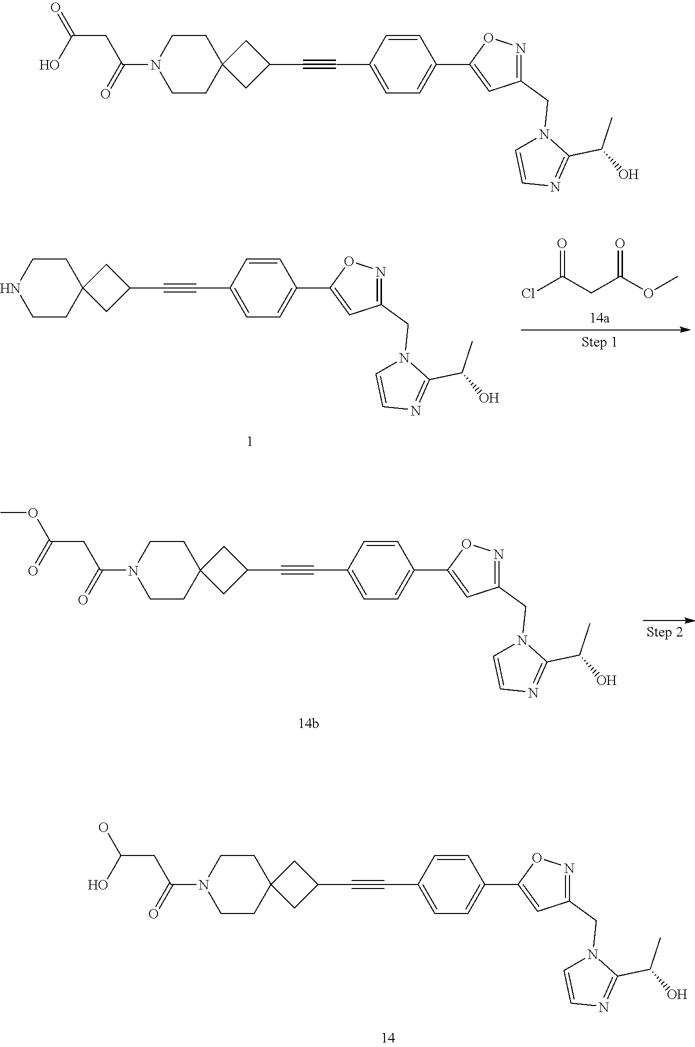

### Step 1

### Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoate

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (100 mg, 240.09 μmol), methyl 3-chloro-3-oxopropanoate 14a (32.8 mg, 240.09 μmol, commercially available), and triethylamine (72.9 mg, 720.26 μmol) were respectively added to N,N-dimethylformamide (3 mL), and reacted at room temperature for 8 hours. After the reaction was basically completed, the reaction solution was concentrated, and subjected to preparative liquid separation (separation column AKZONOBEL Kromasil; 250×21.2 mm I.D; 5 μm, 20 mL/min; mobile phase A: 0.0500 TFA+H₂O, mobile phase B: CH₃CN) to obtain methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoate 14b (40 mg) with a yield of 32.25%0.

MS m/z (ESI): 517.0 [M+1].

### Step 2

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoic acid

Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoate 14b (5 mg, 9.68 μmol) was dissolved in a mixed solvent of methanol (0.25 mL), tetrahydrofuran (0.25 mL) and water (0.25 mL), added with sodium hydroxide (1.16 mg, 29.04 μmol), and reacted at 80 °C for 4 hours. After the reaction was completed, the reaction solution was added with diluted hydrochloric acid (3 mL) and water (50 mL), and extracted with ethyl acetate (50 mL×3). The combine organic phases were then washed with saturated salt solution (50 mL), and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoic acid 14 (2.70 mg) with a yield of 52.73%.

MS m/z (ESI): 503.0 [M+1].

### Example 15

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanamide

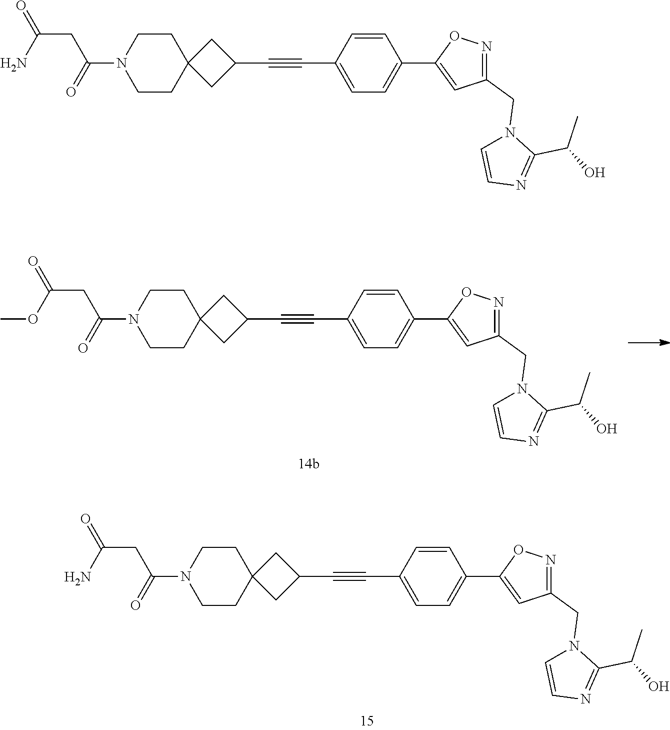 [SMILES: C[C@H](O)c1nccn1Cc1cc(-c2ccc(C#CC3CC4(CCN(C(=O)CC(N)=O)CC4)C3)cc2)on1 ; COC(=O)CC(=O)N1CCC2(CC1)CC(C#Cc1ccc(-c3cc(Cn4ccnc4[C@H](C)O)no3)cc1)C2 ; C[C@H](O)c1nccn1Cc1cc(-c2ccc(C#CC3CC4(CCN(C(=O)CC(N)=O)CC4)C3)cc2)on1]

Methyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanoate 14b (20 mg, 38.72 μmol) was dissolved in methanol (1 mL). Then dropwise added with ammonia water (0.8 mL), and reacted at room temperature for 36 hours. After the reaction was basically completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanamide 15 (2.66 mg) with a yield of 10.60%.

MS m/z (ESI): 502.0 [M+1].

### Example 16

### (S)-2-amino-1-(2-((4-(3-((2-(1-hydroxyethyl)-1H-imidazol-1-yl)methyl)isoxazol-5-yl)phenyl)eth ynyl)-7-azaspiro[3.5]nonan-7-yl)ethan-1-one

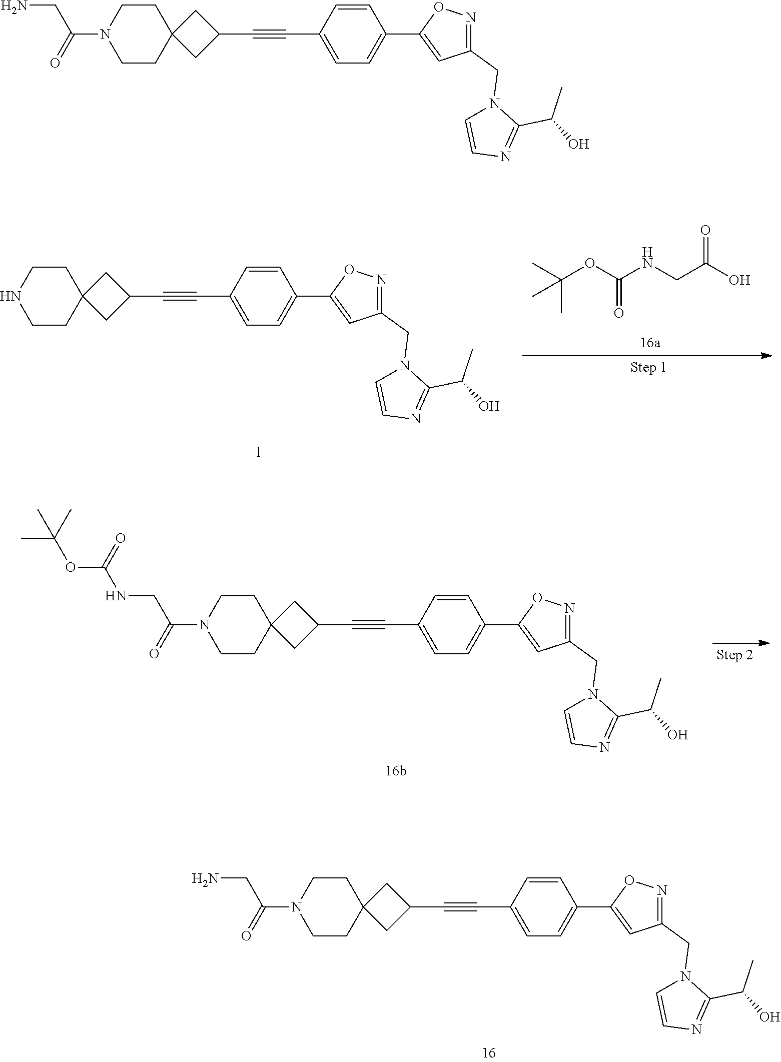 [SMILES: C[C@H](O)c1nccn1Cc1cc(-c2ccc(C#CC3CC4(CCN(C(=O)CN)CC4)C3)cc2)on1 ; C[C@H](O)c1nccn1Cc1cc(-c2ccc(C#CC3CC4(CCNCC4)C3)cc2)on1 ; CC(C)(C)OC(=O)NCC(=O)O ; C[C@H](O)c1nccn1Cc1cc(-c2ccc(C#CC3CC4(CCN(C(=O)CNC(=O)OC(C)(C)C)CC4)C3)cc2)on1 ; C[C@H](O)c1nccn1Cc1cc(-c2ccc(C#CC3CC4(CCN(C(=O)CN)CC4)C3)cc2)on1]

### Step 1

### Tert-butyl (S)-(2-(2-((4-(3-((2-(1-hydroxyethyl)-1H-imidazol-1-yl)methyl)isoxazol-5-yl)phenyl)ethynyl)-7-azaspiro[3.5]nonan-7-yl)-2-oxoethyl)carbamate

2-((tert-butoxycarbonyl)amino)acetic acid 16a (31.54 mg, 180.07 μmol, commercially available), N,N-diisopropylethylamine (62.06 mg, 480.18 μmol), and O-(7-azabenzotriazole-1-yl)-N,N,N′,N′-tetramethyluronium hexafluorophosphate (68.47 mg, 180.07 μmol) were dissolved in N,N-dimethylformamide (2 mL), stirred for 5 minutes, and then added with (S)-1-(1-((5-(4-((7-azaspiro[3.5]nonan-2-yl)ethynyl)phenyl)isoxazol-3-yl)methyl)-1H-imidazol-2-yl)ethan-1-ol 1 (50.00 mg, 120.04 μmol), and reacted overnight at room temperature. After the reaction was completed, the reaction solution was added with water (50 mL) and extracted with ethyl acetate (50 mL×3). The organic phases were combined, and washed with saturated sodium chloride solution (20 mL). The organic phases were dried width anhydrous sodium sulfate, filtered, and concentrated to obtain a crude product, and then the crude product was subjected to preparative liquid separation (separation column AKZONOBEL Kromasil; 250×21.2 mm ID; 5 m, 20 mL/min; mobile phase A: 0.05% TFA+H₂O, mobile phase B: CH₃CN) to obtain tert-butyl (S)-(2-(2-((4-(3-((2-(1-hydroxyethyl)-1H-imidazol-1-yl)methyl)isoxazol-5-yl)phenyl)ethynyl)-7-azaspiro[3.5]nonan-7-yl)-2-oxoethyl)carbamate 16b (20 mg) with a yield of 29.04%.

MS m/z (ESI): 574.3 [M+1].

### Step 2

### (S)-2-amino-1-(2-((4-(3-((2-(1-hydroxyethyl)-1H-imidazol-1-yl)methyl)isoxazol-5-yl)phenyl)eth ynyl)-7-azaspiro[3.5]nonan-7-yl)ethan-1-one

Tert-butyl (S)-(2-(2-((4-(3-((2-(1-hydroxyethyl)-1H-imidazol-1-yl)methyl)isoxazol-5-yl)phenyl)ethynyl)-7-azaspiro[3.5]nonan-7-yl)-2-oxoethyl)carbamate 16b (20 mg, 34.86 μmol) was dissolved into dichloromethane (2 mL), dropwise added with trifluoroacetic acid (0.3 ml, 2.63 mmol), and reacted at room temperature for 1.5 hours. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-amino-1-(2-((4-(3-((2-(1-hydroxyethyl)-1H-imidazol-1-yl)methyl)isoxazol-5-yl)phenyl)eth ynyl)-7-azaspiro[3.5]nonan-7-yl)ethan-1-one 16 (3 mg) with a yield of 3.91%.

MS m/z (ESI): 474.0 [M+1].

### Example 17

### (S)-2-((4-(3-((2-(1-hydroxyethyl)-1H-imidazol-1-yl)methyl)isoxazol-5-yl)phenyl)ethynyl)-7-azaspiro[3.5]nonane-7-carbaldehyde

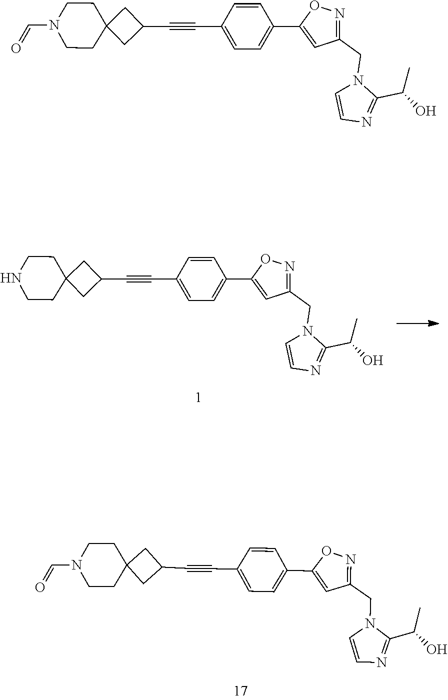 [SMILES: C[C@H](O)c1nccn1Cc1cc(-c2ccc(C#CC3CC4(CCN(C=O)CC4)C3)cc2)on1 ; C[C@H](O)c1nccn1Cc1cc(-c2ccc(C#CC3CC4(CCNCC4)C3)cc2)on1 ; C[C@H](O)c1nccn1Cc1cc(-c2ccc(C#CC3CC4(CCN(C=O)CC4)C3)cc2)on1]

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (30 mg, 56.55 μmol) was dissolved in formamide (2 mL), dropwise added with triethylamine (28.61 mg, 282.73 μmol), and reacted at room temperature for 6 hours. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-((4-(3-((2-(1-hydroxyethyl)-1H-imidazol-1-yl)methyl)isoxazol-5-yl)phenyl)ethynyl)-7-azaspiro[3.5]nonane-7-carbaldehyde 17 (3 mg) with a yield of 9.02%.

MS m/z (ESI): 445.2 [M+1].

### Example 18

### (S)-2-amino-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)propan-1-one

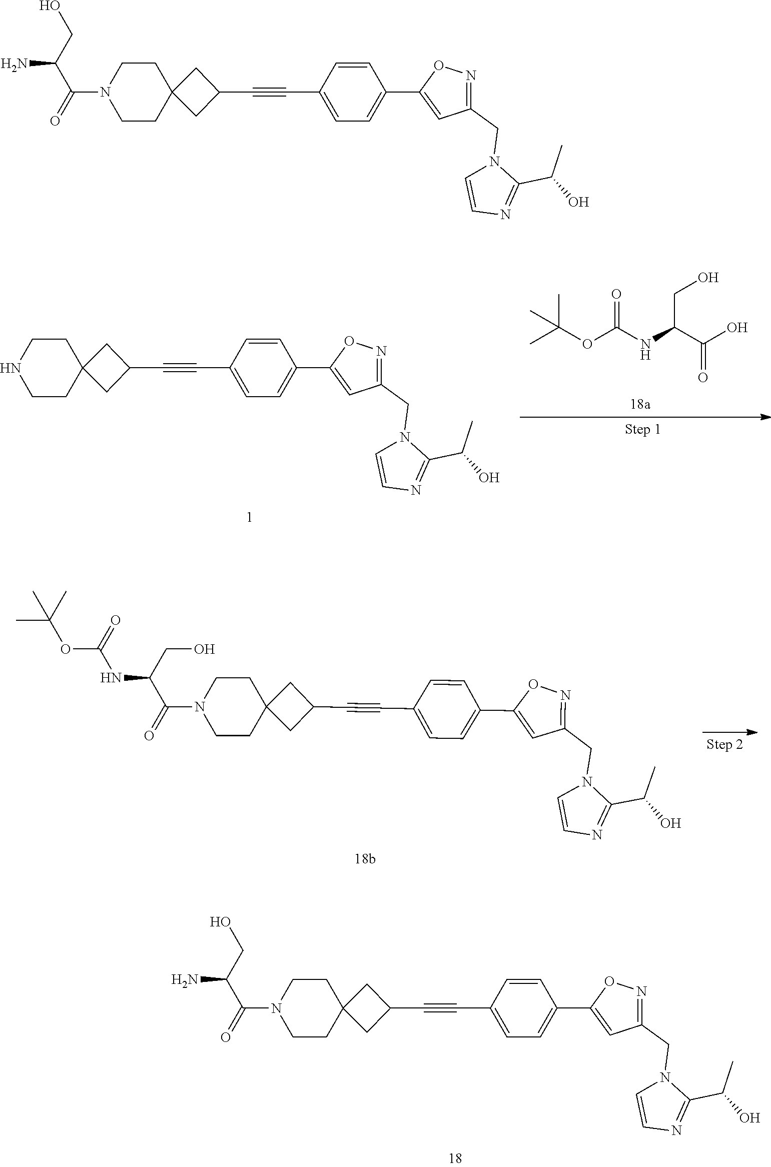 [SMILES: C[C@H](O)c1nccn1Cc1cc(-c2ccc(C#CC3CC4(CCN(C(=O)[C@@H](N)CO)CC4)C3)cc2)on1 ; C[C@H](O)c1nccn1Cc1cc(-c2ccc(C#CC3CC4(CCNCC4)C3)cc2)on1 ; CC(C)(C)OC(=O)N[C@@H](CO)C(=O)O ; C[C@H](O)c1nccn1Cc1cc(-c2ccc(C#CC3CC4(CCN(C(=O)[C@H](CO)NC(=O)OC(C)(C)C)CC4)C3)cc2)on1 ; C[C@H](O)c1nccn1Cc1cc(-c2ccc(C#CC3CC4(CCN(C(=O)[C@@H](N)CO)CC4)C3)cc2)on1]

### Step 1

### Tert-butyl ((S)-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)-1-oxopropan-2-yl)carbamate

(Tert-Butoxycarbonyl)-L-serine 18a (29.01 mg, 141.37 μmol, commercially available), N,N-diisopropylethylamine (60.90 mg, 471.22 μmol), and O-(7-aza-1H-benzotriazole-1-yl)-N,N,N′,N′-tetramethyluronium hexafluorophosphate (53.75 mg, 141.37 μmol) were dissolved in N,N-dimethylformamide (2 mL), stirred for five minutes, and then added with (S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 94.24 μmol), and reacted overnight at room temperature. After the reaction was completed, the reaction solution was quenched by adding water (50 mL), and extracted with ethyl acetate (50 mL×3) thrice. The organic phases were combined, washed with saturated sodium chloride solution (50 mL), dried with anhydrous sodium sulfate, filtered, and concentrated under reduced pressure, and the obtained residue was purified by silica gel column chromatography (eluent: B system) to obtain tert-butyl ((S)-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)-1-oxopropan-2-yl)carbamate 18b (20 mg) with a yield of 35.15%.

MS m/z (ESI): 604.3 [M+1].

### Step 2

### (S)-2-amino-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)propan-1-one

Tert-butyl ((S)-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)-1-oxopropan-2-yl)carbamate 18b (20 mg, 33.13 μmol) was dissolved in dichloromethane (3 mL), dropwise added with trifluoroacetic acid (0.5 g, 4.39 mmol), and reacted at room temperature for 3 hours. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-amino-3-hydroxy-1-(2-((4-(3-((2-((S)-1-hydroxyethyl)-1H-imidazol-1-yl)methyl)isoxazol-5-yl)phenyl)ethynyl)-7-azaspiro[3.5]nonan-7-yl)propan-1-one 18 (5 mg) with a yield of 24.44%.

MS m/z (ESI): 504.0 [M+1].

### Example 19

### (S)-2-((4-(3-((2-(1-hydroxyethyl)-1H-imidazol-1-yl)methyl)isoxazol-5-yl)phenyl)ethynyl)-7-azaspiro[3.5]nonane-7-carboxamide

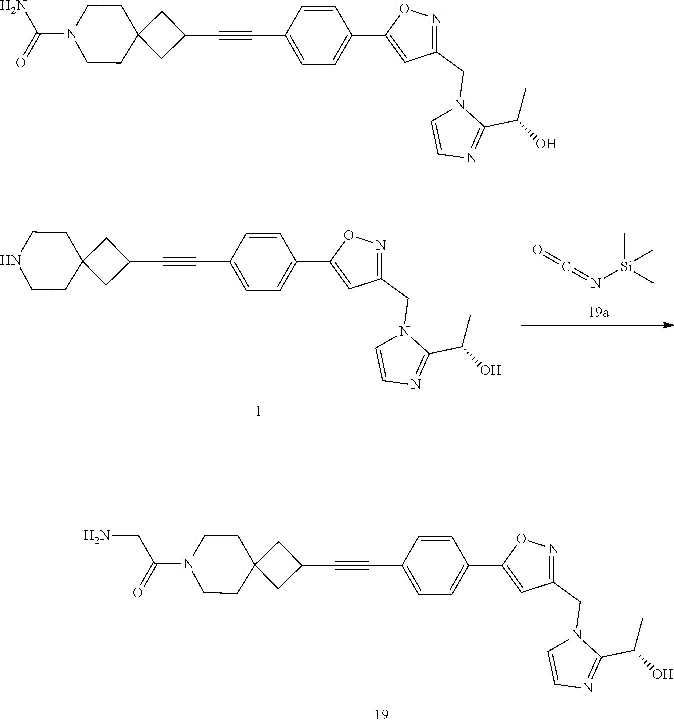

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 120.04 μmol), tetrahydrofuran (2 mL), and triethylamine (36.44 mg, 360.13 μmol) were sequentially added to a reaction flask, and finally added with trimethylsilyl isocyanate 19a (17.98 mg, 156.06 μmol, commercially available), and reacted overnight at room temperature. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-((4-(3-((2-(1-hydroxyethyl)-1H-imidazol-1-yl)methyl)isoxazol-5-yl)phenyl)ethynyl)-7-azaspiro[3.5]nonane-7-carboxamide 19 (10 mg) with a yield of 13.80%.

MS (ESI): 460.2 [M+1].

### Example 20

### (S)-2-(6-((4-(3-((2-(1-hydroxyethyl)-1H-imidazol-1-yl)methyl)isoxazol-5-yl)phenyl)ethynyl)-2-azaspiro[3.3]heptan-2-yl)acetamide

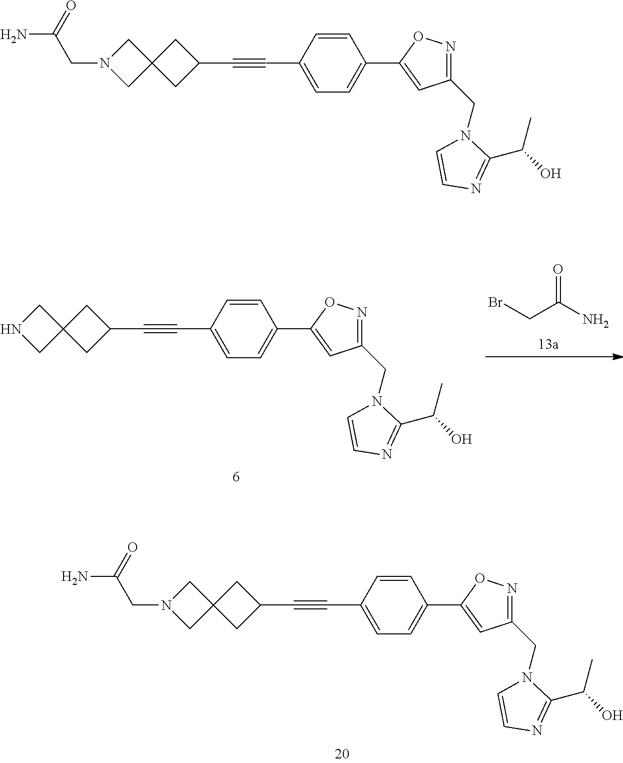 [SMILES: C[C@H](O)c1nccn1Cc1cc(-c2ccc(C#CC3CC4(C3)CN(CC(N)=O)C4)cc2)on1 ; C[C@H](O)c1nccn1Cc1cc(-c2ccc(C#CC3CC4(CNC4)C3)cc2)on1 ; NC(=O)CBr ; C[C@H](O)c1nccn1Cc1cc(-c2ccc(C#CC3CC4(C3)CN(CC(N)=O)C4)cc2)on1]

2-bromoacetamide 13a (30 mg, 217.45 μmol, commercially available), (S)-1-(1-((5-(4-((2-azaspiro[3.3]heptan-6-yl)ethynyl)phenyl)isoxazol-3-yl)methyl)-1H-imidazol-2-yl)ethan-1-ol 6 (84.47 mg, 217.45 μmol), potassium carbonate (90.16 mg, 652.35 μmol), and sodium iodide (32.62 mg, 217.45 μmol) were sequentially added to N,N-dimethylformamide (1.5 mL), and reacted at 80 °C for 4 hours. After the reaction was basically completed, the reaction solution was quenched by adding water (50 mL), and extracted with ethyl acetate (50 mL×3) thrice. The organic phases were combined and concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-(6-((4-(3-((2-(1-hydroxyethyl)-1H-imidazol-1-yl)methyl)isoxazol-5-yl)phenyl)ethynyl)-2-azaspiro[3.3]heptan-2-yl)acetamide 20 (3 mg) with a yield of 2.34%.

MS m/z (ESI): 446.1 [M+1].

### Example 21

### (S)-6-((4-(3-((2-(1-hydroxyethyl)-1H-imidazol-1-yl)methyl)isoxazol-5-yl)phenyl)ethynyl)-2-azaspiro[3.3]heptane-2-carboxamide

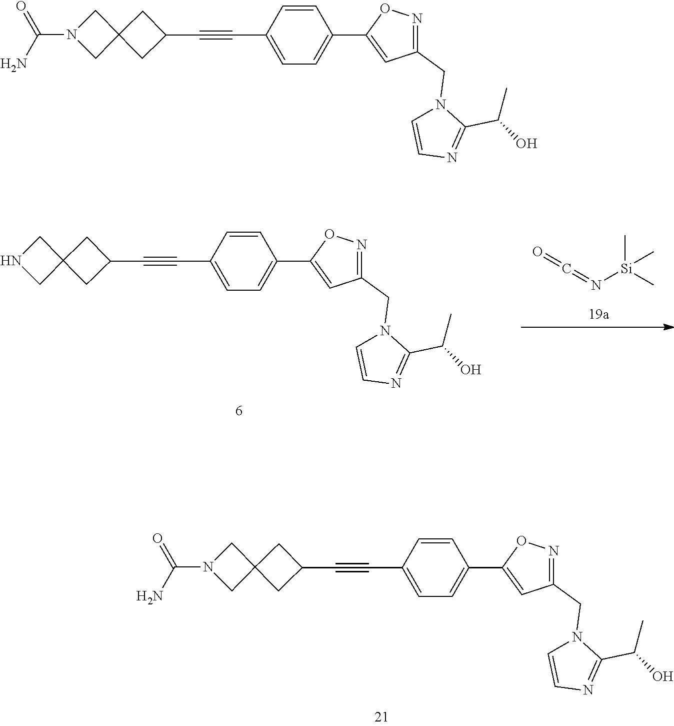

(S)-1-(1-((5-(4-((2-azaspiro[3.3]heptan-6-yl)ethynyl)phenyl)isoxazol-3-yl)methyl)-1H-imidazol-2-yl)ethan-1-ol 6 (35 mg, 90.10 μmol), tetrahydrofuran (2 mL), and triethylamine (27.35 mg, 270.30 μmol) were sequentially added to a reaction flask, and then added with trimethylsilyl isocyanate 19a (13.49 mg, 117.13 μmol, commercially available), and reacted overnight at room temperature. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (hydroxyethyl)-1H-imidazol-1-yl)methyl)isoxazol-5-yl)phenyl)ethynyl))-2-azaspiro[3.3]heptane-2-carboxamide 21 (9 mg) with a yield of 17.40%.

MS m/z (ESI): 432.0 [M+1].

### Example 22

### (S)-6-((4-(3-((2-(1-hydroxyethyl)-1H-imidazol-1-yl)methyl)isoxazol-5-yl)phenyl)ethynyl)-2-azaspiro[3.3]heptane-2-carbaldehyde

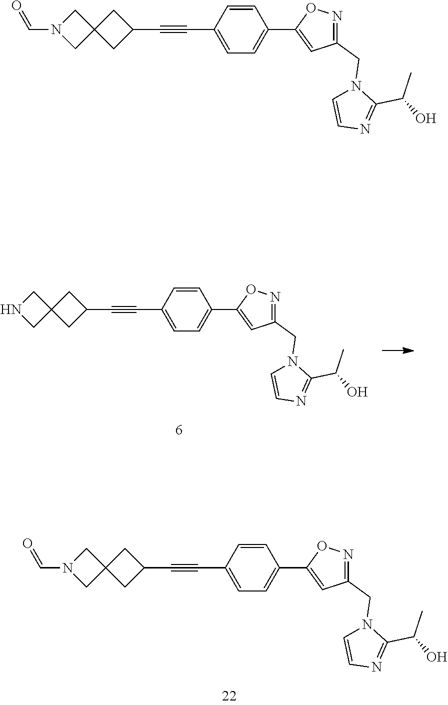 [SMILES: C[C@H](O)c1nccn1Cc1cc(-c2ccc(C#CC3CC4(C3)CN(C=O)C4)cc2)on1 ; C[C@H](O)c1nccn1Cc1cc(-c2ccc(C#CC3CC4(CNC4)C3)cc2)on1 ; C[C@H](O)c1nccn1Cc1cc(-c2ccc(C#CC3CC4(C3)CN(C=O)C4)cc2)on1]

(S)-1-(1-((5-(4-((2-azaspiro[3.3]heptan-6-yl)ethynyl)phenyl)isoxazol-3-yl)methyl)-1H-imidazol-2-yl)ethan-1-ol 6 (35 mg, 90.10 μmol), formamide (2 mL), and triethylamine (27.35 mg, 270.30 μmol) were sequentially added to a single-neck bottle, and reacted overnight at room temperature. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-6-((4-(3-((2-(1-hydroxyethyl)-1H-imidazol-1-yl)methyl)isoxazol-5-yl)phenyl)ethynyl)-2-azaspiro[3.3]heptane-2-carbaldehyde 22 (5 mg) with a yield of 9.94%.

MS m/z (ESI): 417.2 [M+1].

### Example 23

### (S)-2-((4-(3-((2-(1-hydroxyethyl)-1H-imidazol-1-yl)methyl)isoxazol-5-yl)phenyl)ethynyl)-6-azaspiro[3.4]octane-6-carbaldehyde

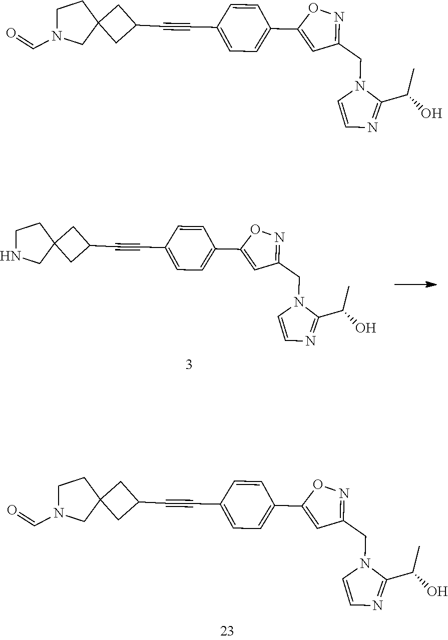 [SMILES: C[C@H](O)c1nccn1Cc1cc(-c2ccc(C#CC3CC4(CCN(C=O)C4)C3)cc2)on1 ; C[C@H](O)c1nccn1Cc1cc(-c2ccc(C#CC3CC4(CCNC4)C3)cc2)on1 ; C[C@H](O)c1nccn1Cc1cc(-c2ccc(C#CC3CC4(CCN(C=O)C4)C3)cc2)on1]

(S)-1-(1-((5-(4-((6-azaspiro[3.4]octan-2-yl)ethynyl)phenyl)isoxazol-3-yl)methyl)-1H-imidazol-2-yl)ethan-1-ol 3 (10 mg, 24.85 μmol) was dissolved in formamide (2 mL), added with triethylamine (25.14 mg, 248.45 μmol), and reacted overnight at room temperature. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-((4-(3-((2-(1-hydroxyethyl)-1H-imidazol-1-yl)methyl)isoxazol-5-yl)phenyl)ethynyl)-6-azaspiro[3.4]octane-6-carbaldehyde 23 (2.40 mg) with a yield of 15.08%.

MS m/z (ESI): 431.3 [M+1].

### Example 24

### (S)-2-((4-(3-((2-(1-hydroxyethyl)-1H-imidazol-1-yl)methyl)isoxazol-5-yl)phenyl)ethynyl)-6-azaspiro[3.4]octane-6-carboxamide

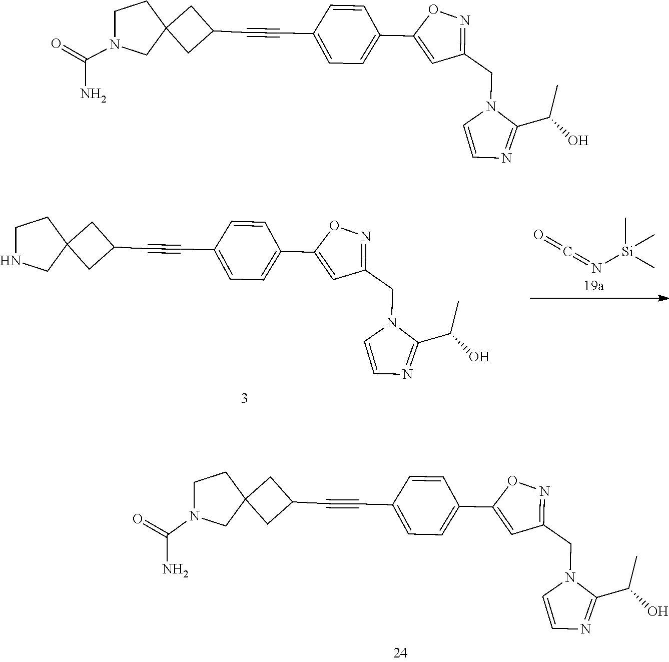

(S)-1-(1-((5-(4-((6-azaspiro[3.4]octan-2-yl)ethynyl)phenyl)isoxazol-3-yl)methyl)-1H-imidazol-2-yl)ethan-1-ol 3 (36.26 mg, 90.10 μmol) was dissolved in tetrahydrofuran (2 mL), added with triethylamine (27.35 mg, 270.30 μmol), trimethylsilyl isocyanate 19a (13.49 mg, 117.13 μmol), and reacted at room temperature for 4 hours. After the reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-((4-(3-((2-(1-hydroxyethyl)-1H-imidazol-1-yl)methyl)isoxazol-5-yl)phenyl)ethynyl)-6-azaspiro[3.4]octane-6-carboxamide 24 (3 mg) with a yield of 5.36%.

MS m/z (ESI): 446.0 [M+1].

### Example 25

### (S)-1-(1-((5-(4-((7-((1H-pyrazol-5-yl)methyl)-7-azaspiro[3.5]nonan-2-yl)ethynyl)phenyl)isoxazol-3-yl)methyl)-1H-imidazol-2-yl)ethan-1-ol

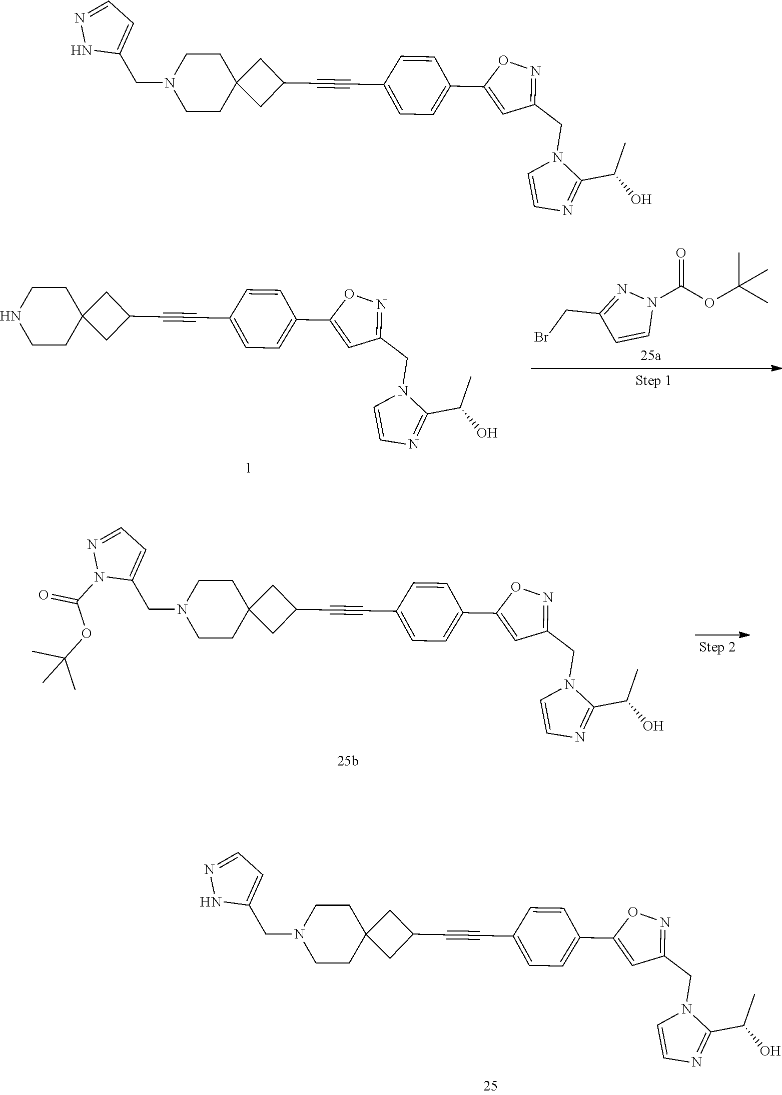 [SMILES: C[C@H](O)c1nccn1Cc1cc(-c2ccc(C#CC3CC4(CCN(Cc5ccn[nH]5)CC4)C3)cc2)on1 ; C[C@H](O)c1nccn1Cc1cc(-c2ccc(C#CC3CC4(CCNCC4)C3)cc2)on1 ; CC(C)(C)OC(=O)n1ccc(CBr)n1 ; C[C@H](O)c1nccn1Cc1cc(-c2ccc(C#CC3CC4(CCN(Cc5ccnn5C(=O)OC(C)(C)C)CC4)C3)cc2)on1 ; C[C@H](O)c1nccn1Cc1cc(-c2ccc(C#CC3CC4(CCN(Cc5ccn[nH]5)CC4)C3)cc2)on1]

### Step 1

### Tert-butyl (S)-5-((2-((4-(3-((2-(1-hydroxyethyl)-1H-imidazol-1-yl)methyl)isoxazol-5-yl)phenyl)ethynyl)-7-azaspiro[3.5]nonan-7-yl)methyl)-1H-pyrazole-1-carboxylate

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 120.04 μmol), tert-butyl 3-(bromomethyl)pyrazole-1-carboxylate 25a (31.35 mg, 120.04 μmol, commercially available), and potassium carbonate (19.91 mg, 144.05 μmol) were sequentially added to N,N-dimethylformamide (1.5 mL), and reacted at 75 °C for 8 hours. After the reaction was completed, the reaction solution was added with water (50 mL), and extracted with ethyl acetate (50 mL×3) trice. The organic phases were combined, dried with anhydrous sodium sulfate, and concentrated, and the obtained residue was purified by silica gel column chromatography (eluent: B system) to obtain a product tert-butyl (S)-5-((2-((4-(3-((2-(1-hydroxyethyl)-1H-imidazol-1-yl)methyl)isoxazol-5-yl)phenyl)ethynyl)-7-azaspiro[3.5]nonan-7-yl)methyl)-1H-pyrazole-1-carboxylate 25b (40 mg) with a yield of 55.84%.

MS m/z (ESI): 497.3 [M−100+1].

### Step 2

### (S)-1-(1-((5-(4-((7-((1H-pyrazol-5-yl)methyl)-7-azaspiro[3.5]nonan-2-yl)ethynyl)phenyl)isoxazol-3-yl)methyl)-1H-imidazol-2-yl)ethan-1-ol

Tert-butyl (S)-5-((2-((4-(3-((2-(1-hydroxyethyl)-1H-imidazol-1-yl)methyl)isoxazol-5-yl)phenyl)ethynyl)-7-azaspiro[3.5]nonan-7-yl)methyl)-1H-pyrazole-1-carboxylate 25b (40 mg, 67.03 μmol) was dissolved in dichloromethane (1 mL), slowly added with trifluoroacetic acid (67.03 μmol, 0.25 mL), and reacted at room temperature for 3 hours. The reaction was completed, the reaction solution was concentrated, and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain Tert-butyl (S)-5-((2-((4-(3-((2-(1-hydroxyethyl)-1H-imidazol-1-yl)methyl)isoxazol-5-yl)phenyl)ethynyl)-7-azaspiro[3.5]nonan-7-yl)methyl)-1H-pyrazole-1-carboxylate 25 (10 mg) with a yield of 23.21%.

MS m/z (ESI): 497.3 [M+1].

### Example 26

### (S)-2-hydroxy-1-(6-((4-(3-((2-(1-hydroxyethyl)-1H-imidazol-1-yl)methyl)isoxazol-5-yl)phenyl)ethynyl)-2-azaspiro[3.3]heptan-2-yl)ethan-1-one

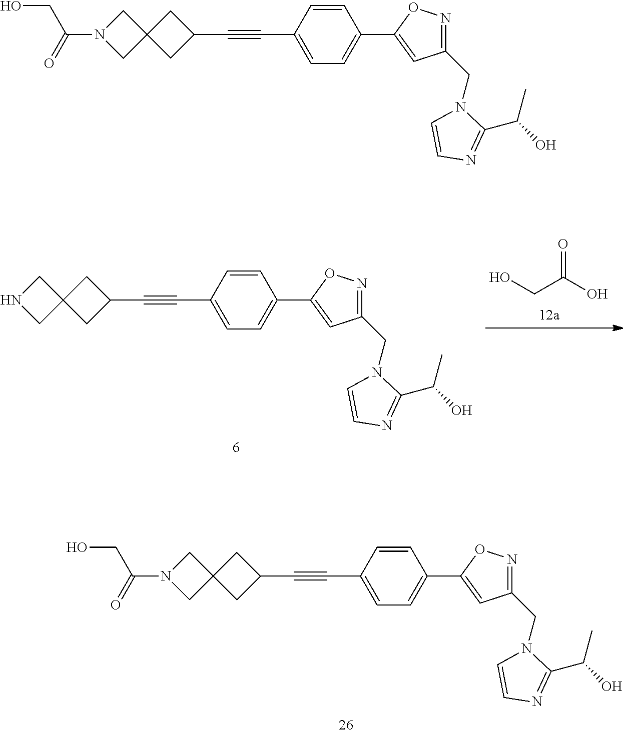 [SMILES: C[C@H](O)c1nccn1Cc1cc(-c2ccc(C#CC3CC4(C3)CN(C(=O)CO)C4)cc2)on1 ; C[C@H](O)c1nccn1Cc1cc(-c2ccc(C#CC3CC4(CNC4)C3)cc2)on1 ; O=C(O)CO ; C[C@H](O)c1nccn1Cc1cc(-c2ccc(C#CC3CC4(C3)CN(C(=O)CO)C4)cc2)on1]

1-(3-dimethylaminopropyl)-3-ethylcarbodiimide hydrochloride (25.91 mg, 135.15 μmol), 1-hydroxybenzotriazole (18.26 mg, 135.15 μmol), and 4-methylmorpholine (20.05 mg, 198.22 μmol), 2-glycolic acid 12a (10.28 mg, 135.15 μmol) were dissolved in dichloromethane (2 mL), reacted for five minutes, added with (S)-1-(1-((5-(4-((2-azaspiro[3.3]heptan-6-yl)ethynyl)phenyl)isoxazol-3-yl)methyl)-1H-imidazol-2-yl)ethan-1-ol 6 (35 mg, 90.10 μmol), and reacted overnight at room temperature. After the reaction was completed, the reaction solution was added with water (50 mL), and extracted with dichloromethane (50 mL×3) thrice. The organic phases were combined, dried with anhydrous sodium sulfate, and concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-hydroxy-1-(6-((4-(3-((2-(1-hydroxyethyl)-1H-imidazol-1-yl)methyl)isoxazol-5-yl)phenyl)ethynyl)-2-azaspiro[3.3]heptan-2-yl)ethan-1-one 26 (2.80 mg) with a yield of 5.27%.

MS m/z (ESI): 447.2 [M+1].

### Example 27

### (S)-1-(1-((5-(4-((7-((1H-imidazol-2-yl)methyl)-7-azaspiro[3.5]nonan-2-yl)ethynyl)phenyl)isoxazol-3-yl)methyl)-1H-imidazol-2-yl)ethan-1-ol

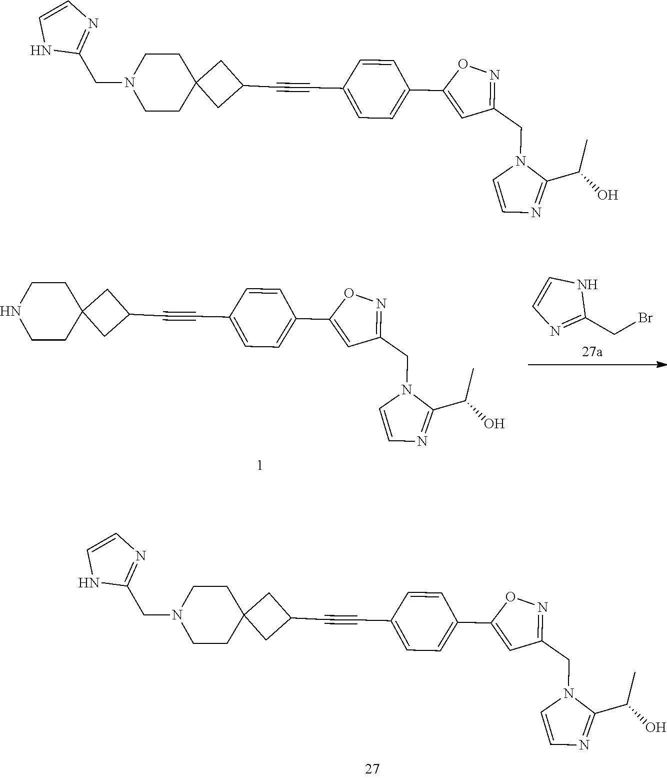 [SMILES: C[C@H](O)c1nccn1Cc1cc(-c2ccc(C#CC3CC4(CCN(Cc5ncc[nH]5)CC4)C3)cc2)on1 ; C[C@H](O)c1nccn1Cc1cc(-c2ccc(C#CC3CC4(CCNCC4)C3)cc2)on1 ; BrCc1ncc[nH]1 ; C[C@H](O)c1nccn1Cc1cc(-c2ccc(C#CC3CC4(CCN(Cc5ncc[nH]5)CC4)C3)cc2)on1]

(S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 120.04 μmol), 2-bromomethyl-1H-imidazole 27a (26.14 mg, 108.04 μmol), sodium iodide (1.99 mg, 12.00 μmol), and potassium carbonate (36.50 mg, 264.10 μmol) were sequentially added to N,N-dimethylformamide (1.5 mL), heated to 65 °C and reacted for 8 hours. After the reaction was basically completed, the reaction solution was filtered, and the filtrate was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(1-((5-(4-((7-((1H-imidazol-2-yl)methyl)-7-azaspiro[3.5]nonan-2-yl)ethynyl)phenyl)isoxazol-3-yl)methyl)-1H-imidazol-2-yl)ethan-1-ol 27 (10 mg) with a yield of 12.96%.

MS (ESI): 497.1 [M+1].

### Example 28

### (S)-2-hydroxy-1-(2-((4-(3-((2-(1-hydroxyethyl)-1H-imidazol-1-yl)methyl)isoxazol-5-yl)phenyl)ethynyl)-6-azaspiro[3.4]octan-6-yl)ethan-1-one

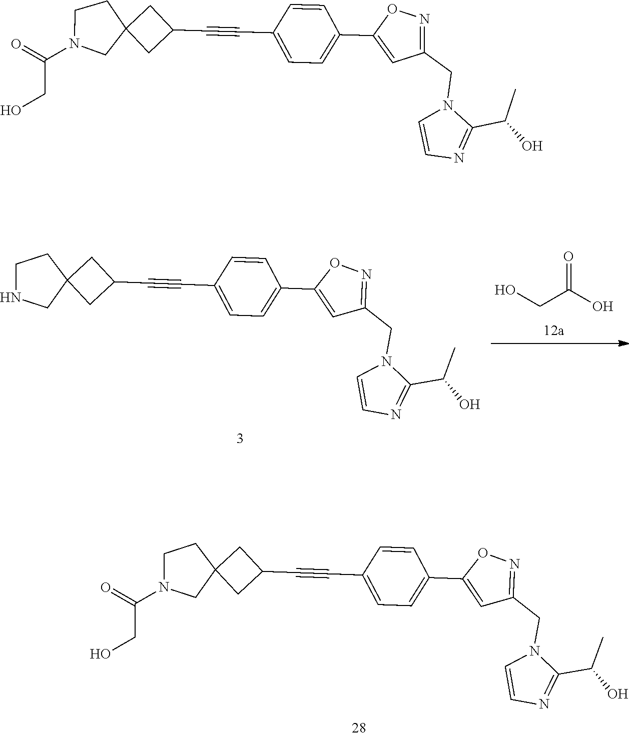 [SMILES: C[C@H](O)c1nccn1Cc1cc(-c2ccc(C#CC3CC4(CCN(C(=O)CO)C4)C3)cc2)on1 ; C[C@H](O)c1nccn1Cc1cc(-c2ccc(C#CC3CC4(CCNC4)C3)cc2)on1 ; O=C(O)CO ; C[C@H](O)c1nccn1Cc1cc(-c2ccc(C#CC3CC4(CCN(C(=O)CO)C4)C3)cc2)on1]

2-glycolic acid 12a (14.17 mg, 186.34 μmol), 1-(3-dimethylaminopropyl)-3-ethylcarbodiimide hydrochloride (35.72 mg, 186.34 μmol), 1-hydroxybenzotriazole (25.18 mg, 186.34 μmol), and 4-methylmorpholine (27.64 mg, 273.30 μmol) were added to dichloromethane solution (2 mL), reacted for five minutes, added with (S)-1-(1-((5-(4-((6-azaspiro[3.4]octan-2-yl)ethynyl)phenyl)isoxazol-3-yl)methyl)-1H-imidazol-2-yl)ethan-1-ol 3 (50 mg, 124.23 μmol), and reacted at room temperature for 16 hours. After the reaction was completed, the reaction solution was added with water (50 mL), and extracted with dichloromethane (50 mL×3) thrice. The organic phases were combined, concentrated and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-hydroxy-1-(2-((4-(3-((2-(1-hydroxyethyl)-1H-imidazol-1-yl)methyl)isoxazol-5-yl)phenyl)ethynyl)-6-azaspiro[3.4]octan-6-yl)ethan-1-one 28 (5 mg) with a yield of 6.30%.

MS m/z (ESI): 461.2 [M+1].

### Example 29

### (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanenitrile

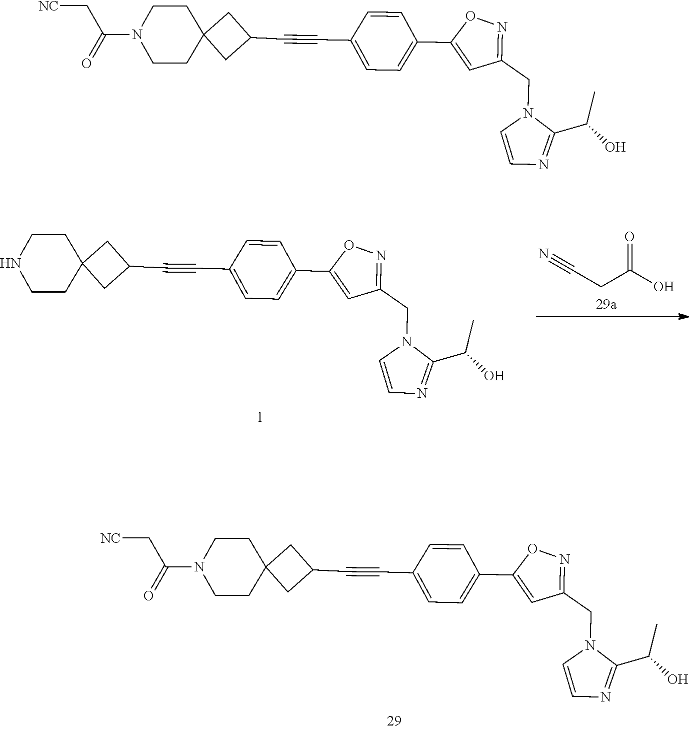 [SMILES: C[C@H](O)c1nccn1Cc1cc(-c2ccc(C#CC3CC4(CCN(C(=O)CC#N)CC4)C3)cc2)on1 ; C[C@H](O)c1nccn1Cc1cc(-c2ccc(C#CC3CC4(CCNCC4)C3)cc2)on1 ; N#CCC(=O)O ; C[C@H](O)c1nccn1Cc1cc(-c2ccc(C#CC3CC4(CCN(C(=O)CC#N)CC4)C3)cc2)on1]

2-cyanoacetic acid 29a (15.32 mg, 180.07 μmol, commercially available), 1-(3-dimethylaminopropyl)-3-ethylcarbodiimide hydrochloride (34.52 mg, 180.07 μmol), 1-hydroxybenzotriazole (24.33 mg, 180.07 μmol), and 4-methylmorpholine (26.71 mg, 264.10 μmol) were added to N,N-dimethylformamide solution (1.5 mL), reacted for five minutes, and added with (S)-1-(1-((5-(4-((7-azaspiro[3.5]nonan-2-yl)ethynyl)phenyl)isoxazol-3-yl)methyl)-1H-imidazol-2-yl)ethan-1-ol 1 (50 mg, 120.04 μmol), and reacted at room temperature for 16 hours. After the reaction was completed, the reaction solution was added with water (50 mL), and extracted with dichloromethane (50 mL×3) thrice. The organic phases were combined, concentrated and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)-3-oxopropanenitrile 29 (3 mg) with a yield of 3.97%.

MS m/z (ESI): 484.2 [M+1].

### Example 30

### (S)-1-(1-((5-(4-((7-(azetidin-3-yl)-7-azaspiro[3.5]nonan-2-yl)ethynyl)phenyl)isoxazol-3-yl)methyl)-1H-imidazol-2-yl)ethan-1-ol

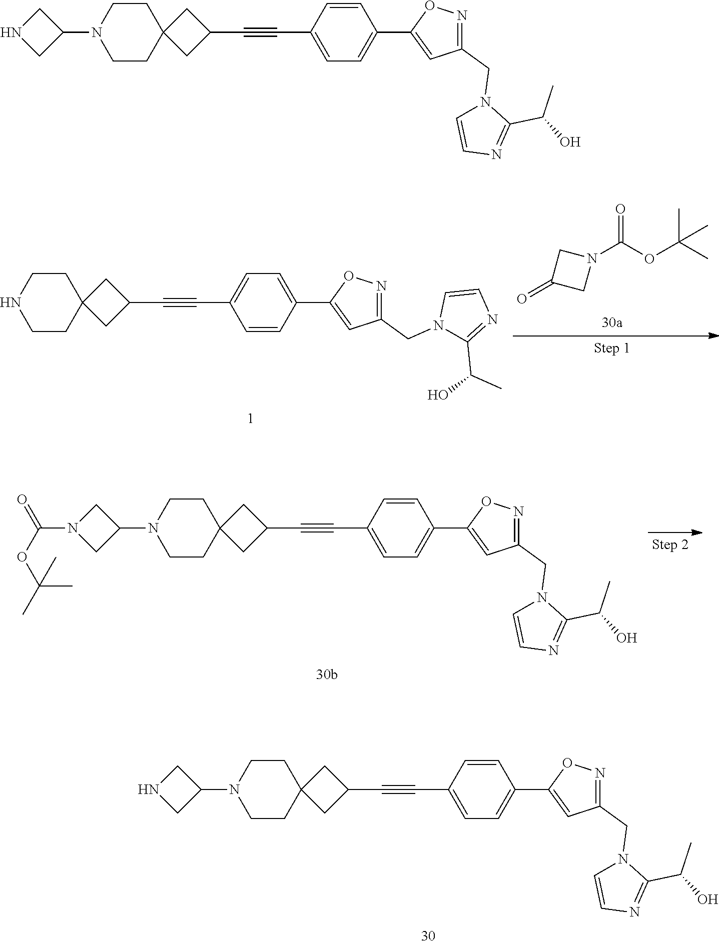

### Step 1

### Tert-butyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)azetidine-1-carboxylate

At room temperature, (S)-1-(1-((5-(4-((7-azaspiro[3.5]nonan-2-yl)ethynyl)phenyl)isoxazol-3-yl)methyl)-1H-imidazol-2-yl)ethan-1-ol 1 (26.81 mg, 64.36 μmol), and tert-butyl 3-oxoazetidine-1-carboxylate 30a (16.53 mg, 96.53 μmol, commercially available) were dissolved in a mixed solution of 1,2-dichloroethane (0.5 mL) and methanol (0.5 mL), dropwise added with acetic acid (20 mg, 64.36 μmol), reacted at 50 °C for 6 hours, slowly dropwise added with sodium cyanoborohydride at room temperature (12.13 mg, 193.07 μmol), and reacted at room temperature for 16 hours. After the reaction was completed, the reaction solution was added with water (1 mL) for quenching, concentrated and subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain tert-butyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)azetidine-1-carboxylate 30b (25 mg) with a yield of 67.95%.

MS m/z (ESI): 572.4 [M+1].

### Step 2

### (S)-1-(1-((5-(4-((7-(azetidin-3-yl)-7-azaspiro[3.5]nonan-2-yl)ethynyl)phenyl)isoxazol-3-yl)methy 1)-1H-imidazol-2-yl)ethan-1-ol

At room temperature, tert-butyl (S)-3-(2-((4-(3-((2-(1-hydroxyethyl)-1H-imidazol-1-yl)methyl)isoxazol-5-yl)phenyl)ethynyl)-7-azaspiro[3.5]nonan-7-yl)azetidine-1-carboxylate 30b (25 mg, 43.73 μmol) was dissolved in dichloromethane (2 mL), dropwise added with trifluoroacetic acid (0.5 g, 4.39 mmol), and reacted at room temperature for 4 hours. After the reaction was completed, the reaction solution was concentrated, and subjected to preparative liquid separation (separation column AKZONOBEL Kromasil; 250×21.2 mm I.D; 5 μm, 20 mL/min; mobile phase A: 0.05% TFA+H₂O, mobile phase B: CH₃CN) to obtain (S)-1-(1-((5-(4-((7-(azetidin-3-yl)-7-azaspiro[3.5]nonan-2-yl)ethynyl)phenyl)isoxazol-3-yl)methyl)-1H-imidazol-2-yl)ethan-1-ol 30 (9 mg) with a yield of 31.63%.

MS m/z (ESI): 472.3 [M+1].

### Example 31

### (S)-2-(6-((4-(3-((2-(1-hydroxyethyl)-1H-imidazol-1-yl)methyl)isoxazol-5-yl)phenyl)ethynyl)-2-azaspiro[3.3]heptan-2-yl)ethan-1-ol

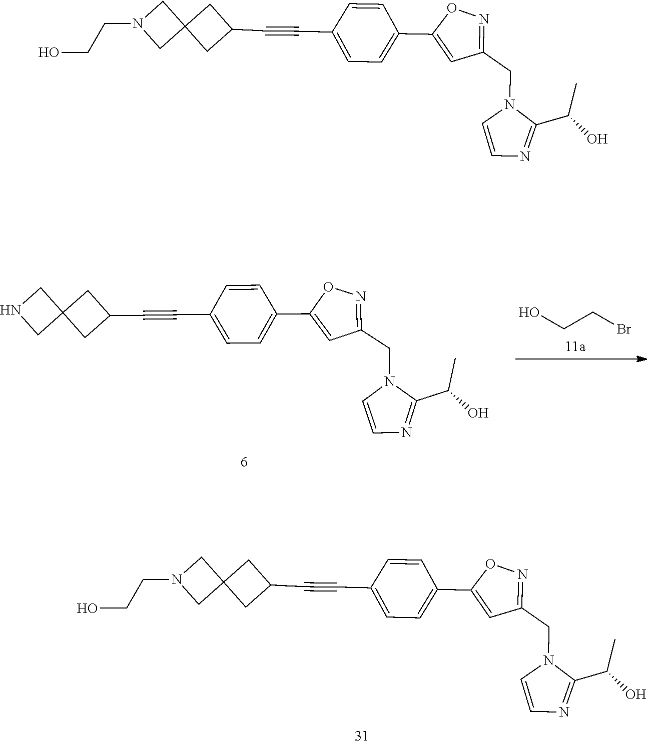

At room temperature, (S)-1-(1-((5-(4-((2-azaspiro[3.3]heptan-6-yl)ethynyl)phenyl)isoxazol-3-yl)methyl)-1H-imidazol-2-yl)ethan-1-ol 6 (25 mg, 49.75 μmol), 2-bromoethanol 11a (6.84 mg, 54.73 μmol, commercially available), and potassium carbonate (27.51 mg, 199.01 μmol) were added to acetonitrile (0.5 mL), and reacted at 60 °C for 12 hours. After the reaction was completed, the reaction solution was filtered, and the filtrate was concentrated and subjected to preparative liquid separation (separation column AKZONOBEL Kromasil; 250×21.2 mm I.D; 5 μm, 20 mL/min; mobile phase A: 0.05% TFA+H₂O, mobile phase B: CH₃CN) to obtain (S)-2-(6-((4-(3-((2-(1-hydroxyethyl)-1H-imidazol-1-yl)methyl)isoxazol-5-yl)phenyl)ethynyl)-2-azaspiro[3.3]heptan-2-yl)ethan-1-ol (8 mg) with a yield of 26.48%.

MS m/z (ESI): 433.0 [M+1].

### Example 32

### (S)-3-(6-((4-(3-((2-(1-hydroxyethyl)-1H-imidazol-1-yl)methyl)isoxazol-5-yl)phenyl)ethynyl)-2-azaspiro[3.3]heptan-2-yl)-3-oxopropanenitrile

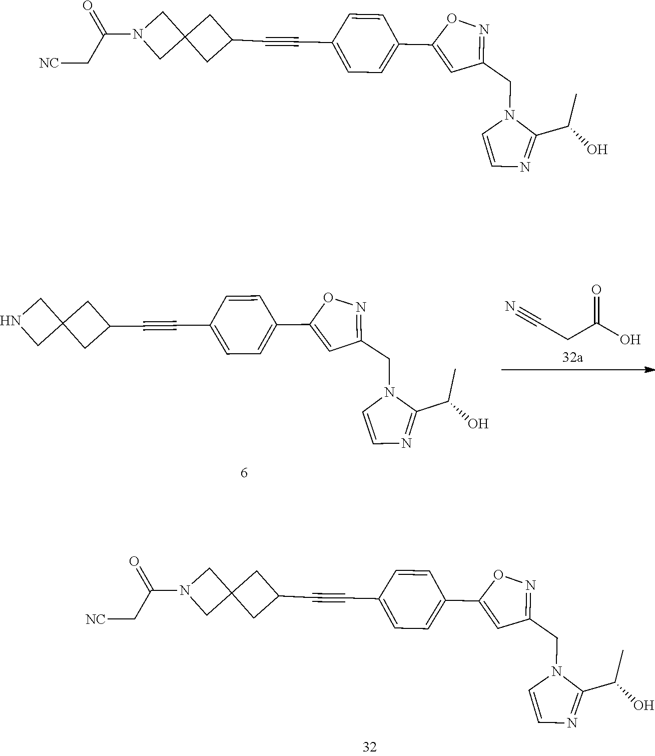 [SMILES: C[C@H](O)c1nccn1Cc1cc(-c2ccc(C#CC3CC4(C3)CN(C(=O)CC#N)C4)cc2)on1 ; C[C@H](O)c1nccn1Cc1cc(-c2ccc(C#CC3CC4(CNC4)C3)cc2)on1 ; N#CCC(=O)O ; C[C@H](O)c1nccn1Cc1cc(-c2ccc(C#CC3CC4(C3)CN(C(=O)CC#N)C4)cc2)on1]

At room temperature, 1-(3-dimethylaminopropyl)-3-ethylcarbodiimide hydrochloride (57.23 mg, 298.52 μmol), 1-hydroxybenzotriazole (40.34 mg, 298.52 μmol), 4-methylmorpholine (84.55 mg, 835.85 μmol), and 2-cyanoacetic acid 32a (30.47 mg, 358.22 μmol, commercially available) were dissolved in dichloromethane (2 mL), reacted for five minutes, and added with (S)-1-(1-((5-(4-((2-azaspiro[3.3]heptan-6-yl)ethynyl)phenyl)isoxazol-3-yl)methyl)-1H-imidazol-2-yl)ethan-1-ol 6 (60 mg, 119.41 μmol), and reacted overnight at room temperature. After the reaction was completed, the reaction solution was added with water (50 mL), and extracted with dichloromethane (50 mL×3). The organic phases were combined, and dried by anhydrous sodium sulfate. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(6-((4-(3-((2-(1-hydroxyethyl)-1H-imidazol-1-yl)methyl)isoxazol-5-yl)phenyl)ethynyl)-2-azaspiro[3.3]heptan-2-yl)-3-oxopropanenitrile 32 (20 mg) with a yield of 27.94%.

MS m/z (ESI): 456.2[M+1].

### Example 33

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-4-yl)methanone

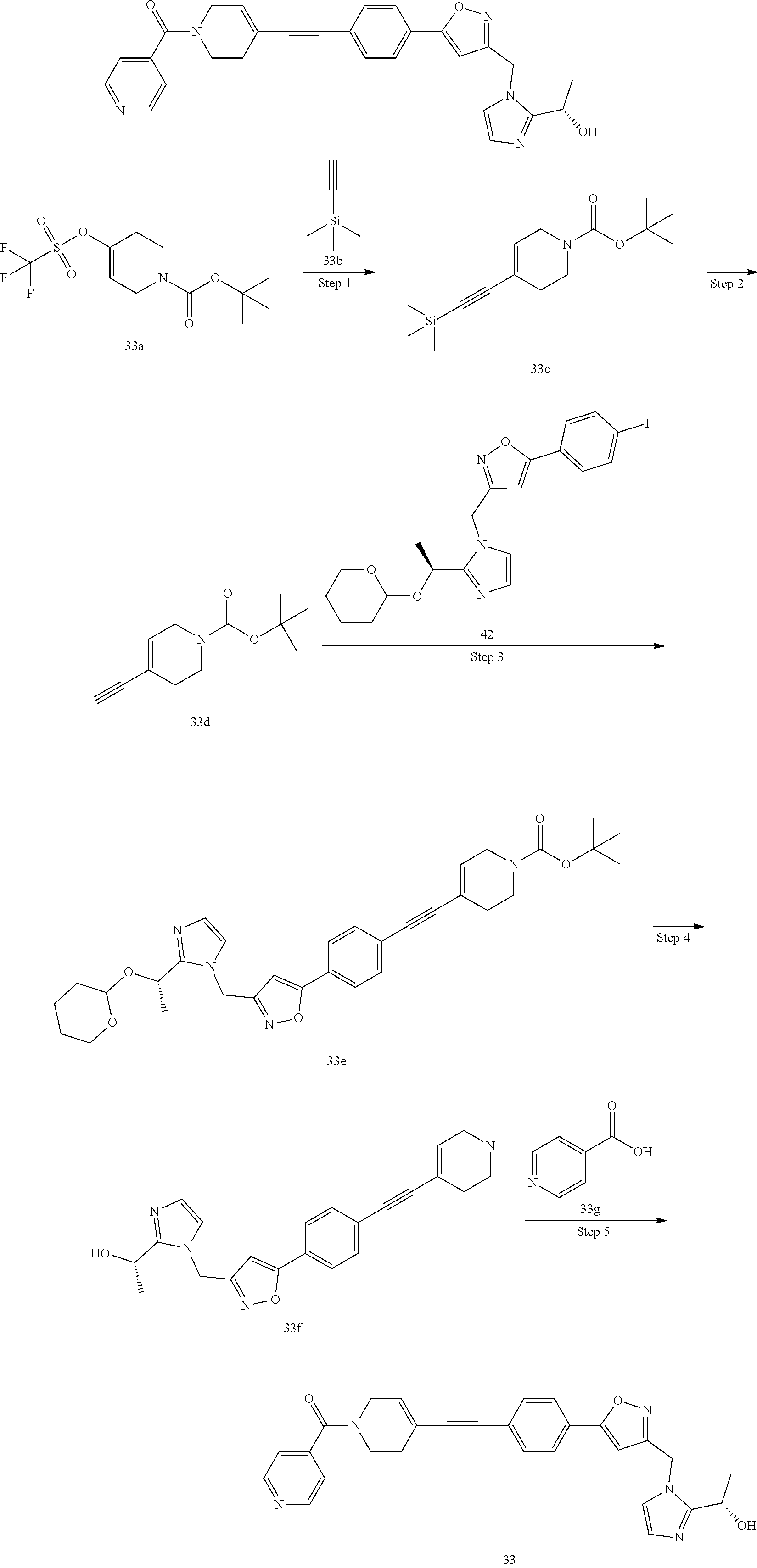

### Step 1

### Tert-butyl 4-((trimethylsilyl)ethynyl)-3,6-dihydropyridine-1(2H)-carboxylate

Tert-butyl 4-(((trifluoromethylsulfonyloxy)-3,6-dihydropyridine-1(2H)-carboxylate 33a (1 g, 3.02 mmol, commercially available), ethynyl trimethylsilane 33b (444.68 mg, 4.53 mmol, 639.83 μL, commercially available), bis(triphenylphosphine) palladium (II) chloride (352.28 mg, 452.75 μmol), triethylamine (305.43 mg, 3.02 mmol), and cuprous iodide (57.48 mg, 301.83 μmol) were sequentially added to toluene solution (3 mL), and continuously stirred at room temperature for 16 hours. The reaction solution was distilled under reduced pressure to remove the organic solvent, added with water (20 mL), and extracted with ethyl acetate (20 mL×3). The organic phases were combined, dried by anhydrous sodium sulfate, and distilled under reduced pressure. The residue was purified by silicagel column chromatography (eluent: system A) to obtain tert-butyl 4-((trimethylsilyl)ethynyl)-3,6-dihydropyridine-1(2H)-carboxylate 33c (820 mg) with a yield of 97.22%.

MS m/z (ESI): 224.1 [M+1-56].

### Step 2

### Tert-butyl 4-ethynyl-3,6-dihydropyridine-1(2H)-carboxylate

Tert-butyl 4-((trimethylsilyl)ethynyl)-3,6-dihydropyridine-1(2H)-carboxylate 33c (800 mg, 2.86 mmol) was added to methanol (2 mL), and then added with potassium fluoride (332.65 mg, 5.73 mmol), and continuously stirred at room temperature for 12 hours. The reaction solution was distilled under reduced pressure to remove the organic solvent, added with water (20 mL), and extracted with ethyl acetate (20 mL×3). The organic phases were combined, dried by anhydrous sodium sulfate, and distilled under reduced pressure. The residue was purified by silicagel column chromatography (eluent: system A) to obtain tert-butyl 4-ethynyl-3,6-dihydropyridine-1(2H)-carboxylate 33d (370 mg) with a yield of 62.36%.

MS m/z (ESI): 152.1[M+1-56].

### Step 3

### tert-butyl 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxylate

5-(4-iodophenyl)-3-((2-((1S)-1-((tetrahydro-2H-pyran-2-yl)oxy)ethyl)-1H-imidazol-1-yl)methyl)isoxazole 1i (800 mg, 1.67 mmol), tert-butyl 4-ethynyl-3,6-dihydropyridine-1(2H)-carboxylate 33d (363.24 mg, 1.75 mmol), allylpalladium chloride dimer (121.84 mg, 333.81 μmol), triethylene diamine (374.44 mg, 3.34 mmol), and tri-tert-butylphosphine (10% in toluene solution) (67.54 mg, 333.81 μmol) were sequentially added to acetonitrile (2 mL), and reacted overnight at room temperature. After the reaction was completed, the reaction solution was added with water (30 mL) for quenching, and extracted with ethyl acetate (100 mL×3). The combined organic phases were washed with saturated salt solution (100 mL), and concentrated under reduced pressure. The obtained residue was purified by silica gel column chromatography (eluent: B system) to obtain tert-butyl 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxylate 33e (660 mg) 33e with a yield of 70.78%.

MS m/z (ESI): 559.2 [M+1].

### Step 4

### (S)-1-(1-((5-(4-((1,2,3,6-tetrahydropyridin-4-yl)ethynyl)phenyl)isoxazol-3-yl)methyl)-1H-imidazol-2-yl)ethan-1-ol

Tert-butyl 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxylate 33e (660 mg, 1.18 mmol) was added to dichloromethane (1.5 mL), and then dropwise added with trifluoroacetic acid (0.5 mL) and continuously stirred at room temperature for one hour. After the reaction was completed, the reaction solution was added with saturated NaHCO₃ solution in an ice bath until no bubbles were generated (pH>7), and extracted with ethyl acetate (20 mL×3). The organic phases were combined, dried by anhydrous sodium sulfate, and distilled under reduced pressure. The residue was purified by silicagel column chromatography (eluent: system B) to obtain (S)-1-(1-((5-(4-((1,2,3,6-tetrahydropyridin-4-yl)ethynyl)phenyl)isoxazol-3-yl)methyl)-1H-imidazol-2-yl)ethan-1-ol 33f (320 mg) with a yield of 72.34%.

MS m/z (ESI): 375.2 [M+1].

### Step 5

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-4-yl)methanone

Isonicotinic acid 33g (32.88 mg, 267.07 μmol, commercially available), (S)-1-(1-((5-(4-((1,2,3,6-tetrahydropyridin-4-yl)ethynyl)phenyl)isoxazol-3-yl)methyl)-1H-imidazol-2-yl)ethan-1-ol 33f (50 mg, 133.53 μmol), N,N-diisopropylethylamine (34.52 mg, 267.07 μmol), and (7-azobenzotriazole)-N,N,N′,N′-tetramethyluronium hexafluorophosphate (50.77 mg, 133.53 μmol) were sequentially added to N,N-dimethylformamide (1 mL), and continuously stirred at room temperature for 16 hours. After the reaction was completed, the reaction solution was added with water (50 mL), and extracted with dichloromethane (50 mL×3). The organic phases were combined, and dried by anhydrous sodium sulfate. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-4-yl)methanone 33 (13.2 mg) with a yield of 15.99%.

MS m/z (ESI): 480.3 [M+1].

### Example 34

### (S)-4-((4-((4-(3-((2-(1-hydroxyethyl)-1H-imidazol-1-yl)methyl)isoxazol-5-yl)phenyl)ethynyl)-3, 6-dihydropyridin-1(2H)-yl)methyl)benzoic acid

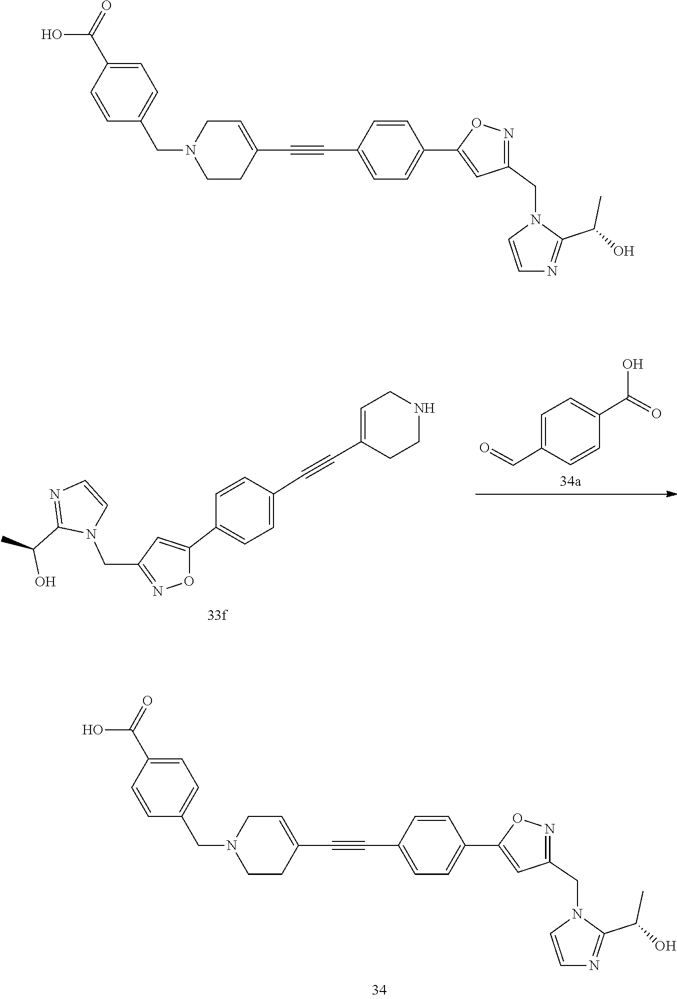 [SMILES: C[C@H](O)c1nccn1Cc1cc(-c2ccc(C#CC3=CCN(Cc4ccc(C(=O)O)cc4)CC3)cc2)on1 ; C[C@H](O)c1nccn1Cc1cc(-c2ccc(C#CC3=CCNCC3)cc2)on1 ; O=Cc1ccc(C(=O)O)cc1 ; C[C@H](O)c1nccn1Cc1cc(-c2ccc(C#CC3=CCN(Cc4ccc(C(=O)O)cc4)CC3)cc2)on1]

4-formylbenzoic acid 34a (24.06 mg, 160.24 μmol, commercially available), (S)-1-(1-((5-(4-((1,2,3,6-tetrahydropyridin-4-yl)ethynyl)phenyl)isoxazol-3-yl)methyl)-1H-imidazol-2-yl)ethan-1-ol 33f (50 mg, 133.53 μmol), and acetic acid (8.02 mg, 133.53 μmol) were sequentially added to dichloromethane (1 mL) and continuously stirred at room temperature for one hour, then added with sodium triacetoxyborohydride (28.30 mg, 133.53 μmol), and stirred at room temperature for 16 hours. After the reaction was completed, the reaction solution was added with water (50 mL), and extracted with dichloromethane (50 mL×3). The organic phases were combined, and dried by anhydrous sodium sulfate. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-((4-((4-(3-((2-(1-hydroxyethyl)-1H-imidazol-1-yl)methyl)isoxazol-5-yl)phenyl)ethynyl)-3, 6-dihydropyridin-1(2H)-yl)methyl)benzoic acid 34 (7.35 mg) with a yield of 8.66%.

MS m/z (ESI): 509.0 [M+1].

### Example 35

### ((1R,5S)-bicyclo[3.1.0]hexan-6-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone

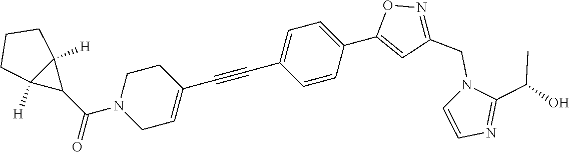 [SMILES: C[C@H](O)c1nccn1Cc1cc(-c2ccc(C#CC3=CCN(C(=O)C4[C@H]5CCC[C@@H]45)CC3)cc2)on1]

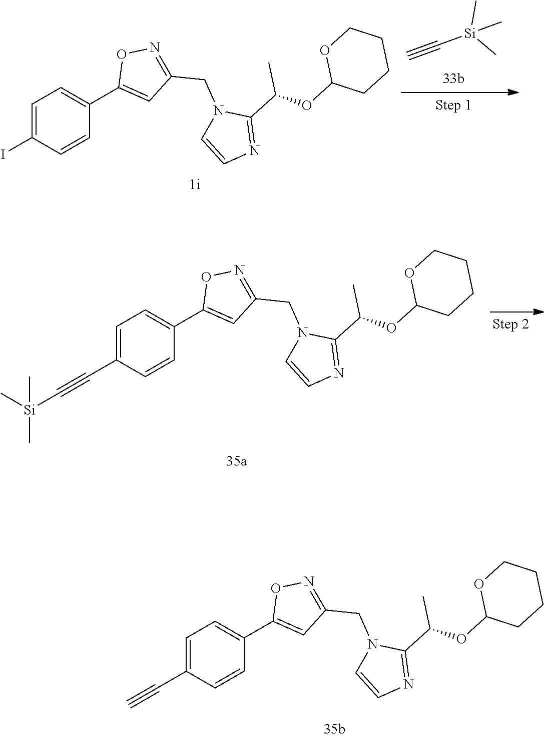

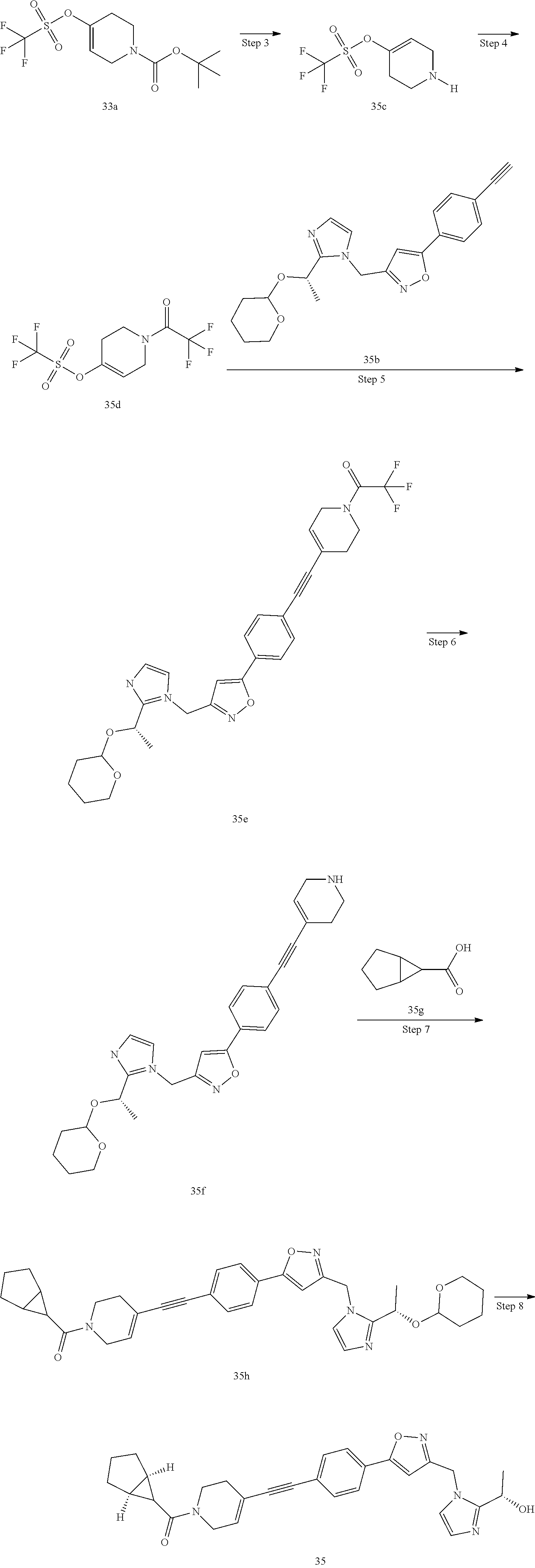

### Step 1

### 3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole

5-(4-iodophenyl)-3-((2-((1S)-1-((tetrahydro-2H-pyran-2-yl)oxy)ethyl)-1H-imidazol-1-yl)methyl)isoxazole 1i (8 g, 16.69 mmol), ethynyl trimethylsilane 33b (3.28 g, 33.38 mmol, 4.72 mL, commercially available), allylpalladium chloride dimer (609.21 mg, 1.67 mmol), triethylene diamine (3.74 g, 33.38 mmol) and tri-tert-butylphosphine (10% toluene solution) (3.38 g, 1.67 mmol) were sequentially added to acetonitrile (20 mL), replaced thrice with argon gas, and continuously stirred at room temperature for 12 hours. The system was concentrated to dryness under reduced pressure, added with water (50 mL), and extracted with ethyl acetate (50 mL×3). The combined organic phases were dried with anhydrous sodium sulfate and distilled under reduced pressure. The residue was purified by silicagel column chromatography (eluent. system A) to obtain 3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole 35a (7.5 g) with a yield of 99.94%.

MS m/z (ESI): 450.3 [M+1].

### Step 2

### 5-(4-ethynylphenyl)-3-((2-((1S)-1-((tetrahydro-2H-pyran-2-yl)oxy)ethyl)-1H-imidazol-1-yl)methyl)isoxazole

3-((2-((1S)-1-((tetrahydro-2H-pyran-2-yl)oxy)ethyl)-1H-imidazol-1-yl)methyl)-5-(4-((trimethylsilyl)ethynyl)phenyl)isoxazole 35a (7.5 g, 16.68 mmol) and potassium fluoride (1.94 g, 33.36 mmol) were sequentially added to methanol (50 mL), and continuously stirred at room temperature for 5 hours. The reaction was completed, the reaction solution was added with water (50 mL), and extracted with ethyl acetate (50 mL×3). The combined organic phases were dried with anhydrous sodium sulfate and distilled under reduced pressure. The residue was purified by silicagel column chromatography (eluent: system A) to obtain 5-(4-ethynylphenyl)-3-((2-((1S)-1-((tetrahydro-2H-pyran-2-yl)oxy)ethyl)-1H-imidazol-1-yl)methyl)isoxazole 35b (5.8 g) with a yield of 92.21%.

MS m/z (ESI): 378.2 [M+1].

### Step 3

### 1,2,3,6-tetrahydropyridin-4-yl trifluoromethanesulfonate

Tert-butyl 4-(((trifluoromethylsulfonyloxy)-3,6-dihydropyridine-1(2H)-carboxylate 33a (1.7 g, 5.13 mmol) was added to dichloromethane (1.5 mL), and then dropwise added with trifluoroacetic acid (0.5 mL), and continuously stirred at room temperature for one hour. After the reaction was completed, the reaction was added with saturated NaHCO₃ solution until no bubbles were generated and pH>7, and then added with water (50 mL), and extracted with dichloromethane (50 mL×3). The organic phases were combined, dried with anhydrous sodium sulfate, and concentrated to obtain 1,2,3,6-tetrahydropyridin-4-yl trifluoromethanesulfonate 35c (1.1 g) with a yield of 92%, The crude product was directly used in the next reaction.

MS m/z (ESI): 232.1 [M+1].

### Step 4

### 1-(2,2,2-trifluoroacetyl)-1,2,3,6-tetrahydropyridin-4-yl trifluoromethanesulfonate

In an ice bath, 1,2,3,6-tetrahydropyridin-4-yl trifluoromethanesulfonate 35c (900 mg, 3.89 mmol), and triethylamine (393.92 mg, 3.89 mmol) were sequentially added to dichloromethane (10 mL), and then dropwise added with trifluoroacetic anhydride (820 mg, 3.89 mmol), gradually heated to room temperature and continuously stirred for 20 minutes. It was detected that all the raw materials were completely reacted. The reaction solution was added with water (50 mL), and extracted with ethyl acetate (50 mL×3). The combined organic phases were dried with anhydrous sodium sulfate and distilled under reduced pressure. The residue was purified by silicagel column chromatography (eluent: system A) to obtain 1-(2,2,2-trifluoroacetyl)-1,2,3,6-tetrahydropyridin-4-yl trifluoromethanesulfonate 35d (1.17 g) with a yield of 91%.

MS m/z (ESI): 328.0 [M+1].

### Step 5

### 2,2,2-trifluoro-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one

In an argon gas atmosphere, 1-(2,2,2-trifluoroacetyl)-1,2,3,6-tetrahydropyridin-4-yl trifluoromethanesulfonate 35d (1.13 g, 3.44 mmol), 5-(4-ethynylphenyl)-3-((2-((1S)-1-((tetrahydro-2H-pyran-2-yl)oxy)ethyl)-1H-imidazol-1-yl)methyl)isoxazole 35b (1 g, 2.65 mmol), cuprous iodide (50.46 mg, 264.95 μmol), bis(triphenylphosphine) palladium (II) chloride (412.30 mg, 529.89 μmol), and triethylamine (268.10 mg, 2.65 mmol) were sequentially added to methylbenzene (5 mL), and continuously stirred at room temperature for 16 hours. After the reaction was completed, the reaction solution was distilled under reduced pressure to remove the organic solvent. The residue was purified by silicagel column chromatography (eluent: system A) to obtain 2,2,2-trifluoro-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 35e (400 mg) with a yield of 27.22%.

MS m/z (ESI): 555.3 [M+1].

### Step 6

### 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole

2,2,2-trifluoro-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 35e (400 mg, 721.29 μmol), and potassium carbonate (797.52 mg, 5.77 mmol) were sequentially added to a mixed solution of methanol (10 mL) and water (0.5 mL), replaced with argon gas thrice, and then stirred at 70 °C for 2 hours. After the reaction was completed, the reaction solution was distilled under reduced pressure to remove the organic solvent. The crude product was purified by silicagel column chromatography (eluent: system B) to obtain 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (180 mg) with a yield of 54.42%.

MS m/z (ESI): 459.3 [M+1].

### Step 7

### Bicyclo[3.1.0]hexan-6-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Bicyclo[3.1.0]hexan-6-carboxylic acid 35g (16.51 mg, 130.85 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), N-methylmorpholine (19.85 mg, 196.27 μmol), 1-hydroxybenzotriazole (17.68 mg, 130.85 μmol), and 1-(3-dimethylaminopropyl)-3-ethylcarbodiimide hydrochloride (25.08 mg, 130.85 μmol) were sequentially added to dichloromethane (1 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain Bicyclo[3.1.0]hexan-6-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 35h (37 mg). The crude product was directly used in the next reaction.

MS m/z (ESI): 567.2 [M+1].

### Step 8

### ((1R,5S)-bicyclo[3.1.0]hexan-6-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone

Bicyclo[3.1.0]hexan-6-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 35h (37 mg, 65.29 μmol) was added to dichloromethane (1.5 mL), and then dropwise added with trifluoroacetic acid (0.5 mL), and continuously stirred at room temperature for half an hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain ((1R,5S)-bicyclo[3.1.0]hexan-6-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone 35 (4.05 mg) with a yield of 9.88%.

MS m/z (ESI): 483.2 [M+1].

### Example 36

### (S)-cyclopropyl(4-((4-(3-((2-(1-hydroxyethyl)-1H-imidazol-1-yl)methyl)isoxazol-5-yl)phenyl)eth ynyl)-3,6-dihydropyridin-1(2H)-yl)methanone

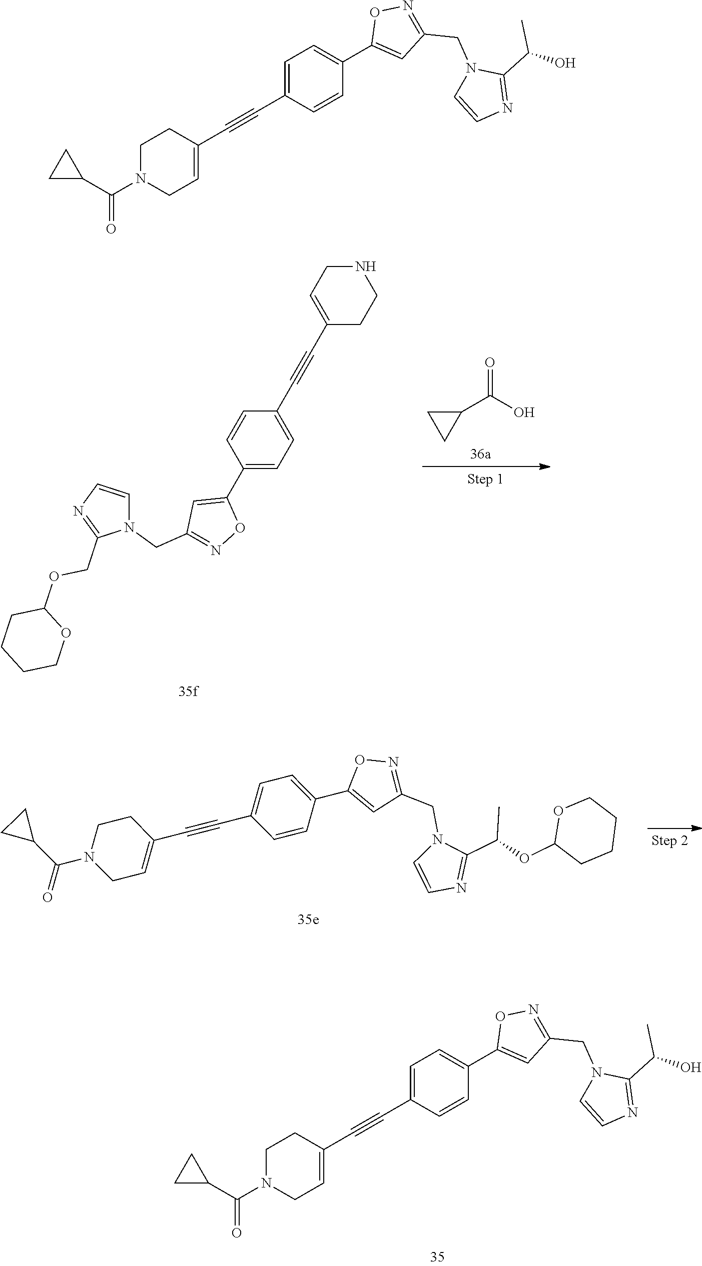 [SMILES: C[C@H](O)c1nccn1Cc1cc(-c2ccc(C#CC3=CCN(C(=O)C4CC4)CC3)cc2)on1 ; C(#Cc1ccc(-c2cc(Cn3ccnc3COC3CCCCO3)no2)cc1)C1=CCNCC1 ; O=C(O)C1CC1 ; C[C@H](OC1CCCCO1)c1nccn1Cc1cc(-c2ccc(C#CC3=CCN(C(=O)C4CC4)CC3)cc2)on1 ; C[C@H](O)c1nccn1Cc1cc(-c2ccc(C#CC3=CCN(C(=O)C4CC4)CC3)cc2)on1]

### Step 1

### Cyclopropyl(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone

Cyclopropanecarboxylic acid 36a (11.26 mg, 130.85 μmol), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), N-methylmorpholine (19.85 mg, 196.27 μmol), 1-hydroxybenzotriazole (17.68 mg, 130.85 μmol), and 1-(3-dimethylaminopropyl)-3-ethylcarbodiimide hydrochloride (25.08 mg, 130.85 μmol) were sequentially added to dichloromethane (1 mL), and continuously stirred at room temperature for six hours. After the reaction was completed, the reaction solution was added with water (50 mL), and extracted with dichloromethane (50 mL×3). The organic phases were combined, dried with anhydrous sodium sulfate, and concentrated to obtain cyclopropyl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 36b (34 mg) with a yield of 98.68%. The crude product was directly used for the next reaction.

MS m/z (ESI): 527.3 [M+1].

### Step 2

### (S)-cyclopropyl(4-((4-(3-((2-(1-hydroxyethyl)-1H-imidazol-1-yl)methyl)isoxazol-5-yl)phenyl)ethynyl)-3,6-dihydropyridin-1(2H)-yl)methanone

Cyclopropyl(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone 36b (34 mg, 64.56 μmol) was added to dichloromethane (1.5 mL), and then dropwise added with trifluoroacetic acid (0.5 mL), and continuously stirred at room temperature for half an hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-cyclopropyl(4-((4-(3-((2-(1-hydroxyethyl)-1H-imidazol-1-yl)methyl)isoxazol-5-yl)phenyl)eth ynyl)-3,6-dihydropyridin-1(2H)-yl)methanone 36 (25.29 mg) with a yield of 69.68%.

MS m/z (ESI): 443.2 [M+1].

### Example 37

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one

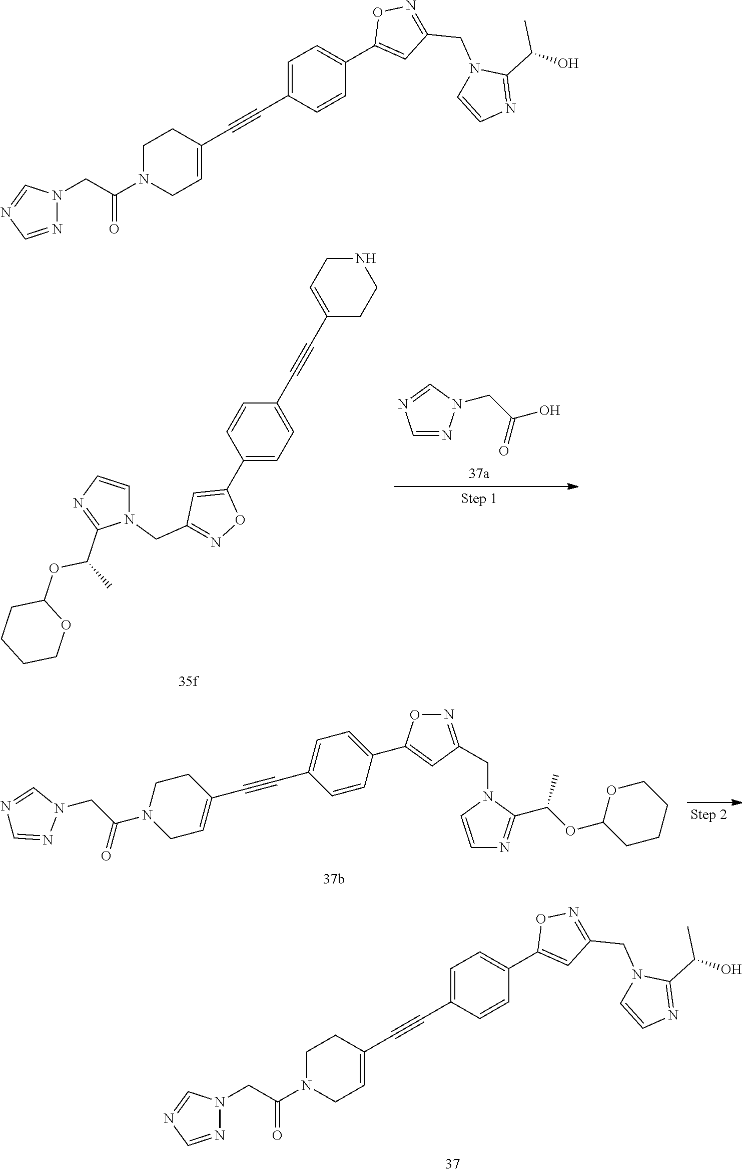 [SMILES: C[C@H](O)c1nccn1Cc1cc(-c2ccc(C#CC3=CCN(C(=O)Cn4cncn4)CC3)cc2)on1 ; C[C@H](OC1CCCCO1)c1nccn1Cc1cc(-c2ccc(C#CC3=CCNCC3)cc2)on1 ; O=C(O)Cn1cncn1 ; C[C@H](OC1CCCCO1)c1nccn1Cc1cc(-c2ccc(C#CC3=CCN(C(=O)Cn4cncn4)CC3)cc2)on1 ; C[C@H](O)c1nccn1Cc1cc(-c2ccc(C#CC3=CCN(C(=O)Cn4cncn4)CC3)cc2)on1]

### Step 1

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one

2-(1H-1,2,4-triazol-1-yl)acetic acid 37a (16.63 mg, 130.85 μmol), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), N-methylmorpholine (19.85 mg, 196.27 μmol), 1-hydroxybenzotriazole (17.68 mg, 130.85 μmol), and 1-(3-dimethylaminopropyl)-3-ethylcarbodiimide hydrochloride (25.08 mg, 130.85 μmol) were sequentially added to dichloromethane (1 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one 37b (37 mg) with a yield of 99.63%, which was directly used in the next reaction.

MS m/z (ESI): 568.4 [M+1].

### Step 2

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one 37b (37 mg, 65.18 μmol) was added to dichloromethane (1.5 mL), and then dropwise added with trifluoroacetic acid (0.5 mL), and continuously stirred at room temperature for half an hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-(1H-1,2,4-triazol-1-yl)ethan-1-one 37 (27.39 mg) with a yield of 69.62%.

MS m/z (ESI): 484.2 [M+1].

### Example 38

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrazin-2-yl)methanone

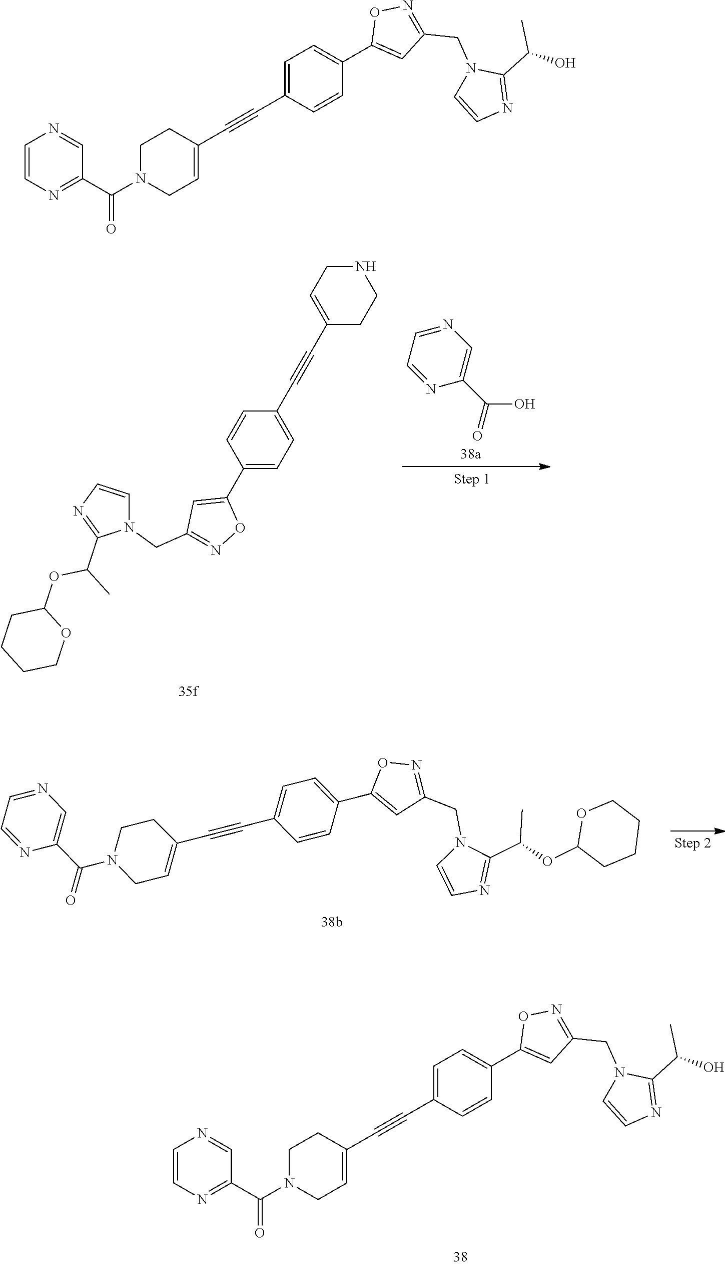 [SMILES: C[C@H](O)c1nccn1Cc1cc(-c2ccc(C#CC3=CCN(C(=O)c4cnccn4)CC3)cc2)on1 ; CC(OC1CCCCO1)c1nccn1Cc1cc(-c2ccc(C#CC3=CCNCC3)cc2)on1 ; O=C(O)c1cnccn1 ; C[C@H](OC1CCCCO1)c1nccn1Cc1cc(-c2ccc(C#CC3=CCN(C(=O)c4cnccn4)CC3)cc2)on1 ; C[C@H](O)c1nccn1Cc1cc(-c2ccc(C#CC3=CCN(C(=O)c4cnccn4)CC3)cc2)on1]

### Step 1

### Pyrazin-2-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Pyrazine-2-carboxylic acid 38a (16.24 mg, 130.85 μmol), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), N-methylmorpholine (19.85 mg, 196.27 μmol), 1-hydroxybenzotriazole (17.68 mg, 130.85 μmol), and 1-(3-dimethylaminopropyl)-3-ethylcarbodiimide hydrochloride (25.08 mg, 130.85 μmol) were sequentially added to dichloromethane (1 mL), and continuously stirred at room temperature for six hours. After the reaction was completed, the reaction solution was added with water (50 mL), and extracted with dichloromethane (50 mL×3). The organic phases were combined, dried with anhydrous sodium sulfate, and concentrated to obtain pyrazin-2-yl(4-((4-(3-((2-((1S)-1-((tetrahydro-2H-pyran-2-yl)oxy)ethyl)-1H-imidazol-1-yl)methy 1)isoxazol-5-yl)phenyl)ethynyl)-3,6-dihydropyridin-1(2H)-yl)methanone 38b (36 mg) with a yield of 97.45%. The crude product was directly used for the next reaction.

MS m/z (ESI): 565.3 [M+1].

### Step 2

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrazin-2-yl)methanone

Pyrazin-2-yl(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone 38b (36 mg, 63.76 μmol) was added to dichloromethane (1.5 mL), and then dropwise added with trifluoroacetic acid (0.5 mL), and continuously stirred at room temperature for half an hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrazin-2-yl)methanone 38 (14.76 mg) with a yield of 38.12%.

MS m/z (ESI): 481.2 [M+1].

### Example 39

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridazin-4-yl)methanone

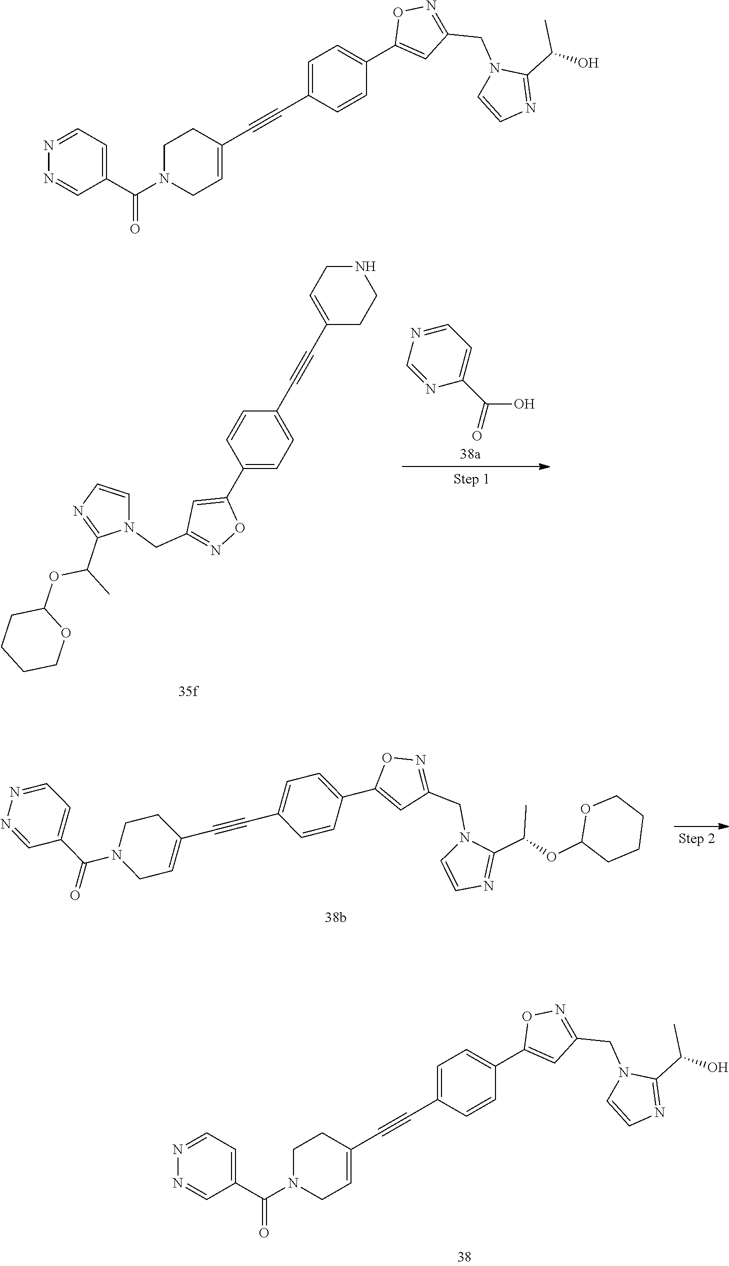 [SMILES: C[C@H](O)c1nccn1Cc1cc(-c2ccc(C#CC3=CCN(C(=O)c4ccnnc4)CC3)cc2)on1 ; CC(OC1CCCCO1)c1nccn1Cc1cc(-c2ccc(C#CC3=CCNCC3)cc2)on1 ; O=C(O)c1ccncn1 ; C[C@H](OC1CCCCO1)c1nccn1Cc1cc(-c2ccc(C#CC3=CCN(C(=O)c4ccnnc4)CC3)cc2)on1 ; C[C@H](O)c1nccn1Cc1cc(-c2ccc(C#CC3=CCN(C(=O)c4ccnnc4)CC3)cc2)on1]

### Step 1

### Pyridazin-4-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Pyridazine-4-carboxylic acid 39a (16.24 mg, 130.85 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), N-methylmorpholine (19.85 mg, 196.27 μmol), 1-hydroxybenzotriazole (17.68 mg, 130.85 μmol), and 1-(3-dimethylaminopropyl)-3-ethylcarbodiimide hydrochloride (25.08 mg, 130.85 μmol) were sequentially added to dichloromethane (1 mL), and continuously stirred at room temperature for six hours. After the reaction was completed, the reaction solution was added with water (50 mL), and extracted with dichloromethane (50 mL×3). The organic phases were combined, dried with anhydrous sodium sulfate, and concentrated to obtain pyridazin-4-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 39b (36 mg) with a yield of 97.45%. The crude product was directly used for the next reaction.

MS m/z (ESI): 565.3 [M+1].

### Step 2

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridazin-4-yl)methanone

Pyridazin-4-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 39b (36 mg, 63.76 μmol) was added to dichloromethane (1.5 mL), and then dropwise added with trifluoroacetic acid (0.5 mL), and continuously stirred at room temperature for half an hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridazin-4-yl)methanone 39 (13.22 mg) with a yield of 33.83%.

MS m/z (ESI): 481.2 [M+1].

### Example 40

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrimidin-5-yl)methanone

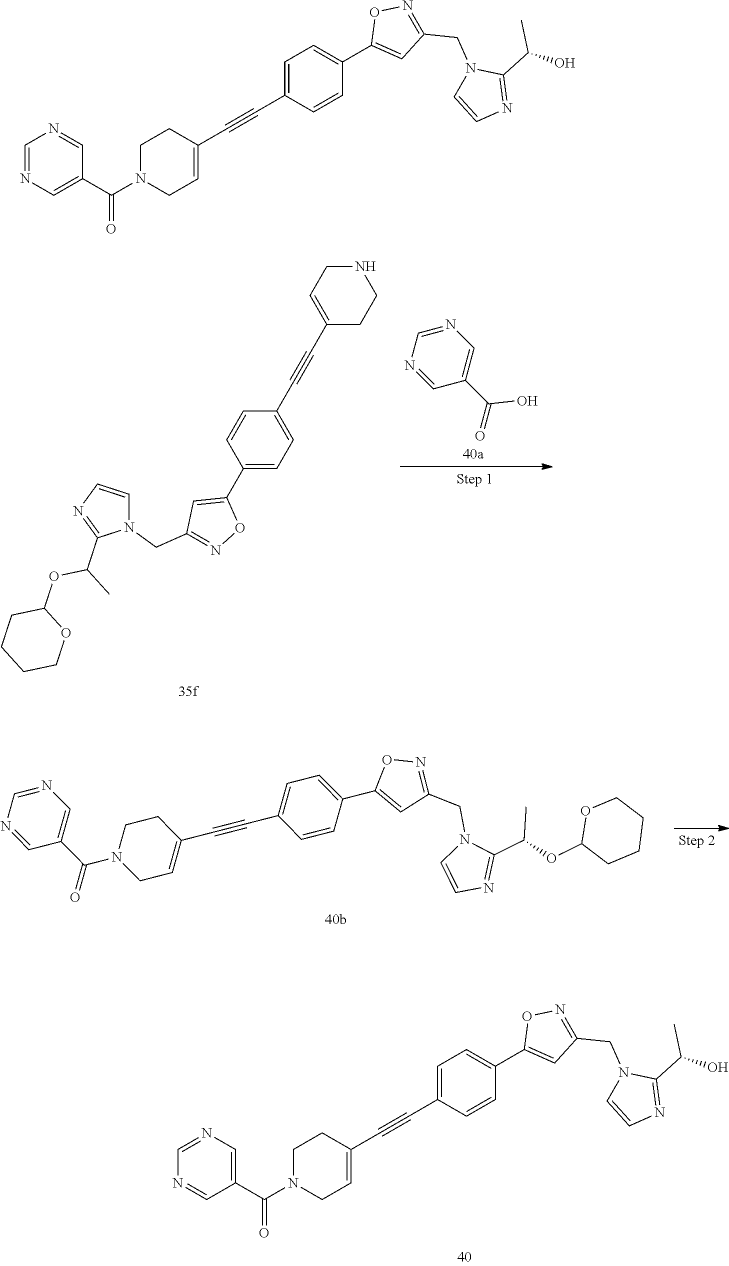 [SMILES: C[C@H](O)c1nccn1Cc1cc(-c2ccc(C#CC3=CCN(C(=O)c4cncnc4)CC3)cc2)on1 ; CC(OC1CCCCO1)c1nccn1Cc1cc(-c2ccc(C#CC3=CCNCC3)cc2)on1 ; O=C(O)c1cncnc1 ; C[C@H](OC1CCCCO1)c1nccn1Cc1cc(-c2ccc(C#CC3=CCN(C(=O)c4cncnc4)CC3)cc2)on1 ; C[C@H](O)c1nccn1Cc1cc(-c2ccc(C#CC3=CCN(C(=O)c4cncnc4)CC3)cc2)on1]

### Step 1

### Pyrimidin-5-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Pyrimidine-5-carboxylic acid 40a (21.65 mg, 174.46 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (40 mg, 87.23 μmol), 1-hydroxybenzotriazole (23.57 mg, 174.46 μmol), N-methylmorpholine (26.47 mg, 261.69 μmol), and 1-(3-dimethylaminopropyl)-3-ethylcarbodiimide hydrochloride (33.44 mg, 174.46 μmol) were sequentially added to dichloromethane (1.5 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain pyrimidin-5-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 40b (49 mg) with a yield of 99.48%, which was directly used in the next reaction.

MS m/z (ESI): 565.3 [M+1].

### Step 2

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrimidin-5-yl)methanone

Pyrimidin-5-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 40b (49 mg, 86.78 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyrimidin-5-yl)methanone 40 (22.3 mg) with a yield of 42.36%.

MS m/z (ESI): 481.2 [M+1]. ¹H NMR (400 MHz, DMSO-d₆) δ 9.30 (s, 1H), 8.94 (s, 2H), 7.88 (d, J=8.2 Hz, 2H), 7.79 (d, J=2.0 Hz, 1H), 7.72 (d, J=2.1 Hz, 1H), 7.62 (d, J=8.1 Hz, 2H), 7.17 (s, 1H), 6.34 (s, 1H), 5.72 (s, 2H), 5.26 (q, J=6.6 Hz, 1H), 4.27 (s, 1H), 4.13 (s, 2H), 3.81 (s, 1H), 2.41 (s, 2H), 1.49 (d, J=6.6 Hz, 3H).

### Example 41

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydrofuran-3-yl)methanone

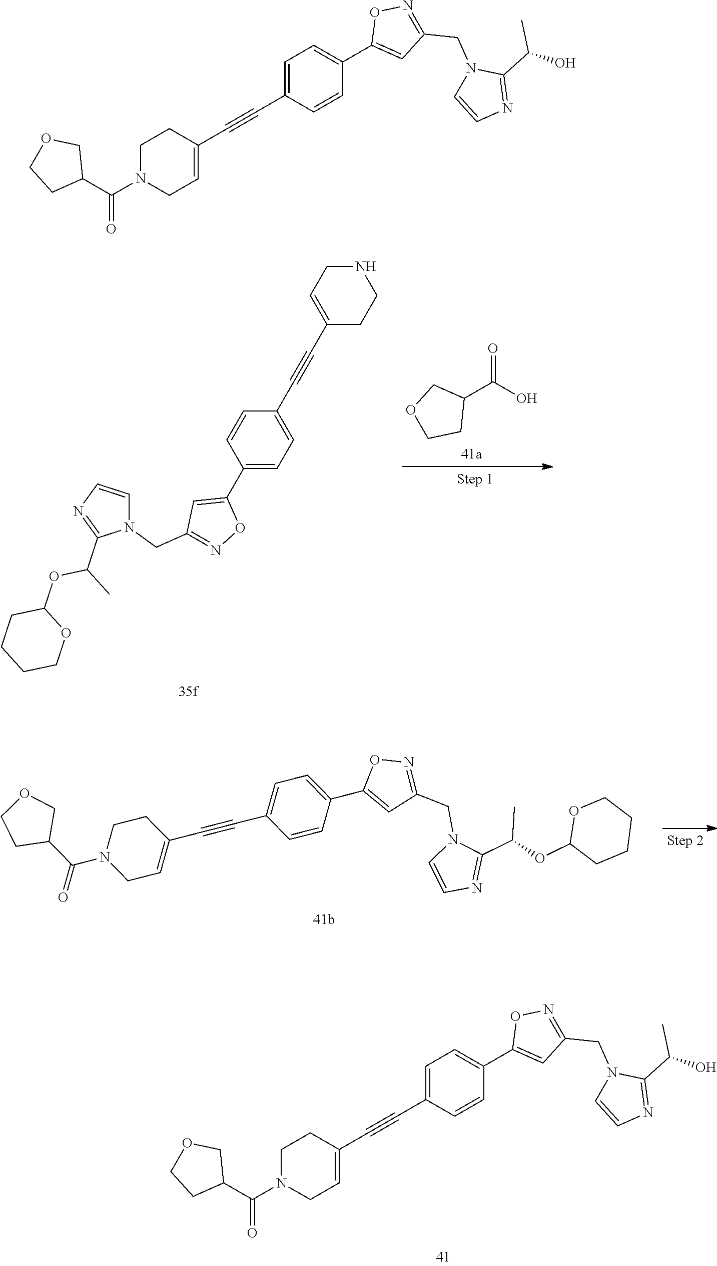 [SMILES: C[C@H](O)c1nccn1Cc1cc(-c2ccc(C#CC3=CCN(C(=O)C4CCOC4)CC3)cc2)on1 ; CC(OC1CCCCO1)c1nccn1Cc1cc(-c2ccc(C#CC3=CCNCC3)cc2)on1 ; O=C(O)C1CCOC1 ; C[C@H](OC1CCCCO1)c1nccn1Cc1cc(-c2ccc(C#CC3=CCN(C(=O)C4CCOC4)CC3)cc2)on1 ; C[C@H](O)c1nccn1Cc1cc(-c2ccc(C#CC3=CCN(C(=O)C4CCOC4)CC3)cc2)on1]

### Step 1

### (4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydrofuran-3-yl)methanone

Tetrahydrofuran-3-carboxylic acid 41a (20.26 mg, 174.46 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (40 mg, 87.23 μmol), 1-hydroxybenzotriazole (23.57 mg, 174.46 μmol), N-methylmorpholine (26.47 mg, 261.69 μmol), and 1-(3-dimethylaminopropyl)-3-ethylcarbodiimide hydrochloride (33.44 mg, 174.46 μmol) were sequentially added to dichloromethane (1.5 mL), and continuously stirred at room temperature for 16 hours. After the reaction was completed, the reaction solution was added with water (50 mL), added with dichloromethane (50 mL×3) for extraction. The organic phases were combined, dried with anhydrous sodium sulfate, and concentrated to obtain (4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydrofuran-3-yl)methanone 41b (48 mg) with a yield of 98.85%. The crude product was directly used in the next reaction.

MS m/z (ESI): 557.3 [M+1].

### Step 2

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydrofuran-3-yl)methanone

(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydrofuran-3-yl)methanone 41b (48 mg, 86.23 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydrofuran-3-yl)methanone 41 (20.51 mg) with a yield of 36.90%.

MS m/z (ESI): 473.3 [M+1].

### Example 42

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(morpholin-3-yl)methanone

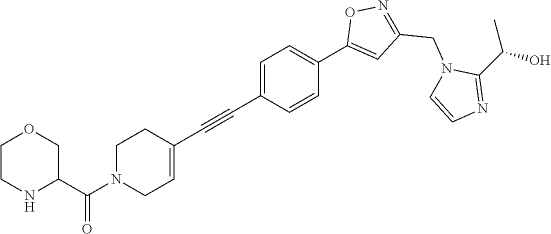 [SMILES: C[C@H](O)c1nccn1Cc1cc(-c2ccc(C#CC3=CCN(C(=O)C4COCCN4)CC3)cc2)on1]

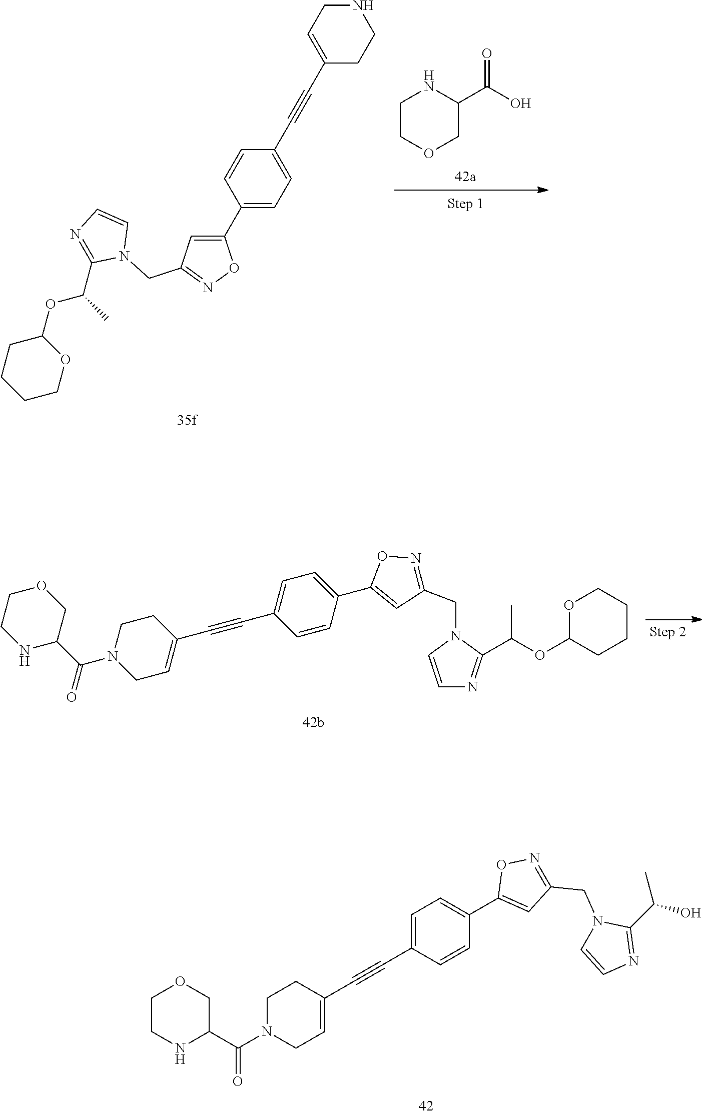 [SMILES: C[C@H](OC1CCCCO1)c1nccn1Cc1cc(-c2ccc(C#CC3=CCNCC3)cc2)on1 ; O=C(O)C1COCCN1 ; CC(OC1CCCCO1)c1nccn1Cc1cc(-c2ccc(C#CC3=CCN(C(=O)C4COCCN4)CC3)cc2)on1 ; C[C@H](O)c1nccn1Cc1cc(-c2ccc(C#CC3=CCN(C(=O)C4COCCN4)CC3)cc2)on1]

### Step 1

### Morpholin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Morpholine-3-carboxylic acid 42a (22.88 mg, 174.46 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (40 mg, 87.23 μmol), 1-hydroxybenzotriazole (23.57 mg, 174.46 μmol), N-methylmorpholine (26.47 mg, 261.69 μmol), and 1-(3-dimethylaminopropyl)-3-ethylcarbodiimide hydrochloride (33.44 mg, 174.46 μmol) were sequentially added to dichloromethane (1.5 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain morpholin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 42b (45 mg) with a yield of 90.24%. The crude product was directly used in the next reaction.

MS m/z (ESI): 572.3 [M+1].

### Step 2

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(morpholin-3-yl)methanone

Morpholin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 42b (45 mg, 78.72 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(morpholin-3-yl)methanone 42 (10.35 mg) with a yield of 20.33%.

MS m/z (ESI): 488.3 [M+1].

### Example 43

### (S)-1-(1-((5-(4-((1-(2-(1H-1,2,4-triazol-1-yl)ethyl)-1,2,3,6-tetrahydropyridin-4-yl)ethynyl)phenyl)isoxazol-3-yl)methyl)-1H-imidazol-2-yl)ethan-1-ol

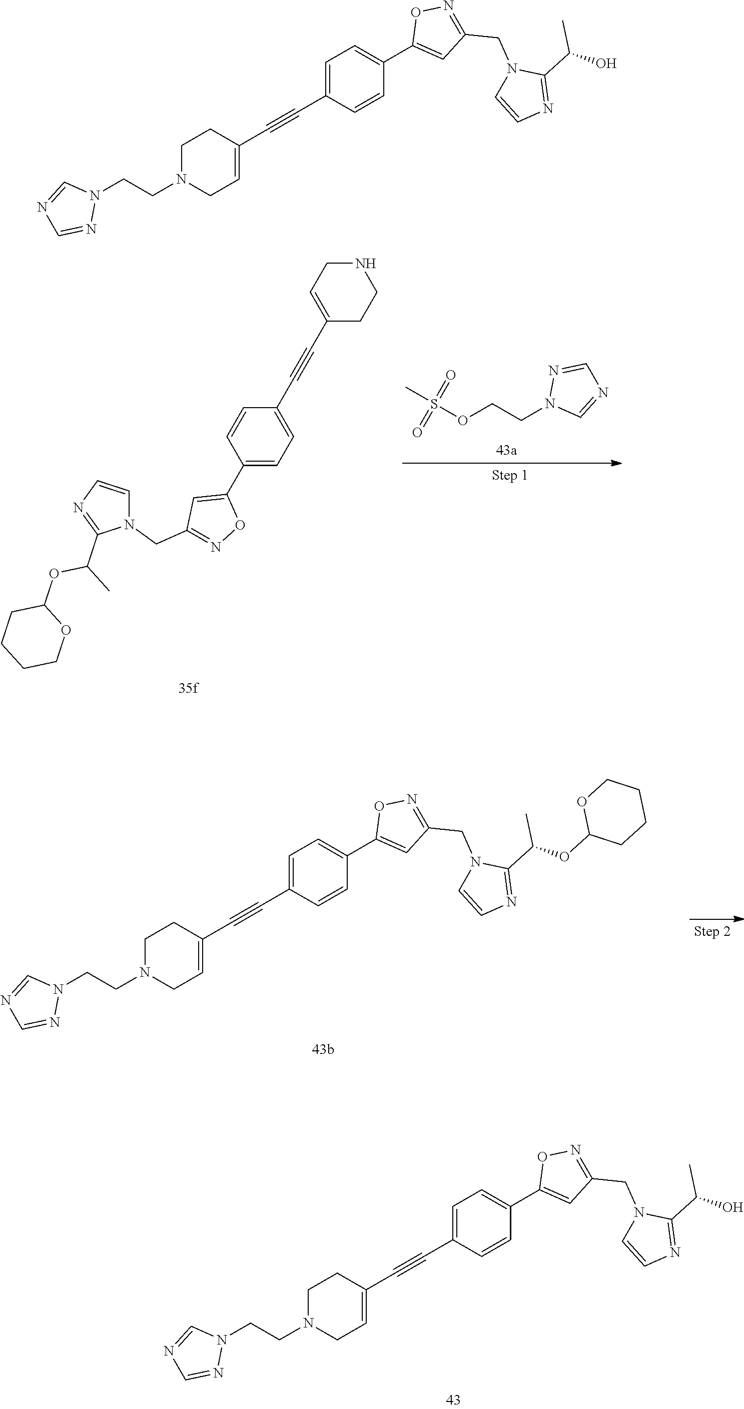 [SMILES: C[C@H](O)c1nccn1Cc1cc(-c2ccc(C#CC3=CCN(CCn4cncn4)CC3)cc2)on1 ; CC(OC1CCCCO1)c1nccn1Cc1cc(-c2ccc(C#CC3=CCNCC3)cc2)on1 ; CS(=O)(=O)OCCn1cncn1 ; C[C@H](OC1CCCCO1)c1nccn1Cc1cc(-c2ccc(C#CC3=CCN(CCn4cncn4)CC3)cc2)on1 ; C[C@H](O)c1nccn1Cc1cc(-c2ccc(C#CC3=CCN(CCn4cncn4)CC3)cc2)on1]

### Step 1

### 5-(4-((1-(2-(1H-1,2,4-triazol-1-yl)ethyl)-1,2,3,6-tetrahydropyridin-4-yl)ethynyl)phenyl)-3-((2-((1S)-1-((tetrahydro-2H-pyran-2-yl)oxy)ethyl)-1H-imidazol-1-yl)methyl)isoxazole

2-(1H-1,2,4-triazol-1-yl) ethyl methanesulfonate 43a (25.02 mg, 130.85 μmol), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (30 mg, 65.42 μmol), and potassium carbonate (9.04 mg, 65.42 μmol) were added to acetonitrile (2 mL) in sequence and continuously stirred at 60 °C for 16 hours. After the reaction was completed, the reaction solution was added with water (50 mL), and extracted with dichloromethane (50 mL×3). The organic phases were combined, dried with anhydrous sodium sulfate, and concentrated to obtain 5-(4-((1-(2-(1H-1,2,4-triazol-1-yl)ethyl)-1,2,3,6-tetrahydropyridin-4-yl)ethynyl)phenyl)-3-((2-((1 S)-1-((tetrahydro-2H-pyran-2-yl)oxy)ethyl)-1H-imidazol-1-yl)methyl)isoxazole 43b (35 mg) with a yield of 96.63%. The crude product was directly used for the next reaction.

MS m/z (ESI): 554.4 [M+1].

### Step 2

### (S)-1-(1-((5-(4-((1-(2-(1H-1,2,4-triazol-1-yl)ethyl)-1,2,3,6-tetrahydropyridin-4-yl)ethynyl)phenyl)isoxazol-3-yl)methyl)-1H-imidazol-2-yl)ethan-1-ol

5-(4-((1-(2-(1H-1,2,4-triazol-1-yl)ethyl)-1,2,3,6-tetrahydropyridin-4-yl)ethynyl)phenyl)-3-((2-((1S)-1-((tetrahydro-2H-pyran-2-yl)oxy)ethyl)-1H-imidazol-1-yl)methyl)isoxazole 43b (35 mg, 63.22 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm I.D; 5 μm, 20 mL/min; mobile phase A: 0.05% TFA+H₂O, mobile phase B: CH₃CN) to obtain (S)-1-(1-((5-(4-((1-(2-(1H-1,2,4-triazol-1-yl)ethyl)-1,2,3,6-tetrahydropyridin-4-yl)ethynyl)phenyl)isoxazol-3-yl)methyl)-1H-imidazol-2-yl)ethan-1-ol 43 (10.69 mg) with a yield of 28.98%.

MS m/z (ESI): 470.3 [M+1].

### Example 44

### (S)-3-(4-((4-(3-((2-(1-hydroxyethyl)-1H-imidazol-1-yl)methyl)isoxazol-5-yl)phenyl)ethynyl)-3,6-dihydropyridin-1(2H)-yl)propanenitrile

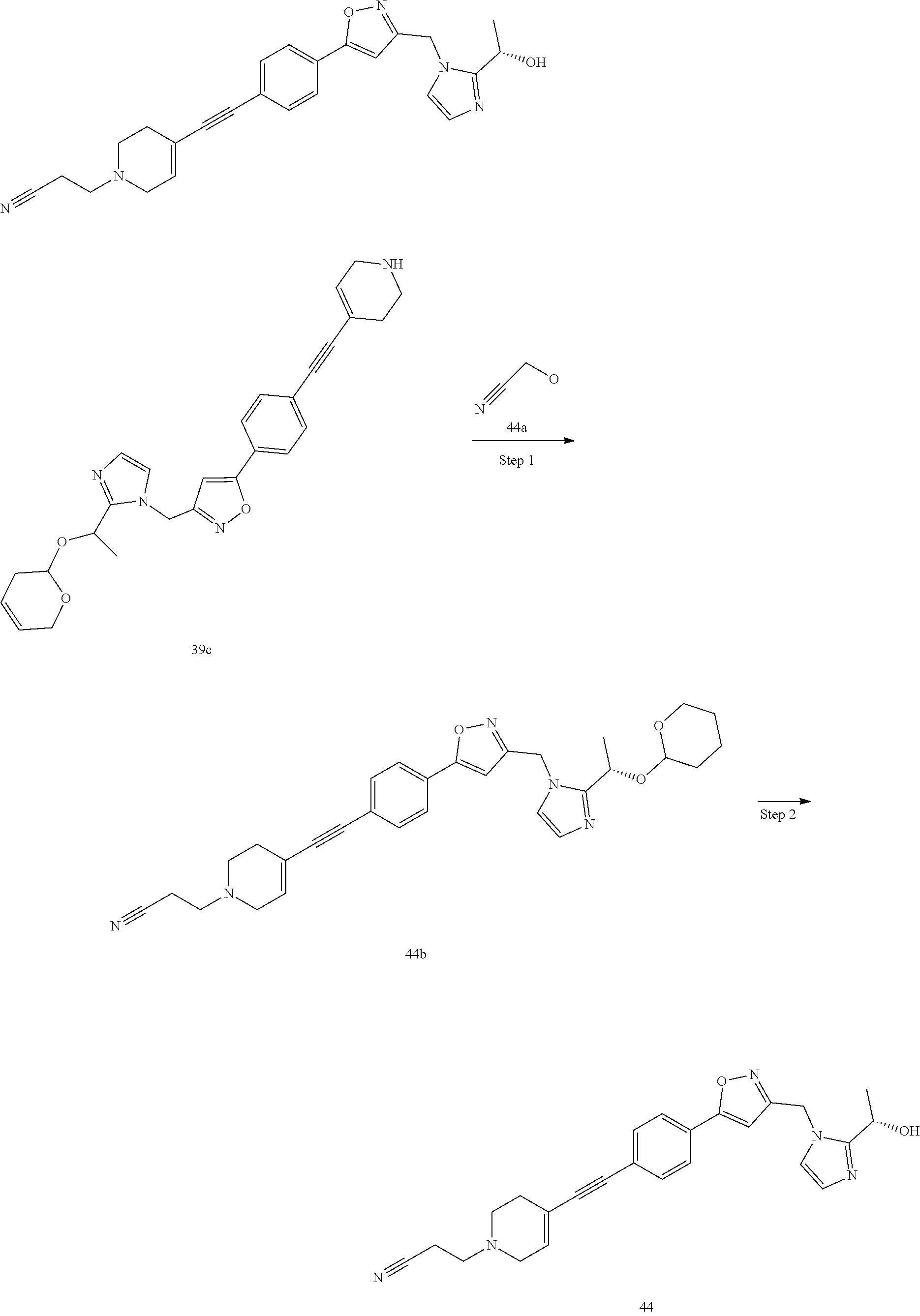

### Step 1

### 3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)propanenitrile

Acrylonitrile 44a (13.89 mg, 261.69 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (60 mg, 130.85 μmol), and potassium carbonate (36.17 mg, 261.69 μmol) were sequentially added to acetonitrile (1 mL) and water (0.5 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain 3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)propanenitrile 44b (66 mg) with a yield of 98.59%. The crude product was directly used in the next reaction.

MS m/z (ESI): 512.3 [M+1].

### Step 2

### (S)-3-(4-((4-(3-((2-(1-hydroxyethyl)-1H-imidazol-1-yl)methyl)isoxazol-5-yl)phenyl)ethynyl)-3,6-dihydropyridin-1(2H)-yl)propanenitrile

3-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)propanenitrile 44b (66 mg, 129.00 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-3-(4-((4-(3-((2-(1-hydroxyethyl)-1H-imidazol-1-yl)methyl)isoxazol-5-yl)phenyl)ethynyl)-3,6-dihydropyridin-1(2H)-yl)propanenitrile 44 (32.38 mg) with a yield of 46.35%.

MS m/z (ESI): 428.2 [M+1].

### Example 45

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-3-yl)methanone

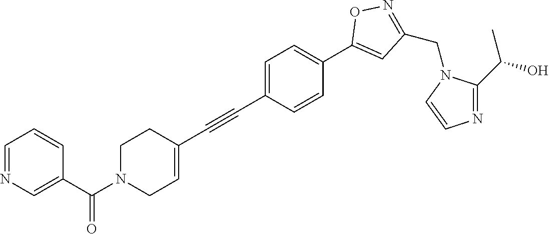 [SMILES: C[C@H](O)c1nccn1Cc1cc(-c2ccc(C#CC3=CCN(C(=O)c4cccnc4)CC3)cc2)on1]

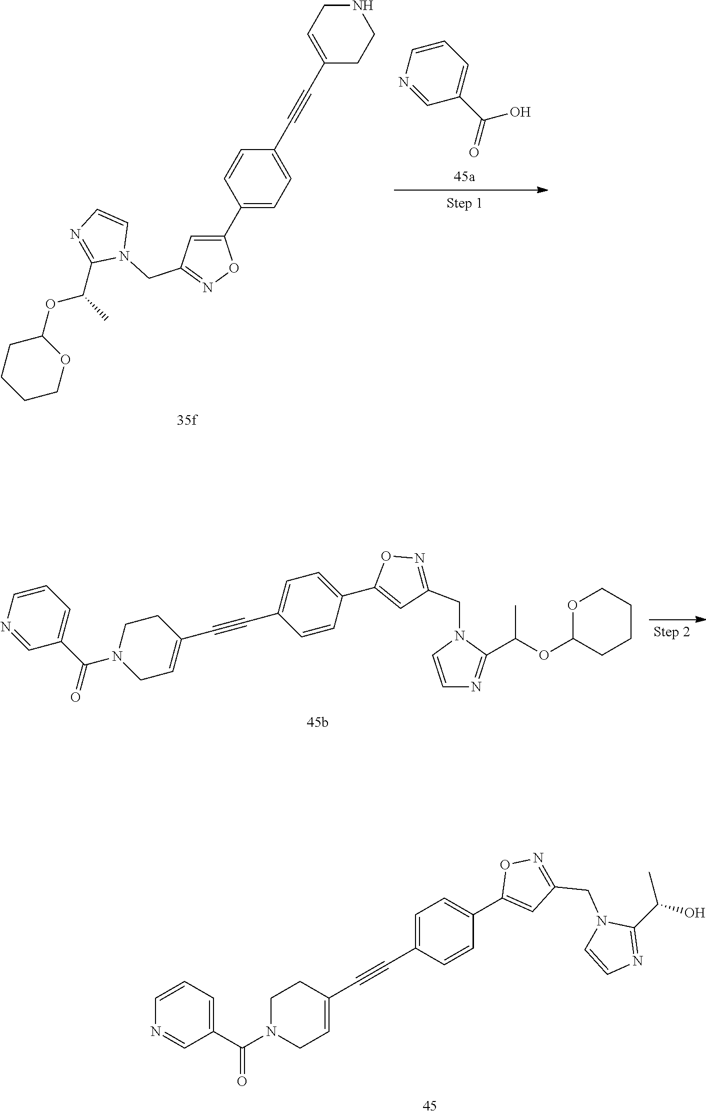

### Step 1

### Pyridin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

Nicotinic acid 45a (21.48 mg, 174.46 μmol, commercially available), 3-((2-((1S)-1-((tetrahydro-2H-pyran-2-yl)oxy)ethyl)-1H-imidazol-1-yl)methyl)-5-(4-((1,2,3,6-tetrahydropyridin-4-yl)ethynyl)phenyl)isoxazole 35f (40 mg, 87.23 μmol), 1-hydroxybenzotriazole (23.57 mg, 174.46 μmol), N-methylmorpholine (26.47 mg, 261.69 μmol), and 1-(3-dimethylaminopropyl)-3-ethylcarbodiimide hydrochloride (33.44 mg, 174.46 μmol) were sequentially added to dichloromethane (1.5 mL), and continuously stirred at room temperature for 16 hours. After the reaction was completed, the reaction solution was added with water (50 mL), and extracted with dichloromethane (50 mL×3). The organic phases were combined, dried with anhydrous sodium sulfate, and concentrated to obtain pyridin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 45b (49 mg) with a yield of 99.66%. The crude product was directly used for the next reaction.

MS m/z (ESI): 564.3 [M+1].

### Step 2

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-3-yl)methanone

Pyridin-3-yl(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone 45b (49 mg, 86.93 μmol) was added to dichloromethane (1.5 mL), and then dropwise added with trifluoroacetic acid (0.5 mL) and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain Pyridin-3-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 45 (37.08 mg) with a yield of 70.42%0.

MS m/z (ESI): 480.2 [M+1].

Examples 46-56 were synthesized according to the synthesis methods of Examples 1-45 of the present invention, and the structures and characterization data can be seen in the following table.

| No. of | | | MS m/z |
|---|---|---|---|
| example | Structure | Name | (ESI) |
| 46 | 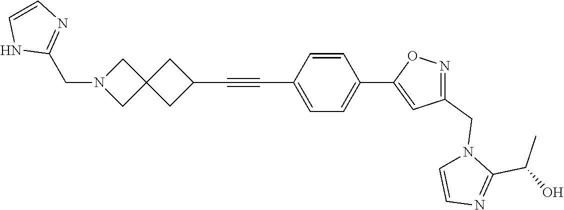 [SMILES: C[C@H](O)c1nccn1Cc1cc(-c2ccc(C#CC3CC4(C3)CN(Cc3ncc[nH]3)C4)cc2)on1] | (S)-1-(1-((5-(4-((2- ((1H-imidazol-2-yl) methyl)-2-azaspiro3.3] heptan-6-yl)ethynyl) phenyl)isoxazol-3-yl) methyl)-1H-imidazol-2- yl)ethan-1-ol | 469.3 [M + 1] |
| 47 | 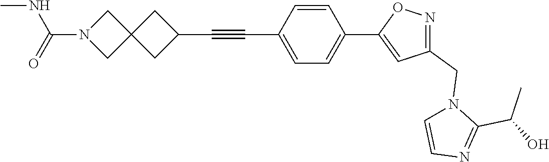 [SMILES: CNC(=O)N1CC2(CC(C#Cc3ccc(-c4cc(Cn5ccnc5[C@H](C)O)no4)cc3)C2)C1] | (S)-6-((4-(3-((2-(1- hydroxyethyl)-1H- imidazol-1-yl)methyl) isoxazol-5-yl)phenyl) ethynyl)-N-methyl-2- azaspiro[3.3]heptane-2- carboxamide | 446.3 [M + 1] |
| 48 | 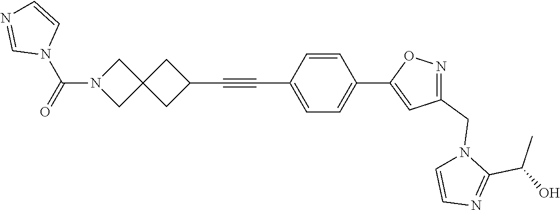 [SMILES: C[C@H](O)c1nccn1Cc1cc(-c2ccc(C#CC3CC4(C3)CN(C(=O)n3ccnc3)C4)cc2)on1] | (S)-(6-((4-(3-((2-(1- hydroxyethyl)-1H- imidazol-1-yl)methyl) isoxazol-5-yl)phenyl) ethynyl)-2-azaspiro[3.3] heptan-2-yl)(1H-imidazol- 1-yl)methanone | 483.2 [M + 1] |
| 49 | 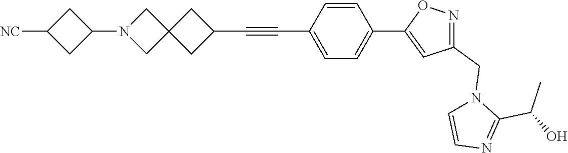 [SMILES: C[C@H](O)c1nccn1Cc1cc(-c2ccc(C#CC3CC4(C3)CN(C3CC(C#N)C3)C4)cc2)on1] | (S)-3-(6-((4-(3-((2-(1- hydroxyethyl)-1H- imidazol-1-yl)methyl) isoxazol-5-yl)phenyl) ethynyl)-2-azaspiro[3.3] heptan-2-yl)cyclobutane- 1-carbonitrile | 468.1 [M + 1] |
| 50 | 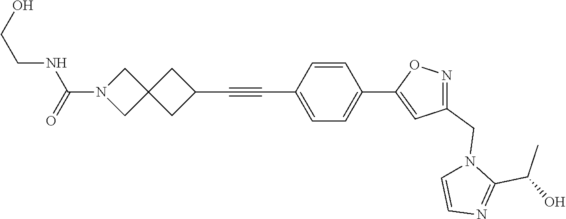 [SMILES: C[C@H](O)c1nccn1Cc1cc(-c2ccc(C#CC3CC4(C3)CN(C(=O)NCCO)C4)cc2)on1] | (S)-N-(2-hydroxyethyl)- 6-((4-(3-((2-(1- hydroxyethyl)-1H- imidazol-1-yl)methyl) isoxazol-5-yl)phenyl) ethynyl)-2-azaspiro[3.3] heptane-2-carboxamide | 476.1 [M + 1] |
| 51 | 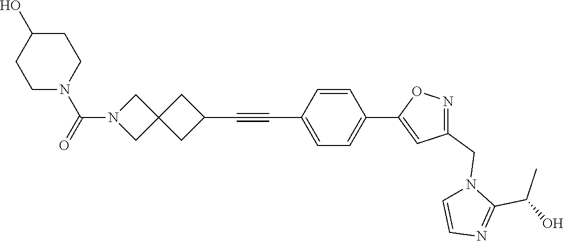 [SMILES: C[C@H](O)c1nccn1Cc1cc(-c2ccc(C#CC3CC4(C3)CN(C(=O)N3CCC(O)CC3)C4)cc2)on1] | (S)-(6-((4-(3-((2-(1- hydroxyethyl)-1H- imidazol-1-yl)methyl) isoxazol-5-yl)phenyl) ethynyl)-2-azaspiro[3.3] heptan-2-yl)(4- hydroxypiperidin-1-yl) methanone | 516.3 [M + 1] |
| 52 | 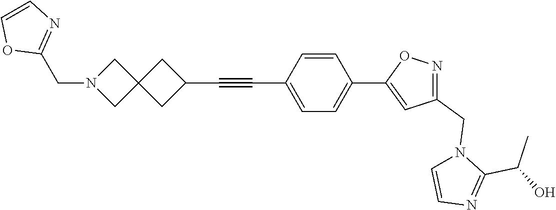 [SMILES: C[C@H](O)c1nccn1Cc1cc(-c2ccc(C#CC3CC4(C3)CN(Cc3ncco3)C4)cc2)on1] | (S)-1-(1-((5-(4-((2- (oxazol-2-ylmethyl)-2- azaspiro[3.3]heptan-6-yl) ethynyl)phenyl)isoxazol- 3-yl)methyl)-1H-imidazol- 2-yl)ethan-1-ol | 470.2 [M + 1] |
| 53 | 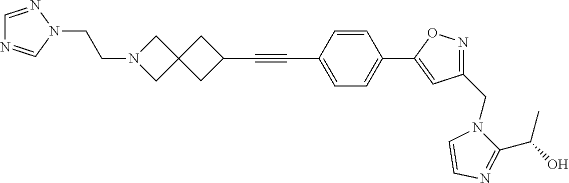 [SMILES: C[C@H](O)c1nccn1Cc1cc(-c2ccc(C#CC3CC4(C3)CN(CCn3cncn3)C4)cc2)on1] | (S)-1-(1-((5-(4-((2-(2- (1H-1,2,4-triazol-1-yl) ethyl)-2-azaspiro[3.3] heptan-6-yl)ethynyl) phenyl)isoxazol-3-yl) methyl)-1H-imidazol-2- yl)ethan-1-ol | 484.3 [M + 1] |
| 54 | 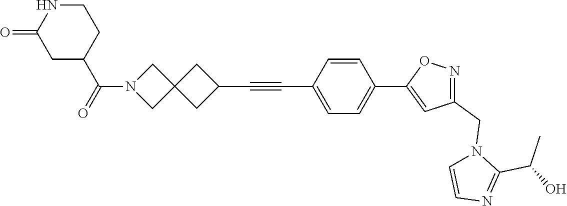 [SMILES: C[C@H](O)c1nccn1Cc1cc(-c2ccc(C#CC3CC4(C3)CN(C(=O)C3CCNC(=O)C3)C4)cc2)on1] | 4-(6-((4-(3-((2-((S)-1- hydroxyethyl)-1H- imidazol-1-yl)methyl) isoxazol-5-yl)phenyl) ethynyl)-2-azaspiro[3.3] heptane-2-carbonyl) piperidin-2-one | 514.2 [M + 1] |
| 55 | 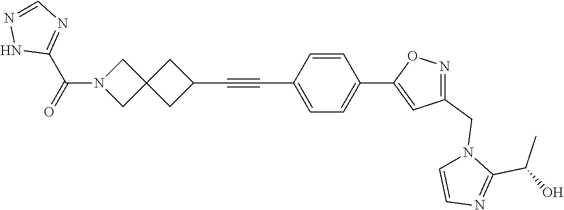 [SMILES: C[C@H](O)c1nccn1Cc1cc(-c2ccc(C#CC3CC4(C3)CN(C(=O)c3ncn[nH]3)C4)cc2)on1] | (S)-(6-((4-(3-((2-(1- hydroxyethyl)-1H- imidazol-1-yl)methyl) isoxazol-5-yl)phenyl) ethynyl)-2-azaspiro[3.3] heptan-2-yl)(1H-1,2,4- triazol-5-yl)methanone | 484.2 [M + 1] |
| 56 | 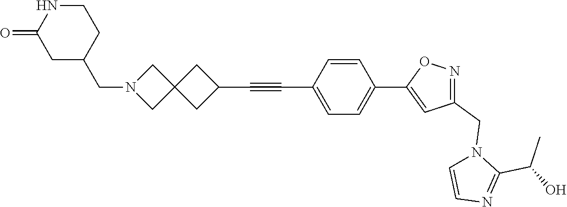 [SMILES: C[C@H](O)c1nccn1Cc1cc(-c2ccc(C#CC3CC4(C3)CN(CC3CCNC(=O)C3)C4)cc2)on1] | 4-((6-((4-(3-((2-((S)-1- hydroxyethyl)-1H- imidazol-1-yl)methyl) isoxazol-5-yl)phenyl) ethynyl)-2-azaspiro[3.3] heptan-2-yl)methyl) piperidin-2-one | 500.3 [M + 1] |

### Example 57

### (S)-4-((4-(3-((2-(1-hydroxyethyl)-1H-imidazol-1-yl)methyl)isoxazol-5-yl)phenyl)ethynyl)-3,6-dihydropyridine-1(2H)-carbaldehyde

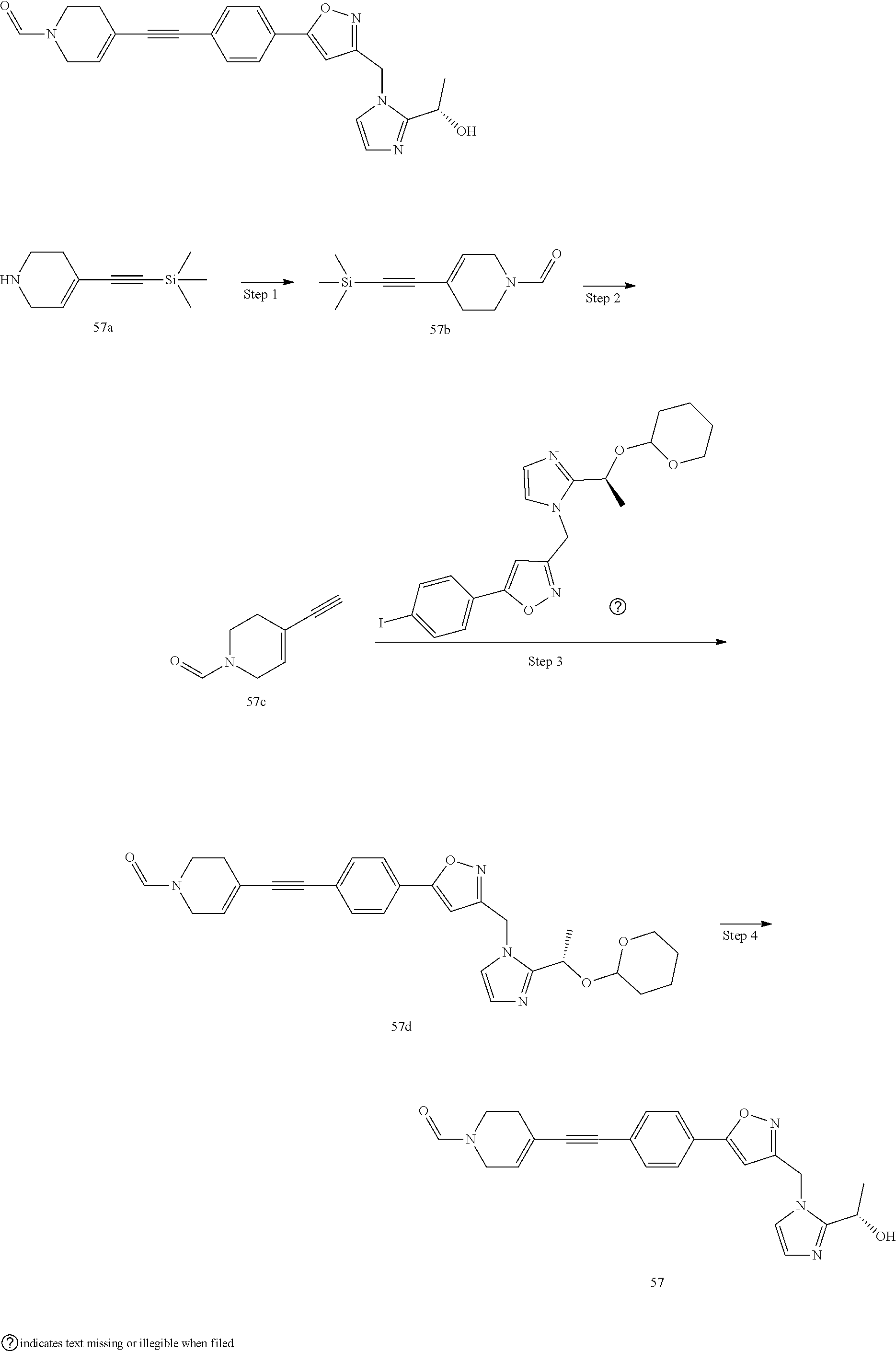

### Step 1

### 4-((trimethylsilyl)ethynyl)-3,6-dihydropyridine-1(2H)-carbaldehyde

4-((trimethylsilyl)ethynyl-1,2,3,6-tetrahydropyridine 57a (250 mg, 1.16 mmol, commercially available), and triethylamine (468.92 mg, 4.63 mmol, 642.35 μL) were sequentially added to formamide (5.13 mL), continuously stirred at room temperature for 12 hours, added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), dried with anhydrous sodium sulfate, filtered, and concentrated under reduced pressure to dryness to obtain 4-((trimethylsilyl)ethynyl)-3,6-dihydropyridine-1(2H)-carbaldehyde 57b (220 mg), which was directly used for the next reaction.

MS m/z (ESI): 208.0 [M+1].

### Step 2

### 4-ethynyl-3,6-dihydropyridine-1(2H)-carbaldehyde

4-((trimethylsilyl)ethynyl)-3,6-dihydropyridine-1(2H)-carbaldehyde 57b (220 mg, 1.06 mmol), and potassium fluoride (308.23 mg, 5.31 mmol) were sequentially added to methanol (5 mL), and continuously stirred at room temperature for 3 hours. The system was concentrated to dryness under reduced pressure, and added with acetonitrile (3 mL) for filtering, and the filtrate was concentrated under reduced pressure to dryness to obtain 4-ethynyl-3,6-dihydropyridine-1(2H)-carbaldehyde 57c (140 mg), which was directly used for the next reaction.

MS m/z (ESI): 136.0 [M+1].

### Step 3

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carbaldehyde

5-(4-iodophenyl)-3-((2-((1S)-1-((tetrahydro-2H-pyran-2-yl)oxy)ethyl)-1H-imidazol-1-yl)methyl)isoxazole 1i (250 mg, 495.50 μmol), 4-ethynyl-3,6-dihydropyridine-1(2H)-carbaldehyde 57c (118.19 mg, 743.26 μmol), triethylene diamine (111.16 mg, 991.01 μmol) and allylpalladium(II) chloride dimer (36.17 mg, 99.10 μmol) were sequentially added to acetonitrile (2.0 mL), replaced thrice with argon gas, continuously stirred at room temperature for 3 hours, and then added with ethyl acetate (30 mL) and water (15 mL) for liquid separation. The aqueous phases were extracted with ethyl acetate (30 mL×2)). The combined organic phases were washed with saturated sodium chloride solution (30 mL×2), dried with anhydrous sodium sulfate, filtered and concentrated to dryness under reduced pressure. The residue was separated and purified by silicagel column chromatography (eluent: system A) to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carbaldehyde 57d (240 mg), with a yield of 89.59%.

MS m/z (ESI): 487.3 [M+1].

### Step 4

### (S)-4-((4-(3-((2-(1-hydroxyethyl)-1H-imidazol-1-yl)methyl)isoxazol-5-yl)phenyl)ethynyl)-3,6-dihydropyridine-1(2H)-carbaldehyde

4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carbaldehyde 57d (220 mg, 452.15 μmol), and trifluoroacetic acid (0.5 mL) were added to dichloromethane (2 mL), and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)-3,6-dihydropyridine-1(2H)-carbaldehyde 57 (59.7 mg), with a yield of 25.05%.

MS m/z (ESI): 402.9 [M+1]. ¹H NMR (400 MHz, DMSO-d₆) δ 8.09 (s, 1H), 7.91-7.85 (m, 2H), 7.79 (d, J=2.0 Hz, 1H), 7.71 (d, J=2.0 Hz, 1H), 7.61 (d, J=8.2 Hz, 2H), 7.18 (s, 1H), 6.32-6.21 (m, 1H), 5.73 (s, 2H), 5.27 (q, J=6.6 Hz, 1H), 4.04 (dq, J=20.9, 3.0 Hz, 2H), 3.55 (t, J=5.7 Hz, 2H), 2.36 (dq, J=5.8, 2.9 Hz, 1H), 2.26 (h, J=2.7 Hz, 1H), 1.50 (d, J=6.6 Hz, 3H).

### Example 58

### (S)-4-((4-(3-((2-(1-hydroxyethyl)-1H-imidazol-1-yl)methyl)isoxazol-5-yl)phenyl)ethynyl)-3,6-dihydropyridine-1(2H)-carboxamide

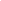

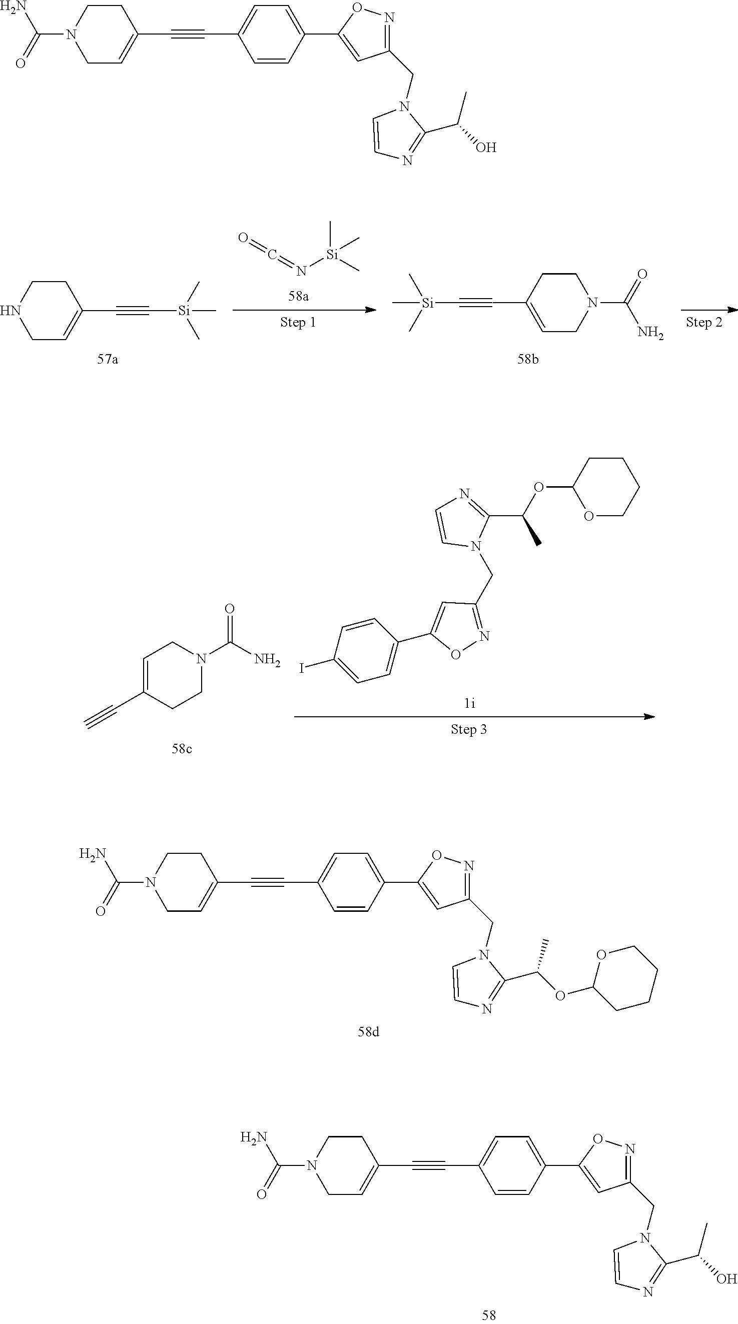

### Step 1

### 4-((trimethylsilyl)ethynyl)-3,6-dihydropyridine-1(2H)-carboxamide

4-((trimethylsilyl)ethynyl-1,2,3,6-tetrahydropyridine 57a (50 mg, 278.81 μmol), and triethylamine (84.64 mg, 836.43 μmol, 115.94 μL) were sequentially added to tetrahydrofuran (1.95 mL), then added with isocyanatotrimethylsilane 58a (41.76 mg, 362.45 μmol, 49.13 μL, commercially available), and reacted at room temperature for 16 hours. After the reaction was completed, the reaction solution was concentrated to obtain 4-((trimethylsilyl)ethynyl)-3,6-dihydropyridine-1(2H)-carboxamide 58b (50 mg), which was directly used for the next reaction.

MS m/z (ESI): 223.0 [M+1].

### Step 2

### 4-ethynyl-3,6-dihydropyridine-1(2H)-carboxamide

4-((trimethylsilyl)ethynyl)-3,6-dihydropyridine-1(2H)-carboxamide 58b (50 mg, 224.86 μmol), and potassium fluoride (65.32 mg, 1.12 mmol) were sequentially added into methanol (2 mL), and continuously stirred at room temperature for 12 hours. The system was concentrated to dryness under reduced pressure, and added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), and concentrated under reduced pressure to obtain 4-ethynyl-3,6-dihydropyridine-1(2H)-carboxamide 58c (20 mg), which was directly used for the next reaction.

MS m/z (ESI): 151.1 [M+1].

### Step 3

### 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxamide

In an argon gas atmosphere, 5-(4-iodophenyl)-3-((2-((1S)-1-((tetrahydro-2H-pyran-2-yl)oxy)ethyl)-1H-imidazol-1-yl)methyl)isoxazole 1i (50 mg, 104.32 μmol), 4-ethynyl-3,6-dihydropyridine-1(2H)-carboxamide 58c (23.50 mg, 156.47 μmol), triethylene diamine (23.40 mg, 208.63 μmol), allylpalladium(II) chloride dimer (7.62 mg, 20.86 μmol), and tri-tert-butylphosphine (10% in toluene solution) (42.21 mg, 20.86 μmol, 10% purity) were sequentially added to acetonitrile (2 mL), replaced with argon gas, reacted at room temperature for 4 hours, and then added with ethyl acetate (30 mL) and water (15 mL) for liquid separation. The aqueous phases were extracted with ethyl acetate (30 mL×2). The combined organic phases were washed with saturated sodium chloride solution (30 mL×2), dried with anhydrous sodium sulfate, filtered and concentrated to dryness under reduced pressure.

The residue was separated and purified by silicagel column chromatography (eluent: system A) to obtain 4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxamide 58d (40 mg), with a yield of 76.45%.

MS m/z (ESI): 502.3 [M+1].

### Step 4

### (S)-4-((4-(3-((2-(1-hydroxyethyl)-1H-imidazol-1-yl)methyl)isoxazol-5-yl)phenyl)ethynyl)-3,6-dihydropyridine-1(2H)-carboxamide

4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridine-1(2H)-carboxamide 58d (40 mg, 79.75 μmol), and trifluoroacetic acid (0.5 mL) were sequentially added to dichloromethane (2 mL), and continuously stirred at room temperature for one hour. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-4-((4-(3-((2-(1-hydroxyethyl)-1H-imidazol-1-yl)methyl)isoxazol-5-yl)phenyl)ethynyl)-3,6-dihydropyridine-1(2H)-carboxamide 58 (13.5 mg), with a yield of 30.26%.

MS m/z (ESI): 418.2 [M+1].

### Example 59

### (S)-2-(4-((4-(3-((2-(1-hydroxyethyl)-1H-imidazol-1-yl)methyl)isoxazol-5-yl)phenyl)ethynyl)-3,6-dihydropyridin-1(2H)-yl)acetamide

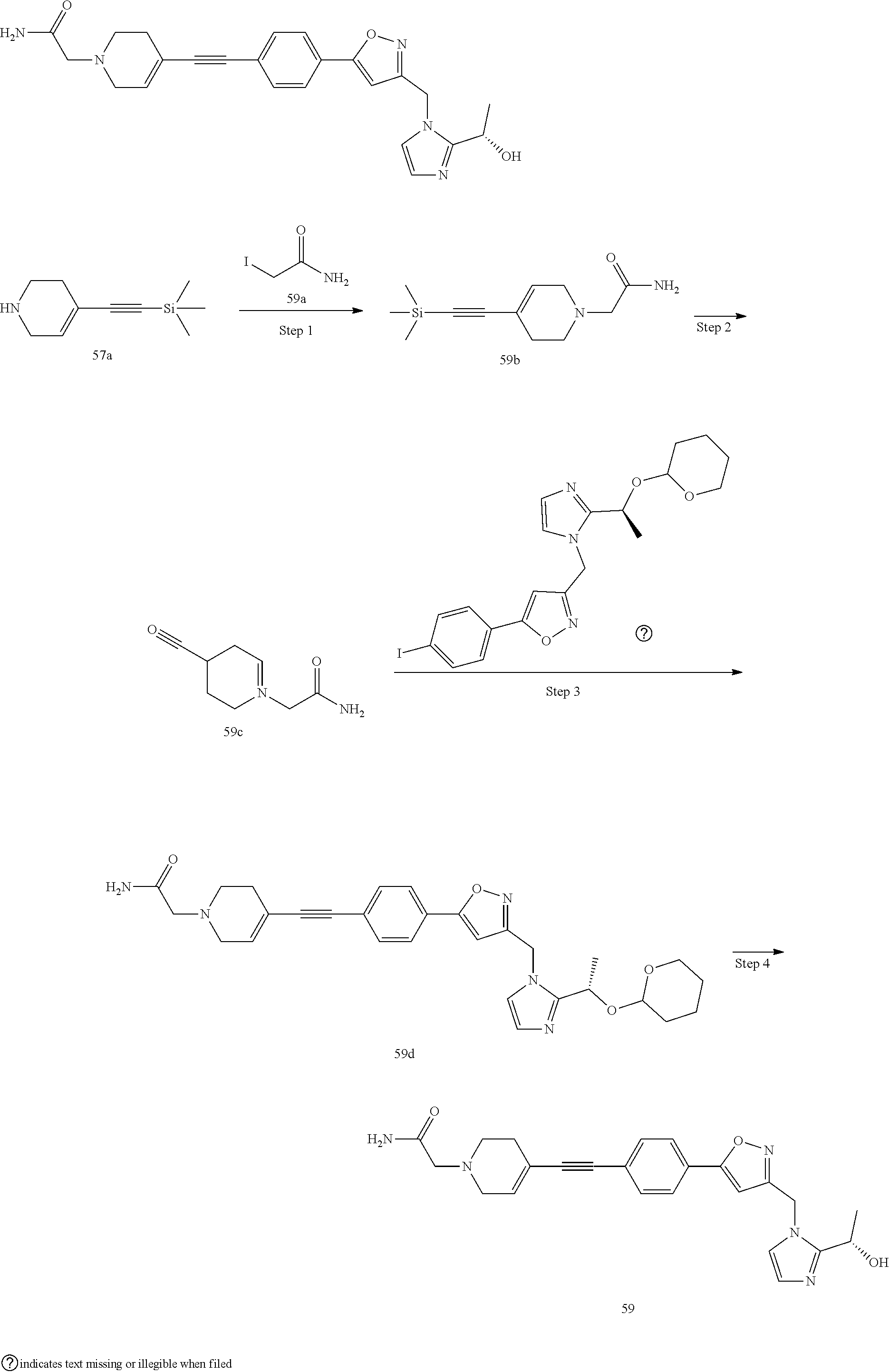

### Step 1

### 2-(4-((trimethylsilyl)ethynyl)-3,6-dihydropyridin-1(2H)-yl)acetamide

2-iodoacetamide 59a (123.77 mg, 669.14 μmol, commercially available), 4-((trimethylsilyl)ethynyl-1,2,3,6-tetrahydropyridine 57a (100 mg, 557.62 μmol), and cesium carbonate (363.37 mg, 1.12 mmol) were sequentially added to acetonitrile (2 mL), and continuously stirred at room temperature for 12 hours. The reaction solution was distilled under reduced pressure to remove the organic solvent, and then added with ethyl acetate (50 mL) and (30 mL) for liquid separation. The aqueous phases were extracted with ethyl acetate (30 mL×2). The organic phases were combined, dried with anhydrous sodium sulfate, distilled under reduced pressure to remove the organic solvent and obtain 2-(4-((trimethylsilyl)ethynyl)-3,6-dihydropyridin-1(2H)-yl)acetamide 59b (130 mg), which was directly used for the next reaction.

MS m/z (ESI): 237.2 [M+1].

### Step 2

### 2-(4-ethynyl-3,6-dihydropyridin-1(2H)-yl)acetamide

2-(4-((trimethylsilyl)ethynyl)-3,6-dihydropyridin-1(2H)-yl)acetamide 59b (130 mg, 549.95 μmol), and potassium fluoride (95.86 mg, 1.65 mmol) were sequentially added to methanol (2 mL), and continuously stirred at room temperature for 16 hours. The reaction solution was distilled under reduced pressure to remove the organic solvent and obtain 2-(4-ethynyl-3,6-dihydropyridin-1(2H)-yl)acetamide 59c (80 mg), which was directly used for the next reaction.

MS m/z (ESI): 165.2 [M+1].

### Step 3

### 2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)acetamide

5-(4-iodophenyl)-3-((2-((1S)-1-((tetrahydro-2H-pyran-2-yl)oxy)ethyl)-1H-imidazol-1-yl)methyl)isoxazole 1i (150 mg, 312.95 μmol), 2-(4-ethynyl-3,6-dihydropyridin-1(2H)-yl)acetamide 59c (56.53 mg, 344.24 μmol), triethylene diamine (70.21 mg, 625.90 μmol), allylpalladium(II) chloride dimer (22.85 mg, 62.59 μmol) and tri-tert-butylphosphine (10% in toluene solution) (12.66 mg, 62.59 μmol) were sequentially added to acetonitrile (2 mL), replaced with argon gas, and reacted at room temperature for 4 hours. The reaction solution was distilled under reduced pressure to remove the organic solvent. The residue was separated and purified by silicagel column chromatography (eluent: system A) to obtain 2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)acetamide 59d (108 mg), with a yield of 66.93%.

MS m/z (ESI): 516.3 [M+1].

### Step 4

### (S)-2-(4-((4-(3-((2-(1-hydroxyethyl)-1H-imidazol-1-yl)methyl)isoxazol-5-yl)phenyl)ethynyl)-3,6-dihydropyridin-1(2H)-yl)acetamide

2-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)acetamide 59d (50 mg, 96.97 μmol), and trifluoroacetic acid (0.5 mL) were sequentially added to dichloromethane (1.5 mL), and continuously stirred at room temperature for 3 hours. The reaction solution was concentrated to obtain a crude product, and the crude product was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-(4-((4-(3-((2-(1-hydroxyethyl)-1H-imidazol-1-yl)methyl)isoxazol-5-yl)phenyl)ethynyl)-3,6-dihydropyridin-1(2H)-yl)acetamide 59 (26.0 mg), with a yield of 43.60%.

MS m/z (ESI): 431.9 [M+1].

### Example 60

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(morpholino)methanone

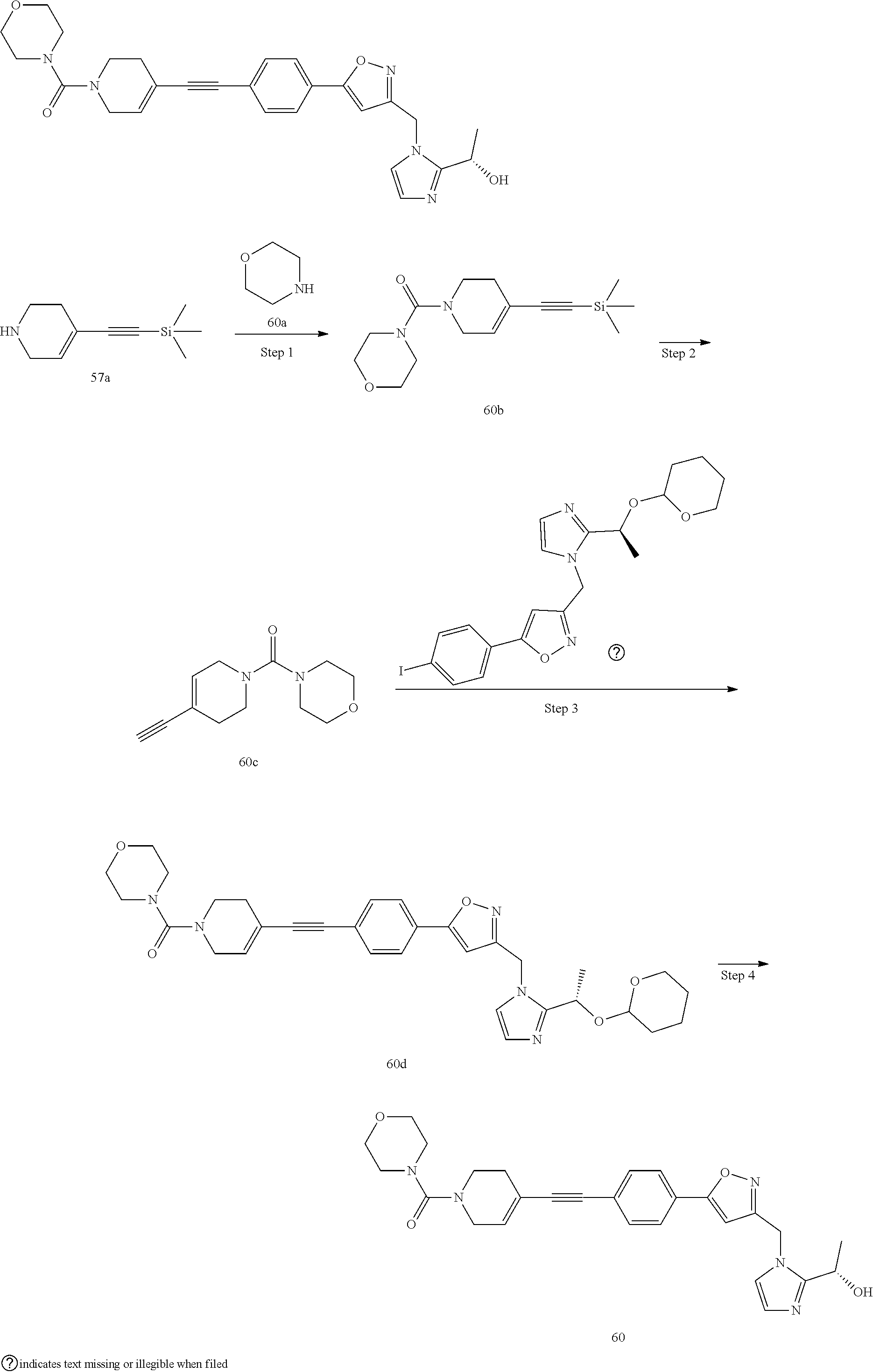

### Step 1

### Morpholino(4-((trimethylsilyl)ethynyl)-3,6-dihydropyridin-1(2H)-yl)methanone

4-((trimethylsilyl)ethynyl-1,2,3,6-tetrahydropyridine 57a (62.50 mg, 278.81 μmol), morpholine 60a (48.58 mg, 557.62 μmol, 49.07 μL), triphosgene (99.28 mg, 334.57 μmol) and triethylamine (169.28 mg, 1.67 mmol, 235.11 μL) were sequentially added to tetrahydrofuran (1.72 mL), continuously stirred at room temperature for 12 hours, and then added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), dried with dried with anhydrous sodium sulfate, filtered and concentrated to dryness under reduced pressure. The residue was separated and purified by silicagel column chromatography (eluent: system A) to obtain morpholino(4-((trimethylsilyl)ethynyl)-3,6-dihydropyridin-1(2H)-yl)methanone 60b (40 mg), with a yield of 49.06%.

MS m/z (ESI): 293.0 [M+1].

### Step 2

### (4-ethynyl-3,6-dihydropyridin-1(2H)-yl)(morpholino)methanone

Morpholino(4-((trimethylsilyl)ethynyl)-3,6-dihydropyridin-1(2H)-yl)methanone 60b (40 mg, 136.78 μmol), potassium fluoride (39.73 mg, 683.88 μmol) were sequentially added to methanol (1 mL), and continuously stirred at room temperature for 4 hours. The system was concentrated to dryness under reduced pressure, and the residue was added with acetonitrile (10 mL), and filtered. The filtrate was concentrated to dryness to obtain (4-ethynyl-3,6-dihydropyridin-1(2H)-yl)(morpholino)methanone 60c (20 mg), which was directly used for the next reaction.

MS m/z (ESI): 221.0 [M+1].

### Step 3

### Morpholino(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

5-(4-iodophenyl)-3-((2-((1S)-1-((tetrahydro-2H-pyran-2-yl)oxy)ethyl)-1H-imidazol-1-yl)methyl)isoxazole 1i (25 mg, 49.55 μmol), (4-ethynyl-3,6-dihydropyridin-1(2H)-yl)(morpholino)methanone 60c (18.19 mg, 74.33 μmol), triethylene diamine (11.12 mg, 99.10 μmol) and allylpalladium(II) chloride dimer (3.62 mg, 9.91 μmol) were sequentially added to acetonitrile (2 mL), replaced thrice with argon gas, and continuously stirred at room temperature for 12 hours. The system was concentrated to dryness under reduced pressure, and the residue was separated and purified by silica column chromatography (eluent: system A) to obtain morpholino(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 60d (20 mg), with a yield of 70.61%.

MS m/z (ESI): 572.0 [M+1].

### Step 4

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(morpholino)methanone

Morpholino(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 60d (20 mg, 34.99 μmol), and trifluoroacetic acid (0.3 mL) were sequentially added to dichloromethane (1 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(morpholino)methanone 60 (2.5 mg), with a yield of 11.11%.

MS m/z (ESI): 487.9 [M+1].

### Example 61

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-2-yl)methanone

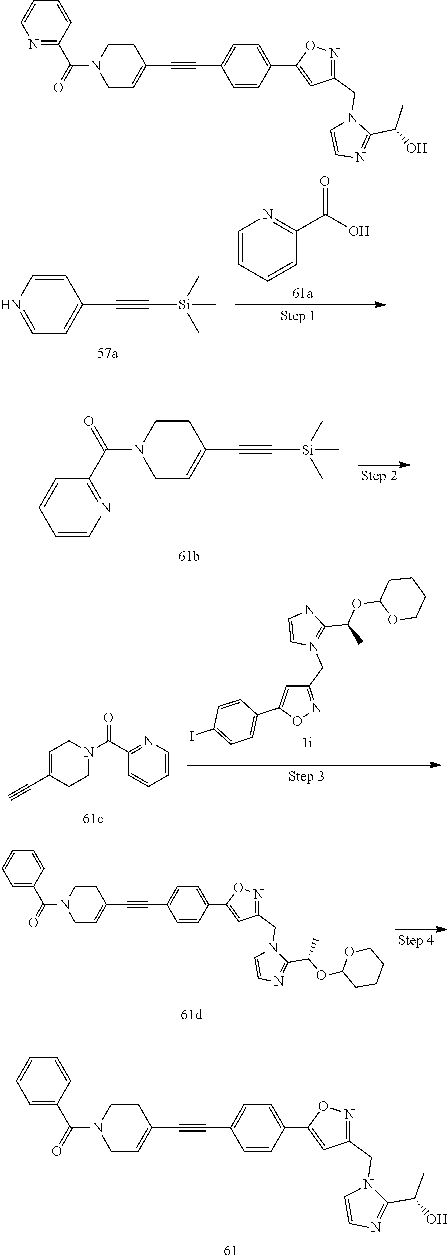

### Step 1

### Pyridin-2-yl(4-((trimethylsilyl)ethynyl)-3,6-dihydropyridin-1(2H)-yl)methanone

4-((trimethylsilyl)ethynyl-1,2,3,6-tetrahydropyridine 57a (50 mg, 278.81 μmol), pyridine carboxylic acid 61a (51.49 mg, 418.22 μmol, commercially available), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (2.0 mL), and reacted at room temperature for 4 hours.

The raw materials reacted completed detected by mass spectrometry, and the system was concentrated to dryness under reduced pressure to obtain pyridin-2-yl(4-((trimethylsilyl)ethynyl)-3,6-dihydropyridin-1(2H)-yl)methanone 61b (55 mg), which was directly used for the next reaction.

MS m/z (ESI): 285.1 [M+1].

### Step 2

### (4-ethynyl-3,6-dihydropyridin-1(2H)-yl)(pyridin-2-yl)methanone

Pyridin-2-yl(4-((trimethylsilyl)ethynyl)-3,6-dihydropyridin-1(2H)-yl)methanone 61b (55 mg, 193.37 μmol), and potassium fluoride (56.17 mg, 966.85 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain (4-ethynyl-3,6-dihydropyridin-1(2H)-yl)(pyridin-2-yl)methanone 61c (30 mg), which was directly used for the next reaction.

MS m/z (ESI): 213.0 [M+1].

### Step 3

### Pyridin-2-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), (4-ethynyl-3,6-dihydropyridin-1(2H)-yl)(pyridin-2-yl)methanone 61c (21.03 mg, 89.19 μmol), triethylene diamine (13.34 mg, 118.92 μmol) and allylpalladium(II) chloride dimer (4.34 mg, 11.89 μmol) were sequentially added to acetonitrile (2 mL), replaced thrice with argon gas, and continuously stirred at room temperature for 12 hours. The system was concentrated to dryness under reduced pressure, and the residue was separated and purified by silica column chromatography (eluent: system A) to obtain pyridin-2-yl(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 61d (25 mg), with a yield of 74.59%.

MS m/z (ESI): 563.9 [M+1].

### Step 4

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-2-yl)methanone

Pyridin-2-yl(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone 61d (25 mg, 44.35 μmol), trifluoroacetic acid (0.5 mL) were sequentially added to dichloromethane (2 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(pyridin-2-yl)methanone 61 (15.2 mg), with a yield of 56.00%.

MS m/z (ESI): 480.2 [M+1].

### Example 62

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one

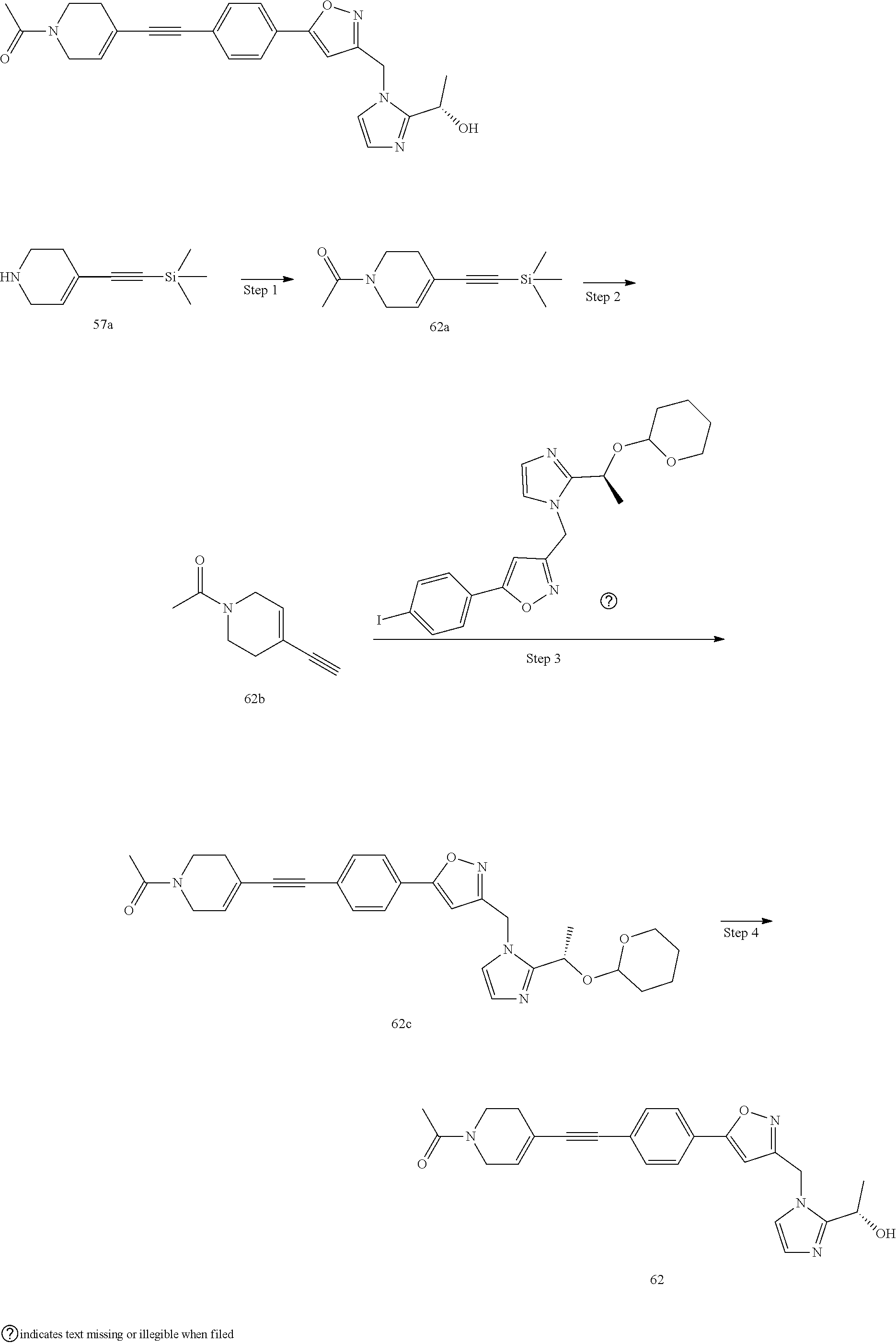

### Step 1

### 1-(4-((trimethylsilyl)ethynyl)-3,6-dihydropyridin-1(2H)-yl)ethan-1-one

4-((trimethylsilyl)ethynyl-1,2,3,6-tetrahydropyridine 57a (50 mg, 278.81 μmol), acetic anhydride (42.70 mg, 418.22 μmol, 39.53 μL) and triethylamine (84.64 mg, 836.43 μmol, 116.26 μL) were sequentially added to dichloromethane (1 mL), and continuously stirred at room temperature for 12 hours. The system was concentrated to dryness under reduced pressure, and the residue was separated and purified by silicagel column chromatography (eluent: system A) to obtain 1-(4-((trimethylsilyl)ethynyl)-3,6-dihydropyridin-1(2H)-yl)ethan-1-one 62a (50 mg), with a yield of 81.01%.

MS m/z (ESI): 222.0 [M+1].

### Step 2

### 1-(4-ethynyl-3,6-dihydropyridin-1(2H)-yl)ethan-1-one

1-(4-((trimethylsilyl)ethynyl)-3,6-dihydropyridin-1(2H)-yl)ethan-1-one 62a (50 mg, 225.87 μmol), and potassium fluoride (65.61 mg, 1.13 mmol) were sequentially added into methanol (1 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain 1-(4-ethynyl-3,6-dihydropyridin-1(2H)-yl)ethan-1-one 62b (30 mg), which was directly used for the next reaction.

MS m/z (ESI): 150.0 [M+1].

### Step 3

### 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), 1-(4-ethynyl-3,6-dihydropyridin-1(2H)-yl)ethan-1-one 62b (14.78 mg, 89.19 μmol), triethylene diamine (13.34 mg, 118.92 μmol) and allylpalladium(II) chloride dimer (4.34 mg, 11.89 μmol) were sequentially added to acetonitrile (2 mL), replaced thrice with argon gas, and continuously stirred at room temperature for 12 hours. The system was concentrated to dryness under reduced pressure, and the residue was separated and purified by silicagel column chromatography (eluent: system A) to obtain 1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 62c (25 mg), with a yield of 83.99%.

MS m/z (ESI): 500.9 [M+1].

### Step 4

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one

1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 62c (25 mg, 49.94 μmol), and trifluoroacetic acid (0.5 mL) were sequentially added to dichloromethane (2 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one 62 (6.8 mg), with a yield of 24.38%.

MS m/z (ESI): 417.2 [M+1].

### Example 63

### (S)-2-hydroxy-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one

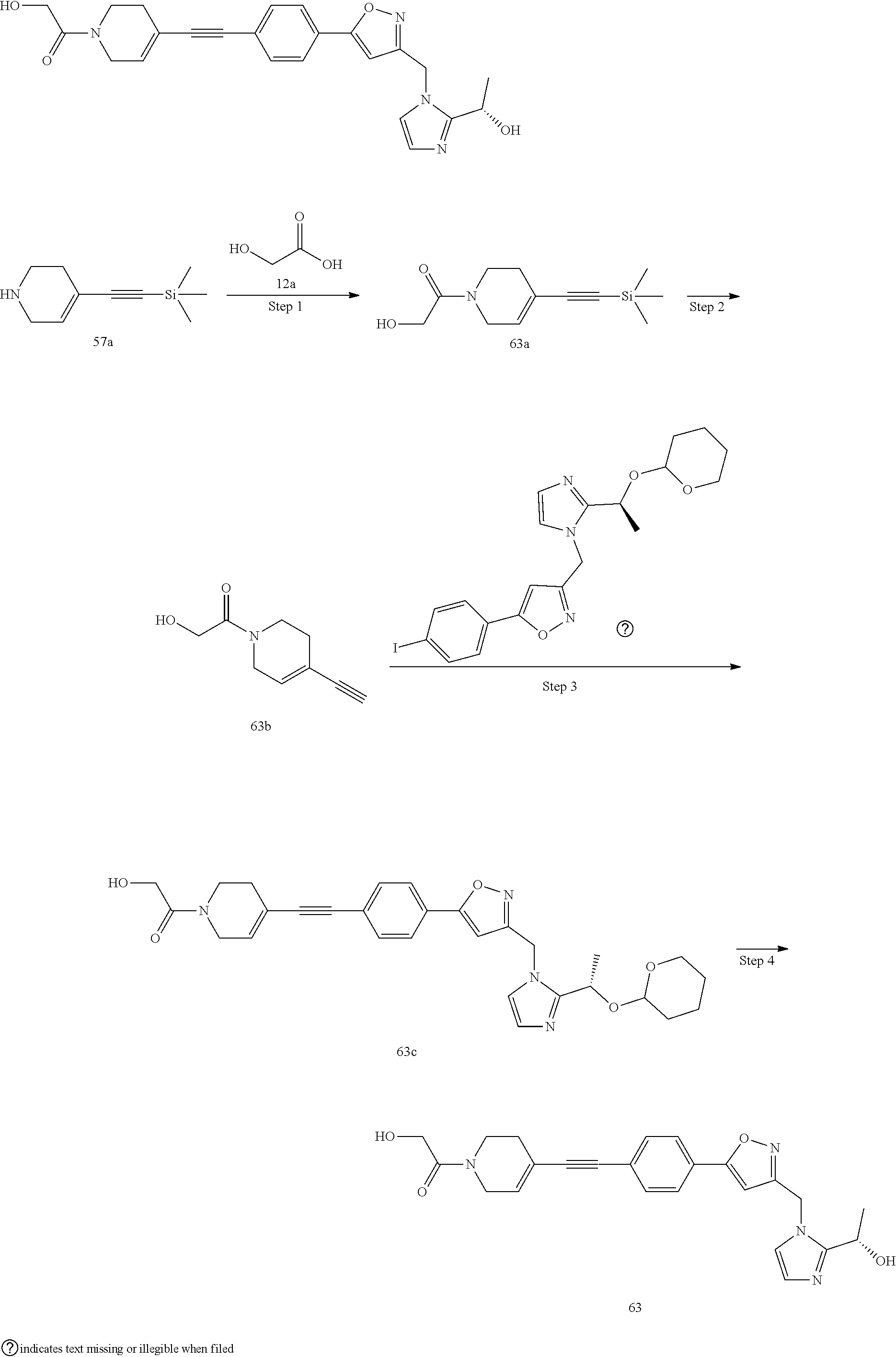

### Step 1

### 2-hydroxy-1-(4-((trimethylsilyl)ethynyl)-3,6-dihydropyridin-1(2H)-yl)ethan-1-one

4-((trimethylsilyl)ethynyl-1,2,3,6-tetrahydropyridine 57a (50 mg, 278.81 μmol), 2-glycolic acid 12a (42.41 mg, 557.62 μmol), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (2.0 mL), and continuously stirred at room temperature for 4 hours. The raw materials reacted completed detected by mass spectrometry, and the system was concentrated to dryness under reduced pressure to obtain 2-hydroxy-1-(4-((trimethylsilyl)ethynyl)-3,6-dihydropyridin-1(2H)-yl)ethan-1-one 63a (40 mg), which was directly used for the next reaction.

MS m/z (ESI): 237.9 [M+1].

### Step 2

### 1-(4-ethynyl-3,6-dihydropyridin-1(2H)-yl)-2-hydroxyethan-1-one

2-hydroxy-1-(4-((trimethylsilyl)ethynyl)-3,6-dihydropyridin-1(2H)-yl)ethan-1-one 63a (40 mg, 168.51 μmol), potassium fluoride (48.95 mg, 842.57 μmol) were sequentially added to methanol (1 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain 1-(4-ethynyl-3,6-dihydropyridin-1(2H)-yl)-2-hydroxyethan-1-one 63b (20 mg), which was directly used for the next reaction.

MS m/z (ESI): 166.0 [M+1].

### Step 3

### 2-hydroxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), 1-(4-ethynyl-3,6-dihydropyridin-1(2H)-yl)-2-hydroxyethan-1-one 63b (16.37 mg, 89.19 μmol), triethylene diamine (13.34 mg, 118.92 μmol), and allylpalladium(II) chloride dimer (4.34 mg, 11.89 μmol) were sequentially added to acetonitrile (2 mL), replaced thrice with argon gas, and continuously stirred at room temperature for 12 hours. The system was concentrated to dryness under reduced pressure, and the residue was separated and purified by silica gel column chromatography (eluent: system A) to obtain 2-hydroxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 63c (25 mg), with a yield of 81.39%.

MS m/z (ESI): 516.9 [M+1].

### Step 4

### (S)-2-hydroxy-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one

2-hydroxy-1-(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)ethan-1-one 63c (25 mg, 48.39 μmol), and trifluoroacetic acid (0.5 mL) were sequentially added to dichloromethane (2 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-2-hydroxy-1-(4-((4-(3-((2-(1-hydroxyethyl)-1H-imidazol-1-yl)methyl)isoxazol-5-yl)phenyl)ethynyl)-3,6-dihydropyridin-1(2H)-yl)ethan-1-one 63 (1.3 mg), with a yield of 4.62%.

MS m/z (ESI): 433.2 [M+1]. ¹H NMR (400 MHz, DMSO-d₆) δ 7.89-7.86 (m, 2H), 7.76-7.75 (d, J=4.0 Hz, 1H), 7.67-7.66 (d, J=4.0 Hz, 1H), 7.62-7.60 (m, 3H), 7.17-7.16 (d, J=4.0 Hz, 1H), 6.27-6.24 (m, 1H), 5.73 (s, 2H), 5.24 (q, J=6.6 Hz, 1H), 4.41-4.04 (m, 4H), 3.64-3.61 (m, 1H), 3.50-3.47 (m, 1H), 2.35-2.28 (m, 2H), 1.50 (d, J=6.6 Hz, 3H).

### Example 64

### (1,4-dioxan-2-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone

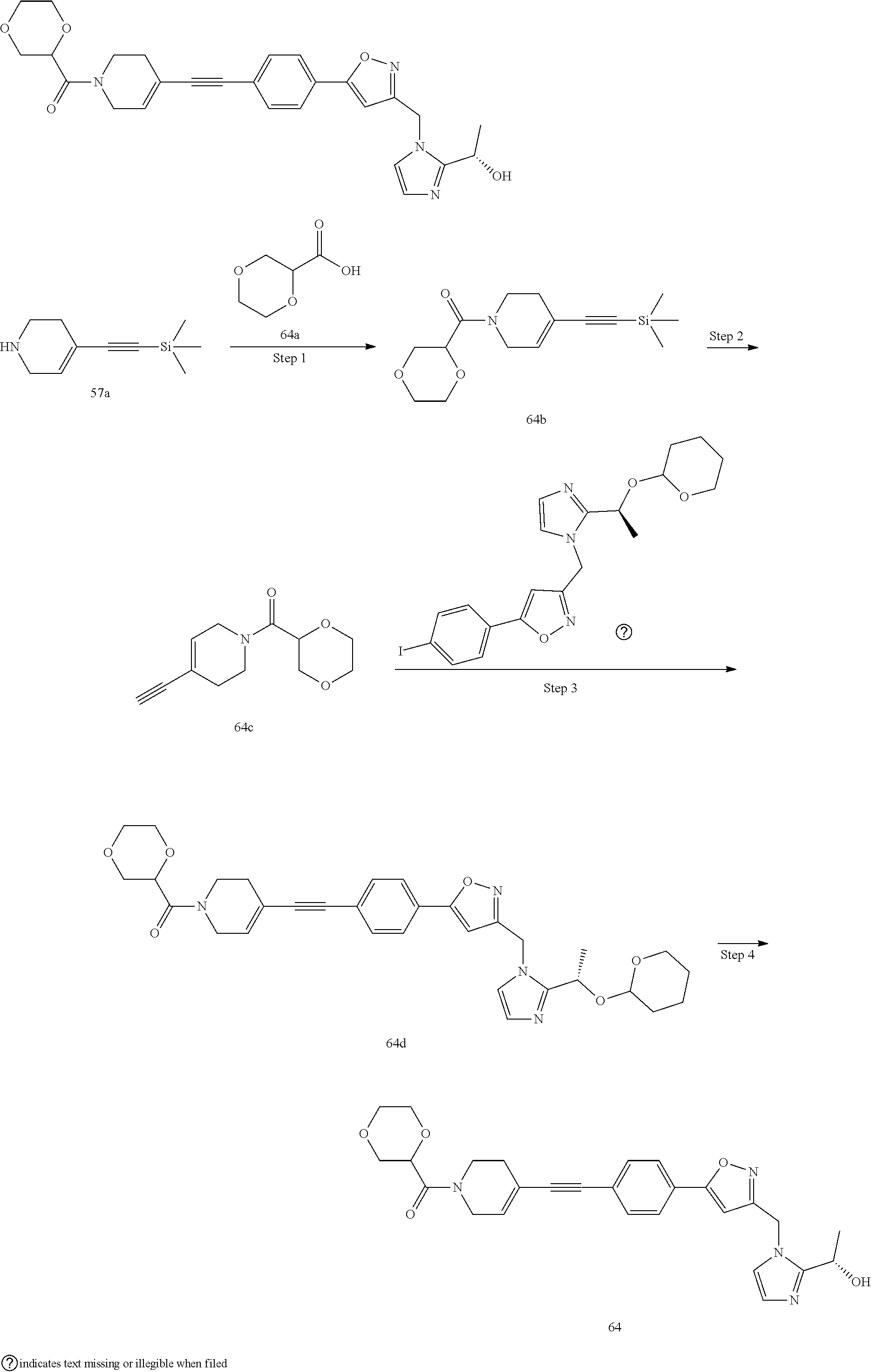

### Step 1

### (1,4-dioxan-2-yl)(4-((trimethylsilyl)ethynyl)-3,6-dihydropyridin-1(2H)-yl)methanone

4-((trimethylsilyl)ethynyl-1,2,3,6-tetrahydropyridine 57a (50 mg, 278.81 μmol), 1,4-dioxane-2-carboxylic acid 64a (55.25 mg, 418.22 μmol), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (2.0 mL), and continuously stirred at room temperature for 4 hours. The raw materials reacted completed detected by mass spectrometry, and the system was concentrated to dryness under reduced pressure to obtain (1,4-dioxan-2-yl)(4-((trimethylsilyl)ethynyl)-3,6-dihydropyridin-1(2H)-yl)methanone 64b (45 mg), which was directly used for the next reaction.

MS m/z (ESI): 294.0 [M+1].

### Step 2

### (1,4-dioxan-2-yl)(4-ethynyl-3,6-dihydropyridin-1(2H)-yl)methanone

(1,4-dioxan-2-yl)(4-((trhimethylsilyl)ethynyl-3,6-dihydropyridin-1(2H)-yl)methanone 64b (45 mg, 153.36 μmol), potassium fluoride (8.91 mg, 153.36 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain (1,4-dioxan-2-yl)(4-ethynyl-3,6-dihydropyridin-1(2H)-yl)methanone 64c (25 mg), which was directly used for the next reaction.

MS m/z (ESI): 222.1 [M+1].

### Step 3

### (1,4-dioxan-2-yl)(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), (1,4-dioxan-2-yl)(4-ethynyl-3,6-dihydropyridin-1(2H)-yl)methanone 64c (21.93 mg, 89.19 μmol), triethylene diamine (13.34 mg, 118.92 μmol) and allylpalladium(II) chloride dimer (4.34 mg, 11.89 μmol) were sequentially added to acetonitrile (2 mL), replaced thrice with argon gas, and stirred at room temperature for 12 hours. The system was concentrated to dryness under reduced pressure, and the residue was separated and purified by silica gel column chromatography (eluent: system A) to obtain (1,4-dioxan-2-yl)(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)methanone 64d (25 mg), with a yield of 73.42%.

MS m/z (ESI): 573.0 [M+1].

### Step 4

### (1,4-dioxan-2-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone

(1,4-dioxan-2-yl)(4-((3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 64d (25 mg, 43.66 μmol), and trifluoroacetic acid (0.5 mL) were sequentially added to dichloromethane (2 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (1,4-dioxan-2-yl)(4-((4-(3-((2-((S)-1-hydroxyethyl)-1H-imidazol-1-yl)methyl)isoxazol-5-yl)phenyl)ethynyl)-3,6-dihydropyridin-1(2H)-yl)methanone 64 (3.8 mg), with a yield of 14.33%.

MS m/z (ESI): 488.9 [M+1].

### Example 65

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydro-2H-pyran-2-yl)methanone

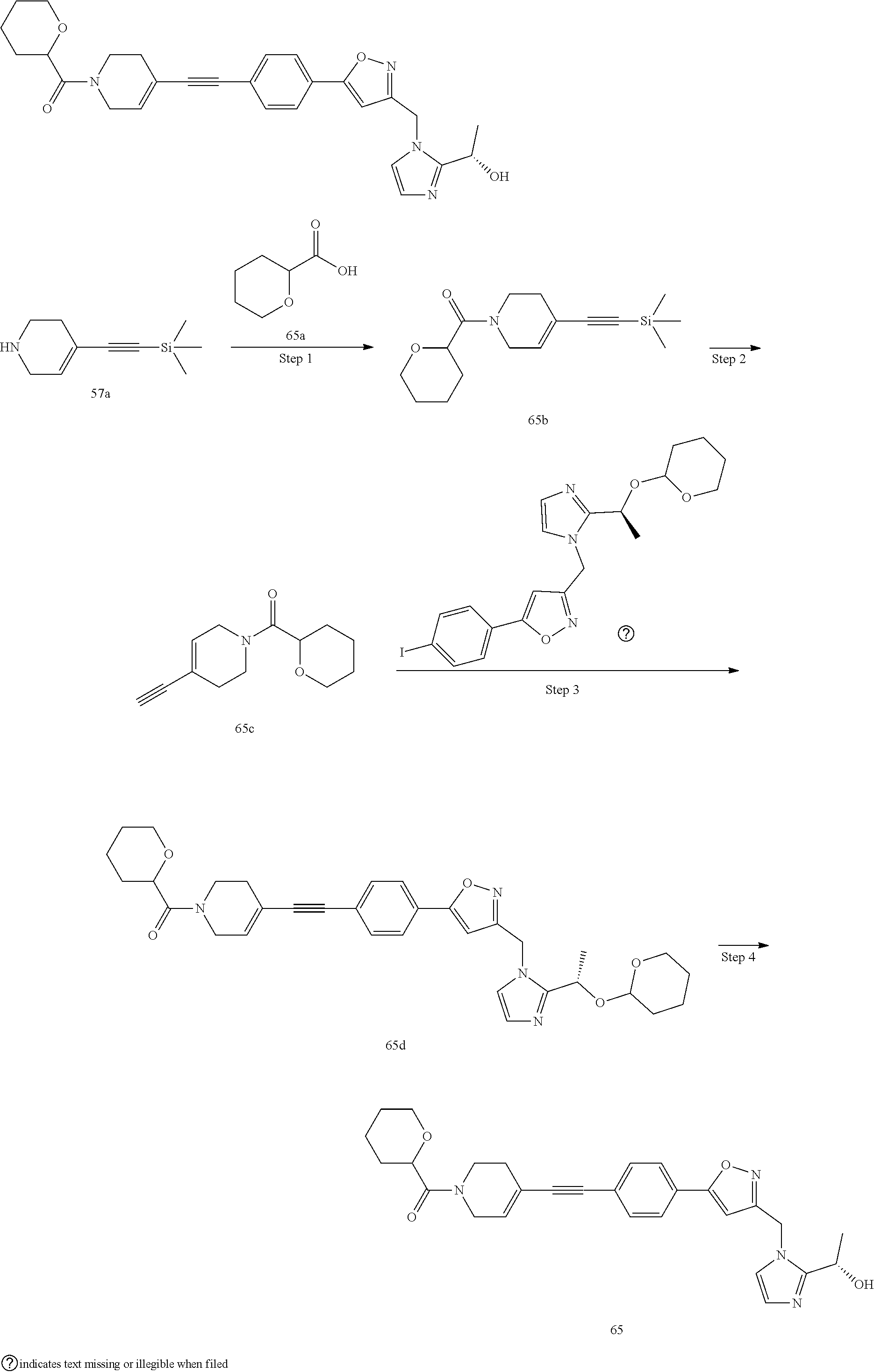

### Step 1

### (Tetrahydro-2H-pyran-2-yl)(4-((trimethylsilyl)ethynyl)-3,6-dihydropyridin-1(2H)-yl)methanone

4-((trimethylsilyl)ethynyl-1,2,3,6-tetrahydropyridine 57a (50 mg, 278.81 μmol), tetrahydro-2H-pyran-2-carboxylic acid 65a (54.43 mg, 418.22 μmol, 47.74 μL, commercially available), O-(7-azabenzotriazole-1-yl)-N,N,N′,N′-tetramethyluronium hexafluorophosphate (127.21 mg, 334.57 μmol) and N,N-diisopropylethylamine (108.10 mg, 836.43 μmol, 145.69 μL) were sequentially added to dichloromethane (1.95 mL), and continuously stirred at room temperature for 4 hours. The raw materials reacted completed detected by mass spectrometry, and the system was concentrated to dryness under reduced pressure to obtain (tetrahydro-2H-pyran-2-yl)(4-((trimethylsilyl)ethynyl)-3,6-dihydropyridin-1(2H)-yl)methanone 65b (60 mg), which was directly used for the next reaction.

MS m/z (ESI): 292.0 [M+1].

### Step 2

### (4-ethynyl-3,6-dihydropyridin-1(2H)-yl)(tetrahydro-2H-pyran-2-yl)methanone

(Tetrahydro-2H-pyran-2-yl)(4-((trimethylsilyl)ethynyl)-3,6-dihydropyridin-1(2H)-yl)methan one 65b (60 mg, 205.86 μmol), and potassium fluoride (59.80 mg, 1.03 mmol) were sequentially added to methanol (2 mL), and continuously stirred at room temperature for 3 hours. The system was concentrated to dryness under reduced pressure, and added with acetonitrile (3 mL for dissolving, and then filtered. The filtrate was concentrated to dryness under reduced pressure to obtain (4-ethynyl-3,6-dihydropyridin-1(2H)-yl)(tetrahydro-2H-pyran-2-yl)methanone 65c (30 mg). The crude product was directly used in the next reaction.

MS m/z (ESI): 220.1 [M+1].

### Step 3

### (Tetrahydro-2H-pyran-2-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), (4-ethynyl-3,6-dihydropyridin-1(2H)-yl)(tetrahydro-2H-pyran-2-yl)methanone 65c (21.73 mg, 89.19 μmol), triethylene diamine (13.34 mg, 118.92 μmol) and allylpalladium chloride dimer (4.34 mg, 11.89 μmol) were sequentially added to acetonitrile (2 mL), replaced thrice with argon gas, and continuously stirred at room temperature for 12 hours. The system was concentrated to dryness under reduced pressure, and the residue was separated and purified by silica gel column chromatography (eluent: system A) to obtain (tetrahydro-2H-pyran-2-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 65d (20 mg), with a yield of 58.94%.

MS m/z (ESI): 570.9 [M+1].

### Step 4

### (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydro-2H-pyran-2-yl)methanone

(Tetrahydro-2H-pyran-2-yl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 65d (20 mg, 35.05 μmol), trifluoroacetic acid (0.5 mL) were sequentially added to dichloromethane (2 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (4-((4-(3-((2-((S)-1-hydroxyethyl)-1H-imidazol-1-yl)methyl)isoxazol-5-yl)phenyl)ethynyl)-3,6-dihydropyridin-1(2H)-yl)(tetrahydro-2H-pyran-2-yl)methanone 65 (4.9 mg), with a yield of 22.44%.

MS m/z (ESI): 486.9 [M+1].

### Example 66

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(tetrahydro-2H-pyran-4-yl)methanone

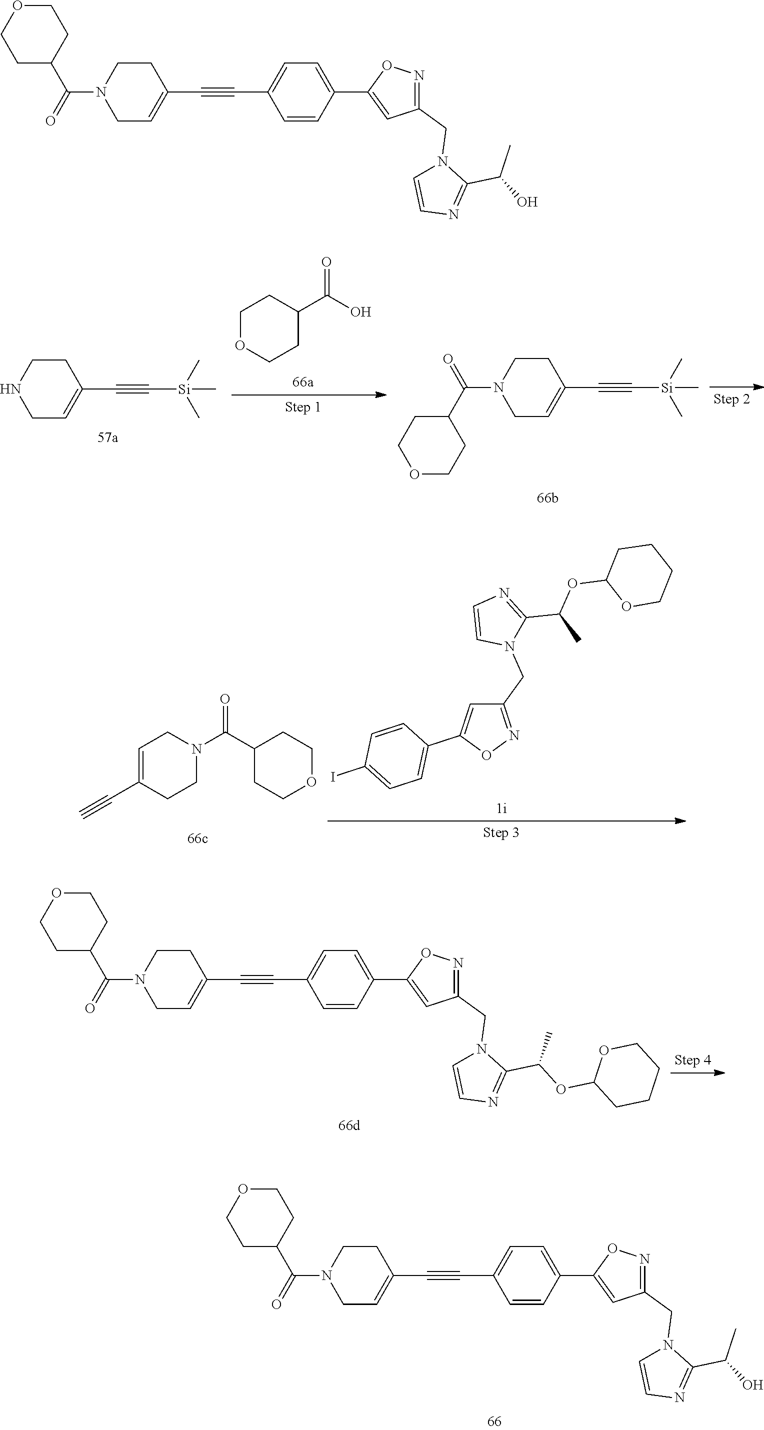

### Step 1

### (Tetrahydro-2H-pyran-4-yl)(4-((trimethylsilyl)ethynyl)-3,6-dihydropyridin-1(2H)-yl)methanone

4-((trimethylsilyl)ethynyl-1,2,3,6-tetrahydropyridine 57a (50 mg, 278.81 μmol), tetrahydro-2H-pyran-4-carboxylic acid 66a (54.43 mg, 418.22 μmol), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (1.85 mL), and continuously stirred at room temperature for 4 hours. The raw materials reacted completed detected by mass spectrometry, and the system was concentrated to dryness under reduced pressure to obtain (tetrahydro-2H-pyran-4-yl)(4-((trimethylsilyl)ethynyl)-3,6-dihydropyridin-1(2H)-yl)methanone 66b (50 mg), which was directly used for the next reaction.

MS m/z (ESI). 292.0 [M+1].

### Step 2

### (4-ethynyl-3,6-dihydropyridin-1(2H)-yl)(tetrahydro-2H-pyran-4-yl)methanone

(Tetrahydro-2H-pyran-4-yl)(4-((trimethylsilyl)ethynyl)-3,6-dihydropyridin-1(2H)-yl)methan one 66b (50 mg, 171.55 μmol), and potassium fluoride (49.84 mg, 857.75 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain (4-ethynyl-3,6-dihydropyridin-1(2H)-yl)(tetrahydro-2H-pyran-4-yl)methanone 66c (25 mg), which was directly used for the next reaction.

MS m/z (ESI): 220.0 [M+1].

### Step 3

### (4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydro-2H-pyran-4-yl)methanone

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), (4-ethynyl-3,6-dihydropyridin-1(2H)-yl)(tetrahydro-2H-pyran-4-yl)methanone 66c (23.01 mg, 89.19 μmol), triethylene diamine (13.34 mg, 118.92 μmol) and allylpalladium(II) chloride dimer (4.34 mg, 11.89 μmol) were sequentially added to acetonitrile (2.0 mL), replaced thrice with argon gas, and continuously stirred at room temperature for 3 hours. The reaction solution was added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), dried with anhydrous sodium sulfate, filtered and concentrated to dryness under reduced pressure. The residue was separated and purified by silicagel column chromatography (eluent: system A) to obtain (4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydro-2H-pyran-4-yl)methanone 66d (25 mg), with a yield of 73.68%.

MS m/z (ESI): 571.0 [M+1].

### Step 4

### (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(tetrahydro-2H-pyran-4-yl)methanone

(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)(tetrahydro-2H-pyran-4-yl)methanone 66d (20 mg, 35.05 μmol), and trifluoroacetic acid (0.5 mL) were sequentially added to dichloromethane (1 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(4-((4-(3-((2-(1-hydroxyethyl)-1H-imidazol-1-yl)methyl)isoxazol-5-yl)phenyl)ethynyl)-3,6-dihydropyridin-1(2H)-yl)(tetrahydro-2H-pyran-4-yl)methanone 66 (15.8 mg), with a yield of 71.39%.

MS m/z (ESI): 486.9 [M+1].

### Example 67

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-3-methoxypropan-1-one

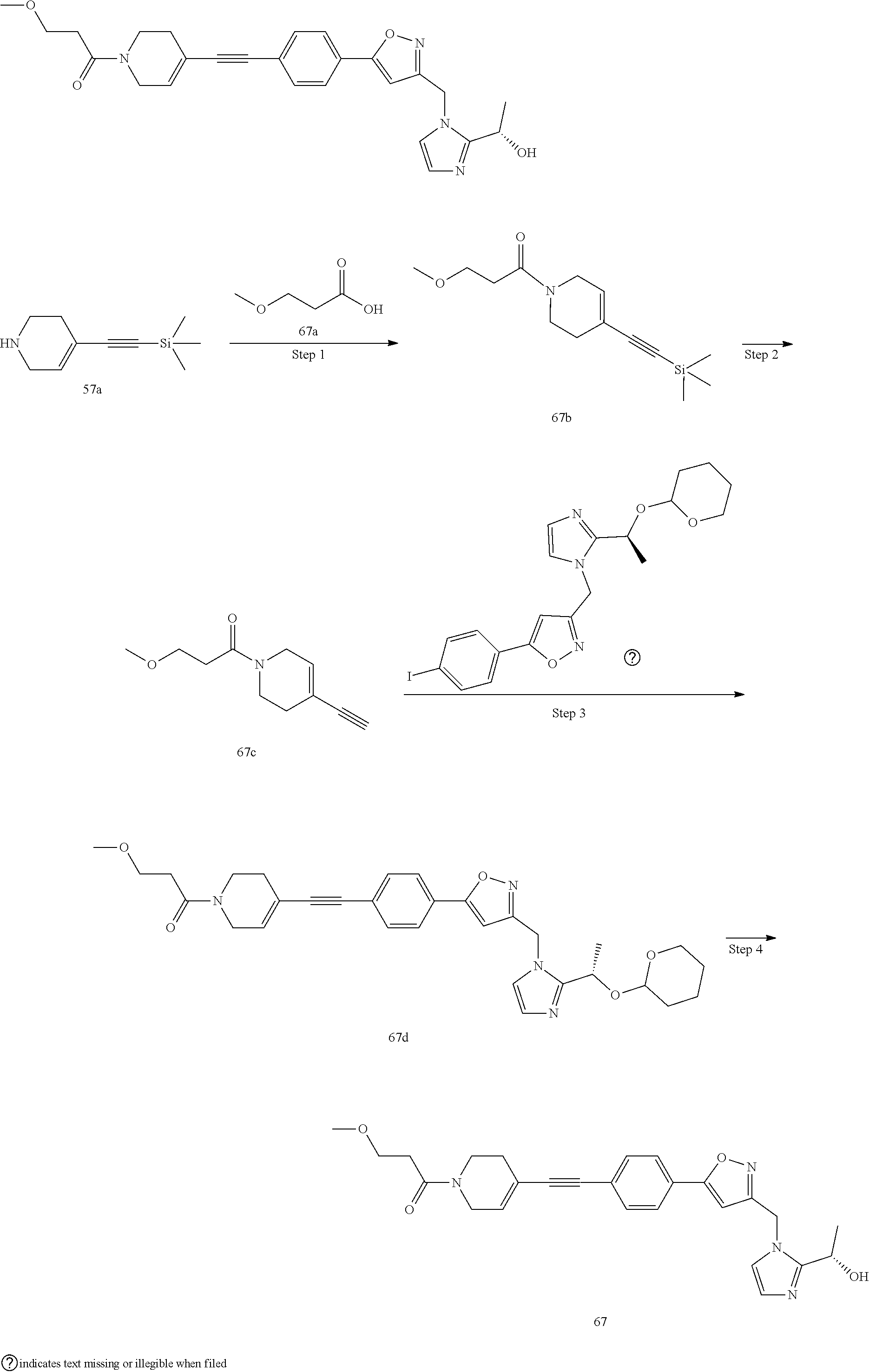

### Step 1

### 3-methoxy-1-(4-((trimethylsilyl)ethynyl)-3,6-dihydropyridin-1(2H)-yl)propan-1-one

4-((trimethylsilyl)ethynyl-1,2,3,6-tetrahydropyridine 57a (50 mg, 278.81 μmol), 3-methoxypropanoic acid 67a (43.54 mg, 418.22 μmol, 39.29 μL, commercially available), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (2 mL), and continuously stirred at room temperature for 4 hours. The raw materials reacted completed detected by mass spectrometry, and the system was concentrated to dryness under reduced pressure to obtain 3-methoxy-1-(4-((trimethylsilyl)ethynyl)-3,6-dihydropyridin-1(2H)-yl)propan-1-one 67b (50 mg), which was directly used for the next reaction.

MS m/z (ESI): 266.0 [M+1].

### Step 2

### 1-(4-ethynyl-3,6-dihydropyridin-1(2H)-yl)-3-methoxypropan-1-one

3-methoxy-1-(4-((trimethylsilyl)ethynyl)-3,6-dihydropyridin-1(2H)-yl)propan-1-one 67b (50 mg, 188.38 μmol), and potassium fluoride (54.72 mg, 941.89 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain 1-(4-ethynyl-3,6-dihydropyridin-1(2H)-yl)-3-methoxypropan-1-one 67c (20 mg), which was directly used for the next reaction.

MS m/z (ESI): 194.0 [M+1].

### Step 3

### 3-methoxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)propan-1-one

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), 1-(4-ethynyl-3,6-dihydropyridin-1(2H)-yl)-3-methoxypropan-1-one 67c (20.28 mg, 89.19 μmol), triethylene diamine (13.34 mg, 118.92 μmol), and allylpalladium(II) chloride dimer (4.34 mg, 11.89 μmol) were sequentially added to acetonitrile (2.0 mL), replaced thrice with argon gas, and continuously stirred at room temperature for 3 hours. The reaction solution was added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), dried with anhydrous sodium sulfate, filtered and concentrated to dryness under reduced pressure. The residue was separated and purified by silicagel column chromatography (eluent: system A) to obtain 3-methoxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)propan-1-one 67d (25 mg), with a yield of 77.20%.

MS m/z (ESI): 545.0 [M+1].

### Step 4

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-3-methoxypropan-1-one

3-methoxy-1-(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)propan-1-one 67d (20 mg, 36.72 μmol), and trifluoroacetic acid (0.5 mL) were sequentially added to dichloromethane (1 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-3-methoxypropan-1-one 67 (14.0 mg), with a yield of 64.70%.

MS m/z (ESI): 461.0 [M+1].

### Example 68

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-methoxyethan-1-one

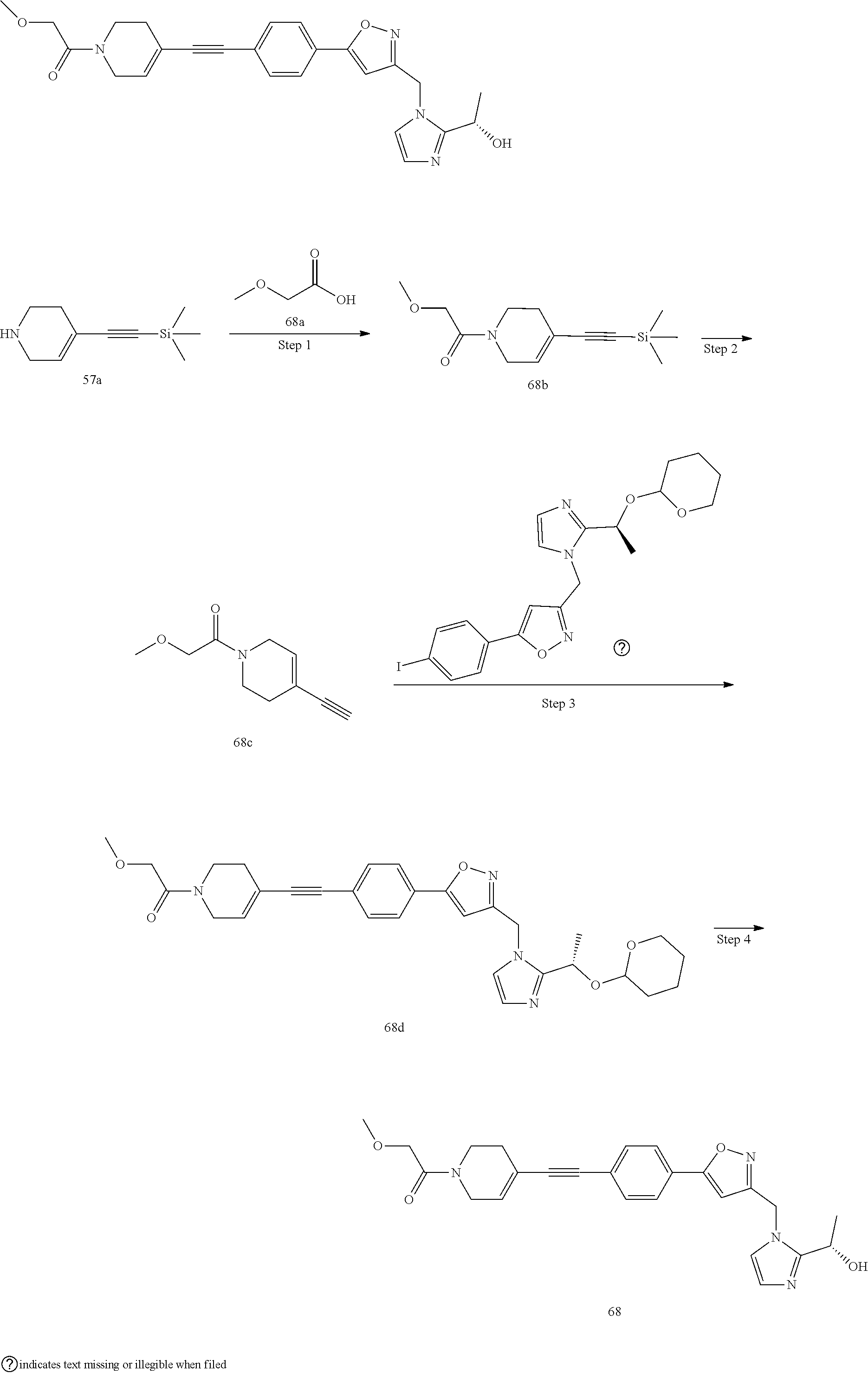

### Step 1

### 2-methoxy-1-(4-((trimethylsilyl)ethynyl)-3,6-dihydropyridin-1(2H)-yl)ethan-1-one

4-((trimethylsilyl)ethynyl-1,2,3,6-tetrahydropyridine 57a (50 mg, 278.81 μmol), 2-methoxyacetic acid 68a (37.67 mg, 418.22 μmol, commercially available), O-(7-azabenzotriazole-1-yl)-N,N,N′,N′-tetramethyluronium hexafluorophosphate (127.21 mg, 334.57 μmol), and N,N-diisopropylethylamine (108.10 mg, 836.43 μmol, 145.69 μL) were sequentially added to dichloromethane (2 mL), and continuously stirred at room temperature for 4 hours. The raw materials reacted completed detected by mass spectrometry, and the system was concentrated to dryness under reduced pressure to obtain 2-methoxy-1-(4-((trimethylsilyl)ethynyl)-3,6-dihydropyridin-1(2H)-yl)ethan-1-one 68b (50 mg), which was directly used for the next reaction.

MS m/z (ESI): 252.0 [M+1].

### Step 2

### 1-(4-ethynyl-3,6-dihydropyridin-1(2H)-yl)-2-methoxyethan-1-one

2-methoxy-1-(4-((trimethylsilyl)ethynyl)-3,6-dihydropyridin-1(2H)-yl)ethan-1-one 68b (50 mg, 198.89 μmol), and potassium fluoride (57.78 mg, 994.45 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain 1-(4-ethynyl-3,6-dihydropyridin-1(2H)-yl)-2-methoxyethan-1-one 68c (20 mg), which was directly used for the next reaction.

MS m/z (ESI): 180.0 [M+1].

### Step 3

### 2-methoxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), 1-(4-ethynyl-3,6-dihydropyridin-1(2H)-yl)-2-methoxyethan-1-one 68c (18.81 mg, 89.19 μmol), triethylene diamine (13.34 mg, 118.92 μmol), and allylpalladium(II) chloride dimer (4.34 mg, 11.89 μmol) were sequentially added to acetonitrile (2.0 mL), replaced thrice with argon gas, and continuously stirred at room temperature for 3 hours. The reaction solution was added with ethyl acetate (30 mL) and water (15 mL). The organic phases were separated, and the aqueous phases were extracted with ethyl acetate (30 mL×2). The combined organic phases were washed with saturated sodium chloride solution (30 mL×2), dried with anhydrous sodium sulfate, filtered and concentrated to dryness under reduced pressure. The residue was separated and purified by silicagel column chromatography (eluent: system A) to obtain 2-methoxy-1-(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)ethan-1-one 68d (25 mg), with a yield of 79.24%.

MS m/z (ESI): 531.0 [M+1].

### Step 4

### (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-methoxyethan-1-one

2-methoxy-1-(4-((4-(3-((2-((1S)-1-((tetrahydro-2H-pyran-2-yl)oxy)ethyl)-1H-imidazol-1-yl) methyl)isoxazol-5-yl)phenyl)ethynyl)-3,6-dihydropyridin-1(2H)-yl)ethan-1-one 68d (20 mg, 37.69 μmol), and trifluoroacetic acid (0.5 mL) were sequentially added to dichloromethane (1 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-1-(4-((4-(3-((2-(1-hydroxyethyl)-1H-imidazol-1-yl)methyl)isoxazol-5-yl)phenyl)ethynyl)-3,6-dihydropyridin-1(2H)-yl)-2-methoxyethan-1-one 68 (13.0 mg), with a yield of 58.89%.

MS m/z (ESI): 446.9 [M+1].

### Example 69

### (S)-(1-hydroxycyclopropyl)(4-((4-(3-((2-(1-hydroxyethyl)-1H-imidazol-1-yl)methyl)isoxazol-5-yl)phenyl)ethynyl)-3,6-dihydropyridin-1(2H)-yl)methanone

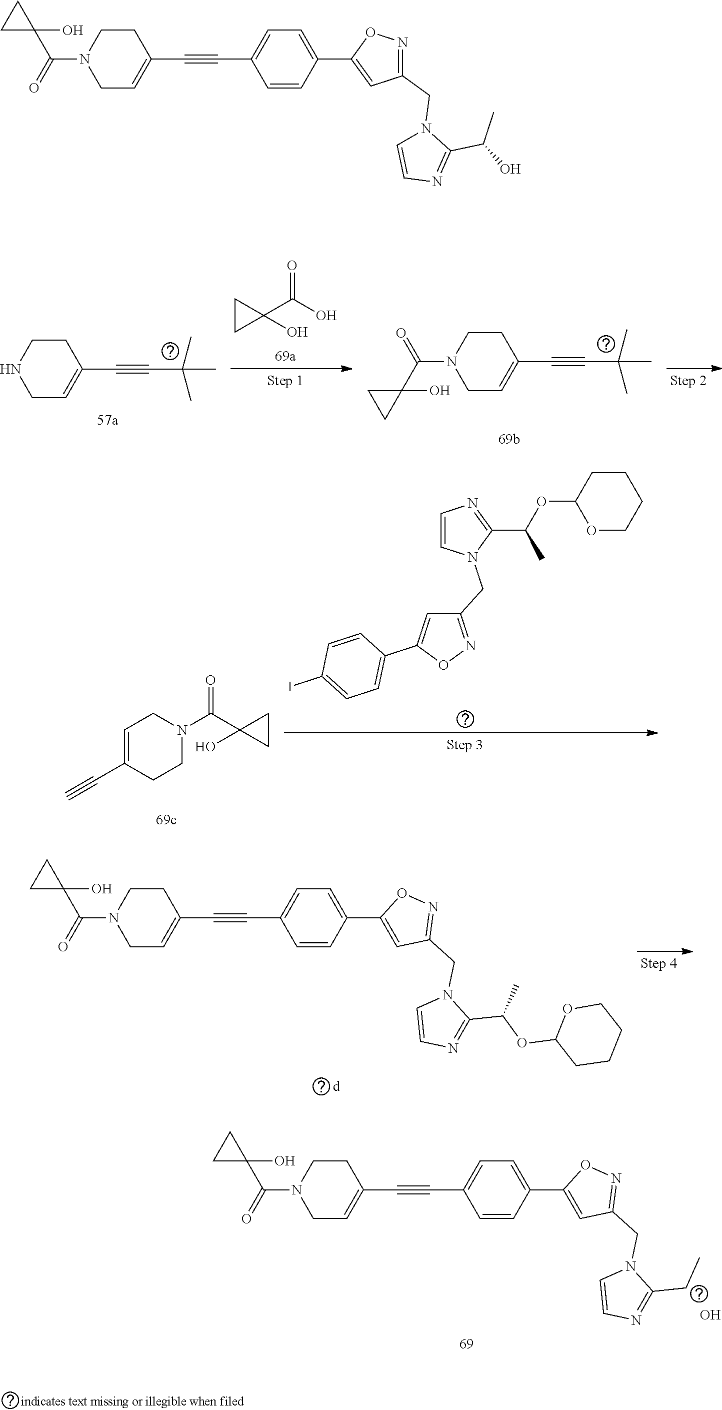

### Step 1

### (1-hydroxycyclopropyl)(4-((trimethylsilyl)ethynyl)-3,6-dihydropyridin-1(2H)-yl)methanone

4-((trimethyl silyl)ethynyl-1,2,3,6-tetrahydropyridine 57a (50 mg, 278.81 μmol), 1-hydroxycyclopropane-1-carboxylic acid 69a (42.69 mg, 418.22 μmol, commercially available), O-(7-aza-benzotriazole-1-yl)-N,N,N′,N′-tetramethyluronium hexafluorophosphate (127.21 mg, 334.57 μmol), and N,N-diisopropyl ethyl amine (108.10 mg, 836.43 μmol, 145.69 μL) were sequentially added to dichloromethane (2 mL), and reacted at room temperature for 4 hours. The raw materials reacted completed detected by mass spectrometry, and the system was concentrated to dryness under reduced pressure to obtain (1-hydroxycyclopropyl)(4-((trimethylsilyl)ethynyl)-3,6-dihydropyridin-1(2H)-yl)methanone 69b (50 mg), which was directly used for the next reaction.

MS m/z (ESI): 263.9 [M+1].

### Step 2

### (4-ethynyl-3,6-dihydropyridin-1(2H)-yl)(1-hydroxycyclopropyl)methanone

(1-hydroxycyclopropyl)(4-((trimethylsilyl)ethynyl)-3,6-dihydropyridin-1(2H)-yl)methanone 69b (50 mg, 189.82 μmol), potassium fluoride (55.14 mg, 949.10 μmol) were sequentially added to methanol (2 mL), and continuously stirred at room temperature for 3 hours. The system was concentrated to dryness under reduced pressure, and added with acetonitrile (3 mL) for dissolving, and then filtered. The filtrate was concentrated to dryness under reduced pressure to obtain (4-ethynyl-3,6-dihydropyridin-1(2H)-yl)(1-hydroxycyclopropyl)methanone 69c (30 mg), which was directly used for the next reaction.

MS m/z (ESI): 191.9 [M+1].

### Step 3

### (1-hydroxycyclopropyl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone

5-(4-iodophenyl)-3-((2-((1S)-1-((tetrahydro-2H-pyran-2-yl)oxy)ethyl)-1H-imidazol-1-yl)methyl)isoxazole 1i (30 mg, 59.46 μmol), (4-ethynyl-3,6-dihydropyridin-1(2H)-yl)(1-hydroxycyclopropyl)methanone 69c (20.07 mg, 89.19 μmol), triethylene diamine (13.34 mg, 118.92 μmol) and allylpalladium(II) chloride dimer (4.34 mg, 11.89 μmol) were sequentially added to acetonitrile (2.0 mL), replaced thrice with argon gas, and continuously stirred at room temperature for 3 hours. The reaction solution was added with ethyl acetate (30 mL) and water (15 mL) for liquid separation. The aqueous phases were extracted with ethyl acetate (30 mL×2). The combined organic phases were washed with saturated sodium chloride solution (30 mL×2), dried with dried with anhydrous sodium sulfate, filtered and concentrated to dryness under reduced pressure. The residue was separated and purified by silicagel column chromatography (eluent: system A) to obtain (1-hydroxycyclopropyl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 69d (25 mg), with a yield of 77.48%.

MS m/z (ESI): 543.0 [M+1].

### Step 4

### (S)-(1-hydroxycyclopropyl)(4-((4-(3-((2-(1-hydroxyethyl)-1H-imidazol-1-yl)methyl)isoxazol-5-yl)phenyl)ethynyl)-3,6-dihydropyridin-1(2H)-yl)methanone

(1-hydroxycyclopropyl)(4-((4-(3-((2-((1S)-1-((tetrahydro-2H-pyran-2-yl)oxy)ethyl)-1H-imidazol-1-yl)methyl)isoxazol-5-yl)phenyl)ethynyl)-3,6-dihydropyridin-1(2H)-yl)methanone 69d (20 mg, 36.86 μmol), trifluoroacetic acid (0.5 mL) were sequentially added to dichloromethane (1 mL), and continuously stirred at room temperature for one hour. The system was concentrated to dryness under reduced pressure, and the residue was subjected to preparative liquid separation (separation column AKZONOBEL Kromasil; 250×21.2 mm ID; 5 μm, 20 mL/min; mobile phase A: 0.05% TFA+H₂O, mobile phase B: CH₃CN) to obtain (S)-(1-hydroxycyclopropyl)(4-((4-(3-((2-(1-hydroxyethyl)-1H-imidazol-1-yl)methyl)isoxazol-5-yl)phenyl)ethynyl)-3,6-dihydropyridin-1(2H)-yl)methanone 69 (2.2 mg), with a yield of 9.83%.

MS m/z (ESI): 458.9 [M+1].

### Biological Evaluation

### Test Example 1 Determination of the Compounds of the Present Invention on the Inhibition of Enzymatic Activity of LpxC

The following method was used to determine degrees of inhibition of the compounds of the present invention on the enzymatic activity of recombinant Pseudomonas aeruginosa LpxC in vitro.

The test process was briefly described as follows: the tested compound was first dissolved in DMSO to prepare a 10 mM storage solution. The reaction was carried out in a 96-well microplate. Firstly, 20 μL of recombinant Pseudomonas aeruginosa LpxC (purchased from Signalway Antibody, article number AP74647-2) was added into the wells, and the final concentrations were 5 nM respectively. 5 μL of the compound to be tested was added, diluted in 4-fold, with 8 concentration points, and the concentration range was 0.61-10000 nM. 5 μL of LpxC substrate UDP-3-O—(R-3-hydroxydecanoyl)-GlcNAc (purchased from Biosynth Carbosynth, article number mu75071) was added, wherein a final concentration of the substrate was 10 M, and the substrate was incubated at 25 °C for 120 minutes. Subsequently, 20 μL of 2.0 mg/mL fluorescamine (purchased from sigma aldrich, article number F9015, wherein a solvent was dimethylformamide/acetonitrile (1:1)) was added to the reaction system, mixed evenly and reacted for 10 minutes. Finally, 50 μL of 200 mM sodium phosphate buffer solution (pH 8.0) was added to stop the reaction, and a microplate reader (BMG) was used for reading. An excitation wavelength and an emission wavelength were 390 nm and 495 nm respectively. Compared with the fluorescence intensity ratio of a control group (0.1% DMSO), the percentage inhibition rates of the compounds at each concentration were calculated, and the IC₅₀ values of the compounds were obtained by performing nonlinear regression analysis with logarithmic value of the compound concentration-inhibition rate by GraphPad Prism 5 software, which can be seen in Table 1.

TABLE 1
Inhibition results of the compounds of the present
invention on the enzymatic activity of LpxC

| No. of compound | IC₅₀/nM |
|---|---|
| Example 1 | 9.46 |
| Example 4 | 23.38 |
| Example 5 | 7.03 |
| Example 8 | 19.33 |
| Example 9 | 18.8 |
| Example 10 | 22.82 |
| Example 11 | 8.66 |
| Example 12 | 4.06 |
| Example 13 | 5.00 |
| Example 14 | 6.81 |
| Example 15 | 4.42 |
| Example 16 | 10.35 |
| Example 17 | 8.32 |
| Example 18 | 4.42 |
| Example 19 | 6.05 |
| Example 20 | 6.77 |
| Example 21 | 5.11 |
| Example 22 | 6.54 |
| Example 23 | 6.36 |
| Example 24 | 6.90 |
| Example 25 | 7.28 |
| Example 26 | 11.29 |
| Example 27 | 6.08 |
| Example 28 | 14.12 |
| Example 29 | 6.17 |
| Example 30 | 16.2 |
| Example 32 | 22.49 |
| Example 33 | 19.72 |
| Example 34 | 26.73 |
| Example 36 | 19 |
| Example 38 | 22.66 |
| Example 39 | 21.72 |
| Example 40 | 21.42 |
| Example 41 | 22.26 |
| Example 43 | 25.82 |
| Example 44 | 24.88 |
| Example 45 | 12.3 |
| Example 46 | 21.51 |
| Example 47 | 13.13 |
| Example 48 | 27.86 |
| Example 49 | 22.61 |
| Example 50 | 23.45 |
| Example 51 | 15.15 |
| Example 52 | 13.89 |
| Example 54 | 15.49 |
| Example 55 | 14.73 |
| Example 57 | 29.4 |
| Example 58 | 19.2 |
| Example 59 | 23.8 |
| Example 60 | 25.3 |
| Example 61 | 11.5 |
| Example 62 | 18.0 |
| Example 63 | 23.6 |
| Example 64 | 19.1 |
| Example 65 | 15.1 |
| Example 66 | 18.5 |
| Example 67 | 22.9 |
| Example 68 | 21.2 |
| Example 69 | 20.8 |

Conclusion: the IC₅₀ of the preferred compounds of the present invention for inhibiting the enzymic activity of the recombinant Pseudomonas aeruginosa LpxC is less than 100 nM, and the preferred compounds of the present invention have a significant inhibitory effect on the enzymic activity of LpxC.

### Test Example 2. Evaluation of Antibacterial Activity of the Compounds of the Present Invention

The minimum inhibitory concentration (MIC) in vitro was determined according to CLSI's guidelines, and a minimal broth dilution method was used.

The test process was briefly described as follows: the tested compound was dissolved in DMSO to prepare a storage solution of 12.8 mg/mL, and then prepared into 11 two-fold-diluted 100× high-concentration working liquids (a final concentration of the system was 64 μg/mL to 0.06 μg/mL) by DMSO. Stains (K. pneumoniae ATCC13883, K. pneumoniae ATCC51504 and E. coli ATCC 25922) frozen at −80 °C with glycerin were inoculated to a solid agar culture medium, placed into an incubator for culturing for 18-24 hours at 35 °C to finish the preparation work of the strains. A proper amount of solid plate culture was collected, re-suspended in normal saline, and uniformly mixed. The turbidity of the bacterial suspension was adjusted to a proper turbidity by a turbidity meter, containing about 1×10⁸ cfu/mL of bacteria. Then, the turbidity-regulated bacterial suspension was diluted with a test culture medium (type: CAMHIIB, purchased from BD company) to a bacterial concentration of 5×10⁵ cfu/mL, thus finishing the preparation of an inoculation solution. 198 μL of the inoculation solution was inoculated into a 96-well plate, and added with 2 μL 100× high concentration working liquid of the compound, then the 96-well plate was placed at 35 °C for culturing for 18-24 hours. After culturing, the test plate was visually observed, and the minimum drug concentration that completely inhibited the growth of bacterial cells was the minimum inhibitory concentration (MIC) of the compound, as shown in table 2.

TABLE 2
Test results of the antibacterial activities of the compounds of the present invention

| | MIC | MIC | |
|---|---|---|---|
| No. of | (K. PneumoniaeATCC13883)/ | (K. PneumoniaeATCC51504)/ | MIC (E. coli ATCC |
| compound | μg · mL⁻¹ | μg · mL⁻¹ | 25922)/μg · mL⁻¹ |
| Example 12 | 4 | — | 2 |
| Example 13 | 1 | — | 1 |
| Example 17 | 2 | — | 1 |
| Example 19 | 4 | — | 2 |
| Example 20 | 4 | — | 2 |
| Example 21 | 4 | — | 2 |
| Example 22 | 4 | — | 1 |
| Example 23 | 4 | — | 2 |
| Example 25 | 4 | — | 2 |
| Example 26 | 2 | — | 2 |
| Example 27 | 4 | — | 2 |
| Example 29 | 4 | — | 2 |
| Example 33 | 2 | — | 1 |
| Example 34 | 4 | — | 2 |
| Example 35 | — | 2 | 2 |
| Example 36 | — | 0.25 | 1 |
| Example 37 | — | 2 | 2 |
| Example 38 | — | 0.5 | 0.25 |
| Example 39 | — | 1 | 2 |
| Example 40 | — | 0.5 | 2 |
| Example 41 | — | 0.25 | 1 |
| Example 42 | — | 2 | 4 |
| Example 43 | — | 1 | 2 |
| Example 44 | — | 0.5 | 1 |
| Example 45 | — | 0.5 | 1 |
| Example 47 | 2 | — | 2 |
| Example 48 | 2 | — | 2 |
| Example 49 | 2 | — | 1 |
| Example 52 | 2 | 1 | 1 |
| Example 53 | 8 | 2 | 2 |
| Example 55 | — | 2 | 2 |
| Example 57 | — | 1 | 2 |
| Example 58 | — | 1 | 1 |
| Example 59 | — | 0.5 | 2 |
| Example 60 | — | 1 | 2 |
| Example 61 | — | 0.5 | 1 |
| Example 62 | — | 0.5 | 0.5 |
| Example 63 | — | 0.5 | 0.5 |
| Example 64 | — | 0.5 | 1 |
| Example 65 | — | 0.5 | 2 |
| Example 66 | — | 1 | 2 |
| Example 67 | — | 0.5 | 1 |
| Example 68 | — | 1 | 1 |
| Example 69 | — | 2 | 2 |

Conclusion: the in vitro minimum inhibitory degree of the preferred compounds of the present invention against Pneumonia (K. pneumoniae ATCC 13883, K. pneumoniae ATCC 51504) and Escherichia coli (E. coli ATCC 25922) is less than 50 μg/mL, and the preferred compounds of the present invention have good inhibitory effects on both Pneumonia and Escherichia coli.

1. A compound represented by a general formula (I) or a stereoisomer or a tautomer thereof, or a pharmaceutically acceptable salt thereof: 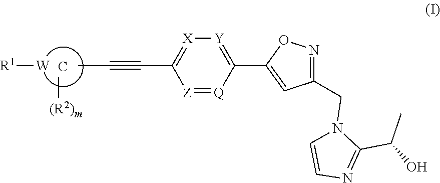
wherein:
W is selected from O, S(O)ᵣ or N;
ring C is selected from 5- to 10-membered spiro-heterocyclyl or monocyclic heterocyclyl, wherein the monocyclic heterocyclyl contains one double bond;
X, Y, Z and Q are each independently selected from CR^{A} or N atoms, and at most two of X, Y, Z and Q are N atoms at the same time;
R^{A} is selected from hydrogen atom, halogen, hydroxyl, cyano, alkyl or alkoxy; wherein the alkyl or the alkoxy is optionally further substituted by one or more substituents selected from halogen, hydroxyl, cyano, alkyl or alkoxy;
R¹ does not exist or is selected from hydrogen atom, hydroxyl, cyano, halogen, alkyl, alkoxy, cycloalkyl, heterocyclyl, aryl, heteroaryl, —C(O)R³, —C(O)NR⁶R⁷ or —S(O)ᵣR⁴; wherein the alkoxy, the alkyl, the cycloalkyl, the heterocyclyl, the aryl, or the heteroaryl is optionally further substituted by one or more R^{B};
R³ and R⁴ are each independently selected from hydrogen atom, hydroxyl, alkyl, cycloalkyl, aryl, heteroaryl or heterocyclyl; wherein the alkyl, the cycloalkyl, the aryl, the heteroaryl or the heterocyclyl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R^{B} is each independently selected from cyano, halogen, alkyl, hydroxyl, cycloalkyl, heterocyclyl, aryl, heteroaryl, —OR⁵, —C(O)R⁵, —C(O)OR %, —NHC(O)R⁵, —NHC(O)OR⁵, —NR⁶R⁷, —C(O)NR⁶R⁷, —CH₂NHC(O)OR⁵, —CH₂NR⁶R⁷ or —S(O)ᵣR⁵; wherein the alkyl, the cycloalkyl, the heterocyclyl, the aryl or the heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
alternatively, two R^{B} form one —C(═O)— together with the same carbon atom to which the two R^{B} are connected;
each R² is the same or different, and is independently selected from hydroxyl, cyano, halogen, alkyl or alkoxy; wherein the alkyl or the alkoxy is optionally further substituted by one or more substituents selected from halogen, hydroxyl, cyano, alkyl or alkoxy;
R⁵ is each independently selected from hydrogen atom, alkyl, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, cyano, alkyl, alkoxy, haloalkyl, haloalkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁶ and R⁷ are each independently selected from hydrogen atom, hydroxyl, halogen, alkyl, alkoxy, cycloalkyl, heterocyclyl, aryl or heteroaryl, wherein the alkyl, the alkoxy, the cycloalkyl, the heterocyclyl, the aryl or the heteroaryl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
alternatively, R⁶ and R⁷ form a 4- to 8-membered heterocyclyl together with atoms to which R⁶ and R⁷ are connected, wherein the 4- to 8-membered heterocyclyl contains one or more N, O or S(O), and the 4- to 8-membered heterocyclyl is optionally further substituted by one or more substituents selected from hydroxyl, halogen, nitro, cyano, alkyl, alkoxy, cycloalkyl, heterocyclyl, aryl, heteroaryl, ═O, —C(O)R⁸, —C(O)OR⁸, —OC(O)R⁸, —NR⁹R¹⁰, —C(O)NR⁹R¹⁰, —SO₂NR⁹R¹⁰ or —NR⁹C(O)R¹⁰;
R⁸, R⁹ and R¹⁰ are independently selected from hydrogen atom, alkyl, amino, cycloalkyl, heterocyclyl, aryl or heteroaryl, wherein the alkyl, the cycloalkyl, the heterocyclyl, the aryl or the heteroaryl is optionally further substituted by one or more substituents selected from hydroxyl, halogen, nitro, amino, cyano, alkyl, alkoxy, cycloalkyl, heterocyclyl, aryl, heteroaryl, carboxyl or carboxylate;
m is 0, 1 or 2; and
r is each independently 0, 1 or 2.

2. The compound or the stereoisomer or the tautomer thereof or the pharmaceutically acceptable salt thereof according to claim 1, wherein the compound or the stereoisomer or the tautomer thereof or the pharmaceutically acceptable salt thereof is a compound represented by general formula (II) or a stereoisomer, a tautomer, or a pharmaceutically acceptable salt thereof, 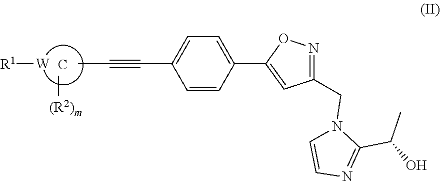
rings C, R¹, R², W and m are as defined in claim 1.

3. The compound or the stereoisomer or the tautomer thereof or the pharmaceutically acceptable salt thereof according to claim 1, wherein W is N.

4. The compound or the stereoisomer or the tautomer thereof or the pharmaceutically acceptable salt thereof according to claim 1, wherein ring C is selected from:

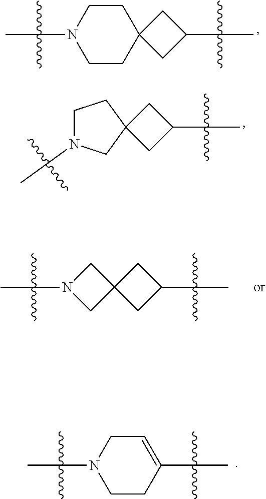

5. The compound or the stereoisomer or the tautomer thereof or the pharmaceutically acceptable salt thereof according to claim 1, wherein:
R¹ is selected from hydrogen atom, hydroxyl, alkyl, cycloalkyl, heterocyclyl, —C(O)R³, —C(O)NR⁶R⁷ or —S(O)ᵣR⁴; wherein the alkyl or the cycloalkyl is optionally further substituted by one or more R^{B};
R^{B} is each independently selected from cyano, hydroxyl, heterocyclyl, aryl, heteroaryl, —OR, —C(O)OR⁵, —NR⁶R⁷, —C(O)NR⁶R⁷ or —S(O)ᵣR⁵; wherein the heterocyclyl or the heteroaryl is optionally further substituted by one or more substituents selected from hydroxyl, cyano, alkoxy, haloalkyl, ═O, —C(O)OR⁵ or —C(O)NR⁹R¹⁰;
alternatively, two R^{B} form one —C(═O)— together with the same carbon atom to which the two R^{B} are connected;
R³ is selected from hydrogen atom, alkyl, cycloalkyl, heteroaryl or heterocyclyl; wherein the alkyl, the cycloalkyl, the heteroaryl or the heterocyclyl is optionally further substituted by one or more substituents selected from hydroxyl, alkoxy, cyano, heteroaryl, ═O, —NR⁹R¹⁰, —C(O)OR⁸ or —C(O)NR⁹R¹⁰;
R⁴ is hydrogen atom or hydroxyl;
R⁵ is each independently selected from hydrogen atom, alkyl or hydroxyalkyl;
R⁶ and R⁷ are each independently selected from hydrogen atom or alkyl, wherein the alkyl is optionally further substituted by one or more substituents selected from hydroxyl, cyano, halogen or alkoxy;
R⁸, R⁹ and R¹⁰ are each independently selected from hydrogen atom or alkyl; and
r is 2.

6. The compound or the stereoisomer or the tautomer thereof or the pharmaceutically acceptable salt thereof according to claim 1, wherein the compound is:

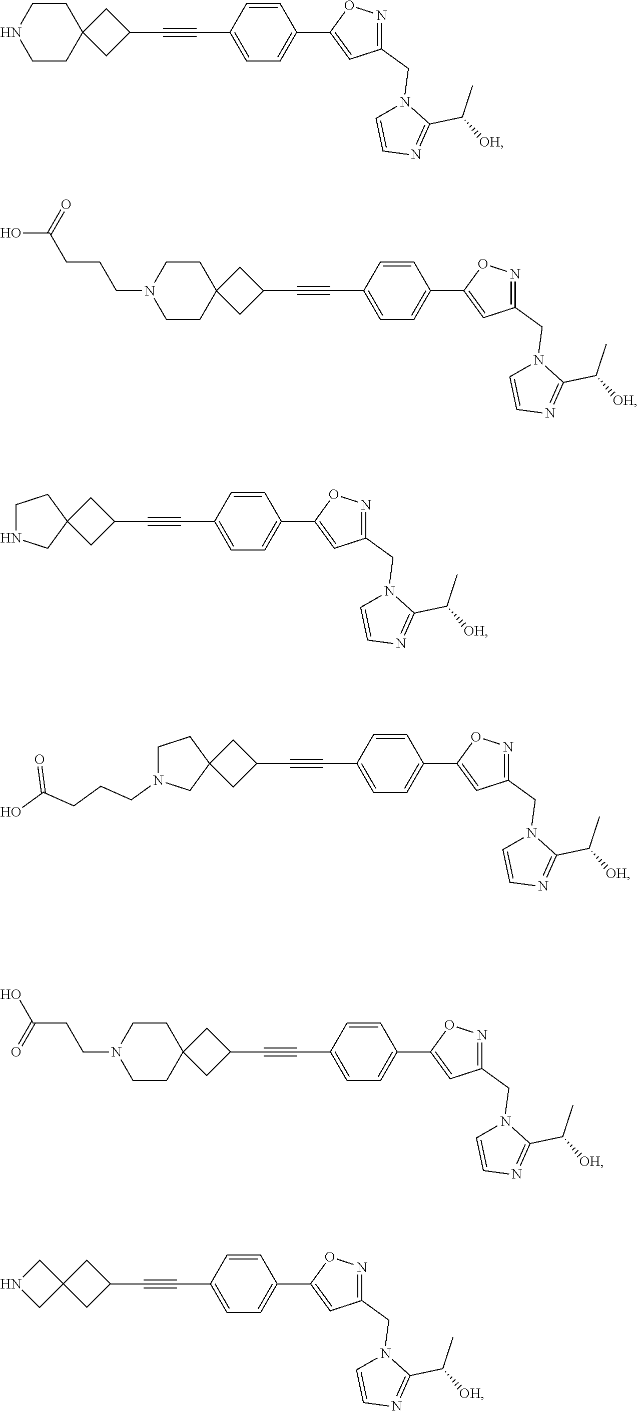 [SMILES: C[C@H](O)c1nccn1Cc1cc(-c2ccc(C#CC3CC4(CCNCC4)C3)cc2)on1 ; C[C@H](O)c1nccn1Cc1cc(-c2ccc(C#CC3CC4(CCN(CCCC(=O)O)CC4)C3)cc2)on1 ; C[C@H](O)c1nccn1Cc1cc(-c2ccc(C#CC3CC4(CCNC4)C3)cc2)on1 ; C[C@H](O)c1nccn1Cc1cc(-c2ccc(C#CC3CC4(CCN(CCCC(=O)O)C4)C3)cc2)on1 ; C[C@H](O)c1nccn1Cc1cc(-c2ccc(C#CC3CC4(CCN(CCC(=O)O)CC4)C3)cc2)on1 ; C[C@H](O)c1nccn1Cc1cc(-c2ccc(C#CC3CC4(CNC4)C3)cc2)on1]

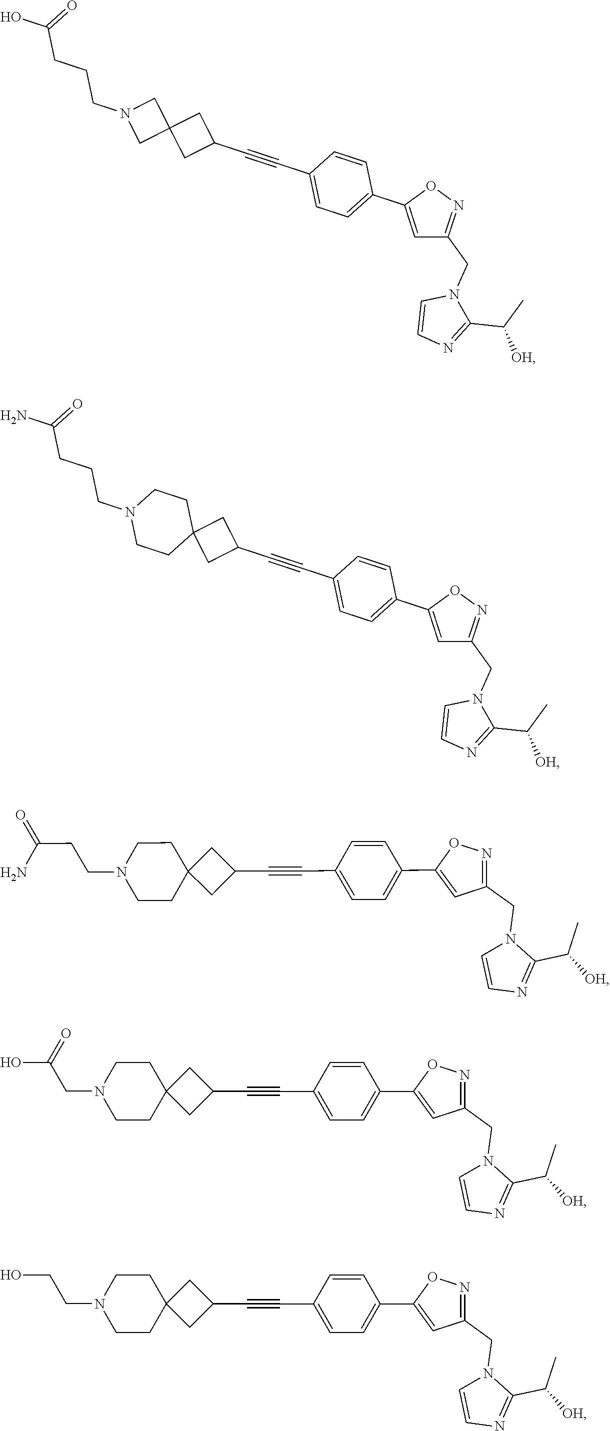 [SMILES: C[C@H](O)c1nccn1Cc1cc(-c2ccc(C#CC3CC4(C3)CN(CCCC(=O)O)C4)cc2)on1 ; C[C@H](O)c1nccn1Cc1cc(-c2ccc(C#CC3CC4(CCN(CCCC(N)=O)CC4)C3)cc2)on1 ; C[C@H](O)c1nccn1Cc1cc(-c2ccc(C#CC3CC4(CCN(CCC(N)=O)CC4)C3)cc2)on1 ; C[C@H](O)c1nccn1Cc1cc(-c2ccc(C#CC3CC4(CCN(CC(=O)O)CC4)C3)cc2)on1 ; C[C@H](O)c1nccn1Cc1cc(-c2ccc(C#CC3CC4(CCN(CCO)CC4)C3)cc2)on1]

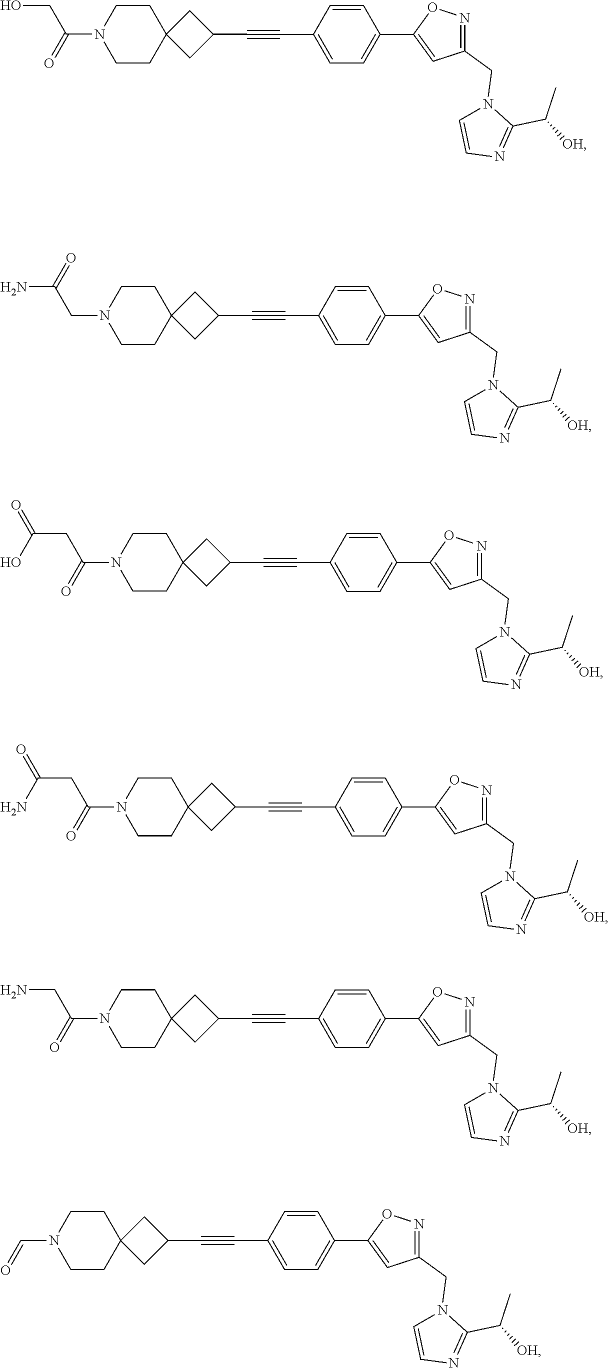 [SMILES: C[C@H](O)c1nccn1Cc1cc(-c2ccc(C#CC3CC4(CCN(C(=O)CO)CC4)C3)cc2)on1 ; C[C@H](O)c1nccn1Cc1cc(-c2ccc(C#CC3CC4(CCN(CC(N)=O)CC4)C3)cc2)on1 ; C[C@H](O)c1nccn1Cc1cc(-c2ccc(C#CC3CC4(CCN(C(=O)CC(=O)O)CC4)C3)cc2)on1 ; C[C@H](O)c1nccn1Cc1cc(-c2ccc(C#CC3CC4(CCN(C(=O)CC(N)=O)CC4)C3)cc2)on1 ; C[C@H](O)c1nccn1Cc1cc(-c2ccc(C#CC3CC4(CCN(C(=O)CN)CC4)C3)cc2)on1 ; C[C@H](O)c1nccn1Cc1cc(-c2ccc(C#CC3CC4(CCN(C=O)CC4)C3)cc2)on1]

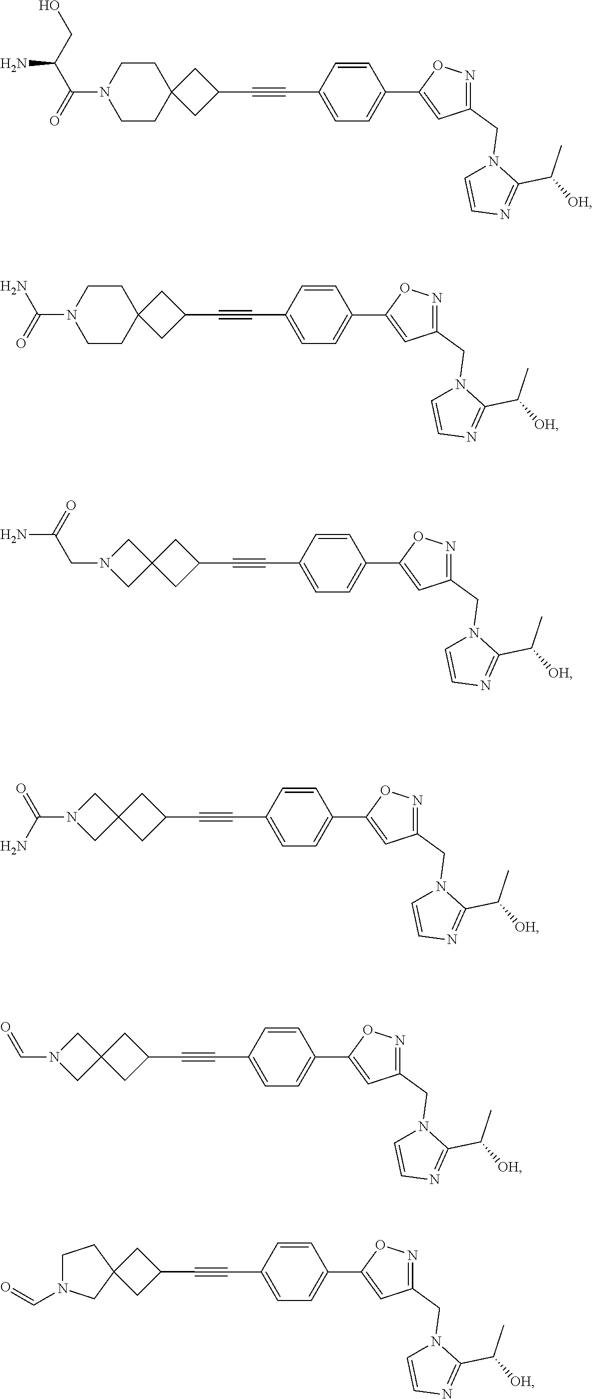 [SMILES: C[C@H](O)c1nccn1Cc1cc(-c2ccc(C#CC3CC4(CCN(C(=O)[C@@H](N)CO)CC4)C3)cc2)on1 ; C[C@H](O)c1nccn1Cc1cc(-c2ccc(C#CC3CC4(CCN(C(N)=O)CC4)C3)cc2)on1 ; C[C@H](O)c1nccn1Cc1cc(-c2ccc(C#CC3CC4(C3)CN(CC(N)=O)C4)cc2)on1 ; C[C@H](O)c1nccn1Cc1cc(-c2ccc(C#CC3CC4(C3)CN(C(N)=O)C4)cc2)on1 ; C[C@H](O)c1nccn1Cc1cc(-c2ccc(C#CC3CC4(C3)CN(C=O)C4)cc2)on1 ; C[C@H](O)c1nccn1Cc1cc(-c2ccc(C#CC3CC4(CCN(C=O)C4)C3)cc2)on1]

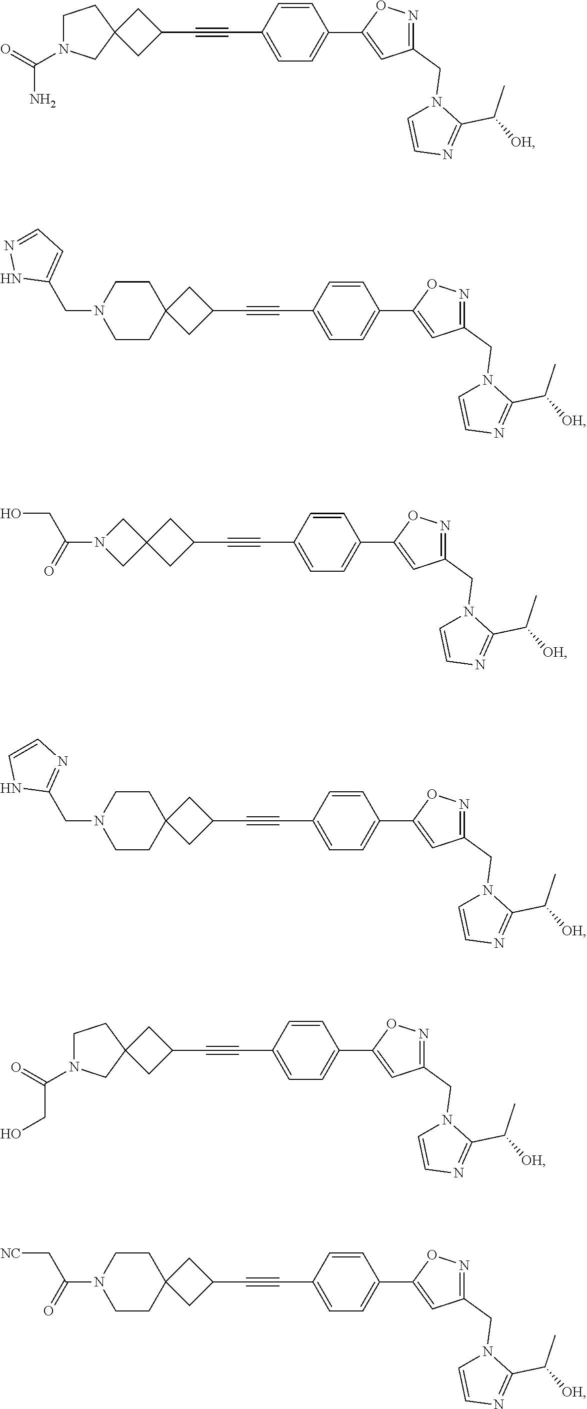 [SMILES: C[C@H](O)c1nccn1Cc1cc(-c2ccc(C#CC3CC4(CCN(C(N)=O)C4)C3)cc2)on1 ; C[C@H](O)c1nccn1Cc1cc(-c2ccc(C#CC3CC4(CCN(Cc5ccn[nH]5)CC4)C3)cc2)on1 ; C[C@H](O)c1nccn1Cc1cc(-c2ccc(C#CC3CC4(C3)CN(C(=O)CO)C4)cc2)on1 ; C[C@H](O)c1nccn1Cc1cc(-c2ccc(C#CC3CC4(CCN(Cc5ncc[nH]5)CC4)C3)cc2)on1 ; C[C@H](O)c1nccn1Cc1cc(-c2ccc(C#CC3CC4(CCN(C(=O)CO)C4)C3)cc2)on1 ; C[C@H](O)c1nccn1Cc1cc(-c2ccc(C#CC3CC4(CCN(C(=O)CC#N)CC4)C3)cc2)on1]

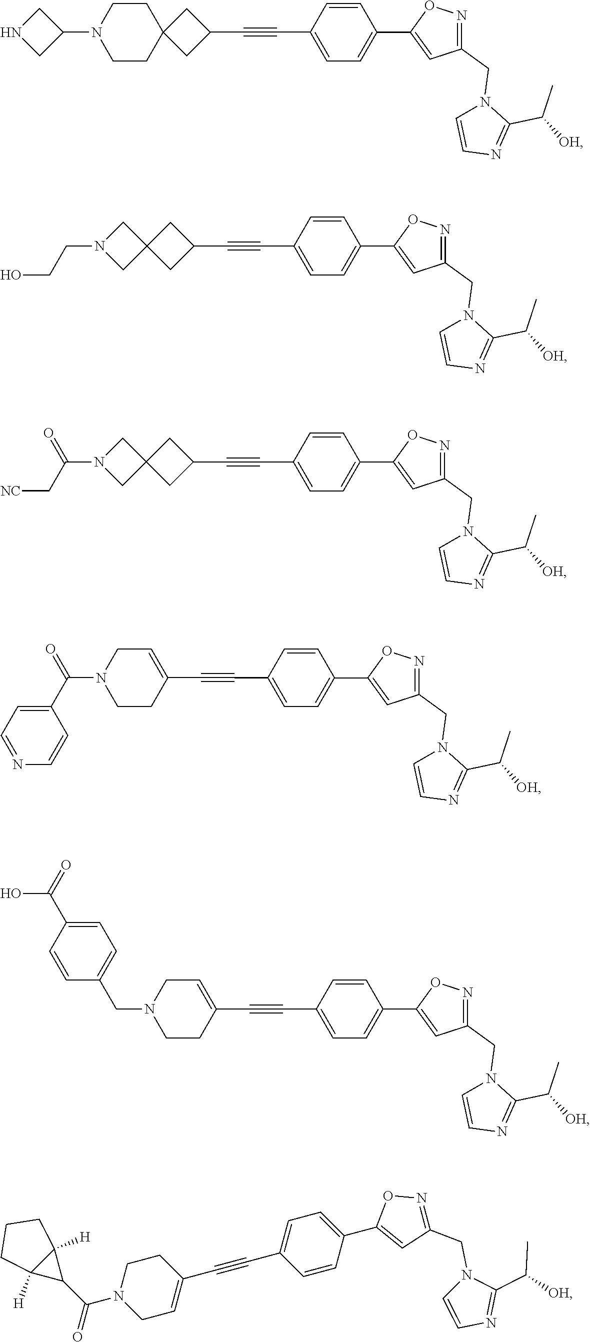 [SMILES: C[C@H](O)c1nccn1Cc1cc(-c2ccc(C#CC3CC4(CCN(C5CNC5)CC4)C3)cc2)on1 ; C[C@H](O)c1nccn1Cc1cc(-c2ccc(C#CC3CC4(C3)CN(CCO)C4)cc2)on1 ; C[C@H](O)c1nccn1Cc1cc(-c2ccc(C#CC3CC4(C3)CN(C(=O)CC#N)C4)cc2)on1 ; C[C@H](O)c1nccn1Cc1cc(-c2ccc(C#CC3=CCN(C(=O)c4ccncc4)CC3)cc2)on1 ; C[C@H](O)c1nccn1Cc1cc(-c2ccc(C#CC3=CCN(Cc4ccc(C(=O)O)cc4)CC3)cc2)on1 ; C[C@H](O)c1nccn1Cc1cc(-c2ccc(C#CC3=CCN(C(=O)C4[C@H]5CCC[C@@H]45)CC3)cc2)on1]

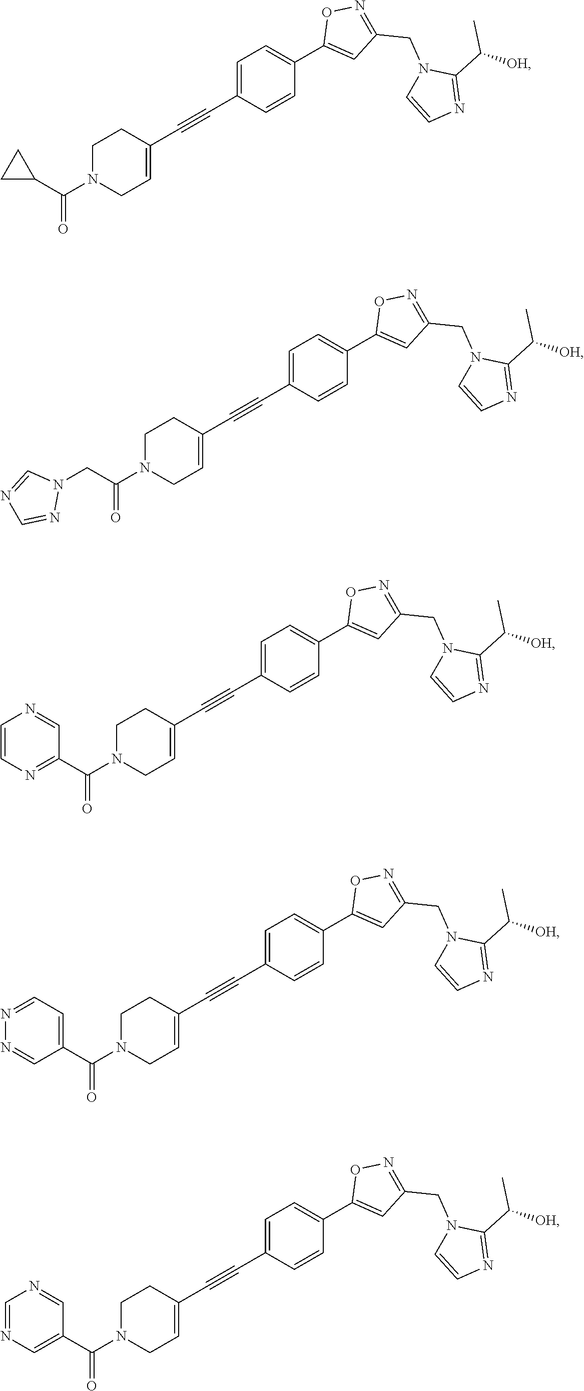 [SMILES: C[C@H](O)c1nccn1Cc1cc(-c2ccc(C#CC3=CCN(C(=O)C4CC4)CC3)cc2)on1 ; C[C@H](O)c1nccn1Cc1cc(-c2ccc(C#CC3=CCN(C(=O)Cn4cncn4)CC3)cc2)on1 ; C[C@H](O)c1nccn1Cc1cc(-c2ccc(C#CC3=CCN(C(=O)c4cnccn4)CC3)cc2)on1 ; C[C@H](O)c1nccn1Cc1cc(-c2ccc(C#CC3=CCN(C(=O)c4ccnnc4)CC3)cc2)on1 ; C[C@H](O)c1nccn1Cc1cc(-c2ccc(C#CC3=CCN(C(=O)c4cncnc4)CC3)cc2)on1]

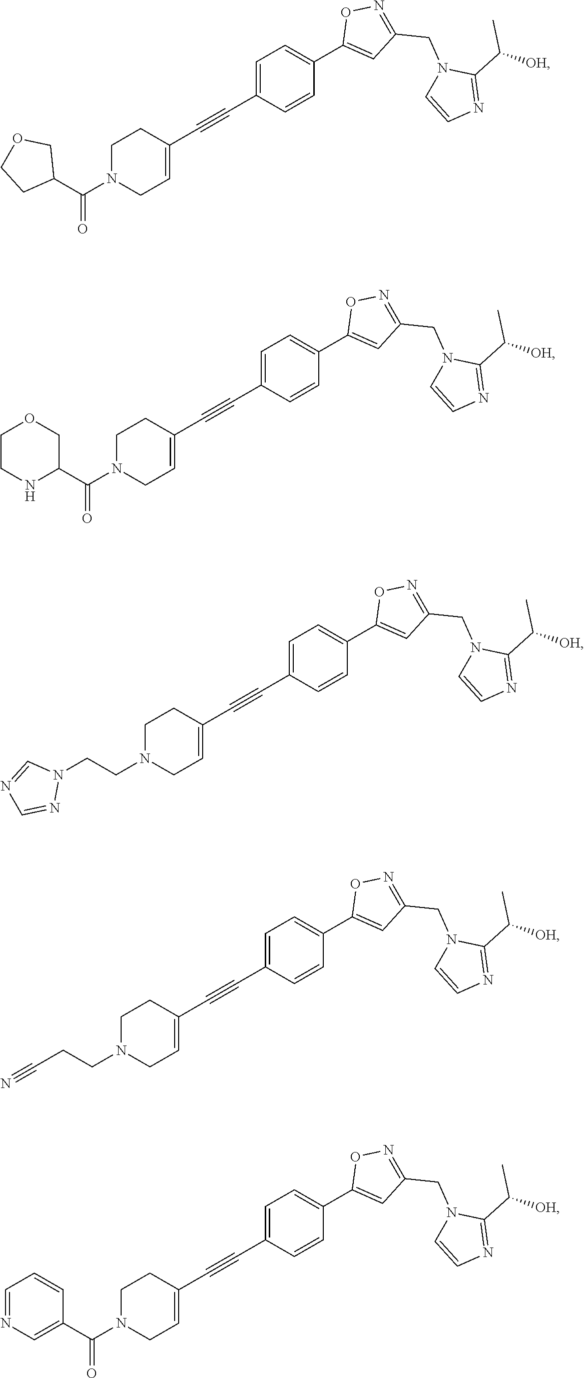 [SMILES: C[C@H](O)c1nccn1Cc1cc(-c2ccc(C#CC3=CCN(C(=O)C4CCOC4)CC3)cc2)on1 ; C[C@H](O)c1nccn1Cc1cc(-c2ccc(C#CC3=CCN(C(=O)C4COCCN4)CC3)cc2)on1 ; C[C@H](O)c1nccn1Cc1cc(-c2ccc(C#CC3=CCN(CCn4cncn4)CC3)cc2)on1 ; C[C@H](O)c1nccn1Cc1cc(-c2ccc(C#CC3=CCN(CCC#N)CC3)cc2)on1 ; C[C@H](O)c1nccn1Cc1cc(-c2ccc(C#CC3=CCN(C(=O)c4cccnc4)CC3)cc2)on1]

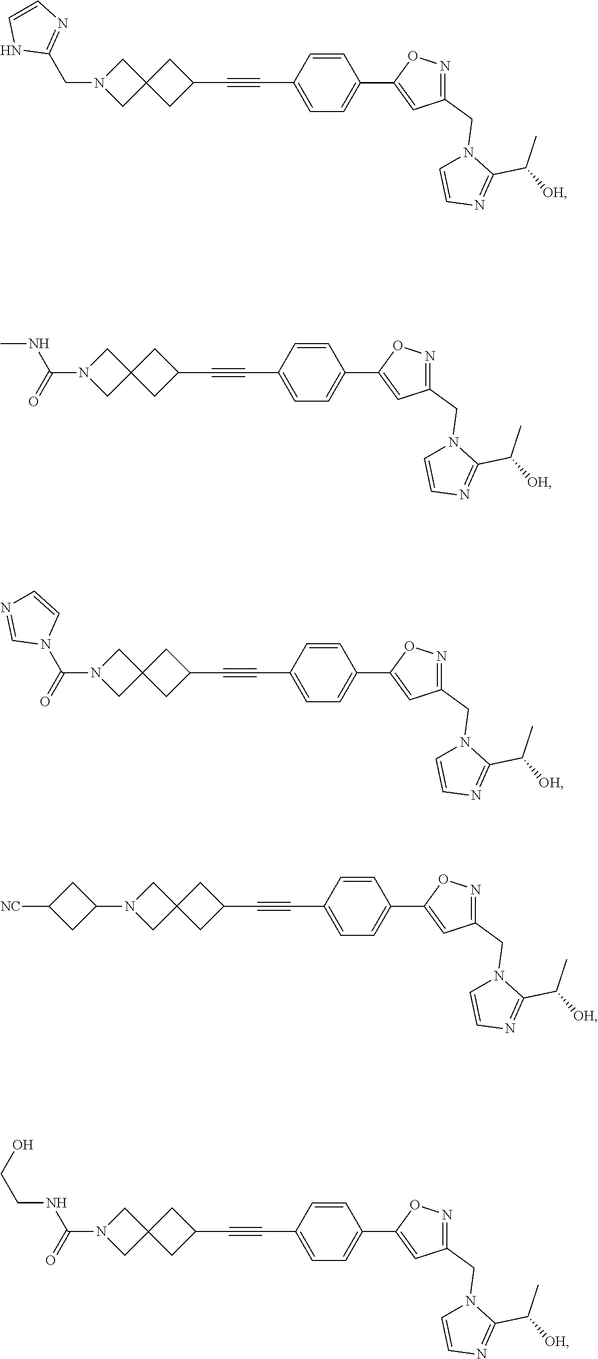 [SMILES: C[C@H](O)c1nccn1Cc1cc(-c2ccc(C#CC3CC4(C3)CN(Cc3ncc[nH]3)C4)cc2)on1 ; CNC(=O)N1CC2(CC(C#Cc3ccc(-c4cc(Cn5ccnc5[C@H](C)O)no4)cc3)C2)C1 ; C[C@H](O)c1nccn1Cc1cc(-c2ccc(C#CC3CC4(C3)CN(C(=O)n3ccnc3)C4)cc2)on1 ; C[C@H](O)c1nccn1Cc1cc(-c2ccc(C#CC3CC4(C3)CN(C3CC(C#N)C3)C4)cc2)on1 ; C[C@H](O)c1nccn1Cc1cc(-c2ccc(C#CC3CC4(C3)CN(C(=O)NCCO)C4)cc2)on1]

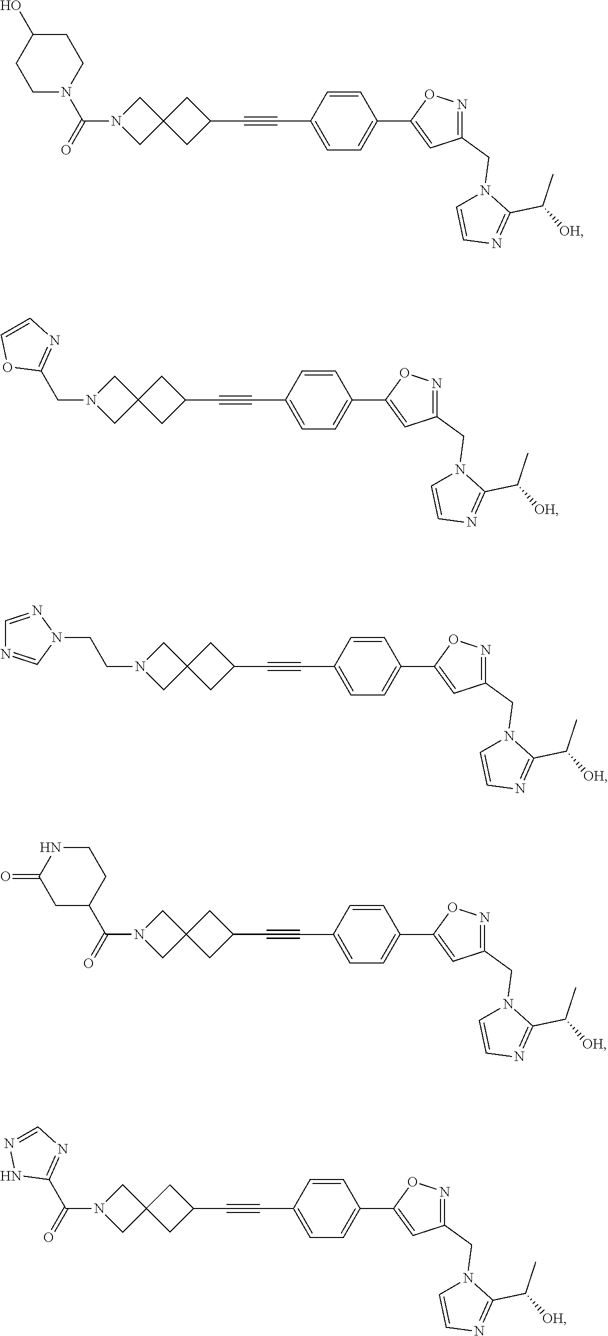 [SMILES: C[C@H](O)c1nccn1Cc1cc(-c2ccc(C#CC3CC4(C3)CN(C(=O)N3CCC(O)CC3)C4)cc2)on1 ; C[C@H](O)c1nccn1Cc1cc(-c2ccc(C#CC3CC4(C3)CN(Cc3ncco3)C4)cc2)on1 ; C[C@H](O)c1nccn1Cc1cc(-c2ccc(C#CC3CC4(C3)CN(CCn3cncn3)C4)cc2)on1 ; C[C@H](O)c1nccn1Cc1cc(-c2ccc(C#CC3CC4(C3)CN(C(=O)C3CCNC(=O)C3)C4)cc2)on1 ; C[C@H](O)c1nccn1Cc1cc(-c2ccc(C#CC3CC4(C3)CN(C(=O)c3ncn[nH]3)C4)cc2)on1]

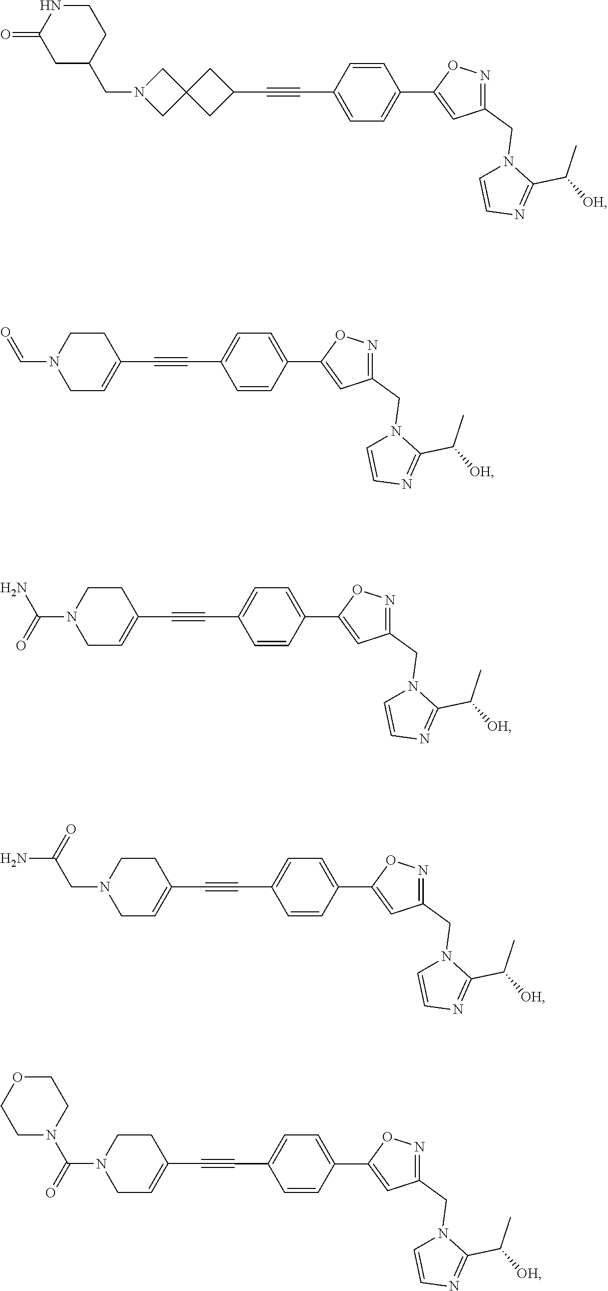 [SMILES: C[C@H](O)c1nccn1Cc1cc(-c2ccc(C#CC3CC4(C3)CN(CC3CCNC(=O)C3)C4)cc2)on1 ; C[C@H](O)c1nccn1Cc1cc(-c2ccc(C#CC3=CCN(C=O)CC3)cc2)on1 ; C[C@H](O)c1nccn1Cc1cc(-c2ccc(C#CC3=CCN(C(N)=O)CC3)cc2)on1 ; C[C@H](O)c1nccn1Cc1cc(-c2ccc(C#CC3=CCN(CC(N)=O)CC3)cc2)on1 ; C[C@H](O)c1nccn1Cc1cc(-c2ccc(C#CC3=CCN(C(=O)N4CCOCC4)CC3)cc2)on1]

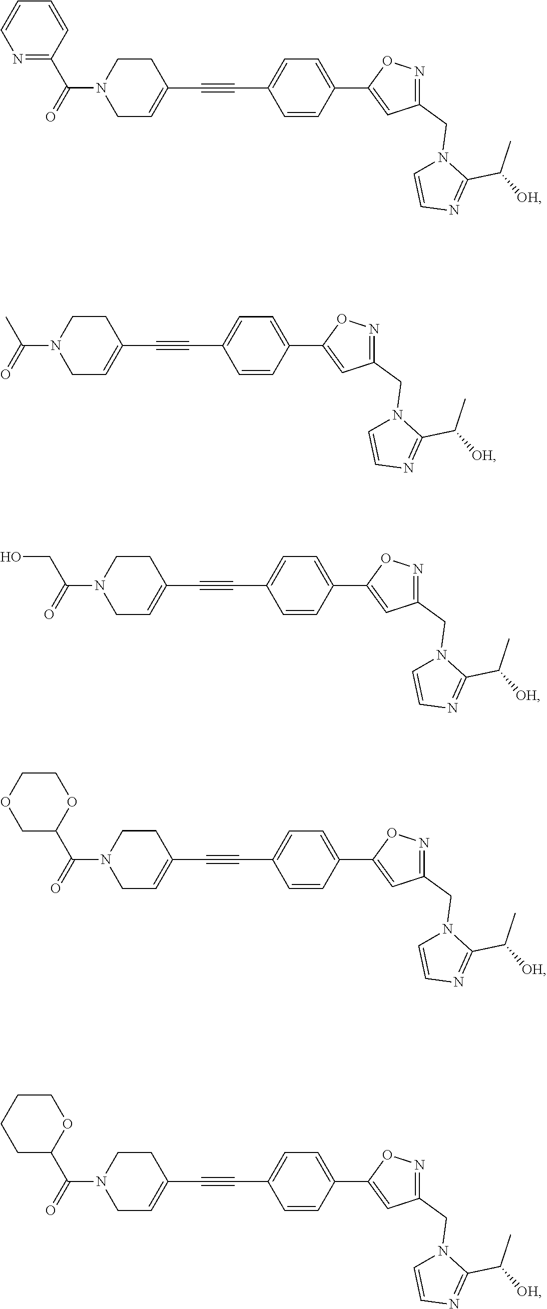 [SMILES: C[C@H](O)c1nccn1Cc1cc(-c2ccc(C#CC3=CCN(C(=O)c4ccccn4)CC3)cc2)on1 ; CC(=O)N1CC=C(C#Cc2ccc(-c3cc(Cn4ccnc4[C@H](C)O)no3)cc2)CC1 ; C[C@H](O)c1nccn1Cc1cc(-c2ccc(C#CC3=CCN(C(=O)CO)CC3)cc2)on1 ; C[C@H](O)c1nccn1Cc1cc(-c2ccc(C#CC3=CCN(C(=O)C4COCCO4)CC3)cc2)on1 ; C[C@H](O)c1nccn1Cc1cc(-c2ccc(C#CC3=CCN(C(=O)C4CCCCO4)CC3)cc2)on1]

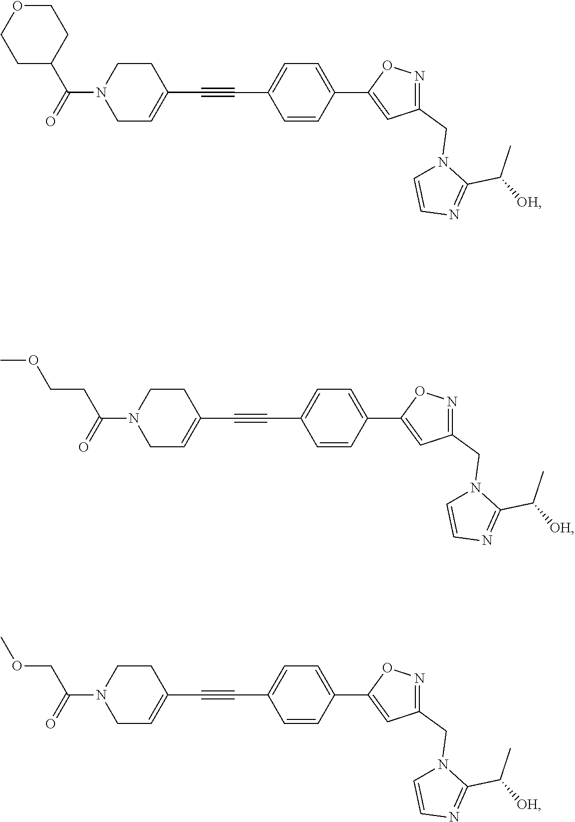 [SMILES: C[C@H](O)c1nccn1Cc1cc(-c2ccc(C#CC3=CCN(C(=O)C4CCOCC4)CC3)cc2)on1 ; COCCC(=O)N1CC=C(C#Cc2ccc(-c3cc(Cn4ccnc4[C@H](C)O)no3)cc2)CC1 ; COCC(=O)N1CC=C(C#Cc2ccc(-c3cc(Cn4ccnc4[C@H](C)O)no3)cc2)CC1]

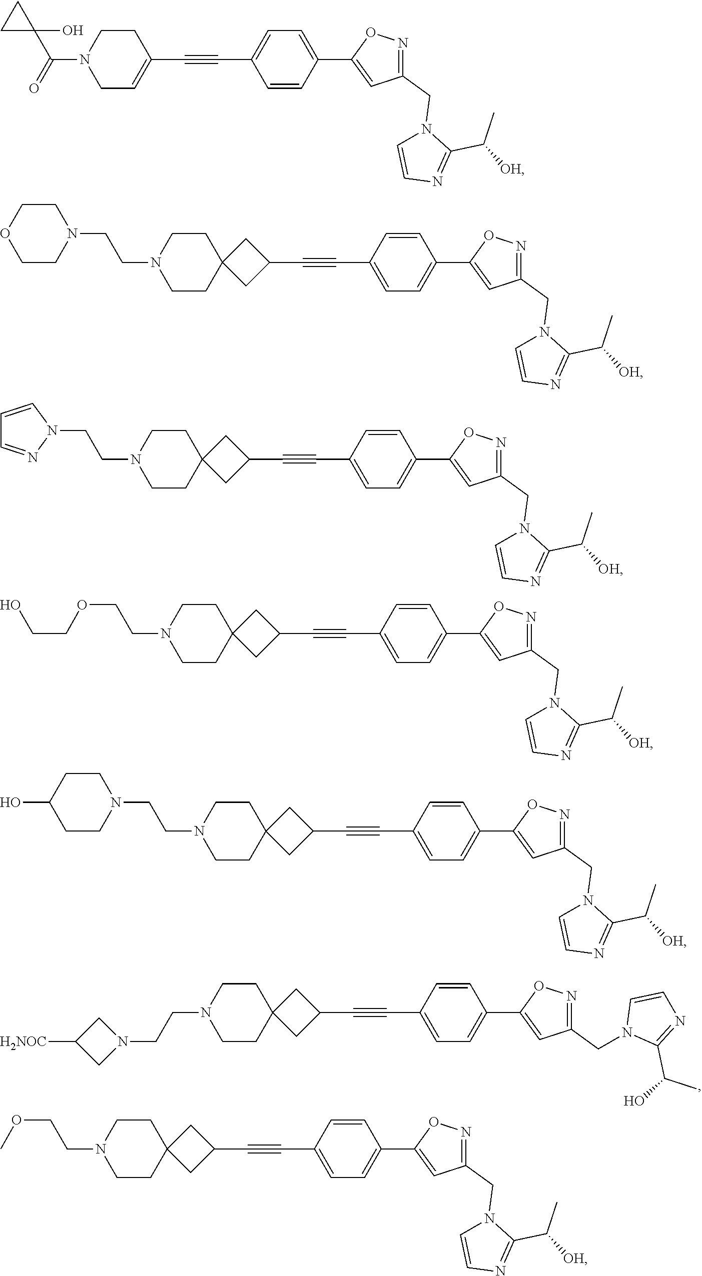

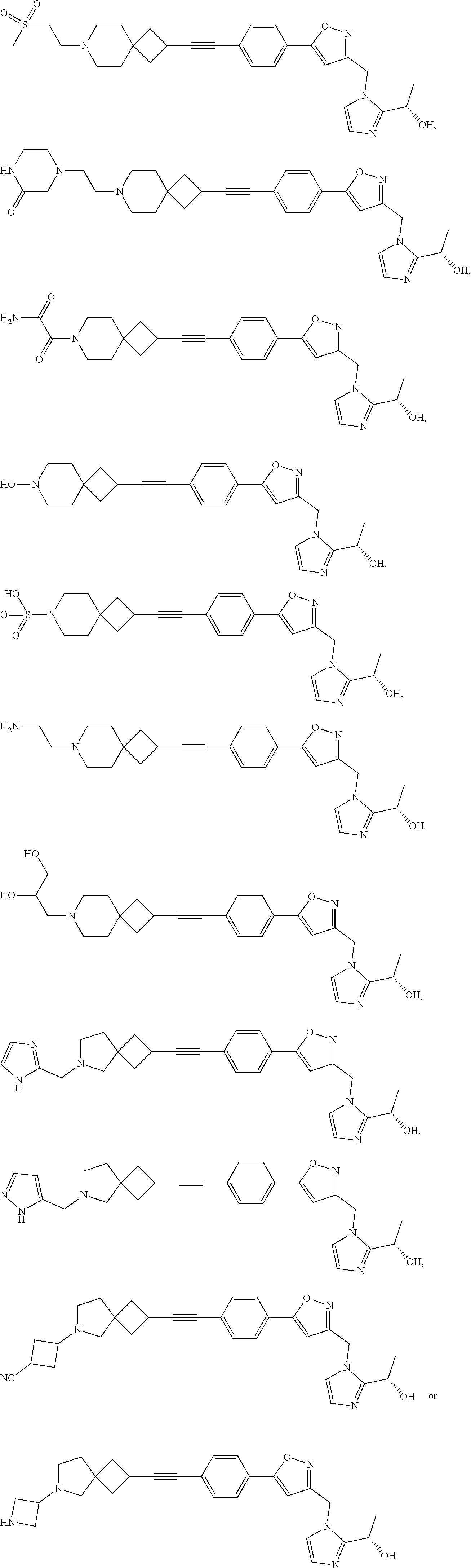

7. A pharmaceutical composition, wherein the pharmaceutical composition comprises an effective dose of the compound or the stereoisomer or the tautomer thereof or the pharmaceutically acceptable salt thereof according to claim 1, and a pharmaceutically acceptable carrier, an excipient or a combination thereof.

8. A method for inhibiting LPXC, comprising administering a therapeutically effective amount of the compound or the stereoisomer or the tautomer thereof or the pharmaceutically acceptable salt thereof according to claim 1 or the pharmaceutical composition comprising the compound or the stereoisomer or the tautomer thereof or the pharmaceutically acceptable salt thereof according to claim 1 to a subject in need thereof.

9. A method for treating a disease mediated by LPXC, comprising administering a therapeutically effective amount of the compound or the stereoisomer or the tautomer thereof or the pharmaceutically acceptable salt thereof according to claim 1, or the pharmaceutical composition comprising the compound or the stereoisomer or the tautomer thereof or the pharmaceutically acceptable salt thereof according to claim 1 to a subject in need thereof, wherein the disease mediated by LPXC is selected from a bacterial infection caused by a Gram-negative bacterium.

10. The method according to claim 9, wherein the Gram-negative bacterium is selected from Escherichia coli, Pseudomonas aeruginosa, Proteusbacillus vulgaris, Shigella dysenteriae, Pneumobacillus, Brucella, Typhoid Bacillus, Acinetobacter, Yersinia, Legionella pneumophila, Bordetella pertussis, Shigella, Pasteurella, Vibrio cholerae or Neisseria Meningitidis.

11. The compound or the stereoisomer or the tautomer thereof or the pharmaceutically acceptable salt thereof according to claim 1, wherein m is 0.

12. The compound or the stereoisomer or the tautomer thereof or the pharmaceutically acceptable salt thereof according to claim 5, wherein the R⁸, R⁹ and R¹⁰ are each independently selected from methyl.

13. The compound or the stereoisomer or the tautomer thereof or the pharmaceutically acceptable salt thereof according to claim 2, wherein W is N.

14. The compound or the stereoisomer or the tautomer thereof or the pharmaceutically acceptable salt thereof according to claim 2, wherein ring C is selected from:

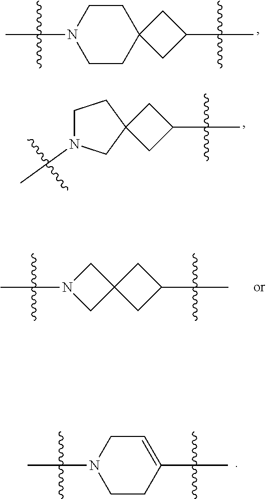

15. The compound or the stereoisomer or the tautomer thereof or the pharmaceutically acceptable salt thereof according to claim 2, wherein:
R¹ is selected from hydrogen atom, hydroxyl, alkyl, cycloalkyl, heterocyclyl, —C(O)R³, —C(O)NR⁶R⁷ or —S(O)ᵣR⁴; wherein the alkyl or the cycloalkyl is optionally further substituted by one or more R^{B};
R^{B} is each independently selected from cyano, hydroxyl, heterocyclyl, aryl, heteroaryl, —OR⁵, —C(O)OR⁵, —NR⁶R⁷, —C(O)NR⁶R⁷ or —S(O)ᵣR⁵; wherein the heterocyclyl or the heteroaryl is optionally further substituted by one or more substituents selected from hydroxyl, cyano, alkoxy, haloalkyl, ═O, —C(O)OR⁸ or —C(O)NR⁹R¹⁰;
alternatively, two R^{B} form one —C(═O)— together with the same carbon atom to which the two R^{B} are connected;
R³ is selected from hydrogen atom, alkyl, cycloalkyl, heteroaryl or heterocyclyl; wherein the alkyl, the cycloalkyl, the heteroaryl or the heterocyclyl is optionally further substituted by one or more substituents selected from hydroxyl, alkoxy, cyano, heteroaryl, ═O, —NR⁹R¹⁰, —C(O)OR⁸ or —C(O)NR⁹R¹⁰;
R⁴ is hydrogen atom or hydroxyl;
R⁵ is each independently selected from hydrogen atom, alkyl or hydroxyalkyl;
R⁶ and R⁷ are each independently selected from hydrogen atom or alkyl, wherein the alkyl is optionally further substituted by one or more substituents selected from hydroxyl, cyano, halogen or alkoxy;
R⁸, R⁹ and R¹⁰ are each independently selected from hydrogen atom or alkyl; and
r is 2.

16. The compound or the stereoisomer or the tautomer thereof or the pharmaceutically acceptable salt thereof according to claim 15, wherein the R⁸, R⁹ and R¹⁰ are each independently selected from methyl.
